SEQUENCE LISTING (152 sequences)

SEQ ID NO: 1 - protein, 727 aa, synthetic construct
  REGION 1..727: note = 3BNC117 human IgG1 antibody
MDWTWRILFLVAAATGTHAQVQLLQSGAAVTKPGASVRVSCEASGYNIRDYFIHWWRQAPGQGLQWVGWINPKTGQPNNPRQFQGRVSLTRHASWDFDTFSFYMDLKALRSDDTAVYFCARQRSDYWDFDVWGSGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDIQMTQSPSSLSASVGDTVTITCQANGYLNWYQQRRGKAPKLLIYDGSKLERGVPSRFSGRRWGQEYNLTINNLQPEDIATYFCQVYEFVVPGTRLDLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGEC

SEQ ID NO: 2 - DNA, 2205 bp, synthetic construct
  misc_feature 1..2205: note = 3BNC117 human IgG1 antibody
  source 1..2205: mol_type = other DNA
ggatccgccaccatggattggacatggagaattctgtttctggtcgccgccgctaccggaactcacgctcaggtgcagctgctgcagtcaggggccgcagtgacaaagccaggggcaagtgtccgcgtgtcatgcgaggccagcggctacaacatccgggactatttcattcactggtggcgacaggcacctggacagggactgcagtgggtggggtggatcaatccaaagaccggccagcccaacaatcctaggcagttccagggcagagtgagcctgacccggcatgcttcctgggactttgatacattctctttttacatggacctgaaggcactgaggagtgacgataccgccgtgtacttctgcgctagacagcggtcagattattgggactttgacgtgtggggcagcggaactcaggtcaccgtgagctccgccagtacaaagggaccttcagtgtttccactggctccctctagtaaaagcacatccggcggaactgccgctctgggatgtctggtgaaggactacttccctgagccagtcaccgtgtcctggaactctggagctctgactagcggggtccacacctttccagcagtgctgcagtcaagcggcctgtactccctgtcctctgtggtcactgtccccagttcaagcctgggaactcagacctatatctgcaacgtgaatcacaagccaagtaataccaaagtcgacaagaaagtggagcccaagtcctgtgataaaacacatacttgccctccctgtccagctcctgaactgctgggcggcccaagcgtgttcctgtttccacccaagcctaaagacactctgatgatttctagaacccctgaggtcacatgcgtggtcgtggacgtgagccacgaggaccccgaagtcaagttcaactggtacgtggatggcgtcgaagtgcataatgccaagaccaaaccccgagaggaacagtacaactctacttatagggtcgtgagtgtcctgaccgtgctgcaccaggactggctgaacggcaaggagtataagtgcaaagtgtccaataaggctctgccagcacccatcgaaaaaacaatttctaaggccaaaggacagcctagagagccacaggtgtacactctgcctccatcccgggacgaactgacaaagaaccaggtctctctgacttgtctggtgaaaggattctatcccagcgatatcgctgtggagtgggaatccaatgggcagcctgagaacaattacaagaccacaccccctgtgctggacagtgatgggtcattctttctgtattccaagctgacagtggacaaatctcggtggcagcagggcaacgtcttttcttgcagtgtgatgcatgaagccctgcacaatcattacacacagaagtcactgagcctgtccccaggaaagcgaggacgaaaaaggagatctgggagtggcgctactaacttctctctgctgaaacaggcaggagatgtggaggaaaatcctgggccaatggtcctgcagacccaggtgtttatctcactgctgctgtggattagcggcgcatatggagacatccagatgacacagagcccttcctctctgtcagcaagcgtcggcgataccgtgacaattacttgtcaggccaacggctacctgaattggtatcagcagcggcgcggaaaggctcccaaactgctgatctacgacggctccaagctggagcgcggagtgcctagccgattctccgggcgaaggtggggacaggagtataacctgactatcaacaatctgcagcctgaagatattgccacctacttctgccaggtgtatgagtttgtcgtgccagggaccaggctggacctgaagagaacagtcgcagcccccagcgtgttcatctttccaccctcagatgagcagctgaaatccggaaccgcctctgtggtgtgcctgctgaacaacttctacccacgcgaagccaaggtccagtggaaagtggacaacgctctgcagtctggcaatagtcaggagtcagtgacagaacaggacagcaaggattccacttattctctgagttcaaccctgacactgtccaaagcagattacgagaagcacaaagtgtatgcctgcgaggtcacccaccaggggctgcggtcaccagtcacaaagtccttcaacagaggggaatgctgataactcgag

SEQ ID NO: 3 - protein, 472 aa, synthetic construct
  REGION 1..472: note = 3BNC117 human IgG1 antibody (heavy chain) (mod1)
MDWTWRILFLVAAATGTHAQVQLLQSGAAVTKPGASVRVSCEASGYNIRDYFIHWWRQAPGQGLQWVGWINPKTGQPNNPRQFQGRVSLTRHASWDFDTFSFYMDLKALRSDDTAVYFCARQRSDYWDFDVWGSGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - DNA, 1440 bp, synthetic construct
  misc_feature 1..1440: note = 3BNC117 human IgG1 antibody (heavy chain) (mod1)
  source 1..1440: mol_type = other DNA
ggatccgccaccatggattggacatggagaattctgtttctggtcgccgccgctaccggaactcacgctcaggtgcagctgctgcagtcaggggccgcagtgacaaagccaggggcaagtgtccgcgtgtcatgcgaggccagcggctacaacatccgggactatttcattcactggtggcgacaggcacctggacagggactgcagtgggtggggtggatcaatccaaagaccggccagcccaacaatcctaggcagttccagggcagagtgagcctgacccggcatgcttcctgggactttgatacattctctttttacatggacctgaaggcactgaggagtgacgataccgccgtgtacttctgcgctagacagcggtcagattattgggactttgacgtgtggggcagcggaactcaggtcaccgtgagctccgccagtacaaagggaccttcagtgtttccactggctccctctagtaaaagcacatccggcggaactgccgctctgggatgtctggtgaaggactacttccctgagccagtcaccgtgtcctggaactctggagctctgactagcggggtccacacctttccagcagtgctgcagtcaagcggcctgtactccctgtcctctgtggtcactgtccccagttcaagcctgggaactcagacctatatctgcaacgtgaatcacaagccaagtaataccaaagtcgacaagaaagtggagcccaagtcctgtgataaaacacatacttgccctccctgtccagctcctgaactgctgggcggcccaagcgtgttcctgtttccacccaagcctaaagacactctgatgatttctagaacccctgaggtcacatgcgtggtcgtggacgtgagccacgaggaccccgaagtcaagttcaactggtacgtggatggcgtcgaagtgcataatgccaagaccaaaccccgagaggaacagtacaactctacttatagggtcgtgagtgtcctgaccgtgctgcaccaggactggctgaacggcaaggagtataagtgcaaagtgtccaataaggctctgccagcacccatcgaaaaaacaatttctaaggccaaaggacagcctagagagccacaggtgtacactctgcctccatcccgggacgaactgacaaagaaccaggtctctctgacttgtctggtgaaaggattctatcccagcgatatcgctgtggagtgggaatccaatgggcagcctgagaacaattacaagaccacaccccctgtgctggacagtgatgggtcattctttctgtattccaagctgacagtggacaaatctcggtggcagcagggcaacgtcttttcttgcagtgtgatgcatgaagccctgcacaatcattacacacagaagtcactgagcctgtccccaggaaagtgataactcgag

SEQ ID NO: 5 - protein, 226 aa, synthetic construct
  REGION 1..226: note = 3BNC117 human IgG1 antibody (light chain) (mod1)
MVLQTQVFISLLLWISGAYGDIQMTQSPSSLSASVGDTVTITCQANGYLNWYQQRRGKAPKLLIYDGSKLERGVPSRFSGRRWGQEYNLTINNLQPEDIATYFCQVYEFVVPGTRLDLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGEC

SEQ ID NO: 6 - DNA, 702 bp, synthetic construct
  misc_feature 1..702: note = 3BNC117 human IgG1 antibody (light chain) (mod1)
  source 1..702: mol_type = other DNA
ggatccgccaccatggtcctgcagacccaggtgtttatctcactgctgctgtggattagcggcgcatatggagacatccagatgacacagagcccttcctctctgtcagcaagcgtcggcgataccgtgacaattacttgtcaggccaacggctacctgaattggtatcagcagcggcgcggaaaggctcccaaactgctgatctacgacggctccaagctggagcgcggagtgcctagccgattctccgggcgaaggtggggacaggagtataacctgactatcaacaatctgcagcctgaagatattgccacctacttctgccaggtgtatgagtttgtcgtgccagggaccaggctggacctgaagagaacagtcgcagcccccagcgtgttcatctttccaccctcagatgagcagctgaaatccggaaccgcctctgtggtgtgcctgctgaacaacttctacccacgcgaagccaaggtccagtggaaagtggacaacgctctgcagtctggcaatagtcaggagtcagtgacagaacaggacagcaaggattccacttattctctgagttcaaccctgacactgtccaaagcagattacgagaagcacaaagtgtatgcctgcgaggtcacccaccaggggctgcggtcaccagtcacaaagtccttcaacagaggggaatgctgataactcgag

SEQ ID NO: 7 - protein, 724 aa, synthetic construct
  REGION 1..724: note = 3BNC117 human IgG4 (mod 2)
MDWTWRILFLVAAATGTHAQVQLLQSGAAVTKPGASVRVSCEASGYNIRDYFIHWWRQAPGQGLQWVGWINPKTGQPNNPRQFQGRVSLTRHASWDFDTFSFYMDLKALRSDDTAVYFCARQRSDYWDFDVWGSGTQVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKRGRKRRSGSGATNFSLLKQAGDVEENPGPMVLQTQVFISLLLWISGAYGDIQMTQSPSSLSASVGDTVTITCQANGYLNWYQQRRGKAPKLLIYDGSKLERGVPSRFSGRRWGQEYNLTINNLQPEDIATYFCQVYEFVVPGTRLDLKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 8 - DNA, 2196 bp, synthetic construct
  misc_feature 1..2196: note = 3BNC117 human IgG4 (mod 2)
  source 1..2196: mol_type = other DNA
ggatccgccaccatggactggacctggcgcatcctgttcctggtggcagcagcaaccggaacacacgcacaggtgcagctgctgcagagcggagcagcagtgacaaagccaggagcatctgtgagggtgagctgcgaggcatccggctacaacatccgcgattacttcatccactggtggaggcaggcaccaggacagggactgcagtgggtgggctggatcaatcctaagaccggccagcctaacaatccaaggcagttccagggacgcgtgagcctgaccaggcacgcctcctgggactttgatacattctctttttacatggacctgaaggccctgcgcagcgacgataccgccgtgtacttctgtgcccggcagaggagcgattattgggactttgacgtgtggggctctggcacccaggtgacagtgagctccgcctccacaaagggaccaagcgtgttcccactggcaccttgcagcaggtccacctctgagagcacagccgccctgggctgtctggtgaaggactacttccctgagccagtgaccgtgagctggaactccggcgccctgaccagcggagtgcacacatttcccgccgtgctgcagtctagcggcctgtactccctgtcctctgtggtgaccgtgcctagctcctctctgggcaccaagacatatacctgcaacgtggaccacaagccaagcaatacaaaggtggataagagagtggagtccaagtacggccctccctgcccttcttgtccagcacctgagttcctgggcggcccttccgtgttcctgtttccacccaagccaaaggacaccctgatgatctctcggacccccgaggtgacatgcgtggtggtggacgtgagccaggaggaccccgaggtgcagttcaactggtacgtggatggcgtggaggtgcacaatgccaagacaaagcccagagaggagcagtttaactctacctaccgggtggtgagcgtgctgacagtgctgcaccaggactggctgaacggcaaggagtataagtgcaaggtgtccaataagggcctgcctagctccatcgagaagaccatctctaaggcaaagggacagccacgcgagccacaggtgtacacactgcctccaagccaggaggagatgaccaagaaccaggtgtccctgacatgtctggtgaagggcttctatccaagcgacatcgccgtggagtgggagtccaatggccagcccgagaacaattacaagaccacaccccctgtgctggactctgatggcagcttctttctgtatagcagactgaccgtggataagtcccggtggcaggagggcaacgtgttttcctgctctgtgatgcacgaggccctgcacaatcactacacccagaagagcctgtccctgtctctgggcaagaggggcagaaagaggagaagcggctccggcgccacaaacttcagcctgctgaagcaggcaggcgacgtggaggagaatccaggacccatggtgctgcagacccaggtgtttatctccctgctgctgtggatctctggcgcctatggcgacatccagatgacacagtccccctctagcctgtctgccagcgtgggcgatacagtgaccatcacatgtcaggccaacggctacctgaattggtatcagcagaggaggggcaaggcaccaaagctgctgatctacgacggcagcaagctggagagaggcgtgccctcccggttctctggcaggagatggggccaggagtataacctgaccatcaacaatctgcagcctgaggatatcgccacatacttctgccaggtgtatgagtttgtggtgcctggcacccgcctggacctgaagaggacagtggccgccccaagcgtgttcatctttccacccagcgatgagcagctgaagtctggcaccgccagcgtggtgtgcctgctgaacaatttctacccaagagaggccaaggtgcagtggaaggtggacaacgccctgcagagcggcaattcccaggagtctgtgaccgagcaggacagcaaggattccacatattccctgtctaacaccctgacactgagcaaggccgattacgagaagcacaaggtgtatgcatgcgaggtgacccaccagggactgtcctctcctgtgacaaagtcctttaatcggggcgagtgttgataactcgag

SEQ ID NO: 9 - protein, 724 aa, synthetic construct
  REGION 1..724: note = 3BNC117 human IgG4 (S228P) (mod 3)
MDWTWRILFLVAAATGTHAQVQLLQSGAAVTKPGASVRVSCEASGYNIRDYFIHWWRQAPGQGLQWVGWINPKTGQPNNPRQFQGRVSLTRHASWDFDTFSFYMDLKALRSDDTAVYFCARQRSDYWDFDVWGSGTQVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKRGRKRRSGSGATNFSLLKQAGDVEENPGPMVLQTQVFISLLLWISGAYGDIQMTQSPSSLSASVGDTVTITCQANGYLNWYQQRRGKAPKLLIYDGSKLERGVPSRFSGRRWGQEYNLTINNLQPEDIATYFCQVYEFVVPGTRLDLKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 10 - DNA, 2196 bp, synthetic construct
  misc_feature 1..2196: note = 3BNC117 human IgG4 (S228P) (mod 3)
  source 1..2196: mol_type = other DNA
ggatccgccaccatggactggacctggcgcatcctgttcctggtggcagcagcaaccggaacacacgcacaggtgcagctgctgcagagcggagcagcagtgacaaagccaggagcatctgtgagggtgagctgcgaggcatccggctacaacatccgcgattacttcatccactggtggaggcaggcaccaggacagggactgcagtgggtgggctggatcaatcctaagaccggccagcctaacaatccaaggcagttccagggacgcgtgagcctgaccaggcacgcctcctgggactttgatacattctctttttacatggacctgaaggccctgcgcagcgacgataccgccgtgtacttctgtgcccggcagaggagcgattattgggactttgacgtgtggggctctggcacccaggtgacagtgagctccgcctccacaaagggaccaagcgtgttcccactggcaccttgcagcaggtccacctctgagagcacagccgccctgggctgtctggtgaaggactacttccctgagccagtgaccgtgagctggaactccggcgccctgaccagcggagtgcacacatttcccgccgtgctgcagtctagcggcctgtactccctgtcctctgtggtgaccgtgcctagctcctctctgggcaccaagacatatacctgcaacgtggaccacaagccaagcaatacaaaggtggataagagagtggagtccaagtacggccctccctgccctccctgtccagcacctgagttcctgggcggcccttccgtgttcctgtttccacccaagccaaaggacaccctgatgatctctcggacccccgaggtgacatgcgtggtggtggacgtgagccaggaggaccccgaggtgcagttcaactggtacgtggatggcgtggaggtgcacaatgccaagacaaagcccagagaggagcagtttaactctacctaccgggtggtgagcgtgctgacagtgctgcaccaggactggctgaacggcaaggagtataagtgcaaggtgtccaataagggcctgcctagctccatcgagaagaccatctctaaggcaaagggacagccacgcgagccacaggtgtacacactgcctccaagccaggaggagatgaccaagaaccaggtgtccctgacatgtctggtgaagggcttctatccaagcgacatcgccgtggagtgggagtccaatggccagcccgagaacaattacaagaccacaccccctgtgctggactctgatggcagcttctttctgtatagcagactgaccgtggataagtcccggtggcaggagggcaacgtgttttcctgctctgtgatgcacgaggccctgcacaatcactacacccagaagagcctgtccctgtctctgggcaagaggggcagaaagaggagaagcggctccggcgccacaaacttcagcctgctgaagcaggcaggcgacgtggaggagaatccaggacccatggtgctgcagacccaggtgtttatctccctgctgctgtggatctctggcgcctatggcgacatccagatgacacagtccccctctagcctgtctgccagcgtgggcgatacagtgaccatcacatgtcaggccaacggctacctgaattggtatcagcagaggaggggcaaggcaccaaagctgctgatctacgacggcagcaagctggagagaggcgtgccctcccggttctctggcaggagatggggccaggagtataacctgaccatcaacaatctgcagcctgaggatatcgccacatacttctgccaggtgtatgagtttgtggtgcctggcacccgcctggacctgaagaggacagtggccgccccaagcgtgttcatctttccacccagcgatgagcagctgaagtctggcaccgccagcgtggtgtgcctgctgaacaatttctacccaagagaggccaaggtgcagtggaaggtggacaacgccctgcagagcggcaattcccaggagtctgtgaccgagcaggacagcaaggattccacatattccctgtctaacaccctgacactgagcaaggccgattacgagaagcacaaggtgtatgcatgcgaggtgacccaccagggactgtcctctcctgtgacaaagtcctttaatcggggcgagtgttgataactcgag

SEQ ID NO: 11 - protein, 469 aa, synthetic construct
  REGION 1..469: note = 3BNC117 human IgG4 (heavy chain) (mod 4)
MDWTWRILFLVAAATGTHAQVQLLQSGAAVTKPGASVRVSCEASGYNIRDYFIHWWRQAPGQGLQWVGWINPKTGQPNNPRQFQGRVSLTRHASWDFDTFSFYMDLKALRSDDTAVYFCARQRSDYWDFDVWGSGTQVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - DNA, 1431 bp, synthetic construct
  misc_feature 1..1431: note = 3BNC117 human IgG4 (heavy chain) (mod 4)
  source 1..1431: mol_type = other DNA
ggatccgccaccatggactggacctggcgcatcctgttcctggtggcagcagcaaccggaacacacgcacaggtgcagctgctgcagagcggagcagcagtgacaaagccaggagcatctgtgagggtgagctgcgaggcatccggctacaacatccgcgattacttcatccactggtggaggcaggcaccaggacagggactgcagtgggtgggctggatcaatcctaagaccggccagcctaacaatccaaggcagttccagggacgcgtgagcctgaccaggcacgcctcctgggactttgatacattctctttttacatggacctgaaggccctgcgcagcgacgataccgccgtgtacttctgtgcccggcagaggagcgattattgggactttgacgtgtggggctctggcacccaggtgacagtgagctccgcctccacaaagggaccaagcgtgttcccactggcaccttgcagcaggtccacctctgagagcacagccgccctgggctgtctggtgaaggactacttccctgagccagtgaccgtgagctggaactccggcgccctgaccagcggagtgcacacatttcccgccgtgctgcagtctagcggcctgtactccctgtcctctgtggtgaccgtgcctagctcctctctgggcaccaagacatatacctgcaacgtggaccacaagccaagcaatacaaaggtggataagagagtggagtccaagtacggccctccctgccctccctgtccagcacctgagttcctgggcggcccttccgtgttcctgtttccacccaagccaaaggacaccctgatgatctctcggacccccgaggtgacatgcgtggtggtggacgtgagccaggaggaccccgaggtgcagttcaactggtacgtggatggcgtggaggtgcacaatgccaagacaaagcccagagaggagcagtttaactctacctaccgggtggtgagcgtgctgacagtgctgcaccaggactggctgaacggcaaggagtataagtgcaaggtgtccaataagggcctgcctagctccatcgagaagaccatctctaaggcaaagggacagccacgcgagccacaggtgtacacactgcctccaagccaggaggagatgaccaagaaccaggtgtccctgacatgtctggtgaagggcttctatccaagcgacatcgccgtggagtgggagtccaatggccagcccgagaacaattacaagaccacaccccctgtgctggactctgatggcagcttctttctgtatagcagactgaccgtggataagtcccggtggcaggagggcaacgtgttttcctgctctgtgatgcacgaggccctgcacaatcactacacccagaagagcctgtccctgtctctgggcaagtgataactcgag

SEQ ID NO: 13 - protein, 226 aa, synthetic construct
  REGION 1..226: note = 3BNC117 human IgG4 (light chain)(mod 4)
MVLQTQVFISLLLWISGAYGDIQMTQSPSSLSASVGDTVTITCQANGYLNWYQQRRGKAPKLLIYDGSKLERGVPSRFSGRRWGQEYNLTINNLQPEDIATYFCQVYEFVVPGTRLDLKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 14 - DNA, 702 bp, synthetic construct
  misc_feature 1..702: note = 3BNC117 human IgG4 (light chain)(mod 4)
  source 1..702: mol_type = other DNA
ggatccgccaccatggtgctgcagacccaggtgtttatctccctgctgctgtggatctctggcgcctatggcgacatccagatgacacagtccccctctagcctgtctgccagcgtgggcgatacagtgaccatcacatgtcaggccaacggctacctgaattggtatcagcagaggaggggcaaggcaccaaagctgctgatctacgacggcagcaagctggagagaggcgtgccctcccggttctctggcaggagatggggccaggagtataacctgaccatcaacaatctgcagcctgaggatatcgccacatacttctgccaggtgtatgagtttgtggtgcctggcacccgcctggacctgaagaggacagtggccgccccaagcgtgttcatctttccacccagcgatgagcagctgaagtctggcaccgccagcgtggtgtgcctgctgaacaatttctacccaagagaggccaaggtgcagtggaaggtggacaacgccctgcagagcggcaattcccaggagtctgtgaccgagcaggacagcaaggattccacatattccctgtctaacaccctgacactgagcaaggccgattacgagaagcacaaggtgtatgcatgcgaggtgacccaccagggactgtcctctcctgtgacaaagtcctttaatcggggcgagtgttgataactcgag

SEQ ID NO: 15 - protein, 744 aa, synthetic construct
  REGION 1..744: note = PGT128 Human IgG1
MDWTWRILFLVAAATGTHAQPQLQESGPTLVEASETLSLTCAVSGDSTAACNSFWGWVRQPPGKGLEWVGSLSHCASYWNRGWTYHNPSLKSRLTLALDTPKNLVFLKLNSVTAADTATYYCARFGGEVLRYTDWPKPAWVDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMAWTPLFLFLLTCCPGGSNSQSALTQPPSASGSPGQSITISCTGTSNNFVSWYQQHAGKAPKLVIYDVNKRPSGVPDRFSGSKSGNTASLTVSGLQTDDEAVYYCGSLVGNWDVIFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 16 - DNA, 2256 bp, synthetic construct
  misc_feature 1..2256: note = PGT128 Human IgG1
  source 1..2256: mol_type = other DNA
ggatccgccaccatggactggacctggagaatcctgttcctggtggcagcagcaacaggaacccacgcacagccacagctgcaggagtccggacccaccctggtggaggcctccgagacactgtctctgacctgcgccgtgagcggcgattccacagcagcctgtaactccttctggggatgggtgcgccagccccctggcaagggcctggagtgggtgggctctctgagccactgcgccagctactggaacaggggctggacctatcacaatccctctctgaagagcagactgaccctggccctggacacacctaagaacctggtgttcctgaagctgaatagcgtgaccgccgccgatacagccacctactattgtgccaggtttggcggcgaggtgctgagatacacagactggccaaagccagcatgggtggatctgtggggaaggggcacactggtgaccgtgagctccgcctccaccaagggaccaagcgtgttcccactggcaccttctagcaagtccacatctggcggcaccgccgccctgggatgcctggtgaaggactacttccctgagccagtgacagtgtcctggaactctggcgccctgacctctggcgtgcacacatttcccgccgtgctgcagtcctctggcctgtacagcctgagctccgtggtgaccgtgccttctagctccctgggcacacagacctatatctgcaacgtgaatcacaagcctagcaatacaaaggtggacaagaaggtggagccaaagtcctgtgataagacacacacctgcccaccctgtccagcacctgagctgctgggcggcccttccgtgttcctgtttcctccaaagccaaaggacaccctgatgatctcccggacacctgaggtgacctgcgtggtggtggacgtgtctcacgaggaccccgaggtgaagtttaactggtacgtggatggcgtggaggtgcacaatgccaagaccaagcccagggaggagcagtacaactctacctatagagtggtgagcgtgctgacagtgctgcaccaggattggctgaacggcaaggagtataagtgcaaggtgagcaataaggccctgccagcccccatcgagaagaccatctccaaggcaaagggacagccacgggagccacaggtgtacacactgcccccttcccgcgacgagctgaccaagaaccaggtgtctctgacatgtctggtgaagggcttctatccaagcgatatcgccgtggagtgggagtccaatggccagcccgagaacaattacaagaccacaccacccgtgctggacagcgatggctccttctttctgtattccaagctgaccgtggacaagtctaggtggcagcagggcaacgtgtttagctgctccgtgatgcacgaggccctgcacaatcactacacccagaagtctctgagcctgtcccctggcaagaggggaaggaagaggagatctggcagcggcgccacaaacttcagcctgctgaagcaggccggcgatgtggaggagaatcctggcccaatggcctggacccctctgttcctgttcctgctgacatgctgtcctggcggctccaactctcagagcgccctgacccagcctccatccgcctctggcagccctggacagagcatcacaatctcctgtacaggcaccagcaacaatttcgtgagctggtaccagcagcacgcaggcaaggcaccaaagctggtcatctacgacgtgaacaagcggccttccggcgtgccagatcgcttctccggctctaagagcggcaatacagcctctctgaccgtgagcggcctgcagaccgacgatgaggccgtgtactattgcggcagcctggtgggcaactgggacgtgatcttcggcggcggaacaaagctgaccgtgctgggacagccaaaggcagcaccttccgtgaccctgtttcccccttctagcgaggagctgcaggccaataaggccaccctggtgtgcctgatcagcgacttctaccctggagcagtgacagtggcatggaaggccgattcctctccagtgaaggccggcgtggagaccacaaccccctctaagcagagcaacaataagtacgccgccagctcctatctgtctctgaccccagagcagtggaagagccacaagtcctattcttgccaggtgacacacgagggctctacagtggagaagaccgtggcccccacagagtgtagctgataactcgag

SEQ ID NO: 17 - protein, 484 aa, synthetic construct
  REGION 1..484: note = PGT128 HUMAN IgG1- (HEAVY CHAIN) (MOD 1)
MDWTWRILFLVAAATGTHAQPQLQESGPTLVEASETLSLTCAVSGDSTAACNSFWGWVRQPPGKGLEWVGSLSHCASYWNRGWTYHNPSLKSRLTLALDTPKNLVFLKLNSVTAADTATYYCARFGGEVLRYTDWPKPAWVDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - DNA, 1476 bp, synthetic construct
  misc_feature 1..1476: note = PGT128 HUMAN IgG1- (HEAVY CHAIN) (MOD 1)
  source 1..1476: mol_type = other DNA
ggatccgccaccatggactggacctggagaatcctgttcctggtggcagcagcaacaggaacccacgcacagccacagctgcaggagtccggacccaccctggtggaggcctccgagacactgtctctgacctgcgccgtgagcggcgattccacagcagcctgtaactccttctggggatgggtgcgccagccccctggcaagggcctggagtgggtgggctctctgagccactgcgccagctactggaacaggggctggacctatcacaatccctctctgaagagcagactgaccctggccctggacacacctaagaacctggtgttcctgaagctgaatagcgtgaccgccgccgatacagccacctactattgtgccaggtttggcggcgaggtgctgagatacacagactggccaaagccagcatgggtggatctgtggggaaggggcacactggtgaccgtgagctccgcctccaccaagggaccaagcgtgttcccactggcaccttctagcaagtccacatctggcggcaccgccgccctgggatgcctggtgaaggactacttccctgagccagtgacagtgtcctggaactctggcgccctgacctctggcgtgcacacatttcccgccgtgctgcagtcctctggcctgtacagcctgagctccgtggtgaccgtgccttctagctccctgggcacacagacctatatctgcaacgtgaatcacaagcctagcaatacaaaggtggacaagaaggtggagccaaagtcctgtgataagacacacacctgcccaccctgtccagcacctgagctgctgggcggcccttccgtgttcctgtttcctccaaagccaaaggacaccctgatgatctcccggacacctgaggtgacctgcgtggtggtggacgtgtctcacgaggaccccgaggtgaagtttaactggtacgtggatggcgtggaggtgcacaatgccaagaccaagcccagggaggagcagtacaactctacctatagagtggtgagcgtgctgacagtgctgcaccaggattggctgaacggcaaggagtataagtgcaaggtgagcaataaggccctgccagcccccatcgagaagaccatctccaaggcaaagggacagccacgggagccacaggtgtacacactgcccccttcccgcgacgagctgaccaagaaccaggtgtctctgacatgtctggtgaagggcttctatccaagcgatatcgccgtggagtgggagtccaatggccagcccgagaacaattacaagaccacaccacccgtgctggacagcgatggctccttctttctgtattccaagctgaccgtggacaagtctaggtggcagcagggcaacgtgtttagctgctccgtgatgcacgaggccctgcacaatcactacacccagaagtctctgagcctgtcccctggcaagtgataactcgag

SEQ ID NO: 19 - protein, 231 aa, synthetic construct
  REGION 1..231: note = PGT128 human IgG1- (light chain) (mod 1)
MAWTPLFLFLLTCCPGGSNSQSALTQPPSASGSPGQSITISCTGTSNNFVSWYQQHAGKAPKLVIYDVNKRPSGVPDRFSGSKSGNTASLTVSGLQTDDEAVYYCGSLVGNWDVIFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 20 - DNA, 717 bp, synthetic construct
  misc_feature 1..717: note = PGT128 human IgG1- (light chain) (mod 1)
  source 1..717: mol_type = other DNA
ggatccgccaccatggcctggacccctctgttcctgttcctgctgacatgctgtcctggcggctccaactctcagagcgccctgacccagcctccatccgcctctggcagccctggacagagcatcacaatctcctgtacaggcaccagcaacaatttcgtgagctggtaccagcagcacgcaggcaaggcaccaaagctggtcatctacgacgtgaacaagcggccttccggcgtgccagatcgcttctccggctctaagagcggcaatacagcctctctgaccgtgagcggcctgcagaccgacgatgaggccgtgtactattgcggcagcctggtgggcaactgggacgtgatcttcggcggcggaacaaagctgaccgtgctgggacagccaaaggcagcaccttccgtgaccctgtttcccccttctagcgaggagctgcaggccaataaggccaccctggtgtgcctgatcagcgacttctaccctggagcagtgacagtggcatggaaggccgattcctctccagtgaaggccggcgtggagaccacaaccccctctaagcagagcaacaataagtacgccgccagctcctatctgtctctgaccccagagcagtggaagagccacaagtcctattcttgccaggtgacacacgagggctctacagtggagaagaccgtggcccccacagagtgtagctgataactcgag

SEQ ID NO: 21 - protein, 741 aa, synthetic construct
  REGION 1..741: note = PGT128 human IgG4 (mod 2)
MDWTWRILFLVAAATGTHAQPQLQESGPTLVEASETLSLTCAVSGDSTAACNSFWGWVRQPPGKGLEWVGSLSHCASYWNRGWTYHNPSLKSRLTLALDTPKNLVFLKLNSVTAADTATYYCARFGGEVLRYTDWPKPAWVDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKRGRKRRSGSGATNFSLLKQAGDVEENPGPMAWTPLFLFLLTCCPGGSNSQSALTQPPSASGSPGQSITISCTGTSNNFVSWYQQHAGKAPKLVIYDVNKRPSGVPDRFSGSKSGNTASLTVSGLQTDDEAVYYCGSLVGNWDVIFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 22 - DNA, 2247 bp, synthetic construct
  misc_feature 1..2247: note = PGT128 human IgG4 (mod 2)
  source 1..2247: mol_type = other DNA
ggatccgccaccatggactggacctggagaatcctgttcctggtggcagcagcaacaggaacccacgcacagccccagctgcaggagtccggccctacactggtggaggccagcgagaccctgtccctgacatgcgccgtgtctggcgatagcaccgcagcatgtaactccttctggggatgggtgcgccagccccctggcaagggcctggagtgggtgggctctctgagccactgcgccagctactggaacaggggctggacctatcacaatccatccctgaagtctagactgacactggccctggacacccccaagaacctggtgttcctgaagctgaatagcgtgacagccgccgatacagccacctactattgtgccaggtttggcggcgaggtgctgagatacaccgactggccaaagccagcatgggtggatctgtggggaaggggcaccctggtgacagtgagctccgccagcacaaagggcccttccgtgtttcctctggccccatgctcccgctctaccagcgagtccacagccgccctgggatgtctggtgaaggactatttccctgagccagtgaccgtgagctggaactccggcgccctgacatccggagtgcacacctttccagccgtgctgcagtctagcggcctgtactctctgtcctctgtggtgacagtgccaagctcctctctgggcacaaagacctatacatgcaacgtggaccacaagccctccaataccaaggtggataagagggtggagtctaagtacggcccaccctgcccaagctgtccagcacctgagttcctgggcggcccaagcgtgttcctgtttcctccaaagcccaaggacaccctgatgatcagcagaacccccgaggtgacatgcgtggtggtggacgtgtcccaggaggaccccgaggtgcagttcaactggtacgtggatggcgtggaggtgcacaatgccaagaccaagccacgggaggagcagtttaattccacataccgcgtggtgtctgtgctgaccgtgctgcaccaggactggctgaacggcaaggagtataagtgcaaggtgtctaataagggcctgcccagctccatcgagaagacaatcagcaaggcaaagggacagccccgggagcctcaggtgtacaccctgcccccttctcaggaggagatgacaaagaaccaggtgagcctgacctgtctggtgaagggcttctatccttccgacatcgccgtggagtgggagtctaatggccagccagagaacaattacaagaccacaccacccgtgctggactctgatggcagcttctttctgtatagccggctgaccgtggataagtcccgctggcaggagggcaacgtgttttcttgcagcgtgatgcacgaggccctgcacaatcactacacacagaagtccctgtctctgagcctgggcaagaggggaaggaagaggagatccggctctggcgccaccaacttcagcctgctgaagcaggcaggcgacgtggaggagaatccaggacccatggcctggacacctctgttcctgtttctgctgacctgctgtccaggcggcagcaactcccagtctgccctgacacagcctccaagcgcctccggctctccaggacagtctatcaccatcagctgtaccggcacatctaacaattttgtgagctggtaccagcagcacgcaggcaaggcacctaagctggtcatctacgacgtgaacaagaggccatccggcgtgcccgatagattcagcggctccaagtctggcaataccgcctccctgacagtgtctggcctgcagacagacgatgaggccgtgtactattgcggcagcctggtgggcaactgggatgtgatcttcggcggcggaaccaagctgacagtgctgggacagcctaaggcagcaccatccgtgaccctgtttcccccttctagcgaggagctgcaggccaataaggccaccctggtgtgcctgatcagcgacttctaccctggagcagtgaccgtggcatggaaggccgattcctctcccgtgaaggccggcgtggagaccacaaccccttccaagcagtctaacaataagtacgccgccagctcctatctgagcctgacacctgagcagtggaagtcccacaagagctattcctgccaggtgacccacgagggctccaccgtggagaagacagtggccccaaccgagtgttcttgataactcgag

SEQ ID NO: 23 - protein, 741 aa, synthetic construct
  REGION 1..741: note = PGT128 human IgG4mut (S228P) (mod3)
MDWTWRILFLVAAATGTHAQPQLQESGPTLVEASETLSLTCAVSGDSTAACNSFWGWVRQPPGKGLEWVGSLSHCASYWNRGWTYHNPSLKSRLTLALDTPKNLVFLKLNSVTAADTATYYCARFGGEVLRYTDWPKPAWVDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKRGRKRRSGSGATNFSLLKQAGDVEENPGPMAWTPLFLFLLTCCPGGSNSQSALTQPPSASGSPGQSITISCTGTSNNFVSWYQQHAGKAPKLVIYDVNKRPSGVPDRFSGSKSGNTASLTVSGLQTDDEAVYYCGSLVGNWDVIFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 24 - DNA, 2247 bp, synthetic construct
  misc_feature 1..2247: note = PGT128 human IgG4mut (S228P) (mod3)
  source 1..2247: mol_type = other DNA
ggatccgccaccatggactggacctggagaatcctgttcctggtggcagcagcaacaggaacccacgcacagccccagctgcaggagtccggccctacactggtggaggccagcgagaccctgtccctgacatgcgccgtgtctggcgatagcaccgcagcatgtaactccttctggggatgggtgcgccagccccctggcaagggcctggagtgggtgggctctctgagccactgcgccagctactggaacaggggctggacctatcacaatccatccctgaagtctagactgacactggccctggacacccccaagaacctggtgttcctgaagctgaatagcgtgacagccgccgatacagccacctactattgtgccaggtttggcggcgaggtgctgagatacaccgactggccaaagccagcatgggtggatctgtggggaaggggcaccctggtgacagtgagctccgccagcacaaagggcccttccgtgtttcctctggccccatgctcccgctctaccagcgagtccacagccgccctgggatgtctggtgaaggactatttccctgagccagtgaccgtgagctggaactccggcgccctgacatccggagtgcacacctttccagccgtgctgcagtctagcggcctgtactctctgtcctctgtggtgacagtgccaagctcctctctgggcacaaagacctatacatgcaacgtggaccacaagccctccaataccaaggtggataagagggtggagtctaagtacggcccaccctgcccaccctgtccagcacctgagttcctgggcggcccaagcgtgttcctgtttcctccaaagcccaaggacaccctgatgatcagcagaacccccgaggtgacatgcgtggtggtggacgtgtcccaggaggaccccgaggtgcagttcaactggtacgtggatggcgtggaggtgcacaatgccaagaccaagccacgggaggagcagtttaattccacataccgcgtggtgtctgtgctgaccgtgctgcaccaggactggctgaacggcaaggagtataagtgcaaggtgtctaataagggcctgcccagctccatcgagaagacaatcagcaaggcaaagggacagccccgggagcctcaggtgtacaccctgcccccttctcaggaggagatgacaaagaaccaggtgagcctgacctgtctggtgaagggcttctatccttccgacatcgccgtggagtgggagtctaatggccagccagagaacaattacaagaccacaccacccgtgctggactctgatggcagcttctttctgtatagccggctgaccgtggataagtcccgctggcaggagggcaacgtgttttcttgcagcgtgatgcacgaggccctgcacaatcactacacacagaagtccctgtctctgagcctgggcaagaggggaaggaagaggagatccggctctggcgccaccaacttcagcctgctgaagcaggcaggcgacgtggaggagaatccaggacccatggcctggacacctctgttcctgtttctgctgacctgctgtccaggcggcagcaactcccagtctgccctgacacagcctccaagcgcctccggctctccaggacagtctatcaccatcagctgtaccggcacatctaacaattttgtgagctggtaccagcagcacgcaggcaaggcacctaagctggtcatctacgacgtgaacaagaggccatccggcgtgcccgatagattcagcggctccaagtctggcaataccgcctccctgacagtgtctggcctgcagacagacgatgaggccgtgtactattgcggcagcctggtgggcaactgggatgtgatcttcggcggcggaaccaagctgacagtgctgggacagcctaaggcagcaccatccgtgaccctgtttcccccttctagcgaggagctgcaggccaataaggccaccctggtgtgcctgatcagcgacttctaccctggagcagtgaccgtggcatggaaggccgattcctctcccgtgaaggccggcgtggagaccacaaccccttccaagcagtctaacaataagtacgccgccagctcctatctgagcctgacacctgagcagtggaagtcccacaagagctattcctgccaggtgacccacgagggctccaccgtggagaagacagtggccccaaccgagtgttcttgataactcgag

SEQ ID NO: 25 - protein, 481 aa, synthetic construct
  REGION 1..481: note = PGT128 human IgG4-heavy chain (mod 4)
MDWTWRILFLVAAATGTHAQPQLQESGPTLVEASETLSLTCAVSGDSTAACNSFWGWVRQPPGKGLEWVGSLSHCASYWNRGWTYHNPSLKSRLTLALDTPKNLVFLKLNSVTAADTATYYCARFGGEVLRYTDWPKPAWVDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 26 - DNA, 1467 bp, synthetic construct
  misc_feature 1..1467: note = PGT128 human IgG4-heavy chain (mod 4)
  source 1..1467: mol_type = other DNA
ggatccgccaccatggactggacctggagaatcctgttcctggtggcagcagcaacaggaacccacgcacagccccagctgcaggagtccggccctacactggtggaggccagcgagaccctgtccctgacatgcgccgtgtctggcgatagcaccgcagcatgtaactccttctggggatgggtgcgccagccccctggcaagggcctggagtgggtgggctctctgagccactgcgccagctactggaacaggggctggacctatcacaatccatccctgaagtctagactgacactggccctggacacccccaagaacctggtgttcctgaagctgaatagcgtgacagccgccgatacagccacctactattgtgccaggtttggcggcgaggtgctgagatacaccgactggccaaagccagcatgggtggatctgtggggaaggggcaccctggtgacagtgagctccgccagcacaaagggcccttccgtgtttcctctggccccatgctcccgctctaccagcgagtccacagccgccctgggatgtctggtgaaggactatttccctgagccagtgaccgtgagctggaactccggcgccctgacatccggagtgcacacctttccagccgtgctgcagtctagcggcctgtactctctgtcctctgtggtgacagtgccaagctcctctctgggcacaaagacctatacatgcaacgtggaccacaagccctccaataccaaggtggataagagggtggagtctaagtacggcccaccctgcccaccctgtccagcacctgagttcctgggcggcccaagcgtgttcctgtttcctccaaagcccaaggacaccctgatgatcagcagaacccccgaggtgacatgcgtggtggtggacgtgtcccaggaggaccccgaggtgcagttcaactggtacgtggatggcgtggaggtgcacaatgccaagaccaagccacgggaggagcagtttaattccacataccgcgtggtgtctgtgctgaccgtgctgcaccaggactggctgaacggcaaggagtataagtgcaaggtgtctaataagggcctgcccagctccatcgagaagacaatcagcaaggcaaagggacagccccgggagcctcaggtgtacaccctgcccccttctcaggaggagatgacaaagaaccaggtgagcctgacctgtctggtgaagggcttctatccttccgacatcgccgtggagtgggagtctaatggccagccagagaacaattacaagaccacaccacccgtgctggactctgatggcagcttctttctgtatagccggctgaccgtggataagtcccgctggcaggagggcaacgtgttttcttgcagcgtgatgcacgaggccctgcacaatcactacacacagaagtccctgtctctgagcctgggcaagtgataactcgag

SEQ ID NO: 27 - protein, 231 aa, synthetic construct; identical to SEQ ID NO: 19
  REGION 1..231: note = PGT128 human IgG4-light chain (mod 4)

SEQ ID NO: 28 - DNA, 717 bp, synthetic construct
  misc_feature 1..717: note = PGT128 human IgG4-light chain (mod 4)
  source 1..717: mol_type = other DNA
ggatccgccaccatggcctggacacctctgttcctgtttctgctgacctgctgtccaggcggcagcaactcccagtctgccctgacacagcctccaagcgcctccggctctccaggacagtctatcaccatcagctgtaccggcacatctaacaattttgtgagctggtaccagcagcacgcaggcaaggcacctaagctggtcatctacgacgtgaacaagaggccatccggcgtgcccgatagattcagcggctccaagtctggcaataccgcctccctgacagtgtctggcctgcagacagacgatgaggccgtgtactattgcggcagcctggtgggcaactgggatgtgatcttcggcggcggaaccaagctgacagtgctgggacagcctaaggcagcaccatccgtgaccctgtttcccccttctagcgaggagctgcaggccaataaggccaccctggtgtgcctgatcagcgacttctaccctggagcagtgaccgtggcatggaaggccgattcctctcccgtgaaggccggcgtggagaccacaaccccttccaagcagtctaacaataagtacgccgccagctcctatctgagcctgacacctgagcagtggaagtcccacaagagctattcctgccaggtgacccacgagggctccaccgtggagaagacagtggccccaaccgagtgttcttgataactcgag

SEQ ID NO: 29 - protein, 750 aa, synthetic construct
  REGION 1..750: note = PG9 IgG1
MDWTWRILFLVAAATGTHAQRLVESGGGVVQPGSSLRLSCAASGFDFSRQGMHWVRQAPGQGLEWVAFIKYDGSEKYHADSVWGRLSISRDNSKDTLYLQMNSLRVEDTATYFCVREAGGPDYRNGYNYYDFYDGYYNYHYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMAWTPLFLFLLTCCPGGSNSQSALTQPASVSGSPGQSITISCNGTSNDVGGYESVSWYQQHPGKAPKVVIYDVSKRPSGVSNRFSGSKSGNTASLTISGLQAEDEGDYYCKSLTSTRRRVFGTGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 30 - DNA, 2274 bp, synthetic construct
  misc_feature 1..2274: note = PG9 IgG1
  source 1..2274: mol_type = other DNA
ggatccgccaccatggactggacttggagaatcctgttcctggtcgccgccgctaccgggactcacgctcagaggctggtggagagtgggggaggggtcgtgcagccaggcagctccctgcgactgagttgcgccgcttcagggttcgacttttcccgacagggaatgcactgggtcaggcaggcacctggacagggactggagtgggtggcttttatcaagtacgacggaagtgaaaaatatcatgccgattcagtgtgggggcggctgtcaattagccgcgacaacagtaaggataccctgtacctgcagatgaatagcctgcgggtcgaggacacagctacttatttctgcgtgcgcgaagcaggcggaccagattaccgaaacgggtataattactatgacttttacgatggctactataactaccactatatggacgtgtggggcaagggaaccacagtcacagtgtctagtgcatcaactaaaggacccagcgtgttccccctggccccttcaagcaagtccacttctgggggcaccgcagccctgggatgtctggtgaaggattacttccctgagccagtcaccgtgagttggaactcaggcgccctgactagcggagtccatacctttcctgctgtgctgcagtcctctgggctgtactccctgagttcagtggtcactgtgccaagctcctctctgggcacccagacatatatctgcaacgtgaatcacaagcctagcaataccaaagtcgacaagaaagtggaaccaaagtcctgtgataaaactcatacctgccctccctgtccagcacctgagctgctgggagggccttccgtgttcctgtttccacccaagccaaaagacacactgatgatttctaggacaccagaagtcacttgcgtggtcgtggacgtgagccacgaggaccccgaagtcaagtttaactggtacgtggatggcgtcgaggtgcataatgccaagaccaaaccccgcgaggaacagtacaactccacatatagggtcgtgtctgtcctgactgtgctgcaccaggactggctgaacggcaaggagtataagtgcaaagtgtccaataaggcactgccagcccccatcgagaaaaccatttctaaggccaaaggacagccaagagaaccccaggtgtacacactgcctccatcccgggacgagctgactaagaaccaggtctctctgacctgtctggtgaaaggcttctatccctctgatatcgctgtggagtgggaaagtaatggacagcctgaaaacaattacaagactacccctcccgtgctggacagcgatgggtccttctttctgtatagcaagctgacagtggacaaatccagatggcagcagggcaacgtctttagttgctcagtgatgcacgaggccctgcacaatcattacacacagaaaagcctgtccctgtctcccggcaagaggggaagaaaaaggagaagtgggtcaggcgcaactaacttcagcctgctgaagcaggccggagatgtggaggaaaatcctgggccaatggcttggacccctctgttcctgttcctgctgacatgctgtcctggcggaagcaactcccagtctgcactgacccagccagcaagcgtgagcgggagcccaggccagagcatcaccatttcctgtaacggcacatccaatgacgtgggcggctacgagtccgtgtcttggtatcagcagcatcctggaaaggccccaaaagtcgtgatctacgatgtcagcaaaagaccttctggggtgagtaaccggttcagtggatcaaagagcgggaataccgcttctctgacaattagtggcctgcaggcagaggacgaaggagattactattgcaagtcactgacaagcactcggcgccgagtcttcggaaccgggacaaagctgactgtgctgggccagcccaaagctgcaccttctgtgaccctgtttccacccagttcagaggaactgcaggctaacaaggccaccctggtgtgcctgatcagcgacttctaccctggcgctgtcactgtggcctggaaggctgatagctccccagtcaaagcaggagtggaaacaactaccccctccaagcagtctaacaacaagtacgccgcttctagttatctgtcactgaccccagagcagtggaagtctcacaaatcctattcttgtcaggtcacccatgaagggagcactgtggagaaaaccgtcgcaccaaccgaatgttcctgataactcgag

SEQ ID NO: 31 - protein, 747 aa, synthetic construct
  REGION 1..747: note = PG9 human IgG4
MDWTWRILFLVAAATGTHAQRLVESGGGVVQPGSSLRLSCAASGFDFSRQGMHWVRQAPGQGLEWVAFIKYDGSEKYHADSVWGRLSISRDNSKDTLYLQMNSLRVEDTATYFCVREAGGPDYRNGYNYYDFYDGYYNYHYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKRGRKRRSGSGATNFSLLKQAGDVEENPGPMAWTPLFLFLLTCCPGGSNSQSALTQPASVSGSPGQSITISCNGTSNDVGGYESVSWYQQHPGKAPKVVIYDVSKRPSGVSNRFSGSKSGNTASLTISGLQAEDEGDYYCKSLTSTRRRVFGTGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 32 - DNA, 2265 bp, synthetic construct
  misc_feature 1..2265: note = PG9 human IgG4
  source 1..2265: mol_type = other DNA
ggatccgccaccatggactggacctggcgcatcctgtttctggtggcagcagcaacaggaacccacgcacagaggctggtggagagcggcggcggcgtggtgcagcctggcagctccctgagactgtcctgcgcagcatctggcttcgacttcagcaggcagggcatgcactgggtgcggcaggcaccaggacagggactggagtgggtggccttcatcaagtacgacggctctgagaagtatcacgcagatagcgtgtggggccgcctgtccatctctagggacaacagcaaggataccctgtacctgcagatgaattccctgcgcgtggaggacacagccacctatttctgcgtgagggaggccggcggaccagattacaggaacggctataattactatgacttttacgatggctactataactaccactatatggacgtgtggggcaagggcaccacagtgacagtgtctagcgcctccaccaagggaccaagcgtgttccctctggcaccatgcagcaggtccacctctgagagcacagccgccctgggctgtctggtgaaggattatttccctgagccagtgaccgtgagctggaattccggcgccctgacatccggagtgcacacctttcctgccgtgctgcagtcctctggcctgtacagcctgagctccgtggtgacagtgccctctagctccctgggcacaaagacctatacatgcaacgtggaccacaagccttccaataccaaggtggataagagagtggagagcaagtacggccctccctgcccatcctgtccagcacctgagttcctgggcggcccaagcgtgttcctgtttccacccaagcccaaggacacactgatgatcagccggacccctgaggtgacatgcgtggtggtggacgtgtcccaggaggaccccgaggtgcagttcaactggtacgtggatggcgtggaggtgcacaatgccaagaccaagcccagagaggagcagtttaacagcacataccgggtggtgtccgtgctgaccgtgctgcaccaggactggctgaacggcaaggagtataagtgcaaggtgtctaataagggcctgccttctagcatcgagaagacaatcagcaaggcaaagggacagccccgggagcctcaggtgtacaccctgcctccatctcaggaggagatgacaaagaaccaggtgagcctgacctgtctggtgaagggcttttatccatctgacatcgccgtggagtgggagagcaatggccagcccgagaacaattacaagaccacaccccctgtgctggactctgatggcagcttctttctgtattcccgcctgactgtggataagtctaggtggcaggagggcaacgtgttttcctgctctgtgatgcacgaggccctgcacaatcactacacacagaagagcctgtccctgtctctgggcaagagaggaaggaagaggagaagcggctccggagcaaccaacttcagcctgctgaagcaggcaggcgacgtggaggagaatccaggacccatggcctggacacctctgttcctgtttctgctgacctgctgtccaggcggctctaacagccagtccgccctgacccagccagcatctgtgagcggctcccctggccagagcatcacaatctcctgtaacggcacctccaatgacgtgggcggctacgagtctgtgagctggtatcagcagcacccaggcaaggcccccaaggtggtcatctacgatgtgagcaagagaccctccggcgtgtctaaccggttctccggctctaagagcggcaataccgcctctctgacaatcagcggactgcaggcagaggacgagggcgattactattgcaagagcctgacctccacacggcgcagggtgttcggaaccggcacaaagctgacagtgctgggacagcctaaggcagcaccatccgtgaccctgtttccaccctcctctgaggagctgcaggccaacaaggccaccctggtgtgcctgatcagcgacttctaccctggagcagtgaccgtggcatggaaggcagatagctccccagtgaaggcaggagtggagaccacaaccccttctaagcagagcaacaataagtacgccgcctctagctatctgtccctgacaccagagcagtggaagtctcacaagtcctattcttgccaggtgacccacgagggctccaccgtggagaagacagtggcccccaccgagtgttcttgataactcgag

SEQ ID NO: 33 - protein, 733 aa, synthetic construct
  REGION 1..733: note = NIH45-46human IgG1 antibody
MDWTWRILFLVAAATGTHAQVRLSQSGGQMKKPGESMRLSCRASGYEFLNCPINWIRLAPGRRPEWMGWLKPRGGAVNYARKFQGRVTMTRDVYSDTAFLELRSLTSDDTAVYFCTRGKYCTARDYYNWDFEHWGRGAPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGEIVLTQSPATLSLSPGETAIISCRTSQSGSLAWYQQRPGQAPRLVIYSGSTRAAGIPDRFSGSRWGADYNLSISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGEC

SEQ ID NO: 34 - DNA, 2223 bp, synthetic construct
  misc_feature 1..2223: note = NIH45-46human IgG1 antibody
  source 1..2223: mol_type = other DNA
ggatccgccaccatggactggacctggcgcatcctgttcctggtggcagcagcaaccggaacacacgcacaggtgaggctgtcccagtctggcggccagatgaagaagcctggagagtctatgaggctgagctgcagggcatccggatacgagttcctgaactgtccaatcaattggatcagactggcccctggcaggagaccagagtggatgggatggctgaagcctcggggcggcgccgtgaactacgcaaggaagtttcagggcagagtgaccatgacacgggacgtgtatagcgataccgccttcctggagctgagatctctgaccagcgacgatacagccgtgtacttttgcacccgcggcaagtattgtacagccagggactactataattgggatttcgagcactggggaaggggcgccccagtgaccgtgagctccgcctccacaaagggacctagcgtgttcccactggcaccttctagcaagagcacctccggcggcacagccgccctgggatgcctggtgaaggactacttccctgagccagtgaccgtgtcctggaactctggcgccctgaccagcggagtgcacacatttccagccgtgctgcagtcctctggcctgtactccctgagctccgtggtgaccgtgccctctagctccctgggcacccagacatatatctgcaacgtgaatcacaagccatctaatacaaaggtggacaagaaggtggagcccaagagctgtgataagacccacacatgccctccctgtccagcacctgagctgctgggcggcccttccgtgttcctgtttccacccaagccaaaggacacactgatgatctccagaacccccgaggtgacatgcgtggtggtggacgtgtctcacgaggaccccgaggtgaagttcaactggtacgtggatggcgtggaggtgcacaatgccaagaccaagccaagagaggagcagtacaactctacctatcgggtggtgagcgtgctgacagtgctgcaccaggattggctgaacggcaaggagtataagtgcaaggtgagcaataaggccctgccagcccccatcgagaagaccatctccaaggccaagggccagccaagagagccccaggtgtacacactgcctccaagccgggacgagctgaccaagaaccaggtgtccctgacatgtctggtgaagggcttctatccttccgatatcgccgtggagtgggagtctaatggccagccagagaacaattacaagaccacaccccctgtgctggacagcgatggctccttctttctgtatagcaagctgaccgtggacaagtcccggtggcagcagggcaacgtgttttcttgcagcgtgatgcacgaggccctgcacaatcactacacccagaagtccctgtctctgagcccaggcaagaggggaagaaagcggcgctccggctctggagccacaaacttcagcctgctgaagcaggccggcgatgtggaggagaatcctggcccaatggtgctgcagacccaggtgtttatctccctgctgctgtggatctctggcgcctacggcgagatcgtgctgacccagtctccagccacactgagcctgtccccaggagagaccgcaatcatctcctgtcgcacatctcagagcggctccctggcatggtaccagcagagaccaggacaggcccctcggctggtcatctactctggcagcacaagggccgcaggcatccccgaccgcttctccggctctaggtggggcgccgattacaacctgagcatctccaatctggagagcggcgactttggcgtgtactattgccagcagtatgagttctttggccagggcaccaaggtgcaggtggacatcaagcgcacagtggccgcccccagcgtgttcatctttccacccagcgatgagcagctgaagtctggcaccgccagcgtggtgtgcctgctgaacaatttctaccctagggaggccaaggtgcagtggaaggtggacaacgccctgcagtctggcaatagccaggagtccgtgaccgagcaggactctaaggatagcacatattccctgtctagcaccctgacactgtccaaggccgattacgagaagcacaaggtgtatgcatgcgaggtgacccaccagggactgcgcagccccgtgacaaagtcctttaacaggggcgagtgttgataactcgag

SEQ ID NO: 35 - protein, 730 aa, synthetic construct
  REGION 1..730: note = NIH45-46human IgG4 antibody
MDWTWRILFLVAAATGTHAQVRLSQSGGQMKKPGESMRLSCRASGYEFLNCPINWIRLAPGRRPEWMGWLKPRGGAVNYARKFQGRVTMTRDVYSDTAFLELRSLTSDDTAVYFCTRGKYCTARDYYNWDFEHWGRGAPVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKRGRKRRSGSGATNFSLLKQAGDVEENPGPMVLQTQVFISLLLWISGAYGEIVLTQSPATLSLSPGETAIISCRTSQSGSLAWYQQRPGQAPRLVIYSGSTRAAGIPDRFSGSRWGADYNLSISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 36 - DNA, 2214 bp, synthetic construct
  misc_feature 1..2214: note = NIH45-46human IgG4 antibody
  source 1..2214: mol_type = other DNA
ggatccgccaccatggactggacctggagaatcctgttcctggtggcagcagcaaccggaacacacgcacaggtgcggctgtcccagtctggcggccagatgaagaagccaggagagtctatgaggctgagctgcagggcatccggatacgagttcctgaactgtcctatcaattggatcaggctggcaccaggcaggagacccgagtggatgggatggctgaagcccaggggcggcgccgtgaactacgcaaggaagtttcagggcagagtgaccatgacacgggacgtgtattccgataccgccttcctggagctgagaagcctgacatccgacgataccgccgtgtacttttgcacacgcggcaagtattgtaccgccagggactactataattgggatttcgagcactggggaagaggcgcccctgtgacagtgagctccgcctccaccaagggcccaagcgtgttccctctggccccatgcagccggtccacatctgagagcaccgccgccctgggatgtctggtgaaggactacttccctgagccagtgaccgtgtcttggaacagcggcgccctgacatctggcgtgcacacctttcccgccgtgctgcagtctagcggcctgtacagcctgtcctctgtggtgacagtgcctagctcctctctgggcaccaagacatatacctgcaacgtggaccacaagccatccaataccaaggtggataagcgcgtggagtctaagtacggccctccctgccctagctgtccagcacctgagttcctgggcggcccaagcgtgttcctgtttccacccaagcccaaggacaccctgatgatctctaggacaccagaggtgacctgcgtggtggtggacgtgagccaggaggaccccgaggtgcagttcaactggtacgtggatggcgtggaggtgcacaatgccaagacaaagcctcgcgaggagcagtttaacagcacatacagggtggtgtccgtgctgaccgtgctgcaccaggattggctgaacggcaaggagtataagtgcaaggtgtccaataagggcctgccaagctccatcgagaagacaatctctaaggcaaagggacagccccgggagcctcaggtgtacaccctgcctccaagccaggaggagatgacaaagaaccaggtgtccctgacctgtctggtgaagggcttctatccctccgacatcgccgtggagtgggagtctaatggccagcctgagaacaattacaagaccacaccccctgtgctggactccgatggctctttctttctgtattccagactgaccgtggataagtctcggtggcaggagggcaacgtgttttcctgctctgtgatgcacgaggccctgcacaatcactacacacagaagagcctgtccctgtctctgggcaagaggggaagaaagcggcgcagcggctccggagcaaccaacttctccctgctgaagcaggcaggcgacgtggaggagaatccaggacccatggtgctgcagacacaggtgtttatctctctgctgctgtggatcagcggcgcctacggcgagatcgtgctgacacagagcccagccaccctgtctctgagccctggagagacagccatcatctcctgtcgcacctcccagtctggcagcctggcatggtaccagcagagacctggacaggccccacggctggtcatctactccggctctaccagggccgcaggcatcccagaccgcttcagcggctccaggtggggcgccgattacaacctgtctatcagcaatctggagagcggcgattttggcgtgtactattgccagcagtatgagttctttggccagggcacaaaggtgcaggtggacatcaagagaaccgtggccgccccaagcgtgttcatctttccaccctccgatgagcagctgaagtctggcacagccagcgtggtgtgcctgctgaacaatttctacccccgggaggccaaggtgcagtggaaggtggacaacgccctgcagagcggcaattcccaggagtctgtgacagagcaggacagcaaggattccacctattccctgtctaacacactgaccctgtccaaggccgattacgagaagcacaaggtgtatgcctgcgaggtgacacaccagggcctgtctagccctgtgaccaagagctttaatcgcggcgagtgttgataactcgag

SEQ ID NO: 37 - protein, 729 aa, synthetic construct
  REGION 1..729: note = VRC01 human IgG1
MDWTWRILFLVAAATGTHAQVQLVQSGGQMKKPGESMRISCRASGYEFIDCTLNWIRLAPGKRPEWMGWLKPRGGAVNYARPLQGRVTMTRDVYSDTAFLELRSLTVDDTAVYFCTRGKNCDYNWDFEHWGRGTPVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGEIVLTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 38 - DNA, 2211 bp, synthetic construct
  misc_feature 1..2211: note = VRC01 human IgG1
  source 1..2211: mol_type = other DNA
ggatccgccaccatggattggacatggaggattctgtttctggtcgccgccgccacaggaactcatgctcaggtgcagctggtgcagtcaggagggcagatgaagaaacctggcgagagtatgcgaatctcatgcagggctagcggatacgaattcatcgactgtaccctgaactggattagactggcacctggaaagcggccagagtggatggggtggctgaaaccaagaggcggagcagtgaattacgccagaccactgcagggacgagtcactatgacccgcgacgtgtatagcgataccgcattcctggaactgcgatccctgacagtcgacgatactgccgtgtacttctgcacaaggggcaagaactgtgactataattgggattttgagcactggggccggggaacaccagtcattgtgagctccgcaagtactaaggggccctcagtgtttcccctggctccttctagtaaaagtacctcagggggcacagccgctctgggatgcctggtgaaggattacttccctgagccagtcacagtgagttggaactcaggcgccctgacatctggagtccatacttttcccgctgtgctgcagtcaagcggactgtacagcctgtcctctgtggtcacagtgcctagttcaagcctggggacacagacttatatctgcaacgtcaatcacaagcccagcaatactaaagtggacaagaaagtggaacctaagtcctgtgataaaacccatacatgccctccctgtccagctcctgagctgctgggagggccaagcgtgttcctgtttccacccaagcctaaagacaccctgatgatttctcggactcctgaagtcacctgcgtggtcgtggacgtgagccacgaggaccccgaagtcaagtttaactggtacgtggatggcgtcgaggtgcataatgccaagacaaaacccagggaggaacagtacaacagcacctatagagtcgtgtccgtcctgacagtgctgcaccaggactggctgaacggaaaggagtataagtgcaaagtgagcaataaggccctgccagctcccatcgagaaaacaatttccaaggcaaaagggcagccacgggaaccccaggtgtacactctgcctccatctcgcgacgagctgactaagaaccaggtcagcctgacctgtctggtgaaagggttctatccaagtgatatcgccgtggagtgggaaagcaatggccagcccgaaaacaattacaagaccacaccccctgtgctggacagcgatggctccttctttctgtattctaagctgaccgtggataaaagtcgctggcagcagggaaacgtctttagctgctccgtgatgcacgaggctctgcacaatcattacacccagaagtctctgagtctgtcacctggcaagcggggacgcaaaaggagaagcgggtccggcgctacaaacttcagcctgctgaagcaggcaggcgacgtggaggaaaatcctggaccaatggtcctgcagacccaggtgttcatctccctgctgctgtggatttctggggcatacggcgaaattgtgctgacccagagccctgggacactgtctctgagtccaggcgagacagccatcatttcctgtcggacttctcagtacggaagtctggcatggtatcagcagcgaccaggacaggctcctcgactggtcatctactcagggagcactagggcagccggcattcctgaccgattctccggatctaggtgggggcctgattacaacctgaccatctcaaatctggaaagcggggactttggcgtgtactattgccagcagtatgagttctttggacaggggaccaaggtccaggtggacatcaaacgcacagtcgctgcaccatccgtgttcatttttccaccctctgatgaacagctgaagtccggcaccgcctctgtggtgtgcctgctgaacaatttctaccccagggaggcaaaggtccagtggaaagtggacaacgccctgcagtctggaaatagtcaggagtcagtgactgaacaggacagcaaggattccacctattctctgtccaacactctgaccctgtccaaagctgattacgagaagcacaaagtgtatgcatgcgaggtcactcaccaggggctgtcatcaccagtcacaaaatccttcaacagaggggaatgctgataactcgag

SEQ ID NO: 39 - protein, 470 aa, synthetic construct
  REGION 1..470: note = VRC01 human IgG1(heavy chain) (mod1)
MDWTWRILFLVAAATGTHAQVQLVQSGGQMKKPGESMRISCRASGYEFIDCTLNWIRLAPGKRPEWMGWLKPRGGAVNYARPLQGRVTMTRDVYSDTAFLELRSLTVDDTAVYFCTRGKNCDYNWDFEHWGRGTPVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - DNA, 1434 bp, synthetic construct
  misc_feature 1..1434: note = VRC01 human IgG1(heavy chain) (mod1)
  source 1..1434: mol_type = other DNA
ggatccgccaccatggattggacatggaggattctgtttctggtcgccgccgccacaggaactcatgctcaggtgcagctggtgcagtcaggagggcagatgaagaaacctggcgagagtatgcgaatctcatgcagggctagcggatacgaattcatcgactgtaccctgaactggattagactggcacctggaaagcggccagagtggatggggtggctgaaaccaagaggcggagcagtgaattacgccagaccactgcagggacgagtcactatgacccgcgacgtgtatagcgataccgcattcctggaactgcgatccctgacagtcgacgatactgccgtgtacttctgcacaaggggcaagaactgtgactataattgggattttgagcactggggccggggaacaccagtcattgtgagctccgcaagtactaaggggccctcagtgtttcccctggctccttctagtaaaagtacctcagggggcacagccgctctgggatgcctggtgaaggattacttccctgagccagtcacagtgagttggaactcaggcgccctgacatctggagtccatacttttcccgctgtgctgcagtcaagcggactgtacagcctgtcctctgtggtcacagtgcctagttcaagcctggggacacagacttatatctgcaacgtcaatcacaagcccagcaatactaaagtggacaagaaagtggaacctaagtcctgtgataaaacccatacatgccctccctgtccagctcctgagctgctgggagggccaagcgtgttcctgtttccacccaagcctaaagacaccctgatgatttctcggactcctgaagtcacctgcgtggtcgtggacgtgagccacgaggaccccgaagtcaagtttaactggtacgtggatggcgtcgaggtgcataatgccaagacaaaacccagggaggaacagtacaacagcacctatagagtcgtgtccgtcctgacagtgctgcaccaggactggctgaacggaaaggagtataagtgcaaagtgagcaataaggccctgccagctcccatcgagaaaacaatttccaaggcaaaagggcagccacgggaaccccaggtgtacactctgcctccatctcgcgacgagctgactaagaaccaggtcagcctgacctgtctggtgaaagggttctatccaagtgatatcgccgtggagtgggaaagcaatggccagcccgaaaacaattacaagaccacaccccctgtgctggacagcgatggctccttctttctgtattctaagctgaccgtggataaaagtcgctggcagcagggaaacgtctttagctgctccgtgatgcacgaggctctgcacaatcattacacccagaagtctctgagtctgtcacctggcaagtgataactcgag

SEQ ID NO: 41 - protein, 230 aa, synthetic construct
  REGION 1..230: note = VRC01 human IgG1(light chain)(mod1)
MVLQTQVFISLLLWISGAYGEIVLTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 42 - DNA, 714 bp, synthetic construct
  misc_feature 1..714: note = VRC01 human IgG1(light chain)(mod1)
  source 1..714: mol_type = other DNA
ggatccgccaccatggtcctgcagacccaggtgttcatctccctgctgctgtggatttctggggcatacggcgaaattgtgctgacccagagccctgggacactgtctctgagtccaggcgagacagccatcatttcctgtcggacttctcagtacggaagtctggcatggtatcagcagcgaccaggacaggctcctcgactggtcatctactcagggagcactagggcagccggcattcctgaccgattctccggatctaggtgggggcctgattacaacctgaccatctcaaatctggaaagcggggactttggcgtgtactattgccagcagtatgagttctttggacaggggaccaaggtccaggtggacatcaaacgcacagtcgctgcaccatccgtgttcatttttccaccctctgatgaacagctgaagtccggcaccgcctctgtggtgtgcctgctgaacaatttctaccccagggaggcaaaggtccagtggaaagtggacaacgccctgcagtctggaaatagtcaggagtcagtgactgaacaggacagcaaggattccacctattctctgtccaacactctgaccctgtccaaagctgattacgagaagcacaaagtgtatgcatgcgaggtcactcaccaggggctgtcatcaccagtcacaaaatccttcaacagaggggaatgctgataactcgag

SEQ ID NO: 43 - protein, 726 aa, synthetic construct
  REGION 1..726: note = VRC01 human IgG4 (mod 2)
MDWTWRILFLVAAATGTHAQVQLVQSGGQMKKPGESMRISCRASGYEFIDCTLNWIRLAPGKRPEWMGWLKPRGGAVNYARPLQGRVTMTRDVYSDTAFLELRSLTVDDTAVYFCTRGKNCDYNWDFEHWGRGTPVIVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKRGRKRRSGSGATNFSLLKQAGDVEENPGPMVLQTQVFISLLLWISGAYGEIVLTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 44 - DNA, 2202 bp, synthetic construct
  misc_feature 1..2202: note = VRC01 human IgG4 (mod 2)
  source 1..2202: mol_type = other DNA
ggatccgccaccatggattggacttggaggattctgtttctggtcgccgccgctactggaacccatgctcaggtgcagctggtgcagtcaggagggcagatgaagaaacctggagagagcatgcgaatctcctgcagggcctctgggtacgaattcatcgactgtacactgaactggattagactggctcctgggaagcggccagagtggatgggctggctgaaaccaagaggcggagcagtgaattacgccagaccactgcagggacgagtcactatgacccgcgacgtgtattctgatactgcttttctggaactgcgaagtctgaccgtcgacgatacagcagtgtacttctgcaccaggggcaagaactgtgactataattgggattttgagcactggggaagagggactcctgtcattgtgagctccgcaagcaccaagggaccatccgtgttcccactggcaccttgcagtcggtcaactagcgagtccaccgccgctctgggatgtctggtgaaggattacttcccagaacccgtcaccgtgagctggaactccggagccctgaccagcggagtccatacatttcctgctgtgctgcagtctagtggactgtactccctgtcaagcgtggtcaccgtcccttcctctagtctggggacaaagacttatacctgcaacgtggaccacaaaccatccaatacaaaggtcgataaacgcgtggagtcaaagtacggccctccctgccctagctgtccagctcctgagttcctgggcggcccaagcgtgttcctgtttccccctaagcctaaagacacactgatgatctctaggacaccagaagtcacttgcgtggtcgtggacgtgagccaggaggaccccgaagtccagttcaactggtacgtggatggcgtcgaggtgcacaatgccaagaccaaacctcgggaggaacagtttaactctacttaccgcgtcgtgagtgtcctgaccgtgctgcatcaggactggctgaacggcaaggagtataagtgcaaagtgtccaataagggactgccatcaagcatcgagaaaaccatttctaaggcaaaaggccagccaagagaaccccaggtgtacacactgccacccagccaggaggaaatgacaaagaaccaggtctccctgacttgtctggtgaaagggttttatccaagcgacattgccgtggagtgggaatccaatggccagcccgagaacaattacaagaccacacctccagtgctggactctgatggcagtttctttctgtattcaaggctgaccgtggataaaagcagatggcaggagggaaacgtcttctcttgcagtgtgatgcacgaagctctgcacaatcattacactcagaagtcactgagcctgtccctgggaaagcgaggacgaaaaaggagatctggcagtggagctaccaacttcagcctgctgaagcaggcaggagacgtggaggaaaatcctgggccaatggtcctgcagactcaggtgttcatcagtctgctgctgtggatttcaggcgcctacggagagattgtgctgactcagtctcccggcaccctgtcactgagccctggagaaaccgccatcattagttgtcgaacatcacagtacgggagcctggcatggtatcagcagcgaccaggacaggctccccgactggtcatctactccggctctacacgggcagccggaattcccgaccgattcagtgggtcaaggtggggccctgattacaacctgactatctccaatctggagtctggcgactttggagtgtactattgccagcagtatgaattctttgggcagggcaccaaggtccaggtggacatcaaaagaacagtcgctgcaccaagcgtgttcatctttcccccttcagatgagcagctgaagagtggaaccgcctcagtggtgtgcctgctgaacaatttctacccccgggaagcaaaggtccagtggaaagtggacaacgccctgcagtcaggcaatagccaggagtccgtgacagaacaggactctaaggatagtacttattcactgtccaacacactgactctgtccaaagctgattacgagaagcacaaagtgtatgcatgcgaggtcacccaccaggggctgtcatcacccgtcaccaagtctttcaataggggcgaatgttgataactcgag

SEQ ID NO: 45 - protein, 726 aa, synthetic construct
  REGION 1..726: note = VRC01 human IgG4 mut (S228P) (mod 3)
MDWTWRILFLVAAATGTHAQVQLVQSGGQMKKPGESMRISCRASGYEFIDCTLNWIRLAPGKRPEWMGWLKPRGGAVNYARPLQGRVTMTRDVYSDTAFLELRSLTVDDTAVYFCTRGKNCDYNWDFEHWGRGTPVIVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKRGRKRRSGSGATNFSLLKQAGDVEENPGPMVLQTQVFISLLLWISGAYGEIVLTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 46 - DNA, 2202 bp, synthetic construct
  misc_feature 1..2202: note = VRC01 human IgG4 mut (S228P) (mod 3)
  source 1..2202: mol_type = other DNA
ggatccgccaccatggattggacttggaggattctgtttctggtcgccgccgctactggaacccatgctcaggtgcagctggtgcagtcaggagggcagatgaagaaacctggagagagcatgcgaatctcctgcagggcctctgggtacgaattcatcgactgtacactgaactggattagactggctcctgggaagcggccagagtggatgggctggctgaaaccaagaggcggagcagtgaattacgccagaccactgcagggacgagtcactatgacccgcgacgtgtattctgatactgcttttctggaactgcgaagtctgaccgtcgacgatacagcagtgtacttctgcaccaggggcaagaactgtgactataattgggattttgagcactggggaagagggactcctgtcattgtgagctccgcaagcaccaagggaccatccgtgttcccactggcaccttgcagtcggtcaactagcgagtccaccgccgctctgggatgtctggtgaaggattacttcccagaacccgtcaccgtgagctggaactccggagccctgaccagcggagtccatacatttcctgctgtgctgcagtctagtggactgtactccctgtcaagcgtggtcaccgtcccttcctctagtctggggacaaagacttatacctgcaacgtggaccacaaaccatccaatacaaaggtcgataaacgcgtggagtcaaagtacggccctccctgccctccctgtccagctcctgagttcctgggcggcccaagcgtgttcctgtttccccctaagcctaaagacacactgatgatctctaggacaccagaagtcacttgcgtggtcgtggacgtgagccaggaggaccccgaagtccagttcaactggtacgtggatggcgtcgaggtgcacaatgccaagaccaaacctcgggaggaacagtttaactctacttaccgcgtcgtgagtgtcctgaccgtgctgcatcaggactggctgaacggcaaggagtataagtgcaaagtgtccaataagggactgccatcaagcatcgagaaaaccatttctaaggcaaaaggccagccaagagaaccccaggtgtacacactgccacccagccaggaggaaatgacaaagaaccaggtctccctgacttgtctggtgaaagggttttatccaagcgacattgccgtggagtgggaatccaatggccagcccgagaacaattacaagaccacacctccagtgctggactctgatggcagtttctttctgtattcaaggctgaccgtggataaaagcagatggcaggagggaaacgtcttctcttgcagtgtgatgcacgaagctctgcacaatcattacactcagaagtcactgagcctgtccctgggaaagcgaggacgaaaaaggagatctggcagtggagctaccaacttcagcctgctgaagcaggcaggagacgtggaggaaaatcctgggccaatggtcctgcagactcaggtgttcatcagtctgctgctgtggatttcaggcgcctacggagagattgtgctgactcagtctcccggcaccctgtcactgagccctggagaaaccgccatcattagttgtcgaacatcacagtacgggagcctggcatggtatcagcagcgaccaggacaggctccccgactggtcatctactccggctctacacgggcagccggaattcccgaccgattcagtgggtcaaggtggggccctgattacaacctgactatctccaatctggagtctggcgactttggagtgtactattgccagcagtatgaattctttgggcagggcaccaaggtccaggtggacatcaaaagaacagtcgctgcaccaagcgtgttcatctttcccccttcagatgagcagctgaagagtggaaccgcctcagtggtgtgcctgctgaacaatttctacccccgggaagcaaaggtccagtggaaagtggacaacgccctgcagtcaggcaatagccaggagtccgtgacagaacaggactctaaggatagtacttattcactgtccaacacactgactctgtccaaagctgattacgagaagcacaaagtgtatgcatgcgaggtcacccaccaggggctgtcatcacccgtcaccaagtctttcaataggggcgaatgttgataactcgag

SEQ ID NO: 47 - protein, 467 aa, synthetic construct
  REGION 1..467: note = VRC01 human IgG4 (heavy chain) (mod4)
MDWTWRILFLVAAATGTHAQVQLVQSGGQMKKPGESMRISCRASGYEFIDCTLNWIRLAPGKRPEWMGWLKPRGGAVNYARPLQGRVTMTRDVYSDTAFLELRSLTVDDTAVYFCTRGKNCDYNWDFEHWGRGTPVIVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 48 - DNA, 1425 bp, synthetic construct
  misc_feature 1..1425: note = VRC01 human IgG4 (heavy chain) (mod4)
  source 1..1425: mol_type = other DNA
ggatccgccaccatggattggacttggaggattctgtttctggtcgccgccgctactggaacccatgctcaggtgcagctggtgcagtcaggagggcagatgaagaaacctggagagagcatgcgaatctcctgcagggcctctgggtacgaattcatcgactgtacactgaactggattagactggctcctgggaagcggccagagtggatgggctggctgaaaccaagaggcggagcagtgaattacgccagaccactgcagggacgagtcactatgacccgcgacgtgtattctgatactgcttttctggaactgcgaagtctgaccgtcgacgatacagcagtgtacttctgcaccaggggcaagaactgtgactataattgggattttgagcactggggaagagggactcctgtcattgtgagctccgcaagcaccaagggaccatccgtgttcccactggcaccttgcagtcggtcaactagcgagtccaccgccgctctgggatgtctggtgaaggattacttcccagaacccgtcaccgtgagctggaactccggagccctgaccagcggagtccatacatttcctgctgtgctgcagtctagtggactgtactccctgtcaagcgtggtcaccgtcccttcctctagtctggggacaaagacttatacctgcaacgtggaccacaaaccatccaatacaaaggtcgataaacgcgtggagtcaaagtacggccctccctgccctccctgtccagctcctgagttcctgggcggcccaagcgtgttcctgtttccccctaagcctaaagacacactgatgatctctaggacaccagaagtcacttgcgtggtcgtggacgtgagccaggaggaccccgaagtccagttcaactggtacgtggatggcgtcgaggtgcacaatgccaagaccaaacctcgggaggaacagtttaactctacttaccgcgtcgtgagtgtcctgaccgtgctgcatcaggactggctgaacggcaaggagtataagtgcaaagtgtccaataagggactgccatcaagcatcgagaaaaccatttctaaggcaaaaggccagccaagagaaccccaggtgtacacactgccacccagccaggaggaaatgacaaagaaccaggtctccctgacttgtctggtgaaagggttttatccaagcgacattgccgtggagtgggaatccaatggccagcccgagaacaattacaagaccacacctccagtgctggactctgatggcagtttctttctgtattcaaggctgaccgtggataaaagcagatggcaggagggaaacgtcttctcttgcagtgtgatgcacgaagctctgcacaatcattacactcagaagtcactgagcctgtccctgggaaagtgataactcgag

SEQ ID NO: 49 - protein, 230 aa, synthetic construct; identical to SEQ ID NO: 41
  REGION 1..230: note = VRC01 human IgG4 (light chain)(mod4)

SEQ ID NO: 50 - DNA, 714 bp, synthetic construct
  misc_feature 1..714: note = VRC01 human IgG4 (light chain)(mod4)
  source 1..714: mol_type = other DNA
ggatccgccaccatggtcctgcagactcaggtgttcatcagtctgctgctgtggatttcaggcgcctacggagagattgtgctgactcagtctcccggcaccctgtcactgagccctggagaaaccgccatcattagttgtcgaacatcacagtacgggagcctggcatggtatcagcagcgaccaggacaggctccccgactggtcatctactccggctctacacgggcagccggaattcccgaccgattcagtgggtcaaggtggggccctgattacaacctgactatctccaatctggagtctggcgactttggagtgtactattgccagcagtatgaattctttgggcagggcaccaaggtccaggtggacatcaaaagaacagtcgctgcaccaagcgtgttcatctttcccccttcagatgagcagctgaagagtggaaccgcctcagtggtgtgcctgctgaacaatttctacccccgggaagcaaaggtccagtggaaagtggacaacgccctgcagtcaggcaatagccaggagtccgtgacagaacaggactctaaggatagtacttattcactgtccaacacactgactctgtccaaagctgattacgagaagcacaaagtgtatgcatgcgaggtcacccaccaggggctgtcatcacccgtcaccaagtctttcaataggggcgaatgttgataactcgag

SEQ ID NO: 51 - protein, 725 aa, synthetic construct
  REGION 1..725: note = VRC01 human IgG2_New
MDWTWRILFLVAAATGTHAQVQLVQSGGQMKKPGESMRISCRASGYEFIDCTLNWIRLAPGKRPEWMGWLKPRGGAVNYARPLQGRVTMTRDVYSDTAFLELRSLTVDDTAVYFCTRGKNCDYNWDFEHWGRGTPVIVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGEIVLTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 52 - DNA, 2199 bp, synthetic construct
  misc_feature 1..2199: note = VRC01 human IgG2_New
  source 1..2199: mol_type = other DNA
ggatccgccaccatggattggacatggaggattctgtttctggtcgccgccgccactggaactcatgctcaggtgcagctggtgcagtctggagggcagatgaagaaaccaggggagtctatgcgaatcagttgcagggcttcaggctacgaattcatcgactgtaccctgaactggattagactggcacctggcaagcgaccagagtggatgggatggctgaaacccagaggcggagccgtgaattacgctagacctctgcagggacgggtcactatgacccgcgacgtgtatagcgatacagcttttctggagctgcgaagcctgaccgtcgacgatacagcagtgtacttctgcactaggggcaagaactgtgactataattgggattttgaacactgggggagaggcaccccagtcattgtgagctccgcatctacaaaaggacctagcgtgttcccactggcaccttgcagccggtccacttctgagagtaccgccgctctgggatgtctggtgaaggattacttccccgaacctgtcacagtgtcttggaacagtggggccctgactagcggcgtccacacctttcccgctgtgctgcagtctagtgggctgtacagcctgtcaagcgtggtcactgtgccatcctctaattttggcacacagacttatacctgcaacgtggaccataagccctccaatacaaaggtcgataaaactgtggagagaaaatgctgcgtggaatgccctccctgtccagcaccacccgtcgcaggaccaagcgtgttcctgtttcctccaaagccaaaagacacactgatgatcagccggacacccgaggtcacttgcgtggtcgtggacgtgagccacgaggaccccgaagtccagttcaactggtacgtggatggcgtcgaagtgcataatgctaagaccaaaccaagggaggaacagttcaactcaacctttagagtcgtgagcgtgctgacagtcgtgcatcaggactggctgaacggaaaggagtataagtgcaaagtgtccaataaggggctgccagcccccatcgagaaaaccatttctaagacaaaaggccagccacgggaaccccaggtgtacacactgcccccttcccgcgaggaaatgactaagaaccaggtctctctgacctgtctggtgaaaggcttttatccctctgacattgccgtggagtgggaaagtaatggacagcctgagaacaattacaagaccacaccacccatgctggactcagatggaagcttctttctgtattccaagctgaccgtggataaatctagatggcagcaggggaacgtcttctcatgcagcgtgatgcacgaggccctgcacaatcattacactcagaagtccctgtctctgagtcctgggaagcggggccgcaaaaggagatcaggaagcggggctaccaacttcagcctgctgaagcaggcaggggacgtggaggaaaatcctggcccaatggtcctgcagactcaggtgttcatcagcctgctgctgtggatttccggagcatacggggagattgtgctgacccagtcacctggaacactgtccctgtctccaggggaaacagccatcattagctgtcgaacttcccagtacggctctctggcatggtatcagcagcgaccaggacaggctcctcgactggtcatctacagtggatcaacccgggcagccgggattcctgaccgattcagcggctccaggtggggacctgattacaacctgacaatcagtaatctggagtcaggagactttggggtgtactattgccagcagtatgaattctttggccagggaacaaaggtccaggtggacatcaaacgcactgtcgctgcacccagcgtgttcatttttcctccatccgatgagcagctgaagagcggaaccgcatccgtggtgtgcctgctgaacaatttctaccctagggaagcaaaggtccagtggaaagtggacaacgccctgcagtccggcaattctcaggagagtgtgactgaacaggactcaaaggatagcacctattccctgagtaacacactgactctgagcaaagctgattacgagaagcacaaagtgtatgcatgcgaagtcacccaccaggggctgagctctccagtcactaagtcttttaatcggggcgaatgctgataactcgag

SEQ ID NO: 53 - protein, 776 aa, synthetic construct
  REGION 1..776: note = VRC01 human IgG3_new
MDWTWRILFLVAAATGTHAQVQLVQSGGQMKKPGESMRISCRASGYEFIDCTLNWIRLAPGKRPEWMGWLKPRGGAVNYARPLQGRVTMTRDVYSDTAFLELRSLTVDDTAVYFCTRGKNCDYNWDFEHWGRGTPVIVSSASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKRGRKRRSGSGATNFSLLKQAGDVEENPGPMVLQTQVFISLLLWISGAYGEIVLTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 54 - DNA, 2352 bp, synthetic construct
  misc_feature 1..2352: note = VRC01 human IgG3_new
  source 1..2352: mol_type = other DNA
ggatccgccaccatggattggacatggaggattctgttcctggtcgccgccgccacaggaacccatgctcaggtgcagctggtgcagtctggagggcagatgaagaaacccggcgagtcaatgcgaatcagctgcagggcttccggatacgaattcatcgattgtaccctgaactggattagactggcaccaggaaagcggcccgagtggatggggtggctgaaaccacgcggcggagcagtgaattacgccagacccctgcagggccgggtcacaatgactcgcgacgtgtattcagataccgcattcctggagctgcgaagcctgactgtcgacgataccgccgtgtacttctgcacaaggggcaaaaactgtgactacaactgggatttcgaacactggggccgcggaaccccagtcattgtgagctccgcatcaacaaaggggcctagcgtgtttcctctggccccatgcagtaggtcaacaagcgggggcactgccgctctgggatgtctggtgaaggactatttcccagagcccgtcaccgtgtcatggaacagcggcgccctgacatccggagtccatacttttcccgctgtgctgcagtctagtggactgtactctctgtcaagcgtggtcacagtcccttcctctagtctggggacccagacatatacttgtaacgtgaatcacaagccaagcaacactaaggtcgacaaaagagtggagctgaaaacccctctgggcgataccacacatacatgcccaagatgtcctgagccaaagtcctgcgacacgcctccaccatgccctcggtgtcccgaacctaaatcttgtgatacaccccctccatgcccacgatgtccagagccaaagagctgcgacactccccctccatgtcctcgatgtcctgctccagaactgctgggagggccctccgtgttcctgtttccccctaagcctaaagataccctgatgatcagtcggaccccagaggtcacatgcgtggtcgtggacgtgtcccacgaggaccccgaagtccagttcaagtggtacgtggacggcgtcgaagtgcataacgccaagacaaaacctagggaggaacagtataattccacttttagagtcgtgtctgtcctgaccgtgctgcaccaggactggctgaacggaaaggagtacaagtgtaaagtgtctaataaggccctgcccgctcctatcgagaaaactattagcaagaccaaagggcagccccgggaacctcaggtgtacaccctgccaccctcccgcgaggaaatgactaagaaccaggtctctctgacctgcctggtgaaagggttctatcccagcgatatcgctgtggagtgggaatcaagcggccagcctgagaacaattacaatactacccctccaatgctggactccgatggctctttctttctgtatagtaagctgacagtggacaaatcaagatggcagcagggaaacattttctcctgttctgtgatgcacgaagccctgcataatcggtttacccagaagagtctgtcactgagcccaggcaagcggggacgcaaaaggagatccgggtctggcgctacaaacttctccctgctgaagcaggcaggcgacgtggaggaaaatccaggacctatggtcctgcagacccaggtgttcatctctctgctgctgtggattagtggggcatacggcgaaattgtgctgacccagtccccagggacactgagtctgtcacccggcgagacagccatcatttcttgccggactagtcagtacggatcactggcatggtatcagcagcgacctggacaggctccacgactggtcatctacagcgggtccactagggcagccggcattcctgaccgattctctggaagtaggtgggggcctgattacaacctgaccatcagcaatctggagtccggggactttggcgtgtactattgtcagcagtatgaattctttggacaggggactaaggtccaggtggatatcaaacgcaccgtcgctgcacccagcgtgttcatctttccccctagtgacgaacagctgaagtctggcaccgccagtgtcgtgtgcctgctgaacaacttctaccctagggaggcaaaggtccagtggaaagtggataacgccctgcagagtggaaattcacaggagagcgtgacagaacaggactccaaggattctacttatagtctgtccaacaccctgacactgagcaaagctgactacgagaagcacaaagtgtatgcatgtgaggtcacccaccaggggctgagctcaccagtcacaaaatccttcaataggggagaatgttgataactcgag

SEQ ID NO: 55 - protein, 489 aa, synthetic construct
  REGION 1..489: note = PGT145 Heavy Chain
MDWTWRILFLVAAATGTHAQVQLVQSGAEVKKPGSSVKVSCKASGNSFSNHDVHWVRQATGQGLEWMGWMSHEGDKTGLAQKFQGRVTITRDSGASTVYMELRGLTADDTAIYYCLTGSKHRLRDYFLYNEYGPNYEEWGDYLATLDVWGHGTAVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - DNA, 1467 bp, synthetic construct
  misc_feature 1..1467: note = PGT145 Heavy Chain
  source 1..1467: mol_type = other DNA
atggattggacatggaggattctgtttctggtcgccgccgccactggaactcacgctcaggtgcagctggtgcagtcaggggccgaagtgaagaagcccggcagctccgtgaaggtgtcctgcaaggcctctggcaactccttctctaatcacgacgtgcactgggtgaggcaggcaaccggacagggcctggagtggatgggctggatgtcccacgagggcgacaagacaggcctggcacagaagtttcagggccgggtgaccatcacacgcgattccggcgcctctaccgtgtatatggagctgagaggcctgaccgccgacgatacagccatctactattgcctgacaggcagcaagcacaggctgagagactacttcctgtataacgagtacggccctaattatgaggagtggggcgactacctggccaccctggacgtgtggggacacggaaccgcagtgacagtgtctagcgccagcacaaagggcccatccgtgtttccactggcaccctcctctaagagcacctccggcggcacagccgccctgggctgtctggtgaaggattacttcccagagccagtgaccgtgagctggaactccggcgccctgacctccggagtgcacacatttccagccgtgctgcagagctccggcctgtatagcctgtctagcgtggtgaccgtgccctcctctagcctgggcacccagacatacatctgcaacgtgaatcacaagccatccaatacaaaggtggacaagaaggtggagcccaagtcttgtgataagacccacacatgccctccctgtcctgcaccagagctgctgggcggcccatccgtgttcctgtttccacccaagcctaaggacaccctgatgatcagccggaccccagaggtgacatgcgtggtggtggacgtgtcccacgaggaccccgaggtgaagttcaactggtacgtggatggcgtggaggtgcacaatgccaagaccaagcctagggaggagcagtataactctacctacagagtggtgagcgtgctgacagtgctgcaccaggactggctgaacggcaaggagtacaagtgcaaggtgtctaataaggccctgcccgcccctatcgagaagaccatcagcaaggcaaagggacagcctcgggagccacaggtgtatacactgcctccatctcgcgacgagctgaccaagaaccaggtgagcctgacatgtctggtgaagggcttctaccctagcgatatcgccgtggagtgggagtccaatggccagccagagaacaattataagaccacaccccctgtgctggactctgatggcagcttctttctgtactccaagctgaccgtggataagtctcgctggcagcagggcaacgtgttcagctgtagcgtgatgcacgaagccctgcacaaccactacacccagaaaagcctgtcactgagccccggaaaa

SEQ ID NO: 57 - protein, 239 aa, synthetic construct
  REGION 1..239: note = PGT145 Kappa Light Chain
MVLQTQVFISLLLWISGAYGEVVITQSPLFLPVTPGEAASLSCKCSHSLQHSTGANYLAWYLQRPGQTPRLLIHLATHRASGVPDRFSGSGSGTDFTLKISRVESDDVGTYYCMQGLHSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - DNA, 717 bp, synthetic construct
  misc_feature 1..717: note = PGT145 Kappa Light Chain
  source 1..717: mol_type = other DNA
atggtgctgcagacccaggtgttcatttcactgctgctgtggatttcaggagcctacggagaagtcgtcattacccagtcccctctgttcctgccagtgaccccaggagaggcagcatctctgagctgcaagtgttcccactctctgcagcacagcacaggcgccaactacctggcctggtatctgcagaggcctggccagaccccaagactgctgatccacctggcaacacacagggcatccggagtgccagaccgcttcagcggctccggctctggaaccgacttcaccctgaagatcagcagggtggagtccgacgatgtgggcacctactattgcatgcagggcctgcactctccctggaccttcggccagggcacaaaggtggagatcaagaggaccgtggcagcaccttccgtgttcatctttcccccttctgacgagcagctgaagtctggcacagccagcgtggtgtgcctgctgaacaacttctaccccagggaggccaaggtgcagtggaaggtggataacgccctgcagagcggcaattcccaggagtctgtgaccgagcaggacagcaaggattccacatattctctgagctccaccctgacactgagcaaggccgactacgagaagcacaaggtgtatgcctgcgaggtcacccatcaggggctgtcaagtccagtcacaaagtccttcaataggggcgaatgc

SEQ ID NO: 59 - protein, 484 aa, synthetic construct
  REGION 1..484: note = PGT151 Heavy Chain
MDWTWRILFLVAAATGTHARVQLVESGGGVVQPGKSVRLSCVVSDFPFSKYPMYWVRQAPGKGLEWVAAISGDAWHVVYSNSVQGRFLVSRDNVKNTLYLEMNSLKIEDTAVYRCARMFQESGPPRLDRWSGRNYYYYSGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - DNA, 1452 bp, synthetic construct
  misc_feature 1..1452: note = PGT151 Heavy Chain
  source 1..1452: mol_type = other DNA
atggactggacttggcgcattctgtttctggtcgcagccgcaaccgggactcacgctcgggtgcagctggtcgaatcaggaggaggagtggtgcagccaggcaagagcgtgcggctgtcctgcgtggtgtctgacttccccttcagcaagtacccaatgtattgggtgcgccaggcaccaggcaagggcctggagtgggtggcagcaatctccggcgatgcatggcacgtggtgtactctaacagcgtgcagggcaggttcctggtgagcagagacaacgtgaagaataccctgtacctggagatgaattccctgaagatcgaggatacagccgtgtataggtgcgccagaatgtttcaggagagcggccctcccaggctggacaggtggagcggaaggaactactattactattccggaatggacgtgtggggacagggaaccacagtgaccgtgagctccgccagcacaaagggcccttccgtgttccctctggccccatctagcaagtccacctctggcggcacagccgccctgggctgtctggtgaaggactacttccctgagccagtgaccgtgtcttggaacagcggcgccctgacctctggagtgcacacatttcccgccgtgctgcagtcctctggcctgtactccctgagctccgtggtgaccgtgccttctagctccctgggcacccagacatatatctgcaacgtgaatcacaagcccagcaatacaaaggtggacaagaaggtggagcctaagtcctgtgataagacccacacatgcccaccctgtccagcacctgagctgctgggcggccctagcgtgttcctgtttcctccaaagccaaaggacaccctgatgatctctagaacccctgaggtgacatgcgtggtggtggacgtgagccacgaggaccccgaggtgaagtttaactggtacgtggatggcgtggaggtgcacaatgccaagacaaagccccgggaggagcagtacaactctacctatcgcgtggtgagcgtgctgacagtgctgcaccaggactggctgaacggcaaggagtataagtgcaaggtgtccaataaggccctgccagcccccatcgagaagaccatctctaaggcaaagggacagcccagggagcctcaggtgtacacactgcccccttccagagacgagctgaccaagaaccaggtgtctctgacatgtctggtgaagggcttctatccatctgatatcgccgtggagtgggagagcaatggccagcccgagaacaattacaagaccacaccacccgtgctggactccgatggctctttctttctgtattccaagctgaccgtggataagtctaggtggcagcagggcaacgtgttcagctgttccgtgatgcacgaagcactgcacaaccattacacccagaagagcctgagcctgagccccggcaag

SEQ ID NO: 61 - protein, 239 aa, synthetic construct
  REGION 1..239: note = PGT151 Kappa Light Chain
MVLQTQVFISLLLWISGAYGDIVMTQTPLSLSVTPGQPASISCKSSESLRQSNGKTSLYWYRQKPGQSPQLLVFEVSNRFSGVSDRFVGSGSGTDFTLRISRVEAEDVGFYYCMQSKDFPLTFGGGTKVDLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - DNA, 717 bp, synthetic construct
  misc_feature 1..717: note = PGT151 Kappa Light Chain
  source 1..717: mol_type = other DNA
atggtgctgcagactcaggtgtttattagcctgctgctgtggatttccggggcctacggggacattgtgatgacccagactcctctgtccctgtctgtgaccccaggacagccagcaagcatctcctgcaagagctccgagagcctgaggcagtccaacggcaagacaagcctgtactggtatagacagaagcctggccagagcccacagctgctggtgttcgaggtgtccaatcggttttccggcgtgtctgacaggttcgtgggctctggcagcggaaccgacttcaccctgaggatctctagagtggaggccgaggacgtgggcttctactattgcatgcagtccaaggatttccccctgaccttcggcggcggcacaaaggtggacctgaagaggaccgtggcagcaccttccgtgttcatctttcccccttctgatgagcagctgaagtctggcacagccagcgtggtgtgcctgctgaacaacttctacccccgcgaggccaaggtgcagtggaaggtggacaacgccctgcagagcggcaattcccaggagtctgtgaccgagcaggacagcaaggattccacatattctctgtctagcaccctgacactgtccaaggccgattacgagaagcacaaggtgtatgcctgtgaggtcacccaccaggggctgtccagtccagtcacaaaatccttcaatcggggagaatgt

SEQ ID NO: 63 - protein, 490 aa, synthetic construct
  REGION 1..490: note = PGDM1400 Heavy Chain
MDWTWRILFLVAAATGTHAQAQLVQSGPEVRKPGTSVKVSCKAPGNTLKTYDLHWVRSVPGQGLQWMGWISHEGDKKVIVERFKAKVTIDWDRSTNTAYLQLSGLTSGDTAVYYCAKGSKHRLRDYALYDDDGALNWAVDVDYLSNLEFWGQGTAVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - DNA, 1470 bp, synthetic construct
  misc_feature 1..1470: note = PGDM1400 Heavy Chain
  source 1..1470: mol_type = other DNA
atggattggacatggaggattctgtttctggtcgccgccgcaaccggaactcacgctcaggctcagctggtgcagtctggacctgaagtgaggaagccaggcacctccgtgaaggtgtcttgcaaggcccccggcaacaccctgaagacatacgatctgcactgggtgagatctgtgccaggacagggcctgcagtggatgggatggatcagccacgagggcgacaagaaagtgatcgtggagaggttcaaggccaaggtgaccatcgattgggacagatccaccaatacagcctacctgcagctgagcggcctgacctccggcgatacagccgtgtactattgcgccaagggcagcaagcacaggctgagagactacgccctgtatgacgatgacggcgccctgaactgggccgtggatgtggactatctgtccaatctggagttctggggacagggaaccgcagtgacagtgagctccgcctctaccaagggccctagcgtgtttccactggccccctctagcaagtctaccagcggcggcacagccgccctgggatgtctggtgaaggactacttcccagagcccgtgacagtgagctggaactccggcgccctgacctctggagtgcacacatttcctgccgtgctgcagtcctctggcctgtacagcctgagctccgtggtgaccgtgccatctagctccctgggcacccagacatatatctgcaacgtgaatcacaagcctagcaatacaaaggtggataagaaggtggagccaaagtcctgtgacaagacccacacatgccctccctgtcctgcaccagagctgctgggcggcccaagcgtgttcctgtttccacccaagcctaaggataccctgatgatctcccggaccccagaggtgacatgcgtggtggtggacgtgagccacgaggaccccgaggtgaagtttaactggtacgtggacggcgtggaggtgcacaatgccaagacaaagccccgggaggagcagtacaactccacctatcgcgtggtgtctgtgctgacagtgctgcaccaggattggctgaacggcaaggagtataagtgcaaggtgagcaataaggccctgcccgcccctatcgagaagaccatctccaaggcaaagggacagcctcgggagccacaggtgtacacactgcctccatcccgcgatgagctgaccaagaaccaggtgtctctgacatgtctggtgaagggcttctatccctctgacatcgccgtggagtgggagagcaatggccagcctgagaacaattacaagaccacaccccctgtgctggattctgacggcagcttctttctgtatagcaagctgaccgtggacaagtcccgctggcagcagggcaacgtgttcagctgttctgtgatgcacgaagcactgcacaaccactacacccagaagtcactgtcactgtcaccaggaaaa

SEQ ID NO: 65 - protein, 239 aa, synthetic construct
  REGION 1..239: note = PGDM1400 Kappa Light Chain
MVLQTQVFISLLLWISGAYGDFVLTQSPHSLSVTPGESASISCKSSHSLIHGDRNNYLAWYVQKPGRSPQLLIYLASSRASGVPDRFSGSGSDKDFTLKISRVETEDVGTYYCMQGRESPWTFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - DNA, 717 bp, synthetic construct
  misc_feature 1..717: note = PGDM1400 Kappa Light Chain
  source 1..717: mol_type = other DNA
atggtgctgcagacccaggtgttcatttcactgctgctgtggatttccggggcatacggggacttcgtgctgactcagagccctcatagcctgtccgtgaccccaggagagtctgccagcatctcctgcaagagctcccacagcctgatccacggcgaccggaacaattacctggcctggtacgtgcagaagccaggccgcagcccccagctgctgatctacctggcctctagcagggcctccggagtgcctgacagattctctggcagcggctccgacaaggacttcaccctgaagatctctagggtggagacagaggatgtgggcacatactattgcatgcagggcagagagtccccttggaccttcggccagggcacaaaggtggacatcaagaggaccgtggcagcaccatccgtgttcatctttcccccttctgatgagcagctgaagtctggcacagccagcgtggtgtgcctgctgaacaacttctacccccgcgaggccaaggtgcagtggaaggtggacaacgccctgcagtctggcaatagccaggagtccgtgaccgagcaggactctaaggatagcacatattccctgtcctctaccctgacactgtccaaggccgattacgagaagcacaaggtgtatgcctgtgaggtcacccaccaggggctgtccagtcccgtcacaaagtccttcaataggggcgaatgc

SEQ ID NO: 67 - protein, 471 aa, synthetic construct
  REGION 1..471: note = 12A21 Heavy Chain
MDWTWRILFLVAAATGTHASQHLVQSGTQVKKPGASVRVSCQASGYTFTNYILHWWRQAPGQGLEWMGLIKPVFGAVNYARQFQGRIQLTRDIYREIAFLDLSGLRSDDTAVYYCARDESGDDLKWHLHPWGQGTQVIVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - DNA, 1413 bp, synthetic construct
  misc_feature 1..1413: note = 12A21 Heavy Chain
  source 1..1413: mol_type = other DNA
atggattggacatggcgcattctgtttctggtcgcagccgctactggaacccacgcatcacagcatctggtgcagtctggaactcaggtgaagaagccaggagcatccgtgcgggtgtcttgccaggcaagcggctacaccttcacaaactatatcctgcactggtggaggcaggcaccaggacagggcctggagtggatgggcctgatcaagcccgtgttcggcgccgtgaattacgcccggcagtttcagggccgcatccagctgaccagggacatctatagagagatcgcctttctggatctgtccggcctgcggtctgacgatacagccgtgtactattgcgcccgcgacgagagcggcgacgatctgaagtggcacctgcacccttggggacagggaacccaggtcatcgtgagcccagcatctacaaagggaccttccgtgttcccactggcacccagctccaagagcacctccggcggcacagccgccctgggctgtctggtgaaggattacttcccagagccagtgaccgtgagctggaactccggcgccctgacctctggagtgcacacatttcccgccgtgctgcagtctagcggcctgtactccctgtcctctgtggtgaccgtgcctagctcctctctgggcacccagacatatatctgcaacgtgaatcacaagccttctaatacaaaggtggacaagaaggtggagccaaagagctgtgataagacccacacatgccctccctgtcctgcaccagagctgctgggcggcccaagcgtgttcctgtttccacccaagcccaaggacaccctgatgatctccaggacccctgaggtgacatgcgtggtggtggacgtgtctcacgaggaccccgaggtgaagtttaactggtacgtggatggcgtggaggtgcacaatgccaagacaaagcccagggaggagcagtacaacagcacctatagagtggtgtccgtgctgacagtgctgcaccaggactggctgaacggcaaggagtataagtgcaaggtgagcaataaggccctgcccgcccctatcgagaagaccatctccaaggccaagggccagcctagggagccacaggtgtacacactgcctccaagcagagacgagctgaccaagaaccaggtgtccctgacatgtctggtgaagggcttttatccatccgatatcgccgtggagtgggagtctaatggccagcccgagaacaattacaagaccacaccccctgtgctggactctgatggcagcttctttctgtattctaagctgaccgtggataagagcagatggcagcagggcaacgtgttcagctgtagcgtgatgcacgaagcactgcacaaccactacacccagaagtcactgtcactgagcccaggaaaa

SEQ ID NO: 69 - protein, 230 aa, synthetic construct
  REGION 1..230: note = 12A21 Kappa Light Chain
MVLQTQVFISLLLWISGAYGDIQMTQSPSSLSASVGDRVTINCQAGQGIGSSLNWYQKKPGRAPKLLVHGASNLQRGVPSRFSGSGFHTTFTLTISSLQPDDVATYFCAVFQWFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - DNA, 690 bp, synthetic construct
  misc_feature 1..690: note = 12A21 Kappa Light Chain
  source 1..690: mol_type = other DNA
atggtgctgcagactcaggtgtttatttcactgctgctgtggatttcaggagcctacggcgacattcagatgactcagagcccctcaagcctgtccgcctctgtgggcgaccgggtgaccatcaactgccaggcaggacagggaatcggcagctccctgaattggtaccagaagaagccaggaagggcaccaaagctgctggtgcacggagcatccaacctgcagaggggcgtgcccagcagattcagcggctccggcttccacaccaccttcaccctgacaatctctagcctgcagcctgacgatgtggccacctatttttgcgccgtgttccagtggtttggccctggcaccaaggtggacatcaagaggacagtggccgccccatccgtgttcatctttcccccttctgatgagcagctgaagtctggcacagccagcgtggtgtgcctgctgaacaatttctacccccgggaggccaaggtgcagtggaaggtggacaacgccctgcagtctggcaatagccaggagtccgtgaccgagcaggactctaaggatagcacatattccctgtcctctaccctgacactgtccaaggccgattacgagaagcacaaggtgtatgcctgtgaggtcacccaccaggggctgtcaagtccagtcacaaaatccttcaatcggggagaatgc

SEQ ID NO: 71 - protein, 471 aa, synthetic construct
  REGION 1..471: note = VRC3401 Heavy Chain
MDWTWRILFLVAAATGTHAQEVLVQSGAEVKKPGASVKVSCRAFGYTFTGNALHWVRQAPGQGLEWLGWINPHSGDTTTSQKFQGRVYMTRDKSINTAFLDVTRLTSDDTGIYYCARDKYYGNEAVGMDVWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - DNA, 1413 bp, synthetic construct
  misc_feature 1..1413: note = VRC3401 Heavy Chain
  source 1..1413: mol_type = other DNA
atggattggacatggcgcattctgtttctggtcgcagccgctaccggaacccacgctcaggaagtgctggtgcagtcaggggccgaagtgaagaagcccggcgcctccgtgaaggtgtcttgcagggccttcggctacaccttcaccggcaacgcactgcactgggtgagacaggcacctggacagggcctggagtggctgggatggatcaatccacactctggcgacaccacaaccagccagaagttccagggcagggtgtatatgacaagagacaagtctatcaacaccgcctttctggatgtgacacggctgaccagcgacgatacaggcatctactattgcgcccgcgacaagtactatggcaatgaggcagtgggaatggacgtgtggggacagggcacatccgtgaccgtgagctccgccagcaccaagggaccttccgtgttcccactggcaccctctagcaagtctacaagcggcggcaccgccgccctgggatgtctggtgaaggattacttcccagagccagtgaccgtgagctggaactccggcgccctgacatctggcgtgcacacctttcctgccgtgctgcagtcctctggcctgtacagcctgagctccgtggtgacagtgccatctagctccctgggcacacagacctatatctgcaacgtgaatcacaagccaagcaataccaaggtggacaagaaggtggagcccaagtcctgtgataagacacacacctgccctccctgtcctgcaccagagctgctgggcggcccaagcgtgttcctgtttccacccaagcctaaggacacactgatgatctcccggacaccagaggtgacctgcgtggtggtggacgtgtctcacgaggaccccgaggtgaagtttaactggtacgtggatggcgtggaggtgcacaatgccaagaccaagccccgggaggagcagtacaactccacatatcgcgtggtgtctgtgctgaccgtgctgcaccaggactggctgaacggcaaggagtataagtgcaaggtgagcaataaggccctgcccgcccctatcgagaagacaatctccaaggccaagggccagcctagggagccacaggtgtacaccctgcctccatccagagacgagctgacaaagaaccaggtgtctctgacctgtctggtgaagggcttctatccctctgatatcgccgtggagtgggagagcaatggccagcctgagaacaattacaagacaacccctcccgtgctggactctgatggcagcttctttctgtatagcaagctgaccgtggataagtcccgctggcagcagggcaacgtgttcagctgttctgtgatgcacgaagcactgcacaaccactacacccagaagtcactgtcactgagcccaggaaag

SEQ ID NO: 73 - protein, 232 aa, synthetic construct
  REGION 1..232: note = VRC3401 Kappa Light Chain
MVLQTQVFISLLLWISGAYGQLTQSPSFLSASVGDKVTITCRASQGVRNELAWYQQKPGKAPNLLIYYASTLQSGVPSRFSATGSGTHFTLTVSSLQPEDFATYFCQHMSSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - DNA, 696 bp, synthetic construct
  misc_feature 1..696: note = VRC3401 Kappa Light Chain
  source 1..696: mol_type = other DNA
atggtgctgcagacccaggtgtttatttcactgctgctgtggattagcggagcctacggccagctgacccagagcccaagtttcctgagcgcctccgtgggcgacaaggtgaccatcacatgcagggcctcccagggcgtgagaaacgagctggcctggtaccagcagaagccaggcaaggcccccaatctgctgatctactatgcctccaccctgcagtctggagtgccctctaggttcagcgccaccggctctggcacacactttaccctgacagtgagctccctgcagcctgaggatttcgccacatacttttgccagcacatgtctagctatccactgaccttcggcggcggcacaaaggtggagatcaagaggaccgtggcagcaccaagcgtgttcatctttccccctagcgacgagcagctgaagagcggcacagcctccgtggtgtgcctgctgaacaacttctaccctcgcgaggccaaggtgcagtggaaggtggataacgccctgcagtccggcaattctcaggagagcgtgaccgagcaggactccaaggattctacatatagcctgtcctctaccctgacactgagcaaggccgactacgagaagcacaaggtgtatgcctgtgaggtcacccaccaggggctgagcagtccagtcaccaagtccttcaatcggggcgaatgt

SEQ ID NO: 75 - protein, 475 aa, synthetic construct
  REGION 1..475: note = IOMA Heavy Chain
MDWTWRILFLVAAATGTHAEVQLVESGAQVKKPGASVTVSCTASGYKFTGYHMHWVRQAPGRGLEWMGWINPFRGAVKYPQNFRGRVSMTRDTSMEIFYMELSRLTSDDTAVYYCAREMFDSSADWSPWRGMVAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - DNA, 1425 bp, synthetic construct
  misc_feature 1..1425: note = IOMA Heavy Chain
  source 1..1425: mol_type = other DNA
atggactggacatggagaatcctgttcctggtcgcagcagcaaccggcacccacgcagaagtgcagctggtcgaaagcggggctcaggtgaagaagccaggagcaagcgtgaccgtgtcctgcacagcatctggctacaagttcaccggctatcacatgcactgggtgcggcaggcacctggaaggggcctggagtggatgggatggatcaaccccttcaggggcgccgtgaagtaccctcagaacttcaggggcagagtgtccatgaccagggacacatctatggagatcttctatatggagctgagcagactgacctccgacgatacagccgtgtactattgcgcccgggagatgtttgacagctccgccgattggagcccctggaggggaatggtggcatggggacagggcaccctggtgacagtgtctagcgcctctaccaagggccctagcgtgtttccactggccccctcctctaagtccacctctggcggcacagccgccctgggctgtctggtgaaggattacttcccagagcccgtgacagtgtcttggaacagcggcgccctgaccagcggagtgcacacatttcctgccgtgctgcagagctccggcctgtactccctgtctagcgtggtgaccgtgccatcctctagcctgggcacccagacatatatctgcaacgtgaatcacaagccttctaatacaaaggtggacaagaaggtggagccaaagagctgtgataagacccacacatgccctccctgtcctgcaccagagctgctgggcggcccatccgtgttcctgtttccacccaagcccaaggacaccctgatgatctccaggaccccagaggtgacatgcgtggtggtggacgtgtctcacgaggaccccgaggtgaagttcaactggtacgtggatggcgtggaggtgcacaatgccaagacaaagcccagggaggagcagtacaacagcacctatagagtggtgtccgtgctgacagtgctgcaccaggactggctgaacggcaaggagtataagtgcaaggtgagcaataaggccctgcccgcccctatcgagaagaccatctccaaggcaaagggacagcctcgggagccacaggtgtacacactgcctccaagccgcgacgagctgaccaagaaccaggtgtccctgacatgtctggtgaagggcttctatccctccgatatcgccgtggagtgggagtctaatggccagcctgagaacaattacaagaccacaccccctgtgctggactccgatggctctttctttctgtattctaagctgaccgtggataagagcagatggcagcagggcaacgtgttcagctgttccgtgatgcacgaagcactgcacaaccattacactcagaagtcactgtcactgtcaccagggaag

SEQ ID NO: 77 - protein, 234 aa, synthetic construct
  REGION 1..234: note = IOMA Lambda Light Chain
MAWTPLFLFLLTCCPGGSNSQSALTQPASVSGSPGQSITISCAGSSRDVGGFDLVSWYQQHPGKAPKLIIYEVNKRPSGISSRFSASKSGNTASLTISGLQEEDEAHYYCYSYADGVAFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 78 - DNA, 702 bp, synthetic construct
  misc_feature 1..702: note = IOMA Lambda Light Chain
  source 1..702: mol_type = other DNA
atggcttggacccctctgtttctgtttctgctgacctgttgccccggaggaagtaattcccagtctgccctgacccagcctgcatcagtgtccggctctcctggccagtccatcacaatctcttgcgcaggcagctccagggacgtgggcggctttgatctggtgtcctggtaccagcagcacccaggcaaggcccccaagctgatcatctatgaggtgaacaagaggccctctggcatctctagcagattcagcgcctccaagtctggcaatacagccagcctgaccatctccggcctgcaggaggaggacgaggcacactactattgctactcctatgcagatggagtggccttcggcggcggcacaaagctgaccgtgctgggacagcctaaggcagcaccatccgtgacactgtttcccccttcctctgaggagctgcaggccaacaaggccaccctggtgtgcctgatcagcgacttctacccaggagcagtgaccgtggcatggaaggcagatagctccccagtgaaggcaggagtggagacaacaaccccttctaagcagagcaacaataagtacgccgcctctagctatctgtctctgacccccgagcagtggaagagccacaagagctattcctgccaggtcacccatgaaggctcaaccgtggagaaaacagtcgcccctaccgaatgctca

SEQ ID NO: 79 - protein, 478 aa, synthetic construct
  REGION 1..478: note = PGT130 Heavy Chain
MDWTWRILFLVAAATGTHAQVQLQESGPGLVKPAETLSLTCSVSGESINTGHYYWGWVRQVPGKGLEWIGHIHYTTAVLHNPSLKSRLTIKIYTLRNQITLRLSNVTAADTAVYHCVRSGGDILYYYEWQKPHWFSPWGPGIHVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - DNA, 1434 bp, synthetic construct
  misc_feature 1..1434: note = PGT130 Heavy Chain
  source 1..1434: mol_type = other DNA
atggactggacttggaggattctgtttctggtcgccgccgctactggaactcacgctcaggtgcagctgcaggaatctggccccggactggtgaagcctgccgagacactgtccctgacatgctctgtgagcggcgagtctatcaacaccggccactactattggggatgggtgcggcaggtgccaggcaagggcctggagtggatcggccacatccactataccacagccgtgctgcacaatccttctctgaagagccgcctgaccatcaagatctacacactgaggaaccagatcaccctgagactgtctaatgtgaccgccgccgacacagccgtgtatcactgcgtgagaagcggcggcgatatcctgtactattacgagtggcagaagccacactggttctccccatggggaccaggcatccacgtgaccgtgagctccgcctctacaaagggccccagcgtgtttcctctggccccatctagcaagtccacctctggcggcacagccgccctgggctgtctggtgaaggactacttcccagagcccgtgaccgtgtcctggaactctggcgccctgacctccggagtgcacacatttcctgccgtgctgcagtcctctggcctgtatagcctgagctccgtggtgaccgtgccatctagctccctgggcacccagacatacatctgcaacgtgaatcacaagccttctaatacaaaggtggacaagaaggtggagccaaagagctgtgataagacccacacatgccctccctgtcctgcaccagagctgctgggcggcccaagcgtgttcctgtttccacccaagcccaaggacaccctgatgatctccaggaccccagaggtgacatgcgtggtggtggacgtgtctcacgaggaccccgaggtgaagttcaactggtacgtggatggcgtggaggtgcacaatgccaagacaaagccccgggaggagcagtataactccacctaccgcgtggtgtctgtgctgacagtgctgcaccaggactggctgaacggcaaggagtacaagtgcaaggtgagcaataaggccctgcccgcccctatcgagaagaccatctccaaggcaaagggacagcccagggagcctcaggtgtatacactgcctccaagcagagacgagctgaccaagaaccaggtgtccctgacatgtctggtgaagggcttctacccctctgatatcgccgtggagtgggagagcaatggccagcctgagaacaattataagaccacaccccctgtgctggacagcgatggctccttctttctgtacagcaagctgaccgtggataagtccaggtggcagcagggcaacgtgttcagctgttccgtgatgcacgaagcactgcacaaccattacacccagaagtccctgtcactgtcacccggcaaa

SEQ ID NO: 81 - protein, 236 aa, synthetic construct
  REGION 1..236: note = PGT130 Lambda Light Chain
MAWTPLFLFLLTCCPGGSNSQSALTQPPSASGSLGQSVTISCNGTSSDIGGWNFVSWYQQFPGRAPRLIIFEVNKRPSGVPGRFSGSKSGNSASLTVSGLQSDDEGQYFCSSLFGRWDVVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 82 - DNA, 708 bp, synthetic construct
  misc_feature 1..708: note = PGT130 Lambda Light Chain
  source 1..708: mol_type = other DNA
atggcatggacacccctgtttctgttcctgctgacctgttgccccggaggctcaaatagccagtctgccctgacccagcccccaagcgcctccggctctctgggccagagcgtgacaatctcctgcaacggcaccagctccgacatcggcggatggaatttcgtgagctggtaccagcagtttccaggcagggcccccagactgatcatcttcgaggtgaacaagcggccttccggagtgccaggccgctttagcggctccaagtctggcaatagcgcctccctgacagtgtctggcctgcagagcgacgatgagggccagtatttctgctctagcctgtttggcaggtgggatgtggtgttcggcggcggaacaaagctgaccgtgctgggacagccaaaggcagcaccttctgtgaccctgtttcccccttcctctgaggagctgcaggccaacaaggccaccctggtgtgcctgatcagcgacttctaccctggagcagtgaccgtggcatggaaggccgatagctccccagtgaaggccggcgtggagacaacaaccccctccaagcagtctaacaataagtacgccgcctctagctatctgtccctgacacccgagcagtggaagtctcacaagtcttatagctgccaggtcacccatgaaggaagcacagtggaaaaaaccgtcgcaccaaccgaatgtagc

SEQ ID NO: 83 - protein, 481 aa, synthetic construct
  REGION 1..481: note = PGT121 Heavy Chain
MDWTWRILFLVAAATGTHAQMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - DNA, 1443 bp, synthetic construct
  misc_feature 1..1443: note = PGT121 Heavy Chain
  source 1..1443: mol_type = other DNA
atggattggacatggaggattctgtttctggtcgccgccgctactggaacccacgctcagatgcagctgcaggagtcaggccccggactggtgaagccctccgagacactgtctctgacatgctctgtgagcggcgcctccatctctgacagctactggtcttggatcaggagaagccctggcaagggcctggagtggatcggctacgtgcacaagtctggcgacaccaactattccccatctctgaagagcagggtgaacctgagcctggatacatccaagaatcaggtgagcctgtccctggtggcagcaaccgcagcagacagcggcaagtactattgcgccagaacactgcacggcaggaggatctacggcatcgtggccttcaacgagtggttcacctacttttatatggacgtgtggggcaatggcacccaggtgacagtgagctccgcctccacaaagggacctagcgtgttcccactggcaccctctagcaagtctaccagcggcggcacagccgccctgggatgtctggtgaaggattatttcccagagccagtgaccgtgagctggaactccggcgccctgaccagcggagtgcacacatttccagccgtgctgcagtcctctggcctgtactccctgagctccgtggtgaccgtgccctctagctccctgggcacccagacatatatctgcaacgtgaatcacaagccatccaatacaaaggtggacaagaaggtggagcccaagtcttgtgataagacccacacatgccctccctgtcctgcaccagagctgctgggcggcccaagcgtgttcctgtttccacccaagcctaaggacaccctgatgatcagccggaccccagaggtgacatgcgtggtggtggacgtgtcccacgaggaccccgaggtgaagttcaactggtacgtggatggcgtggaggtgcacaatgccaagacaaagcccagggaggagcagtacaacagcacctatagagtggtgtccgtgctgacagtgctgcaccaggactggctgaacggcaaggagtataagtgcaaggtgtctaataaggccctgcccgcccctatcgagaagaccatctccaaggcaaagggacagcctcgggagccacaggtgtacacactgcctccaagccgcgacgagctgaccaagaaccaggtgtccctgacatgtctggtgaagggcttctatccttccgatatcgccgtggagtgggagtctaatggccagccagagaacaattacaagaccacaccccctgtgctggactctgatggcagcttctttctgtattctaagctgaccgtggataagagccgctggcagcagggcaacgtgttcagctgttctgtgatgcacgaagcactgcacaaccactacactcagaagtccctgtccctgtcccctggcaag

SEQ ID NO: 85 - protein, 230 aa, synthetic construct
  REGION 1..230: note = PGT121 Lambda Light Chain
MAWTPLFLFLLTCCPGGSNSDISVAPGETARISCGEKSLGSRAVQWYQHRAGQAPSLIIYNNQDRPSGIPERFSGSPDSPFGTTATLTITSVEAGDEADYYCHIWDSRVPTKWVFGGGTTLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 86 - DNA, 690 bp, synthetic construct
  misc_feature 1..690: note = PGT121 Lambda Light Chain
  source 1..690: mol_type = other DNA
atggcttggactcccctgttcctgttcctgctgacttgttgccctggcggctctaactctgacatttccgtcgctcctggcgagactgccaggatcagctgcggagagaagtccctgggctctcgggccgtgcagtggtaccagcacagggcaggacaggcaccatccctgatcatctataacaatcaggacaggccttctggcatcccagagagattcagcggctccccagatagcccctttggcaccacagccacactgaccatcacatccgtggaggcaggcgacgaggcagattactattgccacatctgggactcccgggtgcctaccaagtgggtgttcggcggcggaaccacactgacagtgctgggacagccaaaggcagcaccttctgtgaccctgtttccccctagctccgaggagctgcaggccaacaaggccaccctggtgtgcctgatctccgacttctaccctggagcagtgacagtggcatggaaggccgattctagcccagtgaaggccggcgtggagacaacaacccccagcaagcagtccaacaataagtacgccgcctcctcttatctgtctctgacaccagagcagtggaagagccacaagtcttatagctgccaggtcacccatgaaggaagcaccgtggagaaaacagtcgccccaacagaatgtagc

SEQ ID NO: 87 - protein, 473 aa, synthetic construct
  REGION 1..473: note = 2219 Heavy Chain
MDWTWRILFLVAAATGTHAEIQLEQSGAEVKKSGESLKISCQTSGYSFSDYWIGWVRQMPGKGLEWMGIFYPGDSDSRYSPSFEGQVTMSADRSTNTAHLQWSSLKPSDTALYYCARLGGDYEDSGADAFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - DNA, 1419 bp, synthetic construct
  misc_feature 1..1419: note = 2219 Heavy Chain
  source 1..1419: mol_type = other DNA
atggattggacatggaggattctgtttctggtcgccgccgccactggaactcacgctgagattcagctggagcagagtggagcagaagtcaagaagagcggggagagcctgaagattagttgccagacaagcggatacagtttctctgactactggatcggatgggtgcggcagatgccaggcaagggcctggagtggatgggcatcttctaccccggcgactctgatagcaggtattccccttcttttgagggccaggtgaccatgagcgccgatagatccaccaacacagcccacctgcagtggagctccctgaagccctccgacacagccctgtactattgcgcaaggctgggcggcgattatgaggacagcggagccgacgcctttgatttttgggggcaggggactctggtgaccgtgagcagcgccagcacaaagggcccatccgtgtttccactggcaccctcctctaagagcacctccggcggcacagccgccctgggctgtctggtgaaggattacttcccagagccagtgaccgtgagctggaactccggcgccctgacctccggagtgcacacatttccagccgtgctgcagagctccggcctgtatagcctgtctagcgtggtgaccgtgccctcctctagcctgggcacccagacatacatctgcaacgtgaatcacaagccatccaatacaaaggtggacaagaaggtggagcccaagtcttgtgataagacccacacatgccctccctgtcctgcaccagagctgctgggcggcccatccgtgttcctgtttccacccaagcctaaggacaccctgatgatcagccggaccccagaggtgacatgcgtggtggtggacgtgtcccacgaggaccccgaggtgaagttcaactggtacgtggatggcgtggaggtgcacaatgccaagaccaagcctagggaggagcagtataactctacctacagagtggtgagcgtgctgacagtgctgcaccaggactggctgaacggcaaggagtacaagtgcaaggtgtctaataaggccctgcccgcccctatcgagaagaccatcagcaaggcaaagggacagcctcgggagccacaggtgtatacactgcctccatctcgcgacgagctgaccaagaaccaggtgagcctgacatgtctggtgaagggcttctaccctagcgatatcgccgtggagtgggagtccaatggccagccagagaacaattataagaccacaccccctgtgctggactctgatggcagcttctttctgtactccaagctgaccgtggataagtctcgctggcagcagggcaacgtgttcagctgtagcgtgatgcacgaagccctgcacaaccactacacccagaaaagcctgtcactgagccccggaaaa

SEQ ID NO: 89 - protein, 238 aa, synthetic construct
  REGION 1..238: note = 2219 Lambda Light Chain
MAWTPLFLFLLTCCPGGSNSQSVLTQPPSASGTPGQRISISCSGTSSNVENNYVYWYQHLPGTAPKLLIYRNDHRSSGIPDRFSASKSGTSASLAISGLRPEDEGDYYCAAWDDSRGGPDWVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 90 - DNA, 714 bp, synthetic construct
  misc_feature 1..714: note = 2219 Lambda Light Chain
  source 1..714: mol_type = other DNA
atggcttggacccctctgtttctgtttctgctgacctgttgccccggaggaagtaattcccagtccgtcctgacccagcctccctctgccagtggaacacccggccagcgaatctctatctcctgtagtggaacttcatctaatgtcgagaacaattacgtgtattggtaccagcacctgccaggaaccgcacctaagctgctgatctataggaacgaccacagaagctccggcatcccagatcggttctctgccagcaagtccggcacatctgccagcctggcaatctccggcctgcgccccgaggacgagggcgattactattgcgccgcctgggatgattccagaggaggaccagattgggtgttcggcggcgggactaaactgaccgtgctgggacagcctaaggcagcaccatccgtgacactgtttcccccttcctctgaggagctgcaggccaacaaggccaccctggtgtgcctgatcagcgacttctacccaggagcagtgaccgtggcatggaaggcagatagctccccagtgaaggcaggagtggagacaacaaccccttctaagcagagcaacaataagtacgccgcctctagctatctgtctctgacccccgagcagtggaagagccacaagagctattcctgccaggtcacccatgaaggctcaaccgtggagaaaacagtcgcccctaccgaatgctca

SEQ ID NO: 91 - protein, 476 aa, synthetic construct
  REGION 1..476: note = 3074 Heavy Chain
MDWTWRILFLVAAATGTHAQVQLQESGPGLVKPSETLSLTCTVSGGSISGFHWSWIRQPPGKGLEYIGYIYYSGSTSYNPSLKSRVSMSVDTSRNQFSLELSSVTAADTAVYYCARDFGEYHYDGRGFQCEGFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - DNA, 1428 bp, synthetic construct
  misc_feature 1..1428: note = 3074 Heavy Chain
  source 1..1428: mol_type = other DNA
atggattggacatggaggattctgtttctggtcgccgccgccactggaactcacgctcaggtgcagctgcaggaaagcgggcctggactggtgaagcctagtgaaactctgagcctgacttgtaccgtgagcggaggaagcattagcggcttccactggtcctggatcaggcagccccctggcaagggcctggagtacatcggctatatctactattctggcagcacctcctacaacccctccctgaagtctagggtgtctatgagcgtggacacatctagaaatcagttcagcctggagctgagctccgtgaccgcagcagatacagccgtgtactattgcgcccgggactttggcgagtaccactatgatggccgcggctttcagtgcgagggctttgatctgtgggggcagggaactctggtgaccgtgagcagcgccagcacaaagggcccatccgtgtttccactggcaccctcctctaagagcacctccggcggcacagccgccctgggctgtctggtgaaggattacttcccagagccagtgaccgtgagctggaactccggcgccctgacctccggagtgcacacatttccagccgtgctgcagagctccggcctgtatagcctgtctagcgtggtgaccgtgccctcctctagcctgggcacccagacatacatctgcaacgtgaatcacaagccatccaatacaaaggtggacaagaaggtggagcccaagtcttgtgataagacccacacatgccctccctgtcctgcaccagagctgctgggcggcccatccgtgttcctgtttccacccaagcctaaggacaccctgatgatcagccggaccccagaggtgacatgcgtggtggtggacgtgtcccacgaggaccccgaggtgaagttcaactggtacgtggatggcgtggaggtgcacaatgccaagaccaagcctagggaggagcagtataactctacctacagagtggtgagcgtgctgacagtgctgcaccaggactggctgaacggcaaggagtacaagtgcaaggtgtctaataaggccctgcccgcccctatcgagaagaccatcagcaaggcaaagggacagcctcgggagccacaggtgtatacactgcctccatctcgcgacgagctgaccaagaaccaggtgagcctgacatgtctggtgaagggcttctaccctagcgatatcgccgtggagtgggagtccaatggccagccagagaacaattataagaccacaccccctgtgctggactctgatggcagcttctttctgtactccaagctgaccgtggataagtctcgctggcagcagggcaacgtgttcagctgtagcgtgatgcacgaagccctgcacaaccactacacccagaaaagcctgtcactgagccccggaaaa

SEQ ID NO: 93 - protein, 235 aa, synthetic construct
  REGION 1..235: note = 3074 Lambda Light Chain
MAWTPLFLFLLTCCPGGSNSQSVLTQPPSVSAAPGQKVTISCSGSSSNIGNNMVSWYQQHPGTAPKLLIYENSKRPSGIPDRFSGSRSGTSATLGIIGLQTGDEAEYYCATWDGSLRTVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 94 - DNA, 705 bp, synthetic construct
  misc_feature 1..705: note = 3074 Lambda Light Chain
  source 1..705: mol_type = other DNA
atggcttggacccctctgtttctgtttctgctgacctgttgccccggaggaagtaattcccagagcgtcctgactcagcctccaagcgtgtccgcagcccctgggcagaaggtgaccattagttgtagcggcagcagcagcaatatcggcaacaatatggtgtcttggtaccagcagcacccaggaaccgcacctaagctgctgatctatgagaacagcaagaggccatccggcatccccgacaggttcagcggctccagatctggcaccagcgccacactgggcatcatcggcctgcagacaggcgatgaggccgagtactattgcgctacttgggatgggagcctgcggaccgtctttgggggaggaactaaactgactgtgctgggacagcctaaggcagcaccatccgtgacactgtttcccccttcctctgaggagctgcaggccaacaaggccaccctggtgtgcctgatcagcgacttctacccaggagcagtgaccgtggcatggaaggcagatagctccccagtgaaggcaggagtggagacaacaaccccttctaagcagagcaacaataagtacgccgcctctagctatctgtctctgacccccgagcagtggaagagccacaagagctattcctgccaggtcacccatgaaggctcaaccgtggagaaaacagtcgcccctaccgaatgctca

SEQ ID NO: 95 - protein, 473 aa, synthetic construct
  REGION 1..473: note = 4028 Heavy Chain
MDWTWRILFLVAAATGTHAEVQLVQSGAEVKKPGESLKISCKASGYSFSSYWIAWVRQMPGKGLEWMGFIYPADSDTRYSPSFQGQGTISADKSISTAYLQWSSLKASDTAMYYCAILGFWGANRGGGGMDVWGQGTTVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 96 - DNA, 1419 bp, synthetic construct
  misc_feature 1..1419: note = 4028 Heavy Chain
  source 1..1419: mol_type = other DNA
atggattggacatggaggattctgtttctggtcgccgccgccactggaactcacgctgaagtgcagctggtgcagagcggagccgaggtcaagaagcctggcgaaagcctgaaaatctcatgtaaagcaagcggatactcattcagctcctactggatcgcatgggtgaggcagatgccaggcaagggcctggagtggatgggcttcatctaccccgccgactctgataccagatattccccttcttttcagggccagggcaccatcagcgccgacaagagcatctccacagcctatctgcagtggtctagcctgaaggcctccgatacagccatgtactattgcgccatcctgggcttctggggcgccaatcggggcggaggcgggatggatgtctgggggcagggaaccaccgtcattgtcagcagtgccagcacaaagggcccatccgtgtttccactggcaccctcctctaagagcacctccggcggcacagccgccctgggctgtctggtgaaggattacttcccagagccagtgaccgtgagctggaactccggcgccctgacctccggagtgcacacatttccagccgtgctgcagagctccggcctgtatagcctgtctagcgtggtgaccgtgccctcctctagcctgggcacccagacatacatctgcaacgtgaatcacaagccatccaatacaaaggtggacaagaaggtggagcccaagtcttgtgataagacccacacatgccctccctgtcctgcaccagagctgctgggcggcccatccgtgttcctgtttccacccaagcctaaggacaccctgatgatcagccggaccccagaggtgacatgcgtggtggtggacgtgtcccacgaggaccccgaggtgaagttcaactggtacgtggatggcgtggaggtgcacaatgccaagaccaagcctagggaggagcagtataactctacctacagagtggtgagcgtgctgacagtgctgcaccaggactggctgaacggcaaggagtacaagtgcaaggtgtctaataaggccctgcccgcccctatcgagaagaccatcagcaaggcaaagggacagcctcgggagccacaggtgtatacactgcctccatctcgcgacgagctgaccaagaaccaggtgagcctgacatgtctggtgaagggcttctaccctagcgatatcgccgtggagtgggagtccaatggccagccagagaacaattataagaccacaccccctgtgctggactctgatggcagcttctttctgtactccaagctgaccgtggataagtctcgctggcagcagggcaacgtgttcagctgtagcgtgatgcacgaagccctgcacaaccactacacccagaaaagcctgtcactgagccccggaaaa

SEQ ID NO: 97 - protein, 235 aa, synthetic construct
  REGION 1..235: note = 4028 Lambda Light Chain
MAWTPLFLFLLTCCPGGSNSQSYELTQPPSVSVSPGQTARITCSGDALPEKYAYWYQQKSGQAPVLIIYEDSKRPSGIPERFSGSRSGTMATLTISGAQVDDEADYYCYSTNSGGTFFVFGTGTKVTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 98 - DNA, 705 bp, synthetic construct
  misc_feature 1..705: note = 4028 Lambda Light Chain
  source 1..705: mol_type = other DNA
atggcttggacccctctgtttctgtttctgctgacctgttgccccggaggaagtaattcccagagctacgaactgactcagcctcccagcgtgtccgtgtcccccggccagactgccagaatcacttgcagcggcgatgccctgccagagaagtacgcctattggtaccagcagaagtctggccaggcccccgtgctgatcatctatgaggacagcaagaggccctccggcatccctgagaggttcagcggctccagatctggcaccatggccaccctgacaatcagcggagcacaggtggacgatgaggcagattactattgctactccactaactcaggcgggacatttttcgtgtttgggacagggactaaggtcaccgtgctgggacagcctaaggcagcaccatccgtgacactgtttcccccttcctctgaggagctgcaggccaacaaggccaccctggtgtgcctgatcagcgacttctacccaggagcagtgaccgtggcatggaaggcagatagctccccagtgaaggcaggagtggagacaacaaccccttctaagcagagcaacaataagtacgccgcctctagctatctgtctctgacccccgagcagtggaagagccacaagagctattcctgccaggtcacccatgaaggctcaaccgtggagaaaacagtcgcccctaccgaatgctca

SEQ ID NO: 99 - protein, 473 aa, synthetic construct
  REGION 1..473: note = 2557 Heavy Chain
MDWTWRILFLVAAATGTHAEVQLVESGGEVKQPGQSLKISCKSSGYNFLDSWIGWVRQIPGKGLEWIGIIYPDDSDAHYSPSFEGQVTMSVDKSISTAYLQWTTLQASDTGKYFCTRLYLFEGAQSSNAFDLWGQGTMILVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 100 - DNA, 1419 bp, synthetic construct
  misc_feature 1..1419: note = 2557 Heavy Chain
  source 1..1419: mol_type = other DNA
atggattggacatggaggattctgtttctggtcgccgccgccactggaactcacgctgaagtgcagctggtggaatccgggggcgaggtcaaacagcctgggcagagcctgaagatcagttgtaaaagtagtgggtataatttcctggacagctggatcggatgggtgaggcagatcccaggcaagggcctggagtggatcggcatcatctaccccgacgattccgacgcccactatagcccttccttcgagggccaggtgaccatgtctgtggataagtctatcagcacagcctacctgcagtggaccacactgcaggcctccgataccggcaagtacttctgcacaagactgtatctgtttgagggcgctcagtcaagcaacgcattcgatctgtgggggcagggaactatgattctggtgtcaagcgccagcacaaagggcccatccgtgtttccactggcaccctcctctaagagcacctccggcggcacagccgccctgggctgtctggtgaaggattacttcccagagccagtgaccgtgagctggaactccggcgccctgacctccggagtgcacacatttccagccgtgctgcagagctccggcctgtatagcctgtctagcgtggtgaccgtgccctcctctagcctgggcacccagacatacatctgcaacgtgaatcacaagccatccaatacaaaggtggacaagaaggtggagcccaagtcttgtgataagacccacacatgccctccctgtcctgcaccagagctgctgggcggcccatccgtgttcctgtttccacccaagcctaaggacaccctgatgatcagccggaccccagaggtgacatgcgtggtggtggacgtgtcccacgaggaccccgaggtgaagttcaactggtacgtggatggcgtggaggtgcacaatgccaagaccaagcctagggaggagcagtataactctacctacagagtggtgagcgtgctgacagtgctgcaccaggactggctgaacggcaaggagtacaagtgcaaggtgtctaataaggccctgcccgcccctatcgagaagaccatcagcaaggcaaagggacagcctcgggagccacaggtgtatacactgcctccatctcgcgacgagctgaccaagaaccaggtgagcctgacatgtctggtgaagggcttctaccctagcgatatcgccgtggagtgggagtccaatggccagccagagaacaattataagaccacaccccctgtgctggactctgatggcagcttctttctgtactccaagctgaccgtggataagtctcgctggcagcagggcaacgtgttcagctgtagcgtgatgcacgaagccctgcacaaccactacacccagaaaagcctgtcactgagccccggaaaa

SEQ ID NO: 101 - protein, 233 aa, synthetic construct
  REGION 1..233: note = 2557 Lambda Light Chain
MAWTPLFLFLLTCCPGGSNSQSYLLTQPPSVSVSPGQTASISCSGDKLDDKYVSWYYQRPGQSPVLLMYQDFKRPSGIPERLSGSKSGKTATLTISGTQSLDEGDYYCQAWDASTGVSGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 102 - DNA, 699 bp, synthetic construct
  misc_feature 1..699: note = 2557 Lambda Light Chain
  source 1..699: mol_type = other DNA
atggcttggacccctctgtttctgtttctgctgacctgttgccccggaggaagtaattcccagagctacctgctgacccagcctccttccgtgagcgtgagccctggacagaccgcatcaatttcttgttccggcgataaactggatgacaagtacgtgtcttggtactatcagaggcccggacagagccctgtgctgctgatgtatcaggatttcaagaggccatccggcatccccgagagactgagcggctccaagtctggcaagaccgccacactgaccatcagcggcacacagtccctggacgagggcgattactattgccaggcttgggatgcttcaacaggcgtctctgggggaggaacaaaactgaccgtgctgggacagcctaaggcagcaccatccgtgacactgtttcccccttcctctgaggagctgcaggccaacaaggccaccctggtgtgcctgatcagcgacttctacccaggagcagtgaccgtggcatggaaggcagatagctccccagtgaaggcaggagtggagacaacaaccccttctaagcagagcaacaataagtacgccgcctctagctatctgtctctgacccccgagcagtggaagagccacaagagctattcctgccaggtcacccatgaaggctcaaccgtggagaaaacagtcgcccctaccgaatgctca

SEQ ID NO: 103 - protein, 472 aa, synthetic construct
  REGION 1..472: note = F425-B4e8 Heavy Chain
MDWTWRILFLVAAATGTHAQVQLVQSGGGLVQPGGSLRLSCAAFGFNFSSYVMHWVRQAPGQGLEYLSAISSDGETTYHANSVKGRFTSSRDNSKNTLFLQMGSLRTEDVAVYYCARDRYYETSGSNAFDVWGQGTMVVVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - DNA, 1416 bp, synthetic construct
  misc_feature 1..1416: note = F425-B4e8 Heavy Chain
  source 1..1416: mol_type = other DNA
atggattggacatggaggattctgtttctggtcgccgccgccactggaactcacgctcaggtgcagctggtgcagagcgggggcggcctggtgcagcctggagggtcactgagactgtcctgtgccgcattcgggttcaatttcagctcctacgtgatgcactgggtgaggcaggcaccaggacagggcctggagtacctgtctgccatctctagcgacggcgagacaacatatcacgccaactccgtgaagggccggttcacctcctctcgcgataacagcaagaatacactgtttctgcagatgggctccctgaggaccgaggacgtggccgtgtactattgcgccagggatagatactatgagacaagcggaagcaacgcctttgacgtgtgggggcagggaacaatggtggtggtctcctcagccagcacaaagggcccatccgtgtttccactggcaccctcctctaagagcacctccggcggcacagccgccctgggctgtctggtgaaggattacttcccagagccagtgaccgtgagctggaactccggcgccctgacctccggagtgcacacatttccagccgtgctgcagagctccggcctgtatagcctgtctagcgtggtgaccgtgccctcctctagcctgggcacccagacatacatctgcaacgtgaatcacaagccatccaatacaaaggtggacaagaaggtggagcccaagtcttgtgataagacccacacatgccctccctgtcctgcaccagagctgctgggcggcccatccgtgttcctgtttccacccaagcctaaggacaccctgatgatcagccggaccccagaggtgacatgcgtggtggtggacgtgtcccacgaggaccccgaggtgaagttcaactggtacgtggatggcgtggaggtgcacaatgccaagaccaagcctagggaggagcagtataactctacctacagagtggtgagcgtgctgacagtgctgcaccaggactggctgaacggcaaggagtacaagtgcaaggtgtctaataaggccctgcccgcccctatcgagaagaccatcagcaaggcaaagggacagcctcgggagccacaggtgtatacactgcctccatctcgcgacgagctgaccaagaaccaggtgagcctgacatgtctggtgaagggcttctaccctagcgatatcgccgtggagtgggagtccaatggccagccagagaacaattataagaccacaccccctgtgctggactctgatggcagcttctttctgtactccaagctgaccgtggataagtctcgctggcagcagggcaacgtgttcagctgtagcgtgatgcacgaagccctgcacaaccactacacccagaaaagcctgtcactgagccccggaaaa

SEQ ID NO: 105 - protein, 235 aa, synthetic construct
  REGION 1..235: note = F425 Kappa Light Chain
MVLQTQVFISLLLWISGAYGNSVLTQSPSSLSASVGDRVTITCQASQDISNYLNWYQHKPGKAPKLLIYTASNLETGVPSRFSGGGSGTHFSFTITSLQPEDAATYFCQQYDNLGDLSFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 106 - DNA, 705 bp, synthetic construct
  misc_feature 1..705: note = F425 Kappa Light Chain
  source 1..705: mol_type = other DNA
atggtgctgcagacccaggtgttcatttcactgctgctgtggatttcaggagcctacggaaactctgtgctgacccagtctccatcttctctgagcgcatccgtcggcgaccgcgtgaccatcacttgccaggcttcccaggacatttctaactacctgaattggtatcagcacaagcccggcaaggcccctaagctgctgatctacaccgccagcaacctggagacaggagtgcccagcaggttcagcggcggcggcagcggcacccacttcagctttaccatcacatccctgcagccagaggacgcagcaacatatttttgccagcagtacgataatctgggggacctgtcattcggcggcgggaccaaagtggaaatcaagcggaccgtggcagcaccttccgtgttcatctttcccccttctgacgagcagctgaagtctggcacagccagcgtggtgtgcctgctgaacaacttctaccccagggaggccaaggtgcagtggaaggtggataacgccctgcagagcggcaattcccaggagtctgtgaccgagcaggacagcaaggattccacatattctctgagctccaccctgacactgagcaaggccgactacgagaagcacaaggtgtatgcctgcgaggtcacccatcaggggctgtcaagtccagtcacaaagtccttcaataggggcgaatgc

SEQ ID NO: 107 - protein, 477 aa, synthetic construct
  REGION 1..477: note = 17B Heavy Chain
MDWTWRILFLVAAATGTHAEVQLVESGAEVKKPGSSVKVSCKASGDTFIRYSFTWVRQAPGQGLEWMGRIITILDVAHYAPHLQGRVTITADKSTSTVYLELRNLRSDDTAVYFCAGVYEGEADEGEYDNNGFLKHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 108 - DNA, 1431 bp, synthetic construct
  misc_feature 1..1431: note = 17B Heavy Chain
  source 1..1431: mol_type = other DNA
atggattggacatggaggattctgtttctggtcgccgccgccactggaactcacgctgaagtgcagctggtggaaagcggagccgaagtcaaaaaacctgggtcaagcgtcaaagtcagttgtaaggcaagcggggatacattcatccggtactctttcacctgggtgcgccaggcaccaggacagggcctggagtggatgggcaggatcatcacaatcctggacgtggcacactacgcccctcacctgcagggaagggtgaccatcacagccgataagagcacctccacagtgtatctggagctgaggaacctgagaagcgacgataccgccgtgtacttttgcgcaggcgtgtatgagggagaggcagacgagggcgagtacgacaacaacggatttctgaagcattggggacaggggactctggtgacagtgagcagcgccagcacaaagggcccatccgtgtttccactggcaccctcctctaagagcacctccggcggcacagccgccctgggctgtctggtgaaggattacttcccagagccagtgaccgtgagctggaactccggcgccctgacctccggagtgcacacatttccagccgtgctgcagagctccggcctgtatagcctgtctagcgtggtgaccgtgccctcctctagcctgggcacccagacatacatctgcaacgtgaatcacaagccatccaatacaaaggtggacaagaaggtggagcccaagtcttgtgataagacccacacatgccctccctgtcctgcaccagagctgctgggcggcccatccgtgttcctgtttccacccaagcctaaggacaccctgatgatcagccggaccccagaggtgacatgcgtggtggtggacgtgtcccacgaggaccccgaggtgaagttcaactggtacgtggatggcgtggaggtgcacaatgccaagaccaagcctagggaggagcagtataactctacctacagagtggtgagcgtgctgacagtgctgcaccaggactggctgaacggcaaggagtacaagtgcaaggtgtctaataaggccctgcccgcccctatcgagaagaccatcagcaaggcaaagggacagcctcgggagccacaggtgtatacactgcctccatctcgcgacgagctgaccaagaaccaggtgagcctgacatgtctggtgaagggcttctaccctagcgatatcgccgtggagtgggagtccaatggccagccagagaacaattataagaccacaccccctgtgctggactctgatggcagcttctttctgtactccaagctgaccgtggataagtctcgctggcagcagggcaacgtgttcagctgtagcgtgatgcacgaagccctgcacaaccactacacccagaaaagcctgtcactgagccccggaaaa

SEQ ID NO: 109 - protein, 236 aa, synthetic construct
  REGION 1..236: note = 17B Kappa Light Chain
MVLQTQVFISLLLWISGAYGDIVMTQSPATLSVSPGERATLSCRASESVSSDLAWYQQKPGQAPRLLIYGASTRATGVPARFSGSGSGAEFTLTISSLQSEDFAVYYCQQYNNWPPRYTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 110 - DNA, 708 bp, synthetic construct
  misc_feature 1..708: note = 17B Kappa Light Chain
  source 1..708: mol_type = other DNA
atggtgctgcagacccaggtgttcatttcactgctgctgtggatttcaggagcctacggagatattgtgatgacccagagcccagccaccctgtccgtgagccccggcgagagagccaccctgtcctgtagagcctccgagagcgtgagctccgacctggcatggtaccagcagaagccaggacaggcccctaggctgctgatctatggagcatccaccagggccacaggagtgccagcaagattctccggctctggcagcggagcagagtttaccctgacaatctctagcctgcagtctgaggatttcgccgtgtactattgccagcagtacaataactggcctcctcgctacaccttcgggcagggcacacggctggaaatcaaaagaaccgtggcagcaccttccgtgttcatctttcccccttctgacgagcagctgaagtctggcacagccagcgtggtgtgcctgctgaacaacttctaccccagggaggccaaggtgcagtggaaggtggataacgccctgcagagcggcaattcccaggagtctgtgaccgagcaggacagcaaggattccacatattctctgagctccaccctgacactgagcaaggccgactacgagaagcacaaggtgtatgcctgcgaggtcacccatcaggggctgtcaagtccagtcacaaagtccttcaataggggcgaatgc

SEQ ID NO: 111 - protein, 472 aa, synthetic construct
  REGION 1..472: note = F105 Heavy Chain
MDWTWRILFLVAAATGTHAQVQLQESGPGLVKPSETLSLTCTVSGGSISSHYWSWIRQSPGKGLQWIGYIYYSGSTNYSPSLKSRVTISVETAKNQFSLKLTSMTAADTAVYYCARGPVPAVFYGDYRLDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 112 - DNA, 1416 bp, synthetic construct
  misc_feature 1..1416: note = F105 Heavy Chain
  source 1..1416: mol_type = other DNA
atggattggacatggaggattctgtttctggtcgccgccgccactggaactcacgctcaggtgcagctgcaggaaagcggacccggcctggtgaagccctctgaaactctgtcactgacttgtaccgtctctgggggaagcatcagctcccactactggagctggatcaggcagtccccaggcaagggcctgcagtggatcggctacatctactattctggcagcaccaactattccccctctctgaagtctagagtgaccatcagcgtggagacagccaagaatcagttctccctgaagctgacctctatgacagccgccgacacagccgtgtactattgcgccaggggccccgtgcctgccgtgttttacggagattatcggctggacccttgggggcagggaacactggtgactgtgagtagcgccagcacaaagggcccatccgtgtttccactggcaccctcctctaagagcacctccggcggcacagccgccctgggctgtctggtgaaggattacttcccagagccagtgaccgtgagctggaactccggcgccctgacctccggagtgcacacatttccagccgtgctgcagagctccggcctgtatagcctgtctagcgtggtgaccgtgccctcctctagcctgggcacccagacatacatctgcaacgtgaatcacaagccatccaatacaaaggtggacaagaaggtggagcccaagtcttgtgataagacccacacatgccctccctgtcctgcaccagagctgctgggcggcccatccgtgttcctgtttccacccaagcctaaggacaccctgatgatcagccggaccccagaggtgacatgcgtggtggtggacgtgtcccacgaggaccccgaggtgaagttcaactggtacgtggatggcgtggaggtgcacaatgccaagaccaagcctagggaggagcagtataactctacctacagagtggtgagcgtgctgacagtgctgcaccaggactggctgaacggcaaggagtacaagtgcaaggtgtctaataaggccctgcccgcccctatcgagaagaccatcagcaaggcaaagggacagcctcgggagccacaggtgtatacactgcctccatctcgcgacgagctgaccaagaaccaggtgagcctgacatgtctggtgaagggcttctaccctagcgatatcgccgtggagtgggagtccaatggccagccagagaacaattataagaccacaccccctgtgctggactctgatggcagcttctttctgtactccaagctgaccgtggataagtctcgctggcagcagggcaacgtgttcagctgtagcgtgatgcacgaagccctgcacaaccactacacccagaaaagcctgtcactgagccccggaaaa

SEQ ID NO: 113 - protein, 235 aa, synthetic construct
  REGION 1..235: note = F105 Kappa Light Chain
MVLQTQVFISLLLWISGAYGEIVLTQSPGTLSLSAGERATLSCRASQSVSSRYLAWYQQKPGQAPRLLIYGASSRATGIPDRFSGSGSGTDFTLTISRVEPEDFAVYYCQQYDNSVCTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 114 - DNA, 705 bp, synthetic construct
  misc_feature 1..705: note = F105 Kappa Light Chain
  source 1..705: mol_type = other DNA
atggtgctgcagacccaggtgttcatttcactgctgctgtggatttcaggagcctacggagaaatcgtcctgacccagtcccccggaaccctgagtctgagcgccggagagcgagccaccctgagttgtagagcatcacagagtgtcagctcccggtacctggcctggtatcagcagaagccaggacaggcccctcgcctgctgatctacggagcctctagcagggccaccggcatcccagacagattctccggctctggcagcggcacagacttcaccctgacaatctccagggtggagcccgaggacttcgccgtgtactattgccagcagtatgacaatagcgtctgtaccttcgggcagggcaccaaactggaaatcaagaggaccgtggcagcaccttccgtgttcatctttcccccttctgacgagcagctgaagtctggcacagccagcgtggtgtgcctgctgaacaacttctaccccagggaggccaaggtgcagtggaaggtggataacgccctgcagagcggcaattcccaggagtctgtgaccgagcaggacagcaaggattccacatattctctgagctccaccctgacactgagcaaggccgactacgagaagcacaaggtgtatgcctgcgaggtcacccatcaggggctgtcaagtccagtcacaaagtccttcaataggggcgaatgc

SEQ ID NO: 115 - protein, 476 aa, synthetic construct
  REGION 1..476: note = b12 Heavy Chain
MDWTWRILFLVAAATGTHAQVQLVQSGAEVKKPGASVKVSCQASGYRFSNFVIHWVRQAPGQRFEWMGWINPYNGNKEFSAKFQDRVTFTADTSANTAYMELRSLRSADTAVYYCARVGPYSWDDSPQDNYYMDVWGKGTTVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 116 - DNA, 1428 bp, synthetic construct
  misc_feature 1..1428: note = b12 Heavy Chain
  source 1..1428: mol_type = other DNA
atggattggacatggaggattctgtttctggtcgccgccgccactggaactcacgctcaggtgcagctggtccagtcaggagcagaagtgaagaaacccggagcatcagtgaaagtcagttgtcaggcaagcggatacagatttagcaacttcgtgatccactgggtgcggcaggcaccaggacagaggttcgagtggatgggctggatcaatccttacaacggcaataaggagttctccgccaagtttcaggacagggtgaccttcacagccgatacctctgccaacacagcctacatggagctgaggagcctgagatccgccgacaccgccgtgtactattgcgcccgggtgggaccatattcttgggacgatagccctcaggataactattacatggacgtgtgggggaagggaacaacagtcatcgtgtcatcagccagcacaaagggcccatccgtgtttccactggcaccctcctctaagagcacctccggcggcacagccgccctgggctgtctggtgaaggattacttcccagagccagtgaccgtgagctggaactccggcgccctgacctccggagtgcacacatttccagccgtgctgcagagctccggcctgtatagcctgtctagcgtggtgaccgtgccctcctctagcctgggcacccagacatacatctgcaacgtgaatcacaagccatccaatacaaaggtggacaagaaggtggagcccaagtcttgtgataagacccacacatgccctccctgtcctgcaccagagctgctgggcggcccatccgtgttcctgtttccacccaagcctaaggacaccctgatgatcagccggaccccagaggtgacatgcgtggtggtggacgtgtcccacgaggaccccgaggtgaagttcaactggtacgtggatggcgtggaggtgcacaatgccaagaccaagcctagggaggagcagtataactctacctacagagtggtgagcgtgctgacagtgctgcaccaggactggctgaacggcaaggagtacaagtgcaaggtgtctaataaggccctgcccgcccctatcgagaagaccatcagcaaggcaaagggacagcctcgggagccacaggtgtatacactgcctccatctcgcgacgagctgaccaagaaccaggtgagcctgacatgtctggtgaagggcttctaccctagcgatatcgccgtggagtgggagtccaatggccagccagagaacaattataagaccacaccccctgtgctggactctgatggcagcttctttctgtactccaagctgaccgtggataagtctcgctggcagcagggcaacgtgttcagctgtagcgtgatgcacgaagccctgcacaaccactacacccagaaaagcctgtcactgagccccggaaaa

SEQ ID NO: 117 - protein, 235 aa, synthetic construct
  REGION 1..235: note = b12 Kappa Light Chain
MVLQTQVFISLLLWISGAYGEIVLTQSPGTLSLSPGERATFSCRSSHSIRSRRVAWYQHKPGQAPRLVIHGVSNRASGISDRFSGSGSGTDFTLTITRVEPEDFALYYCQVYGASSYTFGQGTKLERKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 118 - DNA, 705 bp, synthetic construct
  misc_feature 1..705: note = b12 Kappa Light Chain
  source 1..705: mol_type = other DNA
atggtgctgcagacccaggtgttcatttcactgctgctgtggatttcaggagcctacggagagattgtgctgacccagagtcctggaaccctgtccctgagtcctggagaacgggccaccttttcatgccgatcatcccatagtatcaggtctaggagagtggcatggtaccagcacaagccaggacaggcccctcggctggtcatccacggcgtgagcaacagggcaagcggaatctccgaccgcttcagcggctccggctctggcacagacttcaccctgacaatcaccagggtggagcccgaggacttcgccctgtactattgccaggtctacggggcatcctcctatacattcggacagggcacaaaactggaacggaaaagaaccgtggcagcaccttccgtgttcatctttcccccttctgacgagcagctgaagtctggcacagccagcgtggtgtgcctgctgaacaacttctaccccagggaggccaaggtgcagtggaaggtggataacgccctgcagagcggcaattcccaggagtctgtgaccgagcaggacagcaaggattccacatattctctgagctccaccctgacactgagcaaggccgactacgagaagcacaaggtgtatgcctgcgaggtcacccatcaggggctgtcaagtccagtcacaaagtccttcaataggggcgaatgc

SEQ ID NO: 119 - protein, 482 aa, synthetic construct
  REGION 1..482: note = b6 Heavy Chain
MDWTWRILFLVAAATGTHANSQLEESGGGLVKPGGSLRLSCVGSGFTFSSAWMAWVRQAPGRGLEWVGLIKSKADGETTDYATPVKGRFSISRNNLKDTVYLQMDSLRADDTAVYYCATQKPRYFDLLSGQYRRVAGAFDVWGHGTTVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 120 - DNA, 1446 bp, synthetic construct
  misc_feature 1..1446: note = b6 Heavy Chain
  source 1..1446: mol_type = other DNA
atggattggacatggaggattctgtttctggtcgccgccgccactggaactcacgctaactcacagctggaggagagcggcggcggcctggtcaaacctggcgggtccctgcgactgtcttgcgtggggtctggctttactttcagctccgcctggatggcatgggtgaggcaggcaccaggaagaggcctggagtgggtgggcctgatcaagagcaaggccgacggcgagacaacagattacgccacccctgtgaagggccggttctctatcagccgcaacaatctgaaggacacagtgtatctgcagatggattccctgagggccgacgataccgccgtgtactattgcgccacacagaagccccggtactttgacctgctgtctggccagtatcggagagtcgcaggggcttttgatgtctgggggcacggcacaacagtcacagtgtcaccagccagcacaaagggcccatccgtgtttccactggcaccctcctctaagagcacctccggcggcacagccgccctgggctgtctggtgaaggattacttcccagagccagtgaccgtgagctggaactccggcgccctgacctccggagtgcacacatttccagccgtgctgcagagctccggcctgtatagcctgtctagcgtggtgaccgtgccctcctctagcctgggcacccagacatacatctgcaacgtgaatcacaagccatccaatacaaaggtggacaagaaggtggagcccaagtcttgtgataagacccacacatgccctccctgtcctgcaccagagctgctgggcggcccatccgtgttcctgtttccacccaagcctaaggacaccctgatgatcagccggaccccagaggtgacatgcgtggtggtggacgtgtcccacgaggaccccgaggtgaagttcaactggtacgtggatggcgtggaggtgcacaatgccaagaccaagcctagggaggagcagtataactctacctacagagtggtgagcgtgctgacagtgctgcaccaggactggctgaacggcaaggagtacaagtgcaaggtgtctaataaggccctgcccgcccctatcgagaagaccatcagcaaggcaaagggacagcctcgggagccacaggtgtatacactgcctccatctcgcgacgagctgaccaagaaccaggtgagcctgacatgtctggtgaagggcttctaccctagcgatatcgccgtggagtgggagtccaatggccagccagagaacaattataagaccacaccccctgtgctggactctgatggcagcttctttctgtactccaagctgaccgtggataagtctcgctggcagcagggcaacgtgttcagctgtagcgtgatgcacgaagccctgcacaaccactacacccagaaaagcctgtcactgagccccggaaaa

SEQ ID NO: 121 - protein, 235 aa, synthetic construct
  REGION 1..235: note = b6 Kappa Light Chain
MVLQTQVFISLLLWISGAYGEIVLTQSPGTLSLSPGERATLSCRAGQSISSNYLAWYQQKPGQAPRLLIYGASNRATGIPDRFSGSGSGTDFTLSISRLEPEDFAVYYCQQYGTSPYTFGQGTQLDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 122 - DNA, 705 bp, synthetic construct
  misc_feature 1..705: note = b6 Kappa Light Chain
  source 1..705: mol_type = other DNA
atggtgctgcagacccaggtgttcatttcactgctgctgtggatttcaggagcctacggagaaatcgtcctgacccagtcccctggcaccctgtccctgtcccctggagaaagagccaccctgagttgtagagccgggcagagcattagctccaactacctggcctggtatcagcagaagccaggacaggcccctcggctgctgatctacggagcatccaatagggcaaccggcatcccagacagattctctggcagcggctccggaaccgacttcaccctgtctatcagccgcctggagccagaggacttcgccgtgtactattgccagcagtatggcacttcaccttacacttttggacagggaactcagctggacatcaagcgaaccgtggcagcaccttccgtgttcatctttcccccttctgacgagcagctgaagtctggcacagccagcgtggtgtgcctgctgaacaacttctaccccagggaggccaaggtgcagtggaaggtggataacgccctgcagagcggcaattcccaggagtctgtgaccgagcaggacagcaaggattccacatattctctgagctccaccctgacactgagcaaggccgactacgagaagcacaaggtgtatgcctgcgaggtcacccatcaggggctgtcaagtccagtcacaaagtccttcaataggggcgaatgc

SEQ ID NO: 123 - protein, 471 aa, synthetic construct
  REGION 1..471: note = 11A Heavy Chain
MDWTWRILFLVAAATGTHANSQLVESGGGLVKPGTSLSLTCKASGFDFSDNYYICWVRQAPGKGLEWIGCIFTQNVRTYYANWAKGRFTISKTSSTTVTLQMTSLTVADTATYFCARFSDTGPDYGLGNLWGPGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 124 - DNA, 1413 bp, synthetic construct
  misc_feature 1..1413: note = 11A Heavy Chain
  source 1..1413: mol_type = other DNA
atggattggacatggaggattctgtttctggtcgccgccgccactggaactcacgctaatagccagctggtggagagcggcggcggcctggtgaaacctggaacctcactgtccctgacctgtaaggcaagcggatttgacttctccgacaactactatatctgctgggtgcggcaggcaccaggcaagggcctggagtggatcggctgtatcttcacccagaacgtgaggacatactatgccaattgggccaagggcaggttcaccatctctaagacaagctccaccacagtgaccctgcagatgaccagcctgacagtggccgacaccgccacatacttctgcgcccgcttttctgatacaggacctgactacggactgggaaatctgtggggacctgggagcctggtgactgtctcatcagccagcacaaagggcccatccgtgtttccactggcaccctcctctaagagcacctccggcggcacagccgccctgggctgtctggtgaaggattacttcccagagccagtgaccgtgagctggaactccggcgccctgacctccggagtgcacacatttccagccgtgctgcagagctccggcctgtatagcctgtctagcgtggtgaccgtgccctcctctagcctgggcacccagacatacatctgcaacgtgaatcacaagccatccaatacaaaggtggacaagaaggtggagcccaagtcttgtgataagacccacacatgccctccctgtcctgcaccagagctgctgggcggcccatccgtgttcctgtttccacccaagcctaaggacaccctgatgatcagccggaccccagaggtgacatgcgtggtggtggacgtgtcccacgaggaccccgaggtgaagttcaactggtacgtggatggcgtggaggtgcacaatgccaagaccaagcctagggaggagcagtataactctacctacagagtggtgagcgtgctgacagtgctgcaccaggactggctgaacggcaaggagtacaagtgcaaggtgtctaataaggccctgcccgcccctatcgagaagaccatcagcaaggcaaagggacagcctcgggagccacaggtgtatacactgcctccatctcgcgacgagctgaccaagaaccaggtgagcctgacatgtctggtgaagggcttctaccctagcgatatcgccgtggagtgggagtccaatggccagccagagaacaattataagaccacaccccctgtgctggactctgatggcagcttctttctgtactccaagctgaccgtggataagtctcgctggcagcagggcaacgtgttcagctgtagcgtgatgcacgaagccctgcacaaccactacacccagaaaagcctgtcactgagccccggaaaa

SEQ ID NO: 125 - protein, 240 aa, synthetic construct
  REGION 1..240: note = 11A Kappa Light Chain
MVLQTQVFISLLLWISGAYGDIGMTQTPASGEAAVGGTVTIKCQASQRIGSHVSWYQQKPGQRPKLLIYGASNLESGVPSRFSGRGSGTQFTLTISDLECADAATYYCQATYDPYTGGSYGAGFGGGTAVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 126 - DNA, 720 bp, synthetic construct
  misc_feature 1..720: note = 11A Kappa Light Chain
  source 1..720: mol_type = other DNA
atggtgctgcagacccaggtgttcatttcactgctgctgtggatttcaggagcctacggagacatcggaatgacccagacaccagccagcggggaagcagccgtcgggggaacagtgacaatcaagtgccaggccagccagagaatcggcagccacgtgtcctggtaccagcagaagcccggccagaggcctaagctgctgatctatggcgcctccaacctggagtctggcgtgccatctaggttcagcggcagaggcagcggcacccagtttaccctgacaatctccgacctggagtgcgcagatgcagcaacctactattgtcaggccacatacgacccctacactgggggcagctatggagcaggatttgggggaggaacagccgtcgtcgtgaaaaggaccgtggcagcaccttccgtgttcatctttcccccttctgacgagcagctgaagtctggcacagccagcgtggtgtgcctgctgaacaacttctaccccagggaggccaaggtgcagtggaaggtggataacgccctgcagagcggcaattcccaggagtctgtgaccgagcaggacagcaaggattccacatattctctgagctccaccctgacactgagcaaggccgactacgagaagcacaaggtgtatgcctgcgaggtcacccatcaggggctgtcaagtccagtcacaaagtccttcaataggggcgaatgc

SEQ ID NO: 127 - protein, 473 aa, synthetic construct
  REGION 1..473: note = 12N Heavy Chain
MDWTWRILFLVAAATGTHANSQLVESGGDLVKPEGSLTLTCTASGFSFSSGYYVCWVRQAPGKGLDWVACIYVGSSGITDYATWAKGRFTISRTSSTTVTLQMTSLTAADTATYFCARGGVGDYGVISDFRLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 128 - DNA, 1419 bp, synthetic construct
  misc_feature 1..1419: note = 12N Heavy Chain
  source 1..1419: mol_type = other DNA
atggattggacatggaggattctgtttctggtcgccgccgccactggaactcacgctaactcacagctggtggaaagtggaggcgacctggtgaaacccgaaggcagtctgaccctgacctgtaccgcaagtggattctcattcagctccggctactacgtgtgctgggtgcggcaggcaccaggcaagggcctggactgggtggcctgtatctacgtgggctctagcggcatcaccgattatgccacatgggccaagggcaggttcaccatcagcagaacatcctctaccacagtgaccctgcagatgacctccctgacagcagcagacaccgcaacatacttttgcgcccgcggcggcgtgggcgattatggggtcatttctgattttcggctgtgggggcctgggacactggtcaccgtctcatctgccagcacaaagggcccatccgtgtttccactggcaccctcctctaagagcacctccggcggcacagccgccctgggctgtctggtgaaggattacttcccagagccagtgaccgtgagctggaactccggcgccctgacctccggagtgcacacatttccagccgtgctgcagagctccggcctgtatagcctgtctagcgtggtgaccgtgccctcctctagcctgggcacccagacatacatctgcaacgtgaatcacaagccatccaatacaaaggtggacaagaaggtggagcccaagtcttgtgataagacccacacatgccctccctgtcctgcaccagagctgctgggcggcccatccgtgttcctgtttccacccaagcctaaggacaccctgatgatcagccggaccccagaggtgacatgcgtggtggtggacgtgtcccacgaggaccccgaggtgaagttcaactggtacgtggatggcgtggaggtgcacaatgccaagaccaagcctagggaggagcagtataactctacctacagagtggtgagcgtgctgacagtgctgcaccaggactggctgaacggcaaggagtacaagtgcaaggtgtctaataaggccctgcccgcccctatcgagaagaccatcagcaaggcaaagggacagcctcgggagccacaggtgtatacactgcctccatctcgcgacgagctgaccaagaaccaggtgagcctgacatgtctggtgaagggcttctaccctagcgatatcgccgtggagtgggagtccaatggccagccagagaacaattataagaccacaccccctgtgctggactctgatggcagcttctttctgtactccaagctgaccgtggataagtctcgctggcagcagggcaacgtgttcagctgtagcgtgatgcacgaagccctgcacaaccactacacccagaaaagcctgtcactgagccccggaaaa

SEQ ID NO: 129 - protein, 241 aa, synthetic construct
  REGION 1..241: note = 12N Kappa Light Chain
MVLQTQVFISLLLWISGAYGDIVMTQTPASVEVPVGGTVTIKCQASQNIYSHNYLSWYQLKLGQPPKLLIYKASTLESGVPSRFKGSGSGTEFTLTISDLECADAATYYCQFTYGSASTGTYVAFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 130 - DNA, 723 bp, synthetic construct
  misc_feature 1..723: note = 12N Kappa Light Chain
  source 1..723: mol_type = other DNA
atggtgctgcagacccaggtgttcatttcactgctgctgtggatttcaggagcctacggagacattgtgatgactcagacaccagccagcgtggaagtgccagtgggaggaacagtgaccattaagtgccaggccagccagaacatttactctcacaactatctgagctggtaccagctgaagctgggccagccccctaagctgctgatctataaggcatctaccctggagagcggagtgccctccaggttcaagggcagcggctccggcacagagtttaccctgacaatctccgacctggagtgcgcagatgcagcaacctactattgtcagttcacatacggctcagcatcaacaggaacctatgtggcattcggcggagggacagaagtggtggtgaagcggaccgtggcagcaccttccgtgttcatctttcccccttctgacgagcagctgaagtctggcacagccagcgtggtgtgcctgctgaacaacttctaccccagggaggccaaggtgcagtggaaggtggataacgccctgcagagcggcaattcccaggagtctgtgaccgagcaggacagcaaggattccacatattctctgagctccaccctgacactgagcaaggccgactacgagaagcacaaggtgtatgcctgcgaggtcacccatcaggggctgtcaagtccagtcacaaagtccttcaataggggcgaatgc

SEQ ID NO: 131 - protein, 476 aa, synthetic construct
  REGION 1..476: note = 39F Heavy Chain
MDWTWRILFLVAAATGTHAEVQLVQSGAEVKKPGSTVKISCKASGYTFTDFYIQWLRQAPVKGLEWMGLVDPEDGQTRYPQRFQGRVTMTADPSTDTAYMEVSGLDFEDTADYYCATSTLLGGPYDNSGHDAFDIWGQGTKVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 132 - DNA, 1428 bp, synthetic construct
  misc_feature 1..1428: note = 39F Heavy Chain
  source 1..1428: mol_type = other DNA
atggattggacatggaggattctgtttctggtcgccgccgccactggaactcacgctgaagtgcagctggtccagtcaggggcagaggtcaaaaaacccggctccaccgtgaagattagttgtaaagcaagcggctatacatttaccgatttctacatccagtggctgaggcaggcaccagtgaagggcctggagtggatgggcctggtggaccccgaggatggacagacaaggtaccctcagagattccagggccgggtgaccatgacagccgatccaagcaccgacacagcctatatggaggtgtccggcctggactttgaggataccgccgactactattgcgccacctctacactgctgggcggcccatatgacaatagcggacacgacgccttcgacatctgggggcaggggactaaagtgattgtgagtagcgccagcacaaagggcccatccgtgtttccactggcaccctcctctaagagcacctccggcggcacagccgccctgggctgtctggtgaaggattacttcccagagccagtgaccgtgagctggaactccggcgccctgacctccggagtgcacacatttccagccgtgctgcagagctccggcctgtatagcctgtctagcgtggtgaccgtgccctcctctagcctgggcacccagacatacatctgcaacgtgaatcacaagccatccaatacaaaggtggacaagaaggtggagcccaagtcttgtgataagacccacacatgccctccctgtcctgcaccagagctgctgggcggcccatccgtgttcctgtttccacccaagcctaaggacaccctgatgatcagccggaccccagaggtgacatgcgtggtggtggacgtgtcccacgaggaccccgaggtgaagttcaactggtacgtggatggcgtggaggtgcacaatgccaagaccaagcctagggaggagcagtataactctacctacagagtggtgagcgtgctgacagtgctgcaccaggactggctgaacggcaaggagtacaagtgcaaggtgtctaataaggccctgcccgcccctatcgagaagaccatcagcaaggcaaagggacagcctcgggagccacaggtgtatacactgcctccatctcgcgacgagctgaccaagaaccaggtgagcctgacatgtctggtgaagggcttctaccctagcgatatcgccgtggagtgggagtccaatggccagccagagaacaattataagaccacaccccctgtgctggactctgatggcagcttctttctgtactccaagctgaccgtggataagtctcgctggcagcagggcaacgtgttcagctgtagcgtgatgcacgaagccctgcacaaccactacacccagaaaagcctgtcactgagccccggaaaa

SEQ ID NO: 133 - protein, 236 aa, synthetic construct
  REGION 1..236: note = 39F Lambda Light Chain
MAWTPLFLFLLTCCPGGSNSQLPVLTQPPSASGTPGQRVTISCSGSGSSFERNYVYWYQQLPGMAPKLLIYRSDQRLSGVPDRFSGSKSDTSASLAISGLRSEDEATYYCAAWDDSLTLLFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 134 - DNA, 708 bp, synthetic construct
  misc_feature 1..708: note = 39F Lambda Light Chain
  source 1..708: mol_type = other DNA
atggcttggacccctctgtttctgtttctgctgacctgttgccccggaggaagtaattcccagctgcccgtcctgactcagcctcctagcgcaagcggcactcctggacagagggtgaccattagttgtagcggaagcggaagtagtttcgagaggaactacgtgtattggtaccagcagctgcccggcatggcccctaagctgctgatctataggtccgaccagagactgtctggcgtgcccgatcggtttagcggctccaagtctgacaccagcgcctccctggcaatctctggcctgcgcagcgaggatgaggccacatactattgcgctgcttgggatgactctctgacactgctgttcgggggagggacaaaactgactgtgctgggacagcctaaggcagcaccatccgtgacactgtttcccccttcctctgaggagctgcaggccaacaaggccaccctggtgtgcctgatcagcgacttctacccaggagcagtgaccgtggcatggaaggcagatagctccccagtgaaggcaggagtggagacaacaaccccttctaagcagagcaacaataagtacgccgcctctagctatctgtctctgacccccgagcagtggaagagccacaagagctattcctgccaggtcacccatgaaggctcaaccgtggagaaaacagtcgcccctaccgaatgctca

SEQ ID NO: 135 - DNA, 2142 bp, synthetic construct
  misc_feature 1..2142: note = Subtype A consensus Envelope DNA sequence construct
  source 1..2142: mol_type = other DNA
ggatccatggactggacctggattctgttcctggtggccgccgccaccagagtgcacagcagagtgatgggcatccagcggaattgccagcacctgtggagatggggcaccatgatcctgggcatgatcatcatctgctctgccgccgagaacctgtgggtgaccgtgtactacggcgtgcctgtgtggaaggacgccgagaccaccctgttctgcgccagcgacgccaaggcctacgataccgaagtgcacaatgtgtgggccacccacgcctgcgtgcctaccgatcccaacccccaggagatcaacctggagaacgtgaccgaggagttcaacatgtggaagaacaacatggtggagcagatgcacaccgacatcatcagcctgtgggaccagagcctgaagccttgcgtgaagctgacccctctgtgcgtgaccctgaactgcagcaacgtgaacgtgaccaccaacatcatgaagggcgagatcaagaactgcagcttcaacatgaccaccgagctgcgggacaagaagcagaaagtgtacagcctgttctacaagctggacgtggtgcagatcaacaagagcaacagcagcagccagtaccggctgatcaactgcaacaccagcgccatcacccaggcctgccccaaagtgagcttcgagcccatccccatccactactgcgcccctgccggcttcgccatcctgaagtgcaaggacaaggagtttaacggcaccggcccctgcaagaatgtgagcaccgtgcagtgcacccacggcatcaagcccgtggtgtccacccagctgctgctgaacggcagcctggccgaggaggaagtgatgatccggagcgagaacatcaccaacaacgccaagaacatcatcgtgcagctgaccaagcccgtgaagatcaattgcacccggcccaacaacaacacccggaagagcatcagaatcggccctggccaggccttctacgccaccggcgacatcatcggcgatatcaggcaggcccactgcaatgtgagccggaccgagtggaacgagaccctgcagaaagtggccaagcagctgcggaagtacttcaacaacaagaccatcatcttcaccaacagcagcggcggcagactgagaatcaccacccacagcttcaattgtggcggcgagttcttctactgcaatacctccggcctgttcaacagcacctggaacggcaacggcaccaagaagaagaacagcaccgagagcaacgacaccatcaccctgccctgccggatcaagcagatcatcaatatgtggcagagggtgggccaggccatgtacgcccctcccatccagggcgtgatcagatgcgagagcaacatcaccggcctgctgctgaccagagatggcggcgacaacaacagcaagaacgagaccttcagacctggcggcggagacatgagggacaactggcggagcgagctgtacaagtacaaagtggtgaagatcgagcccctgggcgtggcccccaccaaggccaagagaagagtggtggagcgggagaagagagctgtgggcatcggcgccgtgttcctgggcttcctgggagccgccggaagcaccatgggagccgccagcatcaccctgaccgtgcaggccagacagctgctgagcggcattgtgcagcagcagagcaacctgctgagagccatcgaggcccagcagcacctgctgaagctgacagtgtggggcatcaaacagctgcaggcccgcgtgctggccgtggagagatacctgaaggaccagcagctgctgggcatctggggctgcagcggcaagctgatctgcaccaccaacgtgccctggaatagcagctggagcaacaagagccagagcgagatctgggacaacatgacctggctgcagtgggacaaggagatcagcaactacaccgatatcatctacaacctgatcgaggagagccagaaccagcaggagaagaacgagcaggatctgctggccctggacaagtgggccaacctgtggaactggttcgacatcagcaactggctgtggtacatcaagatcttcatcatgattgtgggcggcctgatcggcctgagaatcgtgttcgccgtgctgtctgtgtgactcgag

SEQ ID NO: 136 - protein, 709 aa, synthetic construct
  REGION 1..709: note = Subtype A consensus Envelope protein sequence construct
MDWTWILFLVAAATRVHSRVMGIQRNCQHLWRWGTMILGMIIICSAAENLWVTVYYGVPVWKDAETTLFCASDAKAYDTEVHNVWATHACVPTDPNPQEINLENVTEEFNMWKNNMVEQMHTDIISLWDQSLKPCVKLTPLCVTLNCSNVNVTTNIMKGEIKNCSFNMTTELRDKKQKVYSLFYKLDVVQINKSNSSSQYRLINCNTSAITQACPKVSFEPIPIHYCAPAGFAILKCKDKEFNGTGPCKNVSTVQCTHGIKPVVSTQLLLNGSLAEEEVMIRSENITNNAKNIIVQLTKPVKINCTRPNNNTRKSIRIGPGQAFYATGDIIGDIRQAHCNVSRTEWNETLQKVAKQLRKYFNNKTIIFTNSSGGRLRITTHSFNCGGEFFYCNTSGLFNSTWNGNGTKKKNSTESNDTITLPCRIKQIINMWQRVGQAMYAPPIQGVIRCESNITGLLLTRDGGDNNSKNETFRPGGGDMRDNWRSELYKYKVVKIEPLGVAPTKAKRRVVEREKRAVGIGAVFLGFLGAAGSTMGAASITLTVQARQLLSGIVQQQSNLLRAIEAQQHLLKLTVWGIKQLQARVLAVERYLKDQQLLGIWGCSGKLICTTNVPWNSSWSNKSQSEIWDNMTWLQWDKEISNYTDIIYNLIEESQNQQEKNEQDLLALDKWANLWNWFDISNWLWYIKIFIMIVGGLIGLRIVFAVLSV

SEQ ID NO: 137 - DNA, 2734 bp, synthetic construct
  misc_feature 1..2734: note = Subtype B consensus Envelope DNA sequence construct
  source 1..2734: mol_type = other DNA
ggatccgccaccatggactggacctggattctgttcctggtggccgccgccaccagagtgcacagcagagtgaagggcatccggaagaactaccagcacctgtggagatggggcaccatgctgctgggcatgctgatgatctgttctgccgccgagaagctgtgggtgaccgtgtactacggcgtgcctgtgtggaaggaggccaccaccaccctgttctgcgccagcgacgccaaggcctacgataccgaagtgcacaatgtgtgggccacccacgcctgcgtgcctaccgatcccaaccctcaggaagtggtgctggagaacgtgaccgagaacttcaacatgtggaagaacaacatggtggagcagatgcacgaggacatcatcagcctgtgggaccagagcctgaagccttgcgtgaagctgacccctctgtgcgtgaccctgaactgcaccgacctgagcggcgagaagatggagaagggcgagatcaagaactgcagcttcaacatcaccacctccatccgggacaaagtgcagaaggagtacgccctgttctacaagctggacgtggtgcccatcgacaacgacaacaccagctaccggctgatcagctgcaacaccagcgtgatcacccaggcctgccccaaagtgagcttcgagcccatccccatccactactgcgcccctgccggcttcgccatcctgaagtgcaacgacaagaagttcaacggcaccggcccttgcaccaatgtgagcaccgtgcagtgcacccacggcatcagacccgtggtgtccacccagctgctgctgaacggcagcctggccgaggaagaagtggtgatccggagcgagaatttcaccaacaacgccaagaccatcatcgtgcagctgaacgagagcgtggagatcaactgcacccggcccaacaacaatacccggaagagcatccacatcggccctggccaggccttctacaccaccggcgagatcatcggcgatatcaggcaggcccactgcaatatcagccgggccaagtggaacaacaccctgaagcagatcgtgaagaagctgcgggagcagttcggcaacaagaccatcgtgttcaaccagagcagcggcggcagacctagaatcgtgatgcacagcttcaactgtggcggcgagttcttctactgcaacacaacccagctgttcaacagcacctggaacgtgaacgggacctggaacaacaacaccgagggcaacgacaccatcaccctgccctgccggatcaagcagatcatcaatatgtggcaggaggtgggcaaggccatgtacgcccctcccatcagaggccagatccggtgcagcagcaatatcaccggcctgctgctgaccagagatggcggcaacaataacaccaacgagaccgagatctttagacctggcggcggagacatgagggacaactggcggagcgagctgtacaagtacaaagtggtgaagatcgagcccctgggcgtggcccccaccaaggccaagagaagagtggtgcagcgggagaagagagctgtgggcatcggcgccatgtttctgggctttctgggagccgccggaagcaccatgggagccgccagcatgaccctgaccgtgcaggccagacagctgctgagcggcatcgtgcagcagcagaacaacctgctgagagccatcgaggcccagcagcacctgctgcagctgacagtgtggggcatcaagcagctgcaggcccgcgtgctggccgtggagagatacctgaaggaccagcagctgctgggaatctggggctgcagcggcaagctgatctgcaccaccaccgtgccctggaacgccagctggagcaacaagagcctggacgagatctgggacaacatgacctggatggagtgggagcgggagatcgacaactacaccagcctgatctacaccctgatcgaggagagccagaaccagcaggagaagaacgagcaggagctgctggagctggacaagtgggccagcctgtggaactggttcgacatcaccaactggctgtggtacatcaagatcttcatcatgattgtgggcggcctgatcggcctgagaatcgtgttcgccgtgctgagcatctacccctacgacgtgcccgattacgcctgagaattcgtaagtaagtgtcatatgggagagctcgactagactggacagccaatgacgggtaagagagtgacatttctcactaacctaagacaggagggccgtcaaagctactgcctaatccaatgacgggtaatagtgacaagaaatgtatcactccaacctaagacaggcgcagcctccgagggatgtgtcttttgttttttataattaaaaagggtgacatgtccggagccgtgctgcccggatgatgtcttggcctctgtttgctaccggtatcgatgttaacgtcgaccccgggctcgaggtaagtaagtgtcatatgggagagctcgactagactggacagccaatgacgggtaagagagtgacatttctcactaacctaagacaggagggccgtcaaagctactgcctaatccaatgacgggtaatagtgacaagaaatgtatcactccaacctaagacaggcgcagcctccgagggatgtgtcttttgttttttataattaaaaagggtgacatgtccggagccgtgctgcccggatgatgtcttggcctctgtttgctgcggccgc

SEQ ID NO: 138 - protein, 715 aa, synthetic construct
  REGION 1..715: note = Subtype B consensus Envelope protein sequence construct
MDWTWILFLVAAATRVHSRVKGIRKNYQHLWRWGTMLLGMLMICSAAEKLWVTVYYGVPVWKEATTTLFCASDAKAYDTEVHNVWATHACVPTDPNPQEVVLENVTENFNMWKNNMVEQMHEDIISLWDQSLKPCVKLTPLCVTLNCTDLSGEKMEKGEIKNCSFNITTSIRDKVQKEYALFYKLDVVPIDNDNTSYRLISCNTSVITQACPKVSFEPIPIHYCAPAGFAILKCNDKKFNGTGPCTNVSTVQCTHGIRPVVSTQLLLNGSLAEEEVVIRSENFTNNAKTIIVQLNESVEINCTRPNNNTRKSIHIGPGQAFYTTGEIIGDIRQAHCNISRAKWNNTLKQIVKKLREQFGNKTIVFNQSSGGRPRIVMHSFNCGGEFFYCNTTQLFNSTWNVNGTWNNNTEGNDTITLPCRIKQIINMWQEVGKAMYAPPIRGQIRCSSNITGLLLTRDGGNNNTNETEIFRPGGGDMRDNWRSELYKYKVVKIEPLGVAPTKAKRRVVQREKRAVGIGAMFLGFLGAAGSTMGAASMTLTVQARQLLSGIVQQQNNLLRAIEAQQHLLQLTVWGIKQLQARVLAVERYLKDQQLLGIWGCSGKLICTTTVPWNASWSNKSLDEIWDNMTWMEWEREIDNYTSLIYTLIEESQNQQEKNEQELLELDKWASLWNWFDITNWLWYIKIFIMIVGGLIGLRIVFAVLSIYPYDVPDYA

SEQ ID NO: 139 - DNA, 2140 bp, synthetic construct
  misc_feature 1..2140: note = Subtype C consensus Envelope DNA sequence construct
  source 1..2140: mol_type = other DNA
ggatccgccaccatggattggacctggattctgttcctggtggccgccgccacaagagtgcacagcagagtgcggggcatcctgagaaattgccagcagtggtggatctggggcattctggggttctggatgctgatgatctgcaacgtgatgggcaacctgtgggtgaccgtgtactacggcgtgcctgtgtggaaggaggccaagaccaccctgttctgtgccagcgatgccaaggcctacgagaccgaggtgcacaatgtgtgggccacccacgcctgtgtgcccaccgatcccaaccctcaggagatggtgctggagaacgtgaccgagaacttcaacatgtggaagaacgacatggtggaccagatgcacgaggacatcatcagcctgtgggaccagagcctgaagccttgcgtgaagctgacccctctgtgcgtgaccctgaactgccggaacaacgtgaacaacaacaacaccatgaaggaggagatcaagaactgcagcttcaacatcaccaccgagctgcgggacaagaagcagaaggtgtacgccctgttctaccggctggacatcgtgcccctgaacgagaagaacaacagcaacgactaccggctgatcaactgcaacaccagcgccatcacccaggcctgtcccaaggtgtccttcgaccccatccccatccactattgtgcccctgccggctacgccatcctgaagtgcaacaacaagaccttcaacggcaccggcccctgcaataatgtgagcaccgtgcagtgtacccacggcatcaagcctgtggtgtccacccagctgctgctgaatggcagcctggccgaggaggagattatcatccggagcgagaacctgaccaacaacgccaagaccatcattgtgcacctgaatgagagcgtggagatcgtgtgtacccggcccaacaacaatacccggaagagcatcagaatcggccctggccagaccttttacgccaccggcgacatcatcggcgatatcaggcaggcccactgcaatatcagcgaggagaagtggaacaagaccctgcagcgggtgtccgagaagctgaaggagcacttccccaataagaccatcaagttcgcccctagcagcggcggcagactggagatcaccacccacagcttcaactgcaggggcgagttcttctactgcaataccagcaagctgttcaacagcacctacatgcccaacagcaccaacaataccaacaccaccatcaccctgccctgccggatcaagcagatcatcaatatgtggcaggaagtgggcagagccatgtacgcccctcccatcgagggcaacatcacctgcaagtccaacatcaccggcctgctgctgacaagagatggcggcaagaacgacaccaatgacaccgagaccttcagacctggcggcggagacatgagggacaactggcggagcgagctgtacaagtacaaggtggtggagatcaagcctctgggcgtggcccctaccaaggccaagaggagagtggtggagagggagaagagagccgtgggcatcggcgccgtgtttctgggctttctgggagccgccggatctacaatgggagccgccagcatcacactgaccgtgcaggccagacagctgctgagcggcatcgtgcagcagcagagcaatctgctgagagccatcgaggcccagcagcacatgctgcagctgacagtgtggggcatcaagcagctgcagaccagagtgctggccatcgagcgctacctgaaggatcagcagctgctgggcatctggggctgtagcggcaagctgatctgtaccaccgccgtgccttggaatagcagctggagcaacaagagccaggaggacatctgggacaacatgacctggatgcagtgggaccgggagatcagcaactacaccgacaccatctacaggctgctggaggacagccagaaccagcaggagaagaacgagaaggacctgctggccctggacagctggaagaacctgtggaactggttcgacatcaccaactggctgtggtacatcaagatcttcatcatgattgtgggcggcctgatcggcctgagaatcatcttcgccgtgctgagcatctgatagcggccgc

SEQ ID NO: 140 - protein, 705 aa, synthetic construct
  REGION 1..705: note = Subtype C consensus Envelope protein sequence construct
MDWTWILFLVAAATRVHSRVRGILRNCQQWWIWGILGFWMLMICNVMGNLWVTVYYGVPVWKEAKTTLFCASDAKAYETEVHNVWATHACVPTDPNPQEMVLENVTENFNMWKNDMVDQMHEDIISLWDQSLKPCVKLTPLCVTLNCRNNVNNNNTMKEEIKNCSFNITTELRDKKQKVYALFYRLDIVPLNEKNNSNDYRLINCNTSAITQACPKVSFDPIPIHYCAPAGYAILKCNNKTFNGTGPCNNVSTVQCTHGIKPVVSTQLLLNGSLAEEEIIIRSENLTNNAKTIIVHLNESVEIVCTRPNNNTRKSIRIGPGQTFYATGDIIGDIRQAHCNISEEKWNKTLQRVSEKLKEHFPNKTIKFAPSSGGRLEITTHSFNCRGEFFYCNTSKLFNSTYMPNSTNNTNTTITLPCRIKQIINMWQEVGRAMYAPPIEGNITCKSNITGLLLTRDGGKNDTNDTETFRPGGGDMRDNWRSELYKYKVVEIKPLGVAPTKAKRRVVEREKRAVGIGAVFLGFLGAAGSTMGAASITLTVQARQLLSGIVQQQSNLLRAIEAQQHMLQLTVWGIKQLQTRVLAIERYLKDQQLLGIWGCSGKLICTTAVPWNSSWSNKSQEDIWDNMTWMQWDREISNYTDTIYRLLEDSQNQQEKNEKDLLALDSWKNLWNWFDITNWLWYIKIFIMIVGGLIGLRIIFAVLSI

SEQ ID NO: 141 - DNA, 2089 bp, synthetic construct
  misc_feature 1..2089: note = Subtype D consensus Envelope DNA sequence construct
  source 1..2089: mol_type = other DNA
gggcatcaagcggaattaccagcacctgtggaagtggggcaccatgctgctgggcatgctgatgacctgcagcgtggccgagaacctgtgggtgaccgtgtactacggcgtgcctgtgtggaaggaagccaccaccaccctgttctgcgccagcgatgccaagagctacaagaccgaggcccacaatatctgggccacccacgcctgcgtgcctaccgatcccaaccctcaggagatcgagctggagaacgtgaccgagaacttcaacatgtggaagaacaacatggtggagcagatgcacgaggacatcatcagcctgtgggaccagagcctgaagccttgcgtgaagctgacccctctgtgcgtgaccctgaactgcaccgacggcatgaggaacgacaccaacgataccaacgtgaccatggaggagggcgagatgaagaactgcagcttcaacatcaccaccgaagtgcgggacaagaagaagcaggtgcacgccctgttctacaagctggacgtggtgcccatcgacgacaacaacaccaacaacagcaactaccggctgatcaactgcaacaccagcgccatcacccaggcctgccccaaagtgaccttcgagcccatccccatccactactgcgcccctgccggcttcgccatcctgaagtgcaaggataagaagttcaacggcaccggcccctgcaagaatgtgagcaccgtgcagtgcacccacggcatcagacccgtggtgtccacccagctgctgctgaacggcagcctggccgaggaggagatcatcatccggagcgagaacctgaccaacaacgccaagatcatcattgtgcagctgaacgagagcgtgaccatcaattgcacccggccctacaacaatacccggaagcgcatccccatcggcctgggccaggccttctacaccaccagaggcatcatcggcgacatcagacaggcccactgcaatatcagcggagccgagtggaataagaccctgcagcaggtggccaagaagctgggcgacctgctgaacaagaccaccatcatcttcaagcctagcagcggcggcagacctagaatcaccacccacagcttcaattgtggcggcgagttcttctactgcaataccagccggctgttcaacagcacctggagcaagaacagcaccagcaactccaccaaggagaacaacaccatcaccctgccctgccggatcaagcagatcatcaatatgtggcagggagtgggcaaggccatgtacgcccctcccatcgagggcctgatcaagtgcagcagcaacatcaccggcctgctgctgaccagagatggcggagccaacaactcccacaacgagaccttcagacctggcggcggagacatgagggacaactggcggagcgagctgtacaagtacaaagtggtgaagatcgagcccctgggcgtggcccccaccagagccaagagaagagtggtggagcgggagaagagagccatcggactgggcgccatgttcctgggcttcctgggagccgccggaagcaccatgggagccgccagcctgaccctgaccgtgcaggccagacagctgctgagcggcatcgtgcagcagcagaacaacctgctgagagccattgaggcccagcagcacctgctgcagctgacagtgtggggcattaagcagctgcaggccaggattctggccgtggagcgctacctgaaggatcagcagctgctgggaatctggggctgcagcggcaagcacatctgcaccaccaccgtgccttggaatagcagctggagcaacaagagcctggacgagatctggaacaacatgacctggatggagtgggagagggagatcgacaactacaccggcctgatctacagcctgatcgaggagagccagacccagcaggagaagaacgagcaggagctgctggagctggacaagtgggccagcctgtggaactggttcagcatcacccagtggctgtggtacatcaagatcttcatcatgattgtgggcggcctgatcggcctgagaatcgtgttcgccgtgctgagcctgtgactcgag

SEQ ID NO: 142 - protein, 714 aa, synthetic construct
  REGION 1..714: note = Subtype D consensus Envelope protein sequence construct
MDWTWILFLVAAATRVHSRVRGIKRNYQHLWKWGTMLLGMLMTCSVAENLWVTVYYGVPVWKEATTTLFCASDAKSYKTEAHNIWATHACVPTDPNPQEIELENVTENFNMWKNNMVEQMHEDIISLWDQSLKPCVKLTPLCVTLNCTDGMRNDTNDTNVTMEEGEMKNCSFNITTEVRDKKKQVHALFYKLDVVPIDDNNTNNSNYRLINCNTSAITQACPKVTFEPIPIHYCAPAGFAILKCKDKKFNGTGPCKNVSTVQCTHGIRPVVSTQLLLNGSLAEEEIIIRSENLTNNAKIIIVQLNESVTINCTRPYNNTRKRIPIGLGQAFYTTRGIIGDIRQAHCNISGAEWNKTLQQVAKKLGDLLNKTTIIFKPSSGGRPRITTHSFNCGGEFFYCNTSRLFNSTWSKNSTSNSTKENNTITLPCRIKQIINMWQGVGKAMYAPPIEGLIKCSSNITGLLLTRDGGANNSHNETFRPGGGDMRDNWRSELYKYKVVKIEPLGVAPTRAKRRVVEREKRAIGLGAMFLGFLGAAGSTMGAASLTLTVQARQLLSGIVQQQNNLLRAIEAQQHLLQLTVWGIKQLQARILAVERYLKDQQLLGIWGCSGKHICTTTVPWNSSWSNKSLDEIWNNMTWMEWEREIDNYTGLIYSLIEESQTQQEKNEQELLELDKWASLWNWFSITQWLWYIKIFIMIVGGLIGLRIVFAVLSL

SEQ ID NO: 143 - DNA, 1049 bp, synthetic construct
  misc_feature 1..1049: note = Subtype B consensus Nef-Rev DNA sequence construct
  source 1..1049: mol_type = other DNA
ggatccgccaccatggactggacctggattctgttcctggtggccgctgccaccagagtgcacagcagcaagagaagcgtggtgggttggcctacagtgcgggagaggatgagaagagccgagcctgccgccgatggagtgggcgccgtgtctagagatctggagaagcacggcgccatcaccagcagcaataccgccgccaacaatgccgactgcgcctggctggaggcccaggaggaggaggaagtgggcttccctgtgagagcccaggtggccctgagagccatgacctacaaggccgccgtggatctgagccacttcctgaaggagaagggcggcctggagggcctgatctacagccagaagcggcaggacatcctggatctgtgggtgtaccacacccagggctacttccccgactggcagaattacacccctggccctggcatcagataccctctgaccttcggctggtgcttcaagctggtgcctgtggagcctgagaaagtggaggaggccaacgagggcgagaacaattctgccgcccaccctatgagcctgcacggcatggacgatcccgagagggaagtgctggtgtggaagttcgacagcaggctggccttccaccacatggccagagagctgcaccccgagtactacaaggactgccggggcaggaagagaagaagcgccggcagaagcggcgacagcgacgaggagctgctgaaaacagtgcggctgatcaagttcctgtaccagagcaaccctcctcccagccccgagggcaccagacaggcccggagaaaccggaggaggcggtggagagagaggcagcggcagatcagaagcatcagcgagtggattctgagcacctacctgggcagacccgccgagcccgtgcccctgcagctgccccccctggagagactgaccctggactgcaacgaggactgcggcaccagcggcacccagggagtgggcagcccccagatcctggtggagagccctgccgtgctggagagcggcaccaaggagtgatgagcggccgc

SEQ ID NO: 144 - protein, 341 aa, synthetic construct
  REGION 1..341: note = Subtype B consensus Nef-Rev protein sequence construct
MDWTWILFLVAAATRVHSSKRSVVGWPTVRERMRRAEPAADGVGAVSRDLEKHGAITSSNTAANNADCAWLEAQEEEEVGFPVRAQVALRAMTYKAAVDLSHFLKEKGGLEGLIYSQKRQDILDLWVYHTQGYFPDWQNYTPGPGIRYPLTFGWCFKLVPVEPEKVEEANEGENNSAAHPMSLHGMDDPEREVLVWKFDSRLAFHHMARELHPEYYKDCRGRKRRSAGRSGDSDEELLKTVRLIKFLYQSNPPPSPEGTRQARRNRRRRWRERQRQIRSISEWILSTYLGRPAEPVPLQLPPLERLTLDCNEDCGTSGTQGVGSPQILVESPAVLESGTKE

SEQ ID NO: 145 - DNA, 1863 bp, synthetic construct
  misc_feature 1..1863: note = Gag consensus DNA sequence of subtype A, B, C and D construct
  source 1..1863: mol_type = other DNA
ggatccgccaccatggactggacctggattctgtttctggtcgccgccgccacaagagtgcacagcggcgccagagccagcgtgctgtccggcggcaagctggacgcctgggagaagatcagactgaggcctggcggcaagaagaagtaccggctgaagcaccttgtgtgggccagcagagagctggagagattcgccctgaatcctggcctgctggagaccagcgagggctgtaagcagatcatcggccagctgcagcccgccctgcagaccggcagcgaggagctgagaagcctgtacaacaccgtggccaccctgtactgcgtgcacgagaagatcgaggtgaaggacaccaaggaggccctggacaagatcgaggaggagcagaacaagagcaagcagaaggcccagcaggccgccgccgacaccggcaacagcagccaggtgtcccagaactaccccatcgtgcagaatctgcagggccagatggtgcaccaggccatcagccccagaaccctgaatgcctgggtgaaggtgatcgaggagaaggccttcagccctgaggtgatccctatgttcagcgccctgagcgagggcgccacacctcaggacctgaacaccatgctgaacacagtggggggccaccaggccgccatgcagatgctgaaggataccatcaacgaggaggccgccgagtgggacagactgcaccccgtgcacgccggacctatcgcccctggccagatgagagagcccagaggcagcgacatcgccggcaccacctccaccctgcaagagcagatcggctggatgaccagcaacccccccatccctgtgggcgacatctacaagcggtggatcatcctgggcctgaacaagattgtgaggatgtacagccccgtgtccatcctggatatcaggcagggccccaaggagcccttcagagactacgtggaccggttcttcaagaccctgagagccgagcaggccagccaggacgtgaagaactggatgaccgagaccctgctggtgcagaacgccaaccccgactgtaagaccatcctgagagccctgggccctggcgccaccctggaggagatgatgaccgcctgccagggagtgggcggacccggccacaaggccagagtgctggccgaggccatgagccaggccaccaacagcaacatcatgatgcagcggggcaacttcagaggccccaggaggatcgtgaagtgcttcaactgtggcaaggagggccacatcgccagaaactgtagggcccccaggaagaagggctgctggaagtgtggcaaagaggggcaccagatgaaggactgtaccgagcggcaggccaatttcctggggaagatctggcccagccacaagggcagacccggcaatttcctgcagagcagacctgagcccaccgcccctcccgccgagagcttcggcttcggcgaggagatcacccccagccccaagcaggagcccaaggacagagagctgtaccctctggccagcctgaagagcctgttcggcaacgatcccctgagccagtacccctacgacgtgcccgattacgcctgagaattcgtaagtaagtgtcatatgggagagctcgactagactggacagccaatgacgggtaagagagtgacatttctcactaacctaagacaggagggccgtcaaagctactgcctaatccaatgacgggtaatagtgacaagaaatgtatcactccaacctaagacaggcgcagcctccgagggatgtgtcttttgttttttataattaaaaagggtgacatgtccggagccgtgctgcccggatgatgtcttggcctctgtttgctgcggccgc

SEQ ID NO: 146 - protein, 524 aa, synthetic construct
  REGION 1..524: note = Gag consensus protein sequence of subtype A, B, C and D construct
MDWTWILFLVAAATRVHSGARASVLSGGKLDAWEKIRLRPGGKKKYRLKHLVWASRELERFALNPGLLETSEGCKQIIGQLQPALQTGSEELRSLYNTVATLYCVHEKIEVKDTKEALDKIEEEQNKSKQKAQQAAADTGNSSQVSQNYPIVQNLQGQMVHQAISPRTLNAWVKVIEEKAFSPEVIPMFSALSEGATPQDLNTMLNTVGGHQAAMQMLKDTINEEAAEWDRLHPVHAGPIAPGQMREPRGSDIAGTTSTLQEQIGWMTSNPPIPVGDIYKRWIILGLNKIVRMYSPVSILDIRQGPKEPFRDYVDRFFKTLRAEQASQDVKNWMTETLLVQNANPDCKTILRALGPGATLEEMMTACQGVGGPGHKARVLAEAMSQATNSNIMMQRGNFRGPRRIVKCFNCGKEGHIARNCRAPRKKGCWKCGKEGHQMKDCTERQANFLGKIWPSHKGRPGNFLQSRPEPTAPPAESFGFGEEITPSPKQEPKDRELYPLASLKSLFGNDPLSQYPYDVPDYA

SEQ ID NO: 147 - protein, 692 aa, synthetic construct
  REGION 1..692: note = Subtype A consensus Envelope protein sequence
SRVMGIQRNCQHLWRWGTMILGMIIICSAAENLWVTVYYGVPVWKDAETTLFCASDAKAYDTEVHNVWATHACVPTDPNPQEINLENVTEEFNMWKNNMVEQMHTDIISLWDQSLKPCVKLTPLCVTLNCSNVNVTTNIMKGEIKNCSFNMTTELRDKKQKVYSLFYKLDVVQINKSNSSSQYRLINCNTSAITQACPKVSFEPIPIHYCAPAGFAILKCKDKEFNGTGPCKNVSTVQCTHGIKPVVSTQLLLNGSLAEEEVMIRSENITNNAKNIIVQLTKPVKINCTRPNNNTRKSIRIGPGQAFYATGDIIGDIRQAHCNVSRTEWNETLQKVAKQLRKYFNNKTIIFTNSSGGRLRITTHSFNCGGEFFYCNTSGLFNSTWNGNGTKKKNSTESNDTITLPCRIKQIINMWQRVGQAMYAPPIQGVIRCESNITGLLLTRDGGDNNSKNETFRPGGGDMRDNWRSELYKYKVVKIEPLGVAPTKAKRRVVEREKRAVGIGAVFLGFLGAAGSTMGAASITLTVQARQLLSGIVQQQSNLLRAIEAQQHLLKLTVWGIKQLQARVLAVERYLKDQQLLGIWGCSGKLICTTNVPWNSSWSNKSQSEIWDNMTWLQWDKEISNYTDIIYNLIEESQNQQEKNEQDLLALDKWANLWNWFDISNWLWYIKIFIMIVGGLIGLRIVFAVLSV

SEQ ID NO: 148 - protein, 697 aa, synthetic construct
  REGION 1..697: note = Subtype B consensus Envelope protein sequence
RVKGIRKNYQHLWRWGTMLLGMLMICSAAEKLWVTVYYGVPVWKEATTTLFCASDAKAYDTEVHNVWATHACVPTDPNPQEVVLENVTENFNMWKNNMVEQMHEDIISLWDQSLKPCVKLTPLCVTLNCTDLSGEKMEKGEIKNCSFNITTSIRDKVQKEYALFYKLDVVPIDNDNTSYRLISCNTSVITQACPKVSFEPIPIHYCAPAGFAILKCNDKKFNGTGPCTNVSTVQCTHGIRPVVSTQLLLNGSLAEEEVVIRSENFTNNAKTIIVQLNESVEINCTRPNNNTRKSIHIGPGQAFYTTGEIIGDIRQAHCNISRAKWNNTLKQIVKKLREQFGNKTIVFNQSSGGRPRIVMHSFNCGGEFFYCNTTQLFNSTWNVNGTWNNNTEGNDTITLPCRIKQIINMWQEVGKAMYAPPIRGQIRCSSNITGLLLTRDGGNNNTNETEIFRPGGGDMRDNWRSELYKYKVVKIEPLGVAPTKAKRRVVQREKRAVGIGAMFLGFLGAAGSTMGAASMTLTVQARQLLSGIVQQQNNLLRAIEAQQHLLQLTVWGIKQLQARVLAVERYLKDQQLLGIWGCSGKLICTTTVPWNASWSNKSLDEIWDNMTWMEWEREIDNYTSLIYTLIEESQNQQEKNEQELLELDKWASLWNWFDITNWLWYIKIFIMIVGGLIGLRIVFAVLSIYPYDVPDYA

SEQ ID NO: 149 - protein, 687 aa, synthetic construct
  REGION 1..687: note = Subtype C consensus Envelope protein sequence
RVRGILRNCQQWWIWGILGFWMLMICNVMGNLWVTVYYGVPVWKEAKTTLFCASDAKAYETEVHNVWATHACVPTDPNPQEMVLENVTENFNMWKNDMVDQMHEDIISLWDQSLKPCVKLTPLCVTLNCRNNVNNNNTMKEEIKNCSFNITTELRDKKQKVYALFYRLDIVPLNEKNNSNDYRLINCNTSAITQACPKVSFDPIPIHYCAPAGYAILKCNNKTFNGTGPCNNVSTVQCTHGIKPVVSTQLLLNGSLAEEEIIIRSENLTNNAKTIIVHLNESVEIVCTRPNNNTRKSIRIGPGQTFYATGDIIGDIRQAHCNISEEKWNKTLQRVSEKLKEHFPNKTIKFAPSSGGRLEITTHSFNCRGEFFYCNTSKLFNSTYMPNSTNNTNTTITLPCRIKQIINMWQEVGRAMYAPPIEGNITCKSNITGLLLTRDGGKNDTNDTETFRPGGGDMRDNWRSELYKYKVVEIKPLGVAPTKAKRRVVEREKRAVGIGAVFLGFLGAAGSTMGAASITLTVQARQLLSGIVQQQSNLLRAIEAQQHMLQLTVWGIKQLQTRVLAIERYLKDQQLLGIWGCSGKLICTTAVPWNSSWSNKSQEDIWDNMTWMQWDREISNYTDTIYRLLEDSQNQQEKNEKDLLALDSWKNLWNWFDITNWLWYIKIFIMIVGGLIGLRIIFAVLSI

SEQ ID NO: 150 - protein, 696 aa, synthetic construct
  REGION 1..696: note = Subtype D consensus Envelope protein sequence
RVRGIKRNYQHLWKWGTMLLGMLMTCSVAENLWVTVYYGVPVWKEATTTLFCASDAKSYKTEAHNIWATHACVPTDPNPQEIELENVTENFNMWKNNMVEQMHEDIISLWDQSLKPCVKLTPLCVTLNCTDGMRNDTNDTNVTMEEGEMKNCSFNITTEVRDKKKQVHALFYKLDVVPIDDNNTNNSNYRLINCNTSAITQACPKVTFEPIPIHYCAPAGFAILKCKDKKFNGTGPCKNVSTVQCTHGIRPVVSTQLLLNGSLAEEEIIIRSENLTNNAKIIIVQLNESVTINCTRPYNNTRKRIPIGLGQAFYTTRGIIGDIRQAHCNISGAEWNKTLQQVAKKLGDLLNKTTIIFKPSSGGRPRITTHSFNCGGEFFYCNTSRLFNSTWSKNSTSNSTKENNTITLPCRIKQIINMWQGVGKAMYAPPIEGLIKCSSNITGLLLTRDGGANNSHNETFRPGGGDMRDNWRSELYKYKVVKIEPLGVAPTRAKRRVVEREKRAIGLGAMFLGFLGAAGSTMGAASLTLTVQARQLLSGIVQQQNNLLRAIEAQQHLLQLTVWGIKQLQARILAVERYLKDQQLLGIWGCSGKHICTTTVPWNSSWSNKSLDEIWNNMTWMEWEREIDNYTGLIYSLIEESQTQQEKNEQELLELDKWASLWNWFSITQWLWYIKIFIMIVGGLIGLRIVFAVLSL

SEQ ID NO: 151 - protein, 323 aa, synthetic construct
  REGION 1..323: note = Subtype B consensus Nef-Rev protein sequence
SKRSVVGWPTVRERMRRAEPAADGVGAVSRDLEKHGAITSSNTAANNADCAWLEAQEEEEVGFPVRAQVALRAMTYKAAVDLSHFLKEKGGLEGLIYSQKRQDILDLWVYHTQGYFPDWQNYTPGPGIRYPLTFGWCFKLVPVEPEKVEEANEGENNSAAHPMSLHGMDDPEREVLVWKFDSRLAFHHMARELHPEYYKDCRGRKRRSAGRSGDSDEELLKTVRLIKFLYQSNPPPSPEGTRQARRNRRRRWRERQRQIRSISEWILSTYLGRPAEPVPLQLPPLERLTLDCNEDCGTSGTQGVGSPQILVESPAVLESGTKE

SEQ ID NO: 152 - protein, 506 aa, synthetic construct
  REGION 1..506: note = Gag consensus protein sequence of subtype A, B, C and D
GARASVLSGGKLDAWEKIRLRPGGKKKYRLKHLVWASRELERFALNPGLLETSEGCKQIIGQLQPALQTGSEELRSLYNTVATLYCVHEKIEVKDTKEALDKIEEEQNKSKQKAQQAAADTGNSSQVSQNYPIVQNLQGQMVHQAISPRTLNAWVKVIEEKAFSPEVIPMFSALSEGATPQDLNTMLNTVGGHQAAMQMLKDTINEEAAEWDRLHPVHAGPIAPGQMREPRGSDIAGTTSTLQEQIGWMTSNPPIPVGDIYKRWIILGLNKIVRMYSPVSILDIRQGPKEPFRDYVDRFFKTLRAEQASQDVKNWMTETLLVQNANPDCKTILRALGPGATLEEMMTACQGVGGPGHKARVLAEAMSQATNSNIMMQRGNFRGPRRIVKCFNCGKEGHIARNCRAPRKKGCWKCGKEGHQMKDCTERQANFLGKIWPSHKGRPGNFLQSRPEPTAPPAESFGFGEEITPSPKQEPKDRELYPLASLKSLFGNDPLSQYPYDVPDYA

(19) United States
(12) Patent Application Publication
(10) Pub. No.: US 2024/0124559 A1
(43) Pub. Date: Apr. 18, 2024

(54) DNA ANTIBODY CONSTRUCTS FOR USE AGAINST HIV

(71) Applicants: The Wistar Institute of Anatomy and Biology, Philadelphia, PA (US); INOVIO PHARMACEUTICALS, INC., Plymouth Meeting, PA (US)

(72) Inventors: David Weiner, Merion, PA (US); Megan Wise, Glenside, PA (US)

(21) Appl. No.: 18/526,819

(22) Filed: Dec. 1, 2023

Related U.S. Application Data
(63) Continuation of application No. 16/497,945, filed on Sep. 26, 2019, now Pat. No. 11,845,787.
(60) Provisional application No. 62/477,227, filed on Mar. 27, 2017.
(60) Provisional application No. 62/477,217, filed on Mar. 27, 2017.

(51) Int. Cl. C07K 16/10; A61P 31/18

(52) U.S. Cl. CPC C07K 16/1045; A61P 31/18; C07K 2317/55; C07K 2317/76

(57) ABSTRACT

Disclosed herein is a composition including a recombinant nucleic acid sequence that encodes an antibody to an HIV antigen. Also disclosed herein is a method of generating a synthetic antibody in a subject by administering the composition to the subject. The disclosure also provides a method of preventing and/or treating an HIV infection in a subject using said composition and method of generation.

### CROSS REFERENCE TO RELATED APPLICATIONS

This application is entitled to priority to U.S. Provisional Application No. 62/477,217, filed Mar. 27, 2017, U.S. Provisional Application No. 62/477,227, filed Mar. 27, 2017, and each of which is incorporated by reference herein in its entirety.

### TECHNICAL FIELD

The present invention relates to a composition comprising a recombinant nucleic acid sequence for generating one or more synthetic antibodies, and functional fragments thereof, in vivo, and a method of preventing and/or treating viral infection in a subject by administering said composition.

### BACKGROUND

The strength of broadly neutralizing antibodies (bNabs) to protect or control HIV-1 is well established in animal models. Though these antibodies are powerful and can prevent infection or control viral loads, the cost of production could severely limit the ability to use these antibodies in the field. Additionally, current vaccine approaches have failed to induce similar antibodies.

Thus there is need in the art for improved therapeutics that prevent and/or treat HIV infection. The current invention satisfies this need.

### SUMMARY

The present invention is directed to a nucleic acid molecule encoding one or more synthetic antibodies, wherein the nucleic acid molecule comprises at least one selected from the group consisting of a) a nucleotide sequence encoding an anti-HIV synthetic antibody; and b) a nucleotide sequence encoding a fragment of an anti-HIV synthetic antibody.

In one embodiment, the nucleic acid molecule comprises a nucleotide sequence encoding a cleavage domain.

In one embodiment, the nucleic acid molecule comprises a nucleotide sequence encoding one or more of a variable heavy chain region and a variable light chain region of an anti-HIV antibody.

In one embodiment, the nucleic acid molecule comprises a nucleotide sequence encoding one or more amino acid sequences at least 90% identical to an amino acid sequence as set forth in SEQ ID NO:1, SEQ ID NO:3, SEQ ID NO:5, SEQ ID NO:7, SEQ ID NO:9, SEQ ID NO:11, or SEQ ID NO:13, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39 SEQ ID NO:41, SEQ ID NO:43, SEQ ID NO:45, SEQ ID NO:47, SEQ ID NO:49 SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:111, SEQ ID NO:113, SEQ ID NO:115, SEQ ID NO:117, SEQ ID NO:119, SEQ ID NO:121, SEQ ID NO:123, SEQ ID NO:125, SEQ ID NO:127, SEQ ID NO:129, SEQ ID NO:131, or SEQ ID NO:133.

In one embodiment, the nucleic acid molecule comprises a nucleotide sequence at least 95% identical to SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:17, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO: 60, SEQ ID NO:62, SEQ ID NO:64, SEQ ID NO:66, SEQ ID NO:68,SEQ ID NO: 70, SEQ ID NO:72, SEQ ID NO:74, SEQ ID NO:76, SEQ ID NO:78, SEQ ID NO: 80, SEQ ID NO:82, SEQ ID NO:84, SEQ ID NO:86, SEQ ID NO:88, SEQ ID NO: 90, SEQ ID NO:92, SEQ ID NO:94, SEQ ID NO:96, SEQ ID NO:98, SEQ ID NO: 100, SEQ ID NO:102, SEQ ID NO:104, SEQ ID NO:106, SEQ ID NO:108, SEQ ID NO: 110, SEQ ID NO:112, SEQ ID NO:114, SEQ ID NO:116, SEQ ID NO:118, SEQ ID NO: 120, SEQ ID NO:122, SEQ ID NO:124, SEQ ID NO:126, SEQ ID NO:128, SEQ ID NO: 130, SEQ ID NO:132, or SEQ ID NO:134.

In one embodiment, the nucleic acid molecule comprises a nucleotide sequence encoding a variable heavy chain region and a variable light chain region of an anti-HIV antibody.

In one embodiment, the nucleic acid molecule comprises a nucleotide sequence encoding a heavy chain comprising an amino acid sequence at least 95% identical to SEQ ID NO:3, SEQ ID NO:11, SEQ ID NO:17, SEQ ID NO:25, SEQ ID NO:39, SEQ ID NO:47, SEQ ID NO:55, SEQ ID NO:59, SEQ ID NO:63, SEQ ID NO:67, SEQ ID NO:71, SEQ ID NO:75, SEQ ID NO:79, SEQ ID NO:83, SEQ ID NO:87, SEQ ID NO:91, SEQ ID NO:95, SEQ ID NO:99, SEQ ID NO:103, SEQ ID NO:107, SEQ ID NO:111, SEQ ID NO:115, SEQ ID NO:119, SEQ ID NO:123, SEQ ID NO:127, or SEQ ID NO:131.

In one embodiment, the nucleotide sequence encoding a heavy chain comprises a nucleotide sequence at least 95% identical to SEQ ID NO:4, SEQ ID NO:12, SEQ ID NO:18, SEQ ID NO:26, SEQ ID NO:40, SEQ ID NO:48, SEQ ID NO:56, SEQ ID NO:60, SEQ ID NO:64, SEQ ID NO:68, SEQ ID NO:72, SEQ ID NO:76, SEQ ID NO:80, SEQ ID NO:84, SEQ ID NO:88, SEQ ID NO:92, SEQ ID NO:96, SEQ ID NO:100, SEQ ID NO:104, SEQ ID NO:108, SEQ ID NO:112, SEQ ID NO:116, SEQ ID NO:120, SEQ ID NO:124, SEQ ID NO:128, or SEQ ID NO:132.

In one embodiment, the nucleic acid molecule comprises a nucleotide sequence encoding a light chain comprising an amino acid sequence at least 95% identical to SEQ ID NO:5, SEQ ID NO:13, SEQ ID NO:19, SEQ ID NO:27, SEQ ID NO:41, SEQ ID NO:49, SEQ ID NO:57, SEQ ID NO:61, SEQ ID NO:65, SEQ ID NO:69, SEQ ID NO:73, SEQ ID NO:77, SEQ ID NO:81, SEQ ID NO:85, SEQ ID NO:89, SEQ ID NO:93, SEQ ID NO:97, SEQ ID NO:101, SEQ ID NO:105, SEQ ID NO:109, SEQ ID NO:113, SEQ ID NO:117, SEQ ID NO:121, SEQ ID NO:125, SEQ ID NO:129, or SEQ ID NO:133.

In one embodiment, the nucleotide sequence encoding a light chain comprises a nucleotide sequence at least 95% identical to SEQ ID NO:6, SEQ ID NO:14, SEQ ID NO:20, SEQ ID NO:28, SEQ ID NO:42, SEQ ID NO:50, SEQ ID NO:58, SEQ ID NO:62, SEQ ID NO:66, SEQ ID NO:70, SEQ ID NO:74, SEQ ID NO:76, SEQ ID NO:82, SEQ ID NO:86, SEQ ID NO:90, SEQ ID NO:94, SEQ ID NO:98, SEQ ID NO:102, SEQ ID NO:106, SEQ ID NO:110, SEQ ID NO:114, SEQ ID NO:118, SEQ ID NO:122, SEQ ID NO:126, SEQ ID NO:130, or SEQ ID NO:134.

In one embodiment, the nucleic acid molecule comprises a nucleotide sequence encoding a polypeptide comprising a variable heavy chain region; an IRES element; and a variable light chain region. In one embodiment, the IRES element is one of a viral IRES or an eukaryotic IRES.

In one embodiment, the nucleotide sequence encodes a leader sequence. In one embodiment, the nucleic acid molecule comprises an expression vector.

The invention further provides a composition comprising any of the nucleic acid molecules described herein.

In one embodiment the composition comprises a pharmaceutically acceptable excipient.

The invention further relates to a method of preventing or treating a disease in a subject, the method comprising administering to the subject a nucleic acid molecule or a composition as described herein.

In one embodiment, the disease is an HIV infection.

In one embodiment, the method further comprises administering an antibiotic agent to the subject. In one embodiment, an antibiotic is administered less than 10 days after administration of the nucleic acid molecule or composition.

In one embodiment, the method further comprises administering an antibiotic agent to the subject. In one embodiment an antibiotic is administered less than 10 days after administration of the nucleic acid molecule or composition.

The invention further relates to a method generating an immune response in a subject, the method comprising administering to the subject a nucleic acid molecule or a composition as described herein.

### BRIEF DESCRIPTION OF THE DRAWINGS

FIG. 1, comprising FIG. 1A through FIG. 1B, depicts dMAb concept and design. FIG. 1A depicts a schematic of dMAb design. Antibody sequences go through multiple rounds of optimizations to increase dMAb production level including codon and RNA optimization, amino acid sequence modification, changes to the backbone and formulation. FIG. 1B depicts a diagram of dMAb in vivo production. Plasmid DNA expressing the antibody is injected into the muscle followed by electroporation. Muscle cells then product the functional antibody which is secreted into circulation.

FIG. 2, comprising FIG. 2A through FIG. 2C, depicts experimental results demonstrating in vitro expression of HIV IgG1 dMAb constructs. 293T cells were transfected with different HIV dMAb plasmids. Forty-eight hours later cell media or lysate were harvested and quantified via ELISA. FIG. 2A depicts the expression of HIV dMAB constructs in cell media. FIG. 2B depicts the expression of HIV dMAB constructs in cell lysate. FIG. 2C depicts western blot analysis of media from two constructs to ensure proper processing of the heavy and light chain.

FIG. 3, depicts experimental results demonstrating in vivo expression of HIV dMAb constructs. BalbC mice were injected followed by electroporation with HIV dMAb constructs. Both broadly neutralizing antibodies and non-neutralizing antibodies express in mice.

FIG. 4 depicts experimental results demonstrating how two plasmids expressing the heavy and light chain of the IgG1 on separate plasmids can increase expression levels over a single plasmid expressing both the heavy and light chain IgG1 across multiple HIV dMAbs.

FIG. 5, comprising FIG. 5A through FIG. 5C depicts experimental results demonstrating how different human IgG isotypes can lead to changes in in vivo expression levels of dMAbs. FIG. 5A depicts in vitro expression levels of human IgG1, IgG2, IgG3 and IgG4 isotypes for broadly neutralizing antibody VRC01 for both media and lysate. FIG. 5B depicts expression levels of human IgG isotypes for VRC01 dMAbs in immune incompetent mice over the course of more than a month. FIG. 5C depicts expression levels of human IgG isotypes for VRC01 dMAbs in immune competent balbC mice.

FIG. 6, comprising of FIG. 6A through FIG. 6B depicts experimental results demonstrating the in vitro expression levels of multiple broadly neutralizing antibodies expressed as either human IgG1 or human IgG4 dMAbs. FIG. 6A depicts in vitro expression levels in 293T transfected cell media. FIG. 6B depicts in vitro expression levels in 293T transfected cell lysate.

FIG. 7, comprising of FIG. 7A through FIG. 7B, depicts experimental results demonstrating the increased expression levels in vivo for multiple HIV dMAbs using IgG4 isotype compared to IgG1. FIG. 7A depict in vivo expression levels over time for 5 HIV dMAbs, PG9, PGT128, 3BNC117, NIH45-46 and VRC01 comparing balbC mice injected with plasmids expressing either the IgG1 or IgG4 isoform. FIG. 7B, depicts peak expression levels on Day 7.

FIG. 8, comprising of FIG. 8A through FIG. 8B depicts experimental results demonstrating that the S228P mutation in the IgG4 background does not disrupt expression levels. FIG. 8A depicts expression levels of IgG4 and IgG4mut(S228P) in vitro after 293T cells transfection. FIG. 8B depicts expression levels in vivo in balbC mice injected with either IgG4 or IgG4mut(S228P) plasmids for PGT128, VRC01, or 3BNC117.

FIG. 9 depicts experimental results demonstrating that two plasmid system increases in vivo dMAb levels over that of single plasmid IgG4.

FIG. 10, comprising FIG. 10A through FIG. 10B depicts experimental results demonstrating the long terms expression of dMAb and how modifications can increase expression levels over time. FIG. 10A depicts dMAb expression levels in vivo when balb C mice are immunocompromised and injected with plasmid expressing PGT128 on the IgG1 or IgG4 backbone delivered as either 1 or 2 plasmids. FIG. 10B shows the generalization of IgG4 2 plasmid expression across 5 HIV dMAbs.

FIG. 11, comprising FIG. 11A through FIG. 11B depicts experimental results demonstrating dMAb modifications and optimizations maintains functionality. FIG. 11A depicts reported IC50 concentrations for a panel of tier 2 viruses against numerous bNabs. FIG. 11B depicts neutralization concentration (IC50) for original and modified in vitro produced dMAb to induce functionality after modification.

FIG. 12 depicts experimental results demonstrating that in vivo produced IgG4 HIV dMAbs are functional as measured by neutralization capacity in the TzmBl assay. Serum from balbC mice injected with VRC01, 3BNC117, NIH45-46 or PGT128 are able to neutralize multiple pseudotype viruses to similar levels as reported in the literature.

FIG. 13 depicts expression of multiple HIV dMAb in a single mouse. Mice were injected with single plasmid or two plasmids expressing different antibodies. Expression of hIgG was determined on day 7.

### DETAILED DESCRIPTION

The present invention relates to compositions comprising a recombinant nucleic acid sequence encoding an antibody, a fragment thereof, a variant thereof, or a combination thereof. The composition can be administered to a subject in need thereof to facilitate in vivo expression and formation of a synthetic antibody.

In particular, the heavy chain and light chain polypeptides expressed from the recombinant nucleic acid sequences can assemble into the synthetic antibody. The heavy chain polypeptide and the light chain polypeptide can interact with one another such that assembly results in the synthetic antibody being capable of binding the antigen, being more immunogenic as compared to an antibody not assembled as described herein, and being capable of eliciting or inducing an immune response against the antigen.

Additionally, these synthetic antibodies are generated more rapidly in the subject than antibodies that are produced in response to antigen induced immune response. The synthetic antibodies are able to effectively bind and neutralize a range of antigens. The synthetic antibodies are also able to effectively protect against and/or promote survival of disease.

A sequence listing provided herewith contains a list of sequences including the following:
SEQ ID NO: 1 is the amino acid sequence of 3BNC117 human IgG1 antibody.
SEQ ID NO: 2 is the nucleotide sequence of 3BNC117 human IgG1 antibody.
SEQ ID NO: 3 is the amino acid sequence of 3BNC117 human IgG1 antibody (heavy chain).
SEQ ID NO: 4 is the nucleotide sequence of 3BNC117 human IgG1 antibody (heavy chain).
SEQ ID NO: 5 is the amino acid sequence of 3BNC117 human IgG1 antibody (light chain).
SEQ ID NO: 6 is the nucleotide sequence of 3BNC117 human IgG1 antibody (light chain).
SEQ ID NO: 7 is the amino acid sequence of 3BNC117 human IgG4.
SEQ ID NO: 8 is the nucleotide sequence of 3BNC117 human IgG4.
SEQ ID NO: 9 is the amino acid sequence of 3BNC117 human IgG4 (S228P).
SEQ ID NO: 10 is the nucleotide sequence of 3BNC117 human IgG4 (S228P).
SEQ ID NO: 11 is the amino acid sequence of 3BNC117 human IgG4 (heavy chain).
SEQ ID NO: 12 is the nucleotide sequence of 3BNC117 human IgG4 (heavy chain).
SEQ ID NO: 13 is the amino acid sequence of 3BNC117 human IgG4 (light chain).
SEQ ID NO: 14 is the nucleotide sequence of 3BNC117 human IgG4 (light chain).
SEQ ID NO: 15 is the amino acid sequence of PGT128 human IgG1.
SEQ ID NO: 16 is the nucleotide sequence of PGT128 human IgG1.
SEQ ID NO: 17 is the amino acid sequence of PGT128 human IgG1-(heavy chain).
SEQ ID NO: 18 is the nucleotide sequence of PGT128 human IgG1-(heavy chain).
SEQ ID NO: 19 is the amino acid sequence of PGT128 human IgG1-(light chain).
SEQ ID NO: 20 is the nucleotide sequence of PGT128 human IgG1-(light chain).
SEQ ID NO: 21 is the amino acid sequence of PGT128 human IgG4.
SEQ ID NO: 22 is the nucleotide sequence of PGT128 human IgG4.
SEQ ID NO: 23 is the amino acid sequence of PGT128 human IgG4mut (S228P).
SEQ ID NO: 24 is the nucleotide sequence of PGT128 human IgG4mut (S228P).
SEQ ID NO: 25 is the amino acid sequence of PGT128 human IgG4-heavy chain.
SEQ ID NO: 26 is the nucleotide sequence of PGT128 human IgG4-heavy chain.
SEQ ID NO: 27 is the amino acid sequence of PGT128 human IgG4-light chain.
SEQ ID NO: 28 is the nucleotide sequence of PGT128 human IgG4-light chain.
SEQ ID NO: 29 is the amino acid sequence of PG9 IgG1.
SEQ ID NO: 30 is the nucleotide sequence of PG9 IgG1.
SEQ ID NO: 31 is the amino acid sequence of PG9 human IgG4.
SEQ ID NO: 32 is the nucleotide sequence of PG9 human IgG4.
SEQ ID NO: 33 is the amino acid sequence of NIH45-46 human IgG1 antibody.
SEQ ID NO: 34 is the nucleotide sequence of NIH45-46 human IgG1 antibody.
SEQ ID NO: 35 is the amino acid sequence of NIH45-46 human IgG4 antibody.
SEQ ID NO: 36 is the nucleotide sequence of NIH45-46 human IgG4 antibody.
SEQ ID NO: 37 is the amino acid sequence of VRC01 human IgG1.
SEQ ID NO: 38 is the nucleotide sequence of VRC01 human IgG1.
SEQ ID NO: 39 is the amino acid sequence of VRC01 human IgG1 (heavy chain).
SEQ ID NO: 40 is the nucleotide sequence of VRC01 human IgG1 (heavy chain).
SEQ ID NO: 41 is the amino acid sequence of VRC01 human IgG1 (light chain).
SEQ ID NO: 42 is the nucleotide sequence of VRC01 human IgG1 (light chain).
SEQ ID NO: 43 is the amino acid sequence of VRC01 human IgG4.
SEQ ID NO: 44 is the nucleotide sequence of VRC01 human IgG4.
SEQ ID NO: 45 is the amino acid sequence of VRC01 human IgG4 mut (S228P).
SEQ ID NO: 46 is the nucleotide sequence of VRC01 human IgG4 mut (S228P).
SEQ ID NO: 47 is the amino acid sequence of VRC01 human IgG4 (heavy chain).
SEQ ID NO: 48 is the nucleotide sequence of VRC01 human IgG4 (heavy chain).
SEQ ID NO: 49 is the amino acid sequence of VRC01 human IgG4 (light chain).
SEQ ID NO: 50 is the nucleotide sequence of VRC01 human IgG4 (light chain).
SEQ ID NO: 51 is the amino acid sequence of VRC01 human IgG2.
SEQ ID NO: 52 is the nucleotide sequence of VRC01 human IgG2.
SEQ ID NO: 53 is the amino acid sequence of VRC01 human IgG3
SEQ ID NO: 54 is the nucleotide sequence of VRC01 human IgG3.
SEQ ID NO: 55 is the amino acid sequence of PGT145 Heavy Chain
SEQ ID NO: 56 is the nucleotide sequence of PGT145 Heavy Chain
SEQ ID NO: 57 is the amino acid sequence of PGT145 Kappa
SEQ ID NO: 58 is the nucleotide sequence of PGT145 Kappa
SEQ ID NO: 59 is the amino acid sequence of PGT151 Heavy Chain
SEQ ID NO: 60 is the nucleotide sequence of PGT151 Heavy Chain
SEQ ID NO: 61 is the amino acid sequence of PGT151 Kappa
SEQ ID NO: 62 is the nucleotide sequence of PGT151 Kappa
SEQ ID NO: 63 is the amino acid sequence of PGDM1400 Heavy Chain
SEQ ID NO: 64 is the nucleotide sequence of PGDM1400 Heavy Chain
SEQ ID NO: 65 is the amino acid sequence of PGDM1400 Kappa
SEQ ID NO: 66 is the nucleotide sequence of PGDM1400 Kappa
SEQ ID NO: 67 is the amino acid sequence of 12A21 Heavy Chain
SEQ ID NO: 68 is the nucleotide sequence of 12A21 Heavy Chain
SEQ ID NO: 69 is the amino acid sequence of 12A21 Kappa
SEQ ID NO: 70 is the nucleotide sequence of 12A21 Kappa
SEQ ID NO: 71 is the amino acid sequence of VRC3401 Heavy Chain
SEQ ID NO: 72 is the nucleotide sequence of VRC3401 Heavy Chain
SEQ ID NO: 73 is the amino acid sequence of VRC3401 Kappa
SEQ ID NO: 74 is the nucleotide sequence of VRC3401 Kappa
SEQ ID NO: 75 is the amino acid sequence of IOMA Heavy Chain
SEQ ID NO: 76 is the nucleotide sequence of IOMA Heavy Chain
SEQ ID NO: 77 is the amino acid sequence of IOMA Lambda
SEQ ID NO: 78 is the nucleotide sequence of IOMA Lambda
SEQ ID NO: 79 is the amino acid sequence of PGT130 Heavy Chain
SEQ ID NO: 80 is the nucleotide sequence of PGT130 Heavy Chain
SEQ ID NO: 81 is the amino acid sequence of PGT130 Lambda
SEQ ID NO: 82 is the nucleotide sequence of PGT130 Lambda
SEQ ID NO: 83 is the amino acid sequence of PGT121 Heavy Chain
SEQ ID NO: 84 is the nucleotide sequence of PGT121 Heavy Chain
SEQ ID NO: 85 is the amino acid sequence of PGT121 Lambda
SEQ ID NO: 86 is the nucleotide sequence of PGT121 Lambda
SEQ ID NO: 87 is the amino acid sequence of 2219 Heavy Chain
SEQ ID NO: 88 is the nucleotide sequence of 2219 Heavy Chain
SEQ ID NO: 89 is the amino acid sequence of 2219 Lambda
SEQ ID NO: 90 is the nucleotide sequence of 2219 Lambda
SEQ ID NO: 91 is the amino acid sequence of 3074 Heavy Chain
SEQ ID NO: 92 is the nucleotide sequence of 3074 Heavy Chain
SEQ ID NO: 93 is the amino acid sequence of 3074 Lambda
SEQ ID NO: 94 is the nucleotide sequence of 3074 Lambda
SEQ ID NO: 95 is the amino acid sequence of 4025 Heavy Chain
SEQ ID NO: 96 is the nucleotide sequence of 4025 Heavy Chain
SEQ ID NO: 97 is the amino acid sequence of 4025 Lambda
SEQ ID NO: 98 is the nucleotide sequence of 4025 Lambda
SEQ ID NO: 99 is the amino acid sequence of 2557 Heavy Chain
SEQ ID NO: 100 is the nucleotide sequence of 2557 Heavy Chain
SEQ ID NO: 101 is the amino acid sequence of 2557 Lambda
SEQ ID NO: 102 is the nucleotide sequence of 2557 Lambda
SEQ ID NO: 103 is the amino acid sequence of F425-B4e8 Heavy Chain
SEQ ID NO: 104 is the nucleotide sequence of F425-B4e8 Heavy Chain
SEQ ID NO: 105 is the amino acid sequence of F425-B4e8 Kappa
SEQ ID NO: 106 is the nucleotide sequence of F425-B4e8 Kappa
SEQ ID NO: 107 is the amino acid sequence of 17b Heavy Chain
SEQ ID NO: 108 is the nucleotide sequence of 17b Heavy Chain
SEQ ID NO: 109 is the amino acid sequence of 17b Kappa
SEQ ID NO: 110 is the nucleotide sequence of 17b Kappa
SEQ ID NO: 111 is the amino acid sequence of F105 Heavy Chain
SEQ ID NO: 112 is the nucleotide sequence of F105 Heavy Chain
SEQ ID NO: 113 is the amino acid sequence of F105 Kappa
SEQ ID NO: 114 is the nucleotide sequence of F105 Kappa
SEQ ID NO: 115 is the amino acid sequence of b12 Heavy Chain
SEQ ID NO: 116 is the nucleotide sequence of b12 Heavy Chain
SEQ ID NO: 117 is the amino acid sequence of b12 Kappa
SEQ ID NO: 118 is the nucleotide sequence of b12 Kappa
SEQ ID NO: 119 is the amino acid sequence of B6 Heavy Chain
SEQ ID NO: 120 is the nucleotide sequence of B6 Heavy Chain
SEQ ID NO: 121 is the amino acid sequence of B6 Kappa
SEQ ID NO: 122 is the nucleotide sequence of B6 Kappa
SEQ ID NO: 123 is the amino acid sequence of 11A Heavy Chain
SEQ ID NO: 124 is the nucleotide sequence of 11A Heavy Chain
SEQ ID NO: 125 is the amino acid sequence of 11A Kappa
SEQ ID NO: 126 is the nucleotide sequence of 11A Kappa
SEQ ID NO: 127 is the amino acid sequence of 12N Heavy Chain
SEQ ID NO: 128 is the nucleotide sequence of 12N Heavy Chain
SEQ ID NO: 129 is the amino acid sequence of 12N Kappa
SEQ ID NO: 130 is the nucleotide sequence of 12N Kappa
SEQ ID NO: 131 is the amino acid sequence of 39F Heavy Chain
SEQ ID NO: 132 is the nucleotide sequence of 39F Heavy Chain
SEQ ID NO: 133 is the amino acid sequence of 39F Lambda pF
SEQ ID NO: 134 is the nucleotide sequence of 39F Lambda pF
SEQ ID NO: 135 is the nucleotide sequence of Subtype A Consensus Envelope with an IgE leader.
SEQ ID NO: 136 is the amino acid sequence of Subtype A Consensus Envelope with an IgE leader.
SEQ ID NO: 137 is the nucleotide sequence of Subtype B Consensus Envelope with an IgE leader.
SEQ ID NO: 138 is the amino acid sequence of Subtype B Consensus Envelope with an IgE leader.
SEQ ID NO: 139 is the nucleotide sequence of Subtype C Consensus Envelope with an IgE leader.
SEQ ID NO: 140 is the amino acid sequence of Subtype C Consensus Envelope with an IgE leader.
SEQ ID NO: 141 is the nucleotide sequence of Subtype D Consensus Envelope with an IgE leader.
SEQ ID NO: 142 is the amino acid sequence of Subtype D Consensus Envelope with an IgE leader.
SEQ ID NO: 143 is the nucleotide sequence of Subtype B Consensus Nef-Rev with an IgE leader.
SEQ ID NO: 144 is the amino acid sequence of Subtype B Consensus Nef-Rev with an IgE leader.
SEQ ID NO: 145 is the nucleotide sequence of Gag Consensus DNA sequence of subtype A, B, C and D with an IgE leader.
SEQ ID NO: 146 is the amino acid sequence of Gag Consensus DNA sequence of subtype A, B, C and D with an IgE leader.
SEQ ID NO: 147 is the amino acid sequence of Subtype A Consensus Envelope without an IgE leader.
SEQ ID NO: 148 is the amino acid sequence of Subtype B Consensus Envelope without an IgE leader.
SEQ ID NO: 149 is the amino acid sequence of Subtype C Consensus Envelope without an IgE leader.
SEQ ID NO: 150 is the amino acid sequence of Subtype D Consensus Envelope without an IgE leader.
SEQ ID NO: 151 is the amino acid sequence of Subtype B Consensus Nef-Rev without an IgE leader.
SEQ ID NO: 152 is the amino acid sequence of Gag Consensus DNA sequence of subtype A, B, C and D without an IgE leader.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

“Antibody” may mean an antibody of classes IgG, IgM, IgA, IgD or IgE, or fragments, fragments or derivatives thereof, including Fab, F(ab′)2, Fd, and single chain antibodies, and derivatives thereof. The antibody may be an antibody isolated from the serum sample of mammal, a polyclonal antibody, affinity purified antibody, or mixtures thereof which exhibits sufficient binding specificity to a desired epitope or a sequence derived therefrom.

“Antibody fragment” or “fragment of an antibody” as used interchangeably herein refers to a portion of an intact antibody comprising the antigen-binding site or variable region. The portion does not include the constant heavy chain domains (i.e. CH2, CH3, or CH4, depending on the antibody isotype) of the Fc region of the intact antibody. Examples of antibody fragments include, but are not limited to, Fab fragments, Fab′ fragments, Fab′-SH fragments, F(ab′)2 fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

“Antigen” refers to proteins that have the ability to generate an immune response in a host. An antigen may be recognized and bound by an antibody. An antigen may originate from within the body or from the external environment.

“Coding sequence” or “encoding nucleic acid” as used herein may mean refers to the nucleic acid (RNA or DNA molecule) that comprise a nucleotide sequence which encodes an antibody as set forth herein. The coding sequence may further include initiation and termination signals operably linked to regulatory elements including a promoter and polyadenylation signal capable of directing expression in the cells of an individual or mammal to whom the nucleic acid is administered. The coding sequence may further include sequences that encode signal peptides.

“Complement” or “complementary” as used herein may mean a nucleic acid may mean Watson-Crick (e.g., A-T/U and C-G) or Hoogsteen base pairing between nucleotides or nucleotide analogs of nucleic acid molecules.

“Constant current” as used herein to define a current that is received or experienced by a tissue, or cells defining said tissue, over the duration of an electrical pulse delivered to same tissue. The electrical pulse is delivered from the electroporation devices described herein. This current remains at a constant amperage in said tissue over the life of an electrical pulse because the electroporation device provided herein has a feedback element, preferably having instantaneous feedback. The feedback element can measure the resistance of the tissue (or cells) throughout the duration of the pulse and cause the electroporation device to alter its electrical energy output (e.g., increase voltage) so current in same tissue remains constant throughout the electrical pulse (on the order of microseconds), and from pulse to pulse. In some embodiments, the feedback element comprises a controller.

“Current feedback” or “feedback” as used herein may be used interchangeably and may mean the active response of the provided electroporation devices, which comprises measuring the current in tissue between electrodes and altering the energy output delivered by the EP device accordingly in order to maintain the current at a constant level. This constant level is preset by a user prior to initiation of a pulse sequence or electrical treatment. The feedback may be accomplished by the electroporation component, e.g., controller, of the electroporation device, as the electrical circuit therein is able to continuously monitor the current in tissue between electrodes and compare that monitored current (or current within tissue) to a preset current and continuously make energy-output adjustments to maintain the monitored current at preset levels. The feedback loop may be instantaneous as it is an analog closed-loop feedback.

“Decentralized current” as used herein may mean the pattern of electrical currents delivered from the various needle electrode arrays of the electroporation devices described herein, wherein the patterns minimize, or preferably eliminate, the occurrence of electroporation related heat stress on any area of tissue being electroporated.

“Electroporation,” “electro-permeabilization,” or “electro-kinetic enhancement” (“EP”) as used interchangeably herein may refer to the use of a transmembrane electric field pulse to induce microscopic pathways (pores) in a bio-membrane; their presence allows biomolecules such as plasmids, oligonucleotides, siRNA, drugs, ions, and water to pass from one side of the cellular membrane to the other.

“Endogenous antibody” as used herein may refer to an antibody that is generated in a subject that is administered an effective dose of an antigen for induction of a humoral immune response.

“Feedback mechanism” as used herein may refer to a process performed by either software or hardware (or firmware), which process receives and compares the impedance of the desired tissue (before, during, and/or after the delivery of pulse of energy) with a present value, preferably current, and adjusts the pulse of energy delivered to achieve the preset value. A feedback mechanism may be performed by an analog closed loop circuit.

“Fragment” may mean a polypeptide fragment of an antibody that is function, i.e., can bind to desired target and have the same intended effect as a full length antibody. A fragment of an antibody may be 100% identical to the full length except missing at least one amino acid from the N and/or C terminal, in each case with or without signal peptides and/or a methionine at position 1. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length antibody, excluding any heterologous signal peptide added. The fragment may comprise a fragment of a polypeptide that is 95% or more, 96% or more, 97% or more, 98% or more or 99% or more identical to the antibody and additionally comprise an N terminal methionine or heterologous signal peptide which is not included when calculating percent identity. Fragments may further comprise an N terminal methionine and/or a signal peptide such as an immunoglobulin signal peptide, for example an IgE or IgG signal peptide. The N terminal methionine and/or signal peptide may be linked to a fragment of an antibody.

A fragment of a nucleic acid sequence that encodes an antibody may be 100% identical to the full length except missing at least one nucleotide from the 5′ and/or 3′ end, in each case with or without sequences encoding signal peptides and/or a methionine at position 1. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length coding sequence, excluding any heterologous signal peptide added. The fragment may comprise a fragment that encode a polypeptide that is 95% or more, 96% or more, 97% or more, 98% or more or 99% or more identical to the antibody and additionally optionally comprise sequence encoding an N terminal methionine or heterologous signal peptide which is not included when calculating percent identity. Fragments may further comprise coding sequences for an N terminal methionine and/or a signal peptide such as an immunoglobulin signal peptide, for example an IgE or IgG signal peptide. The coding sequence encoding the N terminal methionine and/or signal peptide may be linked to a fragment of coding sequence.

“Genetic construct” as used herein refers to the DNA or RNA molecules that comprise a nucleotide sequence which encodes a protein, such as an antibody. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed.

“Identical” or “identity” as used herein in the context of two or more nucleic acids or polypeptide sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Impedance” as used herein may be used when discussing the feedback mechanism and can be converted to a current value according to Ohm's law, thus enabling comparisons with the preset current.

“Immune response” as used herein may mean the activation of a host's immune system, e.g., that of a mammal, in response to the introduction of one or more nucleic acids and/or peptides. The immune response can be in the form of a cellular or humoral response, or both.

“Nucleic acid” or “oligonucleotide” or “polynucleotide” as used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

“Operably linked” as used herein may mean that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

A “peptide,” “protein,” or “polypeptide” as used herein can mean a linked sequence of amino acids and can be natural, synthetic, or a modification or combination of natural and synthetic.

“Promoter” as used herein may mean a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which can be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV 40 late promoter and the CMV IE promoter.

“Signal peptide” and “leader sequence” are used interchangeably herein and refer to an amino acid sequence that can be linked at the amino terminus of a protein set forth herein. Signal peptides/leader sequences typically direct localization of a protein. Signal peptides/leader sequences used herein preferably facilitate secretion of the protein from the cell in which it is produced. Signal peptides/leader sequences are often cleaved from the remainder of the protein, often referred to as the mature protein, upon secretion from the cell. Signal peptides/leader sequences are linked at the N terminus of the protein.

“Stringent hybridization conditions” as used herein may mean conditions under which a first nucleic acid sequence (e.g., probe) will hybridize to a second nucleic acid sequence (e.g., target), such as in a complex mixture of nucleic acids. Stringent conditions are sequence dependent and will be different in different circumstances. Stringent conditions may be selected to be about 5-10° C. lower than the thermal melting point (Tₘ) for the specific sequence at a defined ionic strength pH. The Tₘ may be the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may be those in which the salt concentration is less than about 1.0 M sodium ion, such as about 0.01-1.0 M sodium ion concentration (or other salts) at pH 7.0 to 8.3 and the temperature is at least about 30° C. for short probes (e.g., about 10-50 nucleotides) and at least about 60° C. for long probes (e.g., greater than about 50 nucleotides). Stringent conditions may also be achieved with the addition of destabilizing agents such as formamide. For selective or specific hybridization, a positive signal may be at least 2 to 10 times background hybridization. Exemplary stringent hybridization conditions include the following: 50% formamide, 5× SSC, and 1% SDS, incubating at 42° C., or, 5× SSC, 1% SDS, incubating at 65° C., with wash in 0.2× SSC, and 0.1% SDS at 65° C.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc) and a human). In some embodiments, the subject may be a human or a non-human. The subject or patient may be undergoing other forms of treatment.

“Substantially complementary” as used herein may mean that a first sequence is at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or that the two sequences hybridize under stringent hybridization conditions.

“Substantially identical” as used herein may mean that a first and second sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%,or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or more nucleotides or amino acids, or with respect to nucleic acids, if the first sequence is substantially complementary to the complement of the second sequence.

“Synthetic antibody” as used herein refers to an antibody that is encoded by the recombinant nucleic acid sequence described herein and is generated in a subject.

“Treatment” or “treating,” as used herein can mean protecting of a subject from a disease through means of preventing, suppressing, repressing, or completely eliminating the disease. Preventing the disease involves administering a vaccine of the present invention to a subject prior to onset of the disease. Suppressing the disease involves administering a composition of the present invention to a subject after induction of the disease but before its clinical appearance. Repressing the disease involves administering a composition of the present invention to a subject after clinical appearance of the disease.

“Variant” used herein with respect to a nucleic acid may mean (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within ±2 of each other. Both the hyrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A variant may be a nucleic acid sequence that is substantially identical over the full length of the full gene sequence or a fragment thereof. The nucleic acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof. A variant may be an amino acid sequence that is substantially identical over the full length of the amino acid sequence or fragment thereof. The amino acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

“Vector” as used herein may mean a nucleic acid sequence containing an origin of replication. A vector may be a plasmid, bacteriophage, bacterial artificial chromosome or yeast artificial chromosome. A vector may be a DNA or RNA vector. A vector may be either a self-replicating extrachromosomal vector or a vector which integrates into a host genome.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 2. COMPOSITION

The present invention relates to a composition comprising a recombinant nucleic acid sequence encoding an antibody, a fragment thereof, a variant thereof, or a combination thereof. The composition, when administered to a subject in need thereof, can result in the generation of a synthetic antibody in the subject. The synthetic antibody can bind a target molecule (i.e., an antigen) present in the subject. Such binding can neutralize the antigen, block recognition of the antigen by another molecule, for example, a protein or nucleic acid, and elicit or induce an immune response to the antigen.

In one embodiment, the composition comprises a nucleotide sequence encoding a synthetic antibody. In one embodiment, the composition comprises a nucleic acid molecule comprising a first nucleotide sequence encoding a first synthetic antibody and a second nucleotide sequence encoding a second synthetic antibody. In one embodiment, the nucleic acid molecule comprises a nucleotide sequence encoding a cleavage domain.

In one embodiment, the nucleic acid molecule comprises a nucleotide sequence encoding an anti-HIV antibody. In one embodiment, the anti HIV-antibody is an anti-HIV broadly neutralizing antibody.

In one embodiment, the nucleotide sequence encoding an anti HIV-antibody comprises one or more codon optimized nucleic acid sequences encoding an amino acid sequence at least 95% homologous to an amino acid sequence as set forth in SEQ ID NO:1, SEQ ID NO:3, SEQ ID NO:5, SEQ ID NO:7, SEQ ID NO:9, SEQ ID NO:11, SEQ ID NO:13, SEQ ID NO: 15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39 SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:111, SEQ ID NO:113, SEQ ID NO:115, SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, or a fragment thereof.

In one embodiment, the nucleotide sequence encoding an anti-HIV antibody comprises one or more RNA sequence transcribed from one or more DNA sequences encoding an amino acid sequence at least 95% homologous to an amino acid sequence as set forth in SEQ ID NO:1, SEQ ID NO:3, SEQ ID NO:5, SEQ ID NO:7, SEQ ID NO:9, SEQ ID NO:11, SEQ ID NO:13, SEQ ID NO: 15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39 SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:111, SEQ ID NO:113, SEQ ID NO:115, SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, ora fragment thereof.

In one embodiment, the nucleotide sequence encoding an anti-HIV antibody comprises one or more codon optimized nucleic acid sequences at least 95% homologous to a nucleic acid sequence as set forth in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:17, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO: 60, SEQ ID NO:62, SEQ ID NO:64, SEQ ID NO:66, SEQ ID NO:68,SEQ ID NO: 70, SEQ ID NO:72, SEQ ID NO:74, SEQ ID NO:76, SEQ ID NO:78, SEQ ID NO: 80, SEQ ID NO:82, SEQ ID NO:84, SEQ ID NO:86, SEQ ID NO:88, SEQ ID NO: 90, SEQ ID NO:92, SEQ ID NO:94, SEQ ID NO:96, SEQ ID NO:98, SEQ ID NO: 100, SEQ ID NO:102, SEQ ID NO:104, SEQ ID NO:106, SEQ ID NO:108, SEQ ID NO: 110, SEQ ID NO:112, SEQ ID NO:114, SEQ ID NO:116, SEQ ID NO:118, SEQ ID NO: 120, SEQ ID NO:122, SEQ ID NO:124, SEQ ID NO:126, SEQ ID NO:128, SEQ ID NO: 130, SEQ ID NO:132, SEQ ID NO:134, or a fragment thereof.

In one embodiment, the nucleotide sequence encoding an anti-HIV antibody comprises one or more RNA sequence transcribed from one or more DNA sequences at least 95% homologous to a nucleic acid sequence as set forth in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:17, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO: 60, SEQ ID NO:62, SEQ ID NO:64, SEQ ID NO:66, SEQ ID NO:68,SEQ ID NO: 70, SEQ ID NO:72, SEQ ID NO:74, SEQ ID NO:76, SEQ ID NO:78, SEQ ID NO: 80, SEQ ID NO:82, SEQ ID NO:84, SEQ ID NO:86, SEQ ID NO:88, SEQ ID NO: 90, SEQ ID NO:92, SEQ ID NO:94, SEQ ID NO:96, SEQ ID NO:98, SEQ ID NO: 100, SEQ ID NO:102, SEQ ID NO:104, SEQ ID NO:106, SEQ ID NO:108, SEQ ID NO: 110, SEQ ID NO:112, SEQ ID NO:114, SEQ ID NO:116, SEQ ID NO:118, SEQ ID NO: 120, SEQ ID NO:122, SEQ ID NO:124, SEQ ID NO:126, SEQ ID NO:128, SEQ ID NO: 130, SEQ ID NO:132, SEQ ID NO:134, or a fragment thereof.

In one embodiment, the composition comprises a nucleotide sequence encoding a synthetic anti-HIV heavy chain. In one embodiment, the composition comprises a nucleotide sequence encoding a synthetic anti-HIV light chain. In one embodiment, the composition comprises a nucleotide sequence encoding a synthetic anti-HIV antibody. In one embodiment, the sequence encoding a synthetic anti-HIV antibody comprises a first sequence encoding a synthetic anti-HIV heavy chain and a second sequence encoding a synthetic anti-HIV light chain.

In one embodiment, the first nucleotide sequence encodes an amino acid sequence at least 90% homologous to SEQ ID NO:3 and the second nucleotide sequence encodes an amino acid sequence at least 90% homologous to SEQ ID NO:5. In one embodiment, the first nucleotide sequence encodes SEQ ID NO:3 and the second nucleotide sequence encodes SEQ ID NO:5.

In one embodiment, the first nucleotide sequence encodes an amino acid sequence at least 90% homologous to SEQ ID NO:11 and the second nucleotide sequence encodes an amino acid sequence at least 90% homologous to SEQ ID NO:13. In one embodiment, the first nucleotide sequence encodes SEQ ID NO:11 and the second nucleotide sequence encodes SEQ ID NO:13.

In one embodiment, the first nucleotide sequence encodes an amino acid sequence at least 90% homologous to SEQ ID NO:17 and the second nucleotide sequence encodes an amino acid sequence at least 90% homologous to SEQ ID NO:19. In one embodiment, the first nucleotide sequence encodes SEQ ID NO:17 and the second nucleotide sequence encodes SEQ ID NO:19.

In one embodiment, the first nucleotide sequence encodes an amino acid sequence at least 90% homologous to SEQ ID NO:25 and the second nucleotide sequence encodes an amino acid sequence at least 90% homologous to SEQ ID NO:27. In one embodiment, the first nucleotide sequence encodes SEQ ID NO:25 and the second nucleotide sequence encodes SEQ ID NO:27.

In one embodiment, the first nucleotide sequence encodes an amino acid sequence at least 90% homologous to SEQ ID NO:39 and the second nucleotide sequence encodes an amino acid sequence at least 90% homologous to SEQ ID NO:41. In one embodiment, the first nucleotide sequence encodes SEQ ID NO:39 and the second nucleotide sequence encodes SEQ ID NO:41.

In one embodiment, the first nucleotide sequence encodes an amino acid sequence at least 90% homologous to SEQ ID NO:47 and the second nucleotide sequence encodes an amino acid sequence at least 90% homologous to SEQ ID NO:49. In one embodiment, the first nucleotide sequence encodes SEQ ID NO:47 and the second nucleotide sequence encodes SEQ ID NO:49.

In one embodiment, the first nucleotide sequence encodes an amino acid sequence at least 90% homologous to SEQ ID NO:55 and the second nucleotide sequence encodes an amino acid sequence at least 90% homologous to SEQ ID NO:57. In one embodiment, the first nucleotide sequence encodes SEQ ID NO:55 and the second nucleotide sequence encodes SEQ ID NO:57.

In one embodiment, the first nucleotide sequence encodes an amino acid sequence at least 90% homologous to SEQ ID NO:59 and the second nucleotide sequence encodes an amino acid sequence at least 90% homologous to SEQ ID NO:61. In one embodiment, the first nucleotide sequence encodes SEQ ID NO:59 and the second nucleotide sequence encodes SEQ ID NO:61.

In one embodiment, the first nucleotide sequence encodes an amino acid sequence at least 90% homologous to SEQ ID NO:63 and the second nucleotide sequence encodes an amino acid sequence at least 90% homologous to SEQ ID NO:65. In one embodiment, the first nucleotide sequence encodes SEQ ID NO:63 and the second nucleotide sequence encodes SEQ ID NO:65.

In one embodiment, the first nucleotide sequence encodes an amino acid sequence at least 90% homologous to SEQ ID NO:67 and the second nucleotide sequence encodes an amino acid sequence at least 90% homologous to SEQ ID NO:69. In one embodiment, the first nucleotide sequence encodes SEQ ID NO:67 and the second nucleotide sequence encodes SEQ ID NO:69.

In one embodiment, the first nucleotide sequence encodes an amino acid sequence at least 90% homologous to SEQ ID NO:71 and the second nucleotide sequence encodes an amino acid sequence at least 90% homologous to SEQ ID NO:73. In one embodiment, the first nucleotide sequence encodes SEQ ID NO:71 and the second nucleotide sequence encodes SEQ ID NO:73.

In one embodiment, the first nucleotide sequence encodes an amino acid sequence at least 90% homologous to SEQ ID NO:75 and the second nucleotide sequence encodes an amino acid sequence at least 90% homologous to SEQ ID NO:77. In one embodiment, the first nucleotide sequence encodes SEQ ID NO:75 and the second nucleotide sequence encodes SEQ ID NO:77.

In one embodiment, the first nucleotide sequence encodes an amino acid sequence at least 90% homologous to SEQ ID NO:79 and the second nucleotide sequence encodes an amino acid sequence at least 90% homologous to SEQ ID NO:81. In one embodiment, the first nucleotide sequence encodes SEQ ID NO:79 and the second nucleotide sequence encodes SEQ ID NO:81.

In one embodiment, the first nucleotide sequence encodes an amino acid sequence at least 90% homologous to SEQ ID NO:83 and the second nucleotide sequence encodes an amino acid sequence at least 90% homologous to SEQ ID NO:85. In one embodiment, the first nucleotide sequence encodes SEQ ID NO:83 and the second nucleotide sequence encodes SEQ ID NO:85.

In one embodiment, the first nucleotide sequence encodes an amino acid sequence at least 90% homologous to SEQ ID NO:87 and the second nucleotide sequence encodes an amino acid sequence at least 90% homologous to SEQ ID NO:89. In one embodiment, the first nucleotide sequence encodes SEQ ID NO:87 and the second nucleotide sequence encodes SEQ ID NO:89.

In one embodiment, the first nucleotide sequence encodes an amino acid sequence at least 90% homologous to SEQ ID NO:91 and the second nucleotide sequence encodes an amino acid sequence at least 90% homologous to SEQ ID NO:93. In one embodiment, the first nucleotide sequence encodes SEQ ID NO:91 and the second nucleotide sequence encodes SEQ ID NO:93.

In one embodiment, the first nucleotide sequence encodes an amino acid sequence at least 90% homologous to SEQ ID NO:95 and the second nucleotide sequence encodes an amino acid sequence at least 90% homologous to SEQ ID NO:97. In one embodiment, the first nucleotide sequence encodes SEQ ID NO:95 and the second nucleotide sequence encodes SEQ ID NO:97.

In one embodiment, the first nucleotide sequence encodes an amino acid sequence at least 90% homologous to SEQ ID NO:99 and the second nucleotide sequence encodes an amino acid sequence at least 90% homologous to SEQ ID NO:101. In one embodiment, the first nucleotide sequence encodes SEQ ID NO:99 and the second nucleotide sequence encodes SEQ ID NO:101.

In one embodiment, the first nucleotide sequence encodes an amino acid sequence at least 90% homologous to SEQ ID NO:103 and the second nucleotide sequence encodes an amino acid sequence at least 90% homologous to SEQ ID NO: 105. In one embodiment, the first nucleotide sequence encodes SEQ ID NO:103 and the second nucleotide sequence encodes SEQ ID NO: 105.

In one embodiment, the first nucleotide sequence encodes an amino acid sequence at least 90% homologous to SEQ ID NO: 107 and the second nucleotide sequence encodes an amino acid sequence at least 90% homologous to SEQ ID NO: 109. In one embodiment, the first nucleotide sequence encodes SEQ ID NO: 107 and the second nucleotide sequence encodes SEQ ID NO: 109.

In one embodiment, the first nucleotide sequence encodes an amino acid sequence at least 90% homologous to SEQ ID NO:111 and the second nucleotide sequence encodes an amino acid sequence at least 90% homologous to SEQ ID NO: 113. In one embodiment, the first nucleotide sequence encodes SEQ ID NO:111 and the second nucleotide sequence encodes SEQ ID NO: 113.

In one embodiment, the first nucleotide sequence encodes an amino acid sequence at least 90% homologous to SEQ ID NO: 115 and the second nucleotide sequence encodes an amino acid sequence at least 90% homologous to SEQ ID NO: 117. In one embodiment, the first nucleotide sequence encodes SEQ ID NO: 115 and the second nucleotide sequence encodes SEQ ID NO: 117.

In one embodiment, the first nucleotide sequence encodes an amino acid sequence at least 90% homologous to SEQ ID NO: 119 and the second nucleotide sequence encodes an amino acid sequence at least 90% homologous to SEQ ID NO: 121. In one embodiment, the first nucleotide sequence encodes SEQ ID NO: 119 and the second nucleotide sequence encodes SEQ ID NO: 121.

In one embodiment, the first nucleotide sequence encodes an amino acid sequence at least 90% homologous to SEQ ID NO:123 and the second nucleotide sequence encodes an amino acid sequence at least 90% homologous to SEQ ID NO: 125. In one embodiment, the first nucleotide sequence encodes SEQ ID NO:123 and the second nucleotide sequence encodes SEQ ID NO: 125.

In one embodiment, the first nucleotide sequence encodes an amino acid sequence at least 90% homologous to SEQ ID NO: 1207 and the second nucleotide sequence encodes an amino acid sequence at least 90% homologous to SEQ ID NO: 129. In one embodiment, the first nucleotide sequence encodes SEQ ID NO: 1207 and the second nucleotide sequence encodes SEQ ID NO: 129.

In one embodiment, the first nucleotide sequence encodes an amino acid sequence at least 90% homologous to SEQ ID NO:131 and the second nucleotide sequence encodes an amino acid sequence at least 90% homologous to SEQ ID NO: 133. In one embodiment, the first nucleotide sequence encodes SEQ ID NO:131 and the second nucleotide sequence encodes SEQ ID NO: 133.

In one embodiment, the first nucleotide sequence comprises a nucleotide sequence at least 90% homologous to SEQ ID NO:4 and the second nucleotide sequence comprises a nucleotide sequence at least 90% homologous to SEQ ID NO:6. In one embodiment, the first nucleotide sequence comprises a nucleotide sequence as set forth in SEQ ID NO:4 and the second nucleotide sequence comprises a nucleotide sequence as set forth in SEQ ID NO:6.

In one embodiment, the first nucleotide sequence comprises a nucleotide sequence at least 90% homologous to SEQ ID NO:12 and the second nucleotide sequence comprises a nucleotide sequence at least 90% homologous to SEQ ID NO:14. In one embodiment, the first nucleotide sequence comprises a nucleotide sequence as set forth in SEQ ID NO:12 and the second nucleotide sequence comprises a nucleotide sequence as set forth in SEQ ID NO:14.

In one embodiment, the first nucleotide sequence comprises a nucleotide sequence at least 90% homologous to SEQ ID NO:18 and the second nucleotide sequence comprises a nucleotide sequence at least 90% homologous to SEQ ID NO:20. In one embodiment, the first nucleotide sequence comprises a nucleotide sequence as set forth in SEQ ID NO:18 and the second nucleotide sequence comprises a nucleotide sequence as set forth in SEQ ID NO:20.

In one embodiment, the first nucleotide sequence comprises a nucleotide sequence at least 90% homologous to SEQ ID NO:26 and the second nucleotide sequence comprises a nucleotide sequence at least 90% homologous to SEQ ID NO:28. In one embodiment, the first nucleotide sequence comprises a nucleotide sequence as set forth in SEQ ID NO:26 and the second nucleotide sequence comprises a nucleotide sequence as set forth in SEQ ID NO:28.

In one embodiment, the first nucleotide sequence comprises a nucleotide sequence at least 90% homologous to SEQ ID NO:40 and the second nucleotide sequence comprises a nucleotide sequence at least 90% homologous to SEQ ID NO:42. In one embodiment, the first nucleotide sequence comprises a nucleotide sequence as set forth in SEQ ID NO:40 and the second nucleotide sequence comprises a nucleotide sequence as set forth in SEQ ID NO:42.

In one embodiment, the first nucleotide sequence comprises a nucleotide sequence at least 90% homologous to SEQ ID NO:48 and the second nucleotide sequence comprises a nucleotide sequence at least 90% homologous to SEQ ID NO:50. In one embodiment, the first nucleotide sequence comprises a nucleotide sequence as set forth in SEQ ID NO:48 and the second nucleotide sequence comprises a nucleotide sequence as set forth in SEQ ID NO:50.

In one embodiment, the first nucleotide sequence comprises a nucleotide sequence at least 90% homologous to SEQ ID NO:56 and the second nucleotide sequence comprises a nucleotide sequence at least 90% homologous to SEQ ID NO:58. In one embodiment, the first nucleotide sequence comprises a nucleotide sequence as set forth in SEQ ID NO:56 and the second nucleotide sequence comprises a nucleotide sequence as set forth in SEQ ID NO:58.

In one embodiment, the first nucleotide sequence comprises a nucleotide sequence at least 90% homologous to SEQ ID NO:60 and the second nucleotide sequence comprises a nucleotide sequence at least 90% homologous to SEQ ID NO:62. In one embodiment, the first nucleotide sequence comprises a nucleotide sequence as set forth in SEQ ID NO:60 and the second nucleotide sequence comprises a nucleotide sequence as set forth in SEQ ID NO:62.

In one embodiment, the first nucleotide sequence comprises a nucleotide sequence at least 90% homologous to SEQ ID NO:64 and the second nucleotide sequence comprises a nucleotide sequence at least 90% homologous to SEQ ID NO:66. In one embodiment, the first nucleotide sequence comprises a nucleotide sequence as set forth in SEQ ID NO:64 and the second nucleotide sequence comprises a nucleotide sequence as set forth in SEQ ID NO:66.

In one embodiment, the first nucleotide sequence comprises a nucleotide sequence at least 90% homologous to SEQ ID NO:68 and the second nucleotide sequence comprises a nucleotide sequence at least 90% homologous to SEQ ID NO:70. In one embodiment, the first nucleotide sequence comprises a nucleotide sequence as set forth in SEQ ID NO:68 and the second nucleotide sequence comprises a nucleotide sequence as set forth in SEQ ID NO:70.

In one embodiment, the first nucleotide sequence comprises a nucleotide sequence at least 90% homologous to SEQ ID NO:72 and the second nucleotide sequence comprises a nucleotide sequence at least 90% homologous to SEQ ID NO:74. In one embodiment, the first nucleotide sequence comprises a nucleotide sequence as set forth in SEQ ID NO:72 and the second nucleotide sequence comprises a nucleotide sequence as set forth in SEQ ID NO:74.

In one embodiment, the first nucleotide sequence comprises a nucleotide sequence at least 90% homologous to SEQ ID NO:76 and the second nucleotide sequence comprises a nucleotide sequence at least 90% homologous to SEQ ID NO:78. In one embodiment, the first nucleotide sequence comprises a nucleotide sequence as set forth in SEQ ID NO:76 and the second nucleotide sequence comprises a nucleotide sequence as set forth in SEQ ID NO:78.

In one embodiment, the first nucleotide sequence comprises a nucleotide sequence at least 90% homologous to SEQ ID NO:80 and the second nucleotide sequence comprises a nucleotide sequence at least 90% homologous to SEQ ID NO: 82. In one embodiment, the first nucleotide sequence comprises a nucleotide sequence as set forth in SEQ ID NO:80 and the second nucleotide sequence comprises a nucleotide sequence as set forth in SEQ ID NO:82.

In one embodiment, the first nucleotide sequence comprises a nucleotide sequence at least 90% homologous to SEQ ID NO:84 and the second nucleotide sequence comprises a nucleotide sequence at least 90% homologous to SEQ ID NO: 86. In one embodiment, the first nucleotide sequence comprises a nucleotide sequence as set forth in SEQ ID NO:84 and the second nucleotide sequence comprises a nucleotide sequence as set forth in SEQ ID NO:86.

In one embodiment, the first nucleotide sequence comprises a nucleotide sequence at least 90% homologous to SEQ ID NO:88 and the second nucleotide sequence comprises a nucleotide sequence at least 90% homologous to SEQ ID NO:90. In one embodiment, the first nucleotide sequence comprises a nucleotide sequence as set forth in SEQ ID NO:88 and the second nucleotide sequence comprises a nucleotide sequence as set forth in SEQ ID NO:90.

In one embodiment, the first nucleotide sequence comprises a nucleotide sequence at least 90% homologous to SEQ ID NO:92 and the second nucleotide sequence comprises a nucleotide sequence at least 90% homologous to SEQ ID NO:94. In one embodiment, the first nucleotide sequence comprises a nucleotide sequence as set forth in SEQ ID NO:92 and the second nucleotide sequence comprises a nucleotide sequence as set forth in SEQ ID NO:94.

In one embodiment, the first nucleotide sequence comprises a nucleotide sequence at least 90% homologous to SEQ ID NO:96 and the second nucleotide sequence comprises a nucleotide sequence at least 90% homologous to SEQ ID NO:98. In one embodiment, the first nucleotide sequence comprises a nucleotide sequence as set forth in SEQ ID NO:96 and the second nucleotide sequence comprises a nucleotide sequence as set forth in SEQ ID NO:98.

In one embodiment, the first nucleotide sequence comprises a nucleotide sequence at least 90% homologous to SEQ ID NO:100 and the second nucleotide sequence comprises a nucleotide sequence at least 90% homologous to SEQ ID NO:102. In one embodiment, the first nucleotide sequence comprises a nucleotide sequence as set forth in SEQ ID NO:100 and the second nucleotide sequence comprises a nucleotide sequence as set forth in SEQ ID NO:102.

In one embodiment, the first nucleotide sequence comprises a nucleotide sequence at least 90% homologous to SEQ ID NO:104 and the second nucleotide sequence comprises a nucleotide sequence at least 90% homologous to SEQ ID NO: 106. In one embodiment, the first nucleotide sequence comprises a nucleotide sequence as set forth in SEQ ID NO:104 and the second nucleotide sequence comprises a nucleotide sequence as set forth in SEQ ID NO: 106.

In one embodiment, the first nucleotide sequence comprises a nucleotide sequence at least 90% homologous to SEQ ID NO: 108 and the second nucleotide sequence comprises a nucleotide sequence at least 90% homologous to SEQ ID NO: 110. In one embodiment, the first nucleotide sequence comprises a nucleotide sequence as set forth in SEQ ID NO: 108 and the second nucleotide sequence comprises a nucleotide sequence as set forth in SEQ ID NO: 110.

In one embodiment, the first nucleotide sequence comprises a nucleotide sequence at least 90% homologous to SEQ ID NO:112 and the second nucleotide sequence comprises a nucleotide sequence at least 90% homologous to SEQ ID NO: 114. In one embodiment, the first nucleotide sequence comprises a nucleotide sequence as set forth in SEQ ID NO:112 and the second nucleotide sequence comprises a nucleotide sequence as set forth in SEQ ID NO: 114.

In one embodiment, the first nucleotide sequence comprises a nucleotide sequence at least 90% homologous to SEQ ID NO: 116 and the second nucleotide sequence comprises a nucleotide sequence at least 90% homologous to SEQ ID NO: 118. In one embodiment, the first nucleotide sequence comprises a nucleotide sequence as set forth in SEQ ID NO: 116 and the second nucleotide sequence comprises a nucleotide sequence as set forth in SEQ ID NO: 118.

In one embodiment, the first nucleotide sequence comprises a nucleotide sequence at least 90% homologous to SEQ ID NO: 120 and the second nucleotide sequence comprises a nucleotide sequence at least 90% homologous to SEQ ID NO: 122. In one embodiment, the first nucleotide sequence comprises a nucleotide sequence as set forth in SEQ ID NO: 120 and the second nucleotide sequence comprises a nucleotide sequence as set forth in SEQ ID NO: 122.

In one embodiment, the first nucleotide sequence comprises a nucleotide sequence at least 90% homologous to SEQ ID NO:124 and the second nucleotide sequence comprises a nucleotide sequence at least 90% homologous to SEQ ID NO: 126. In one embodiment, the first nucleotide sequence comprises a nucleotide sequence as set forth in SEQ ID NO:124 and the second nucleotide sequence comprises a nucleotide sequence as set forth in SEQ ID NO: 126.

In one embodiment, the first nucleotide sequence comprises a nucleotide sequence at least 90% homologous to SEQ ID NO: 128 and the second nucleotide sequence comprises a nucleotide sequence at least 90% homologous to SEQ ID NO: 130. In one embodiment, the first nucleotide sequence comprises a nucleotide sequence as set forth in SEQ ID NO: 128 and the second nucleotide sequence comprises a nucleotide sequence as set forth in SEQ ID NO: 130.

In one embodiment, the first nucleotide sequence comprises a nucleotide sequence at least 90% homologous to SEQ ID NO:132 and the second nucleotide sequence comprises a nucleotide sequence at least 90% homologous to SEQ ID NO: 134. In one embodiment, the first nucleotide sequence comprises a nucleotide sequence as set forth in SEQ ID NO:132 and the second nucleotide sequence comprises a nucleotide sequence as set forth in SEQ ID NO: 134.

The composition of the invention can treat, prevent and/or protect against any disease, disorder, or condition associated with HIV infection. In certain embodiments, the composition can treat, prevent, and or/protect against viral infection. In certain embodiments, the composition can treat, prevent, and or/protect against condition associated with HIV infection.

In one embodiment, the present invention relates to a composition comprising a recombinant nucleic acid sequence encoding an IgG4 antibody. In one embodiment the IgG4 antibody comprises an amino acid substitution Ser288Pro. In one embodiment, IgG4 amino acid substitution Ser288Pro prevents IgG4 Fab arm switching. The IgG4 synthetic antibody can be for a foreign antigen or a self antigen. For example, IgG4 synthetic antibody can be for a viral antigen, a bacterial antigen, a parasitic antigen, a fungal antigen or a cancer antigen. Examples foreign and self antigens are described in U.S. Patent Application Publication No. US2015/0284448, the contents of which are incorporated herein in its entirety.

The composition comprising a recombinant nucleic acid sequence encoding an IgG4 antibody, when administered to a subject in need thereof, can result in the generation of a synthetic antibody in the subject. The synthetic antibody can bind a target molecule (i.e., an antigen) present in the subject. Such binding can neutralize the antigen, block recognition of the antigen by another molecule, for example, a protein or nucleic acid, and elicit or induce an immune response to the antigen.

The composition can result in the generation of the synthetic antibody in the subject within at least about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 20 hours, 25 hours, 30 hours, 35 hours, 40 hours, 45 hours, 50 hours, or 60 hours of administration of the composition to the subject. The composition can result in generation of the synthetic antibody in the subject within at least about 1 day, 2 days, 3 days, 4 days, 5 days, 6 days, 7 days, 8 days, 9 days, or 10 days of administration of the composition to the subject. The composition can result in generation of the synthetic antibody in the subject within about 1 hour to about 6 days, about 1 hour to about 5 days, about 1 hour to about 4 days, about 1 hour to about 3 days, about 1 hour to about 2 days, about 1 hour to about 1 day, about 1 hour to about 72 hours, about 1 hour to about 60 hours, about 1 hour to about 48 hours, about 1 hour to about 36 hours, about 1 hour to about 24 hours, about 1 hour to about 12 hours, or about 1 hour to about 6 hours of administration of the composition to the subject.

The composition, when administered to the subject in need thereof, can result in the generation of the synthetic antibody in the subject more quickly than the generation of an endogenous antibody in a subject who is administered an antigen to induce a humoral immune response. The composition can result in the generation of the synthetic antibody at least about 1 day, 2 days, 3 days, 4 days, 5 days, 6 days, 7 days, 8 days, 9 days, or 10 days before the generation of the endogenous antibody in the subject who was administered an antigen to induce a humoral immune response.

The composition of the present invention can have features required of effective compositions such as being safe so that the composition does not cause illness or death; being protective against illness; and providing ease of administration, few side effects, biological stability and low cost per dose.

### 3. RECOMBINANT NUCLEIC ACID SEQUENCE

As described above, the composition can comprise a recombinant nucleic acid sequence. The recombinant nucleic acid sequence can encode the antibody, a fragment thereof, a variant thereof, or a combination thereof. The antibody is described in more detail below.

The recombinant nucleic acid sequence can be a heterologous nucleic acid sequence. The recombinant nucleic acid sequence can include one or more heterologous nucleic acid sequences.

The recombinant nucleic acid sequence can be an optimized nucleic acid sequence. Such optimization can increase or alter the immunogenicity of the antibody. Optimization can also improve transcription and/or translation. Optimization can include one or more of the following: low GC content leader sequence to increase transcription; mRNA stability and codon optimization; addition of a kozak sequence (e.g., GCC ACC) for increased translation; addition of an immunoglobulin (Ig) leader sequence encoding a signal peptide; addition of an internal IRES sequence and eliminating to the extent possible cis-acting sequence motifs (i.e., internal TATA boxes).

### a. Recombinant Nucleic Acid Sequence Construct

The recombinant nucleic acid sequence can include one or more recombinant nucleic acid sequence constructs. The recombinant nucleic acid sequence construct can include one or more components, which are described in more detail below.

The recombinant nucleic acid sequence construct can include a heterologous nucleic acid sequence that encodes a heavy chain polypeptide, a fragment thereof, a variant thereof, or a combination thereof. The recombinant nucleic acid sequence construct can include a heterologous nucleic acid sequence that encodes a light chain polypeptide, a fragment thereof, a variant thereof, or a combination thereof. The recombinant nucleic acid sequence construct can also include a heterologous nucleic acid sequence that encodes a protease or peptidase cleavage site. The recombinant nucleic acid sequence construct can also include a heterologous nucleic acid sequence that encodes an internal ribosome entry site (IRES). An IRES may be either a viral IRES or an eukaryotic IRES. The recombinant nucleic acid sequence construct can include one or more leader sequences, in which each leader sequence encodes a signal peptide. The recombinant nucleic acid sequence construct can include one or more promoters, one or more introns, one or more transcription termination regions, one or more initiation codons, one or more termination or stop codons, and/or one or more polyadenylation signals. The recombinant nucleic acid sequence construct can also include one or more linker or tag sequences. The tag sequence can encode a hemagglutinin (HA) tag.

### (1) Heavy Chain Polypeptide

The recombinant nucleic acid sequence construct can include the heterologous nucleic acid encoding the heavy chain polypeptide, a fragment thereof, a variant thereof, or a combination thereof. The heavy chain polypeptide can include a variable heavy chain (VH) region and/or at least one constant heavy chain (CH) region. The at least one constant heavy chain region can include a constant heavy chain region 1 (CH1), a constant heavy chain region 2 (CH2), and a constant heavy chain region 3 (CH3), and/or a hinge region.

In some embodiments, the heavy chain polypeptide can include a VH region and a CH1 region. In other embodiments, the heavy chain polypeptide can include a VH region, a CH1 region, a hinge region, a CH2 region, and a CH3 region.

The heavy chain polypeptide can include a complementarity determining region (“CDR”) set. The CDR set can contain three hypervariable regions of the VH region. Proceeding from N-terminus of the heavy chain polypeptide, these CDRs are denoted “CDR1,” “CDR2,” and “CDR3,” respectively. CDR1, CDR2, and CDR3 of the heavy chain polypeptide can contribute to binding or recognition of the antigen.

In one embodiment, the heavy chain polypeptide comprises an amino acid sequence at least 95% identical to SEQ ID NO:3, SEQ ID NO:11, SEQ ID NO:17, SEQ ID NO:25, SEQ ID NO:39, SEQ ID NO:47, SEQ ID NO:55, SEQ ID NO:59, SEQ ID NO:63, SEQ ID NO:67, SEQ ID NO:71, SEQ ID NO:75, SEQ ID NO:79, SEQ ID NO:83, SEQ ID NO:87, SEQ ID NO:91, SEQ ID NO:95, SEQ ID NO:99, SEQ ID NO:103, SEQ ID NO:107, SEQ ID NO:111, SEQ ID NO:115, SEQ ID NO:119, SEQ ID NO:123, SEQ ID NO:127, or SEQ ID NO:131.

In one embodiment, the heavy chain polypeptide comprises an amino acid sequence as set forth in SEQ ID NO:3, SEQ ID NO:11, SEQ ID NO:17, SEQ ID NO:25, SEQ ID NO:39, SEQ ID NO:47, SEQ ID NO:55, SEQ ID NO:59, SEQ ID NO:63, SEQ ID NO:67, SEQ ID NO:71, SEQ ID NO:75, SEQ ID NO:79, SEQ ID NO:83, SEQ ID NO:87, SEQ ID NO:91, SEQ ID NO:95, SEQ ID NO:99, SEQ ID NO:103, SEQ ID NO:107, SEQ ID NO:111, SEQ ID NO:115, SEQ ID NO:119, SEQ ID NO:123, SEQ ID NO:127, or SEQ ID NO:131.

In one embodiment, the heavy chain polypeptide is encoded by a nucleotide sequence at least 95% identical to SEQ ID NO:4, SEQ ID NO:12, SEQ ID NO:18, SEQ ID NO:26, SEQ ID NO:40, SEQ ID NO:48, SEQ ID NO:56, SEQ ID NO:60, SEQ ID NO:64, SEQ ID NO:68, SEQ ID NO:72, SEQ ID NO:76, SEQ ID NO:80, SEQ ID NO:84, SEQ ID NO:88, SEQ ID NO:92, SEQ ID NO:96, SEQ ID NO:100, SEQ ID NO:104, SEQ ID NO:108, SEQ ID NO:112, SEQ ID NO:116, SEQ ID NO:120, SEQ ID NO:124, SEQ ID NO:128, or SEQ ID NO:132.

In one embodiment, the heavy chain polypeptide is encoded by a nucleotide sequence as set forth in SEQ ID NO:4, SEQ ID NO:12, SEQ ID NO:18, SEQ ID NO:26, SEQ ID NO:40, SEQ ID NO:48, SEQ ID NO:56, SEQ ID NO:60, SEQ ID NO:64, SEQ ID NO:68, SEQ ID NO:72, SEQ ID NO:76, SEQ ID NO:80, SEQ ID NO:84, SEQ ID NO:88, SEQ ID NO:92, SEQ ID NO:96, SEQ ID NO:100, SEQ ID NO:104, SEQ ID NO:108, SEQ ID NO:112, SEQ ID NO:116, SEQ ID NO:120, SEQ ID NO:124, SEQ ID NO:128, or SEQ ID NO:132.

### (2) Light Chain Polypeptide

The recombinant nucleic acid sequence construct can include the heterologous nucleic acid sequence encoding the light chain polypeptide, a fragment thereof, a variant thereof, or a combination thereof. The light chain polypeptide can include a variable light chain (VL) region and/or a constant light chain (CL) region.

The light chain polypeptide can include a complementarity determining region (“CDR”) set. The CDR set can contain three hypervariable regions of the VL region. Proceeding from N-terminus of the light chain polypeptide, these CDRs are denoted “CDR1,” “CDR2,” and “CDR3,” respectively. CDR1, CDR2, and CDR3 of the light chain polypeptide can contribute to binding or recognition of the antigen.

In one embodiment, the light chain polypeptide comprises an amino acid sequence at least 95% identical to SEQ ID NO:5, SEQ ID NO:13, SEQ ID NO:19, SEQ ID NO:27, SEQ ID NO:41, SEQ ID NO:49, SEQ ID NO:57, SEQ ID NO:61, SEQ ID NO:65, SEQ ID NO:69, SEQ ID NO:73, SEQ ID NO:77, SEQ ID NO:81, SEQ ID NO:85, SEQ ID NO:89, SEQ ID NO:93, SEQ ID NO:97, SEQ ID NO:101, SEQ ID NO:105, SEQ ID NO:109, SEQ ID NO:113, SEQ ID NO:117, SEQ ID NO:121, SEQ ID NO:125, SEQ ID NO:129, or SEQ ID NO:133.

In one embodiment, the light chain polypeptide comprises an amino acid sequence as set forth in SEQ ID NO:5, SEQ ID NO:13, SEQ ID NO:19, SEQ ID NO:27, SEQ ID NO:41, SEQ ID NO:49, SEQ ID NO:57, SEQ ID NO:61, SEQ ID NO:65, SEQ ID NO:69, SEQ ID NO:73, SEQ ID NO:77, SEQ ID NO:81, SEQ ID NO:85, SEQ ID NO:89, SEQ ID NO:93, SEQ ID NO:97, SEQ ID NO:101, SEQ ID NO:105, SEQ ID NO:109, SEQ ID NO:113, SEQ ID NO:117, SEQ ID NO:121, SEQ ID NO:125, SEQ ID NO:129, or SEQ ID NO:133.

In one embodiment, the light chain polypeptide is encoded by a nucleotide sequence at least 95% identical to SEQ ID NO:6, SEQ ID NO:14, SEQ ID NO:20, SEQ ID NO:28, SEQ ID NO:42, SEQ ID NO:50, SEQ ID NO:58, SEQ ID NO:62, SEQ ID NO:66, SEQ ID NO:70, SEQ ID NO:74, SEQ ID NO:76, SEQ ID NO:82, SEQ ID NO:86, SEQ ID NO:90, SEQ ID NO:94, SEQ ID NO:98, SEQ ID NO:102, SEQ ID NO:106, SEQ ID NO:110, SEQ ID NO:114, SEQ ID NO:118, SEQ ID NO:122, SEQ ID NO:126, SEQ ID NO:130, or SEQ ID NO:134.

In one embodiment, the light chain polypeptide is encoded by a nucleotide sequence as set forth in SEQ ID NO:6, SEQ ID NO:14, SEQ ID NO:20, SEQ ID NO:28, SEQ ID NO:42, SEQ ID NO:50, SEQ ID NO:58, SEQ ID NO:62, SEQ ID NO:66, SEQ ID NO:70, SEQ ID NO:74, SEQ ID NO:76, SEQ ID NO:82, SEQ ID NO:86, SEQ ID NO:90, SEQ ID NO:94, SEQ ID NO:98, SEQ ID NO:102, SEQ ID NO:106, SEQ ID NO:110, SEQ ID NO:114, SEQ ID NO:118, SEQ ID NO:122, SEQ ID NO:126, SEQ ID NO:130, or SEQ ID NO:134.

### (3) Protease Cleavage Site

The recombinant nucleic acid sequence construct can include heterologous nucleic acid sequence encoding a protease cleavage site. The protease cleavage site can be recognized by a protease or peptidase. The protease can be an endopeptidase or endoprotease, for example, but not limited to, furin, elastase, HtrA, calpain, trypsin, chymotrypsin, trypsin, and pepsin. The protease can be furin. In other embodiments, the protease can be a serine protease, a threonine protease, cysteine protease, aspartate protease, metalloprotease, glutamic acid protease, or any protease that cleaves an internal peptide bond (i.e., does not cleave the N-terminal or C-terminal peptide bond).

The protease cleavage site can include one or more amino acid sequences that promote or increase the efficiency of cleavage. The one or more amino acid sequences can promote or increase the efficiency of forming or generating discrete polypeptides. The one or more amino acids sequences can include a 2A peptide sequence.

### (4) Linker Sequence

The recombinant nucleic acid sequence construct can include one or more linker sequences. The linker sequence can spatially separate or link the one or more components described herein. In other embodiments, the linker sequence can encode an amino acid sequence that spatially separates or links two or more polypeptides.

### (5) Promoter

The recombinant nucleic acid sequence construct can include one or more promoters. The one or more promoters may be any promoter that is capable of driving gene expression and regulating gene expression. Such a promoter is a cis-acting sequence element required for transcription via a DNA dependent RNA polymerase. Selection of the promoter used to direct gene expression depends on the particular application. The promoter may be positioned about the same distance from the transcription start in the recombinant nucleic acid sequence construct as it is from the transcription start site in its natural setting. However, variation in this distance may be accommodated without loss of promoter function.

The promoter may be operably linked to the heterologous nucleic acid sequence encoding the heavy chain polypeptide and/or light chain polypeptide. The promoter may be a promoter shown effective for expression in eukaryotic cells. The promoter operably linked to the coding sequence may be a CMV promoter, a promoter from simian virus 40 (SV40), such as SV40 early promoter and SV40 later promoter, a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human actin, human myosin, human hemoglobin, human muscle creatine, human polyhedrin, or human metalothionein.

The promoter can be a constitutive promoter or an inducible promoter, which initiates transcription only when the host cell is exposed to some particular external stimulus. In the case of a multicellular organism, the promoter can also be specific to a particular tissue or organ or stage of development. The promoter may also be a tissue specific promoter, such as a muscle or skin specific promoter, natural or synthetic. Examples of such promoters are described in US patent application publication no. US20040175727, the contents of which are incorporated herein in its entirety.

The promoter can be associated with an enhancer. The enhancer can be located upstream of the coding sequence. The enhancer may be human actin, human myosin, human hemoglobin, human muscle creatine or a viral enhancer such as one from CMV, FMDV, RSV or EBV. Polynucleotide function enhances are described in U.S. Pat. Nos. 5,593,972, 5,962,428, and WO94/016737, the contents of each are fully incorporated by reference.

### (6) Intron

The recombinant nucleic acid sequence construct can include one or more introns. Each intron can include functional splice donor and acceptor sites. The intron can include an enhancer of splicing. The intron can include one or more signals required for efficient splicing.

### (7) Transcription Termination Region

The recombinant nucleic acid sequence construct can include one or more transcription termination regions. The transcription termination region can be downstream of the coding sequence to provide for efficient termination. The transcription termination region can be obtained from the same gene as the promoter described above or can be obtained from one or more different genes.

### (8) Initiation Codon

The recombinant nucleic acid sequence construct can include one or more initiation codons. The initiation codon can be located upstream of the coding sequence. The initiation codon can be in frame with the coding sequence. The initiation codon can be associated with one or more signals required for efficient translation initiation, for example, but not limited to, a ribosome binding site.

### (9) Termination Codon

The recombinant nucleic acid sequence construct can include one or more termination or stop codons. The termination codon can be downstream of the coding sequence. The termination codon can be in frame with the coding sequence. The termination codon can be associated with one or more signals required for efficient translation termination.

### (10) Polyadenylation Signal

The recombinant nucleic acid sequence construct can include one or more polyadenylation signals. The polyadenylation signal can include one or more signals required for efficient polyadenylation of the transcript. The polyadenylation signal can be positioned downstream of the coding sequence. The polyadenylation signal may be a SV40 polyadenylation signal, LTR polyadenylation signal, bovine growth hormone (bGH) polyadenylation signal, human growth hormone (hGH) polyadenylation signal, or human β-globin polyadenylation signal. The SV40 polyadenylation signal may be a polyadenylation signal from a pCEP4 plasmid (Invitrogen, San Diego, CA).

### (11) Leader Sequence

The recombinant nucleic acid sequence construct can include one or more leader sequences. The leader sequence can encode a signal peptide. The signal peptide can be an immunoglobulin (Ig) signal peptide, for example, but not limited to, an IgG signal peptide and a IgE signal peptide.

### b. Arrangement of the Recombinant Nucleic Acid Sequence Construct

As described above, the recombinant nucleic acid sequence can include one or more recombinant nucleic acid sequence constructs, in which each recombinant nucleic acid sequence construct can include one or more components. The one or more components are described in detail above. The one or more components, when included in the recombinant nucleic acid sequence construct, can be arranged in any order relative to one another. In some embodiments, the one or more components can be arranged in the recombinant nucleic acid sequence construct as described below.

### (1) Arrangement 1

In one arrangement, a first recombinant nucleic acid sequence construct can include the heterologous nucleic acid sequence encoding the heavy chain polypeptide and a second recombinant nucleic acid sequence construct can include the heterologous nucleic acid sequence encoding the light chain polypeptide.

For example, in one embodiment, the first recombinant nucleic acid sequence encodes a heavy chain polypeptide having an amino acid sequence at least 95% homologous to SEQ ID NO:3, SEQ ID NO:11, SEQ ID NO:17, SEQ ID NO:25, SEQ ID NO:39, SEQ ID NO:47, SEQ ID NO:55, SEQ ID NO:59, SEQ ID NO:63, SEQ ID NO:67, SEQ ID NO:71, SEQ ID NO:75, SEQ ID NO:79, SEQ ID NO:83, SEQ ID NO:87, SEQ ID NO:91, SEQ ID NO:95, SEQ ID NO:99, SEQ ID NO:103, SEQ ID NO:107, SEQ ID NO:111, SEQ ID NO:115, SEQ ID NO:119, SEQ ID NO:123, SEQ ID NO:127, or SEQ ID NO:131.

In one embodiment, the first recombinant nucleic acid sequence comprises a nucleic acid sequence at least 95% homologous to SEQ ID NO:4, SEQ ID NO:12, SEQ ID NO:18, SEQ ID NO:26, SEQ ID NO:40, SEQ ID NO:48, SEQ ID NO:56, SEQ ID NO:60, SEQ ID NO:64, SEQ ID NO:68, SEQ ID NO:72, SEQ ID NO:76, SEQ ID NO:80, SEQ ID NO:84, SEQ ID NO:88, SEQ ID NO:92, SEQ ID NO:96, SEQ ID NO:100, SEQ ID NO:104, SEQ ID NO:108, SEQ ID NO:112, SEQ ID NO:116, SEQ ID NO:120, SEQ ID NO:124, SEQ ID NO:128, or SEQ ID NO:132.

In one embodiment, the second recombinant nucleic acid sequence encodes a light chain polypeptide having an amino acid sequence at least 95% homologous to SEQ ID NO:5, SEQ ID NO:13, SEQ ID NO:19, SEQ ID NO:27, SEQ ID NO:41, SEQ ID NO:49, SEQ ID NO:57, SEQ ID NO:61, SEQ ID NO:65, SEQ ID NO:69, SEQ ID NO:73, SEQ ID NO:77, SEQ ID NO:81, SEQ ID NO:85, SEQ ID NO:89, SEQ ID NO:93, SEQ ID NO:97, SEQ ID NO:101, SEQ ID NO:105, SEQ ID NO:109, SEQ ID NO:113, SEQ ID NO:117, SEQ ID NO:121, SEQ ID NO:125, SEQ ID NO:129, or SEQ ID NO:133.

In one embodiment, the second recombinant nucleic acid sequence comprises a nucleic acid sequence at least 95% homologous to SEQ ID NO:6, SEQ ID NO:14, SEQ ID NO:20, SEQ ID NO:28, SEQ ID NO:42, SEQ ID NO:50, SEQ ID NO:58, SEQ ID NO:62, SEQ ID NO:66, SEQ ID NO:70, SEQ ID NO:74, SEQ ID NO:76, SEQ ID NO:82, SEQ ID NO:86, SEQ ID NO:90, SEQ ID NO:94, SEQ ID NO:98, SEQ ID NO:102, SEQ ID NO:106, SEQ ID NO:110, SEQ ID NO:114, SEQ ID NO:118, SEQ ID NO:122, SEQ ID NO:126, SEQ ID NO:130, or SEQ ID NO:134.

The first recombinant nucleic acid sequence construct can be placed in a vector. The second recombinant nucleic acid sequence construct can be placed in a second or separate vector. Placement of the recombinant nucleic acid sequence construct into the vector is described in more detail below.

The first recombinant nucleic acid sequence construct can also include the promoter, intron, transcription termination region, initiation codon, termination codon, and/or polyadenylation signal. The first recombinant nucleic acid sequence construct can further include the leader sequence, in which the leader sequence is located upstream (or 5′) of the heterologous nucleic acid sequence encoding the heavy chain polypeptide. Accordingly, the signal peptide encoded by the leader sequence can be linked by a peptide bond to the heavy chain polypeptide.

The second recombinant nucleic acid sequence construct can also include the promoter, initiation codon, termination codon, and polyadenylation signal. The second recombinant nucleic acid sequence construct can further include the leader sequence, in which the leader sequence is located upstream (or 5′) of the heterologous nucleic acid sequence encoding the light chain polypeptide. Accordingly, the signal peptide encoded by the leader sequence can be linked by a peptide bond to the light chain polypeptide.

Accordingly, one example of arrangement 1 can include the first vector (and thus first recombinant nucleic acid sequence construct) encoding the heavy chain polypeptide that includes VH and CH1, and the second vector (and thus second recombinant nucleic acid sequence construct) encoding the light chain polypeptide that includes VL and CL. A second example of arrangement 1 can include the first vector (and thus first recombinant nucleic acid sequence construct) encoding the heavy chain polypeptide that includes VH, CH1, hinge region, CH2, and CH3, and the second vector (and thus second recombinant nucleic acid sequence construct) encoding the light chain polypeptide that includes VL and CL.

### (2) Arrangement 2

In a second arrangement, the recombinant nucleic acid sequence construct can include the heterologous nucleic acid sequence encoding the heavy chain polypeptide and the heterologous nucleic acid sequence encoding the light chain polypeptide. The heterologous nucleic acid sequence encoding the heavy chain polypeptide can be positioned upstream (or 5′) of the heterologous nucleic acid sequence encoding the light chain polypeptide. Alternatively, the heterologous nucleic acid sequence encoding the light chain polypeptide can be positioned upstream (or 5′) of the heterologous nucleic acid sequence encoding the heavy chain polypeptide.

The recombinant nucleic acid sequence construct can be placed in the vector as described in more detail below.

The recombinant nucleic acid sequence construct can include the heterologous nucleic acid sequence encoding the protease cleavage site and/or the linker sequence. If included in the recombinant nucleic acid sequence construct, the heterologous nucleic acid sequence encoding the protease cleavage site can be positioned between the heterologous nucleic acid sequence encoding the heavy chain polypeptide and the heterologous nucleic acid sequence encoding the light chain polypeptide. Accordingly, the protease cleavage site allows for separation of the heavy chain polypeptide and the light chain polypeptide into distinct polypeptides upon expression. In other embodiments, if the linker sequence is included in the recombinant nucleic acid sequence construct, then the linker sequence can be positioned between the heterologous nucleic acid sequence encoding the heavy chain polypeptide and the heterologous nucleic acid sequence encoding the light chain polypeptide.

The recombinant nucleic acid sequence construct can also include the promoter, intron, transcription termination region, initiation codon, termination codon, and/or polyadenylation signal. The recombinant nucleic acid sequence construct can include one or more promoters. The recombinant nucleic acid sequence construct can include two promoters such that one promoter can be associated with the heterologous nucleic acid sequence encoding the heavy chain polypeptide and the second promoter can be associated with the heterologous nucleic acid sequence encoding the light chain polypeptide. In still other embodiments, the recombinant nucleic acid sequence construct can include one promoter that is associated with the heterologous nucleic acid sequence encoding the heavy chain polypeptide and the heterologous nucleic acid sequence encoding the light chain polypeptide.

The recombinant nucleic acid sequence construct can further include two leader sequences, in which a first leader sequence is located upstream (or 5′) of the heterologous nucleic acid sequence encoding the heavy chain polypeptide and a second leader sequence is located upstream (or 5′) of the heterologous nucleic acid sequence encoding the light chain polypeptide. Accordingly, a first signal peptide encoded by the first leader sequence can be linked by a peptide bond to the heavy chain polypeptide and a second signal peptide encoded by the second leader sequence can be linked by a peptide bond to the light chain polypeptide.

Accordingly, one example of arrangement 2 can include the vector (and thus recombinant nucleic acid sequence construct) encoding the heavy chain polypeptide that includes VH and CH1, and the light chain polypeptide that includes VL and CL, in which the linker sequence is positioned between the heterologous nucleic acid sequence encoding the heavy chain polypeptide and the heterologous nucleic acid sequence encoding the light chain polypeptide.

A second example of arrangement of 2 can include the vector (and thus recombinant nucleic acid sequence construct) encoding the heavy chain polypeptide that includes VH and CH1, and the light chain polypeptide that includes VL and CL, in which the heterologous nucleic acid sequence encoding the protease cleavage site is positioned between the heterologous nucleic acid sequence encoding the heavy chain polypeptide and the heterologous nucleic acid sequence encoding the light chain polypeptide.

A third example of arrangement 2 can include the vector (and thus recombinant nucleic acid sequence construct) encoding the heavy chain polypeptide that includes VH, CH1, hinge region, CH2, and CH3, and the light chain polypeptide that includes VL and CL, in which the linker sequence is positioned between the heterologous nucleic acid sequence encoding the heavy chain polypeptide and the heterologous nucleic acid sequence encoding the light chain polypeptide.

A forth example of arrangement of 2 can include the vector (and thus recombinant nucleic acid sequence construct) encoding the heavy chain polypeptide that includes VH, CH1, hinge region, CH2, and CH3, and the light chain polypeptide that includes VL and CL, in which the heterologous nucleic acid sequence encoding the protease cleavage site is positioned between the heterologous nucleic acid sequence encoding the heavy chain polypeptide and the heterologous nucleic acid sequence encoding the light chain polypeptide.

### c. Expression from the Recombinant Nucleic Acid Sequence Construct

As described above, the recombinant nucleic acid sequence construct can include, amongst the one or more components, the heterologous nucleic acid sequence encoding the heavy chain polypeptide and/or the heterologous nucleic acid sequence encoding the light chain polypeptide. Accordingly, the recombinant nucleic acid sequence construct can facilitate expression of the heavy chain polypeptide and/or the light chain polypeptide.

When arrangement 1 as described above is utilized, the first recombinant nucleic acid sequence construct can facilitate the expression of the heavy chain polypeptide and the second recombinant nucleic acid sequence construct can facilitate expression of the light chain polypeptide. When arrangement 2 as described above is utilized, the recombinant nucleic acid sequence construct can facilitate the expression of the heavy chain polypeptide and the light chain polypeptide.

Upon expression, for example, but not limited to, in a cell, organism, or mammal, the heavy chain polypeptide and the light chain polypeptide can assemble into the synthetic antibody. In particular, the heavy chain polypeptide and the light chain polypeptide can interact with one another such that assembly results in the synthetic antibody being capable of binding the antigen. In other embodiments, the heavy chain polypeptide and the light chain polypeptide can interact with one another such that assembly results in the synthetic antibody being more immunogenic as compared to an antibody not assembled as described herein. In still other embodiments, the heavy chain polypeptide and the light chain polypeptide can interact with one another such that assembly results in the synthetic antibody being capable of eliciting or inducing an immune response against the antigen.

### d. Vector

The recombinant nucleic acid sequence construct described above can be placed in one or more vectors. The one or more vectors can contain an origin of replication. The one or more vectors can be a plasmid, bacteriophage, bacterial artificial chromosome or yeast artificial chromosome. The one or more vectors can be either a self-replication extra chromosomal vector, or a vector which integrates into a host genome.

The one or more vectors can be a heterologous expression construct, which is generally a plasmid that is used to introduce a specific gene into a target cell. Once the expression vector is inside the cell, the heavy chain polypeptide and/or light chain polypeptide that are encoded by the recombinant nucleic acid sequence construct is produced by the cellular-transcription and translation machinery ribosomal complexes. The one or more vectors can express large amounts of stable messenger RNA, and therefore proteins.

### (1) Expression Vector

The one or more vectors can be a circular plasmid or a linear nucleic acid. The circular plasmid and linear nucleic acid are capable of directing expression of a particular nucleotide sequence in an appropriate subject cell. The one or more vectors comprising the recombinant nucleic acid sequence construct may be chimeric, meaning that at least one of its components is heterologous with respect to at least one of its other components.

### (2) Plasmid

The one or more vectors can be a plasmid. The plasmid may be useful for transfecting cells with the recombinant nucleic acid sequence construct. The plasmid may be useful for introducing the recombinant nucleic acid sequence construct into the subject. The plasmid may also comprise a regulatory sequence, which may be well suited for gene expression in a cell into which the plasmid is administered.

The plasmid may also comprise a mammalian origin of replication in order to maintain the plasmid extrachromosomally and produce multiple copies of the plasmid in a cell. The plasmid may be pVAX1, pCEP4 or pREP4 from Invitrogen (San Diego, CA), which may comprise the Epstein Barr virus origin of replication and nuclear antigen EBNA-1 coding region, which may produce high copy episomal replication without integration. The backbone of the plasmid may be pAV0242. The plasmid may be a replication defective adenovirus type 5 (Ad5) plasmid.

The plasmid may be pSE420 (Invitrogen, San Diego, Calif.), which may be used for protein production in Escherichia coli (E. coli). The plasmid may also be pYES2 (Invitrogen, San Diego, Calif.), which may be used for protein production in Saccharomyces cerevisiae strains of yeast. The plasmid may also be of the MAXBAC™ complete baculovirus expression system (Invitrogen, San Diego, Calif), which may be used for protein production in insect cells. The plasmid may also be pcDNAI or pcDNA3 (Invitrogen, San Diego, Calif), which may be used for protein production in mammalian cells such as Chinese hamster ovary (CHO) cells.

### (3) RNA

In one embodiment, the nucleic acid is an RNA molecule. In one embodiment, the RNA molecule is transcribed from a DNA sequence described herein. For example, in some embodiments, the RNA molecule is encoded by a DNA sequence at least 90% homologous to one of SEQ ID NO:1, SEQ ID NO:3, SEQ ID NO:5, SEQ ID NO:7, SEQ ID NO:9, SEQ ID NO:11, SEQ ID NO:13, SEQ ID NO: 15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39 SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:111, SEQ ID NO:113, SEQ ID NO:115, SEQ ID NO:117, SEQ ID NO:119, SEQ ID NO:121, SEQ ID NO:123, SEQ ID NO:125, SEQ ID NO:127, SEQ ID NO:129, SEQ ID NO:131, or SEQ ID NO:133, or a variant thereof or a fragment thereof.

In another embodiment, the nucleotide sequence comprises an RNA sequence transcribed by a DNA sequence encoding a polypeptide sequence of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:17, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO: 60, SEQ ID NO:62, SEQ ID NO:64, SEQ ID NO:66, SEQ ID NO:68,SEQ ID NO: 70, SEQ ID NO:72, SEQ ID NO:74, SEQ ID NO:76, SEQ ID NO:78, SEQ ID NO: 80, SEQ ID NO:82, SEQ ID NO:84, SEQ ID NO:86, SEQ ID NO:88, SEQ ID NO: 90, SEQ ID NO:92, SEQ ID NO:94, SEQ ID NO:96, SEQ ID NO:98, SEQ ID NO: 100, SEQ ID NO:102, SEQ ID NO:104, SEQ ID NO:106, SEQ ID NO:108, SEQ ID NO: 110, SEQ ID NO:112, SEQ ID NO:114, SEQ ID NO:116, SEQ ID NO:118, SEQ ID NO: 120, SEQ ID NO:122, SEQ ID NO:124, SEQ ID NO:126, SEQ ID NO:128, SEQ ID NO: 130, SEQ ID NO:132, or SEQ ID NO:134, or a variant thereof or a fragment thereof.

Accordingly, in one embodiment, the invention provides an RNA molecule encoding one or more of the DMAbs. The RNA may be plus-stranded. Accordingly, in some embodiments, the RNA molecule can be translated by cells without needing any intervening replication steps such as reverse transcription. A RNA molecule useful with the invention may have a 5′ cap (e.g. a 7-methylguanosine). This cap can enhance in vivo translation of the RNA. The 5′ nucleotide of a RNA molecule useful with the invention may have a 5′ triphosphate group. In a capped RNA this may be linked to a 7-methylguanosine via a 5′-to-5′ bridge. A RNA molecule may have a 3′ poly-A tail. It may also include a poly-A polymerase recognition sequence (e.g. AAUAAA) near its 3′ end. A RNA molecule useful with the invention may be single-stranded. A RNA molecule useful with the invention may comprise synthetic RNA. In some embodiments, the RNA molecule is a naked RNA molecule. In one embodiment, the RNA molecule is comprised within a vector.

In one embodiment, the RNA has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of RNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many RNAs is known in the art. In other embodiments, the 5′ UTR can be derived from an RNA virus whose RNA genome is stable in cells. In other embodiments, various nucleotide analogues can be used in the 3′ or 5′ UTR to impede exonuclease degradation of the RNA.

In one embodiment, the RNA has both a cap on the 5′ end and a 3′ poly(A) tail which determine ribosome binding, initiation of translation and stability of RNA in the cell.

In one embodiment, the RNA is a nucleoside-modified RNA. Nucleoside-modified RNA have particular advantages over non-modified RNA, including for example, increased stability, low or absent innate immunogenicity, and enhanced translation

### (4) Circular and Linear Vector

The one or more vectors may be circular plasmid, which may transform a target cell by integration into the cellular genome or exist extrachromosomally (e.g., autonomous replicating plasmid with an origin of replication). The vector can be pVAX, pcDNA3.0, or provax, or any other expression vector capable of expressing the heavy chain polypeptide and/or light chain polypeptide encoded by the recombinant nucleic acid sequence construct.

Also provided herein is a linear nucleic acid, or linear expression cassette (“LEC”), that is capable of being efficiently delivered to a subject via electroporation and expressing the heavy chain polypeptide and/or light chain polypeptide encoded by the recombinant nucleic acid sequence construct. The LEC may be any linear DNA devoid of any phosphate backbone. The LEC may not contain any antibiotic resistance genes and/or a phosphate backbone. The LEC may not contain other nucleic acid sequences unrelated to the desired gene expression.

The LEC may be derived from any plasmid capable of being linearized. The plasmid may be capable of expressing the heavy chain polypeptide and/or light chain polypeptide encoded by the recombinant nucleic acid sequence construct. The plasmid can be pNP (Puerto Rico/34) or pM2 (New Caledonia/99). The plasmid may be WLV009, pVAX, pcDNA3.0, or provax, or any other expression vector capable of expressing the heavy chain polypeptide and/or light chain polypeptide encoded by the recombinant nucleic acid sequence construct.

The LEC can be perM2. The LEC can be perNP. perNP and perMR can be derived from pNP (Puerto Rico/34) and pM2 (New Caledonia/99), respectively.

### (5) Viral Vectors

In one embodiment, viral vectors are provided herein which are capable of delivering a nucleic acid of the invention to a cell. The expression vector may be provided to a cell in the form of a viral vector. Viral vector technology is well known in the art and is described, for example, in Sambrook et al. (2001), and in Ausubel et al. (1997), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

### (6) Method of Preparing the Vector

Provided herein is a method for preparing the one or more vectors in which the recombinant nucleic acid sequence construct has been placed. After the final subcloning step, the vector can be used to inoculate a cell culture in a large scale fermentation tank, using known methods in the art.

In other embodiments, after the final subcloning step, the vector can be used with one or more electroporation (EP) devices. The EP devices are described below in more detail.

The one or more vectors can be formulated or manufactured using a combination of known devices and techniques, but preferably they are manufactured using a plasmid manufacturing technique that is described in a licensed, co-pending U.S. provisional application U.S. Ser. No. 60/939,792, which was filed on May 23, 2007. In some examples, the DNA plasmids described herein can be formulated at concentrations greater than or equal to 10 mg/mL. The manufacturing techniques also include or incorporate various devices and protocols that are commonly known to those of ordinary skill in the art, in addition to those described in U.S. Ser. No. 60/939,792, including those described in a licensed patent, U.S. Pat. No. 7,238,522, which issued on Jul. 3, 2007. The above-referenced application and patent, U.S. Ser. No. 60/939,792 and U.S. Pat. No. 7,238,522, respectively, are hereby incorporated in their entirety.

### 4. ANTIBODY

As described above, the recombinant nucleic acid sequence can encode the antibody, a fragment thereof, a variant thereof, or a combination thereof. The antibody can bind or react with the antigen, which is described in more detail below.

The antibody may comprise a heavy chain and a light chain complementarity determining region (“CDR”) set, respectively interposed between a heavy chain and a light chain framework (“FR”) set which provide support to the CDRs and define the spatial relationship of the CDRs relative to each other. The CDR set may contain three hypervariable regions of a heavy or light chain V region. Proceeding from the N-terminus of a heavy or light chain, these regions are denoted as “CDR1,” “CDR2,” and “CDR3,” respectively. An antigen-binding site, therefore, may include six CDRs, comprising the CDR set from each of a heavy and a light chain V region.

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)₂ fragment, which comprises both antigen-binding sites. Accordingly, the antibody can be the Fab or F(ab′)₂. The Fab can include the heavy chain polypeptide and the light chain polypeptide. The heavy chain polypeptide of the Fab can include the VH region and the CH1 region. The light chain of the Fab can include the VL region and CL region.

The antibody can be an immunoglobulin (Ig). The Ig can be, for example, IgA, IgM, IgD, IgE, and IgG. The immunoglobulin can include the heavy chain polypeptide and the light chain polypeptide. The heavy chain polypeptide of the immunoglobulin can include a VH region, a CH1 region, a hinge region, a CH2 region, and a CH3 region. The light chain polypeptide of the immunoglobulin can include a VL region and CL region.

The antibody can be a polyclonal or monoclonal antibody. The antibody can be a chimeric antibody, a single chain antibody, an affinity matured antibody, a human antibody, a humanized antibody, or a fully human antibody. The humanized antibody can be an antibody from a non-human species that binds the desired antigen having one or more complementarity determining regions (CDRs) from the non-human species and framework regions from a human immunoglobulin molecule.

The antibody can be an IgG1 antibody, an IgG2 antibody, an IgG3 antibody, or an IgG4 antibody. The antibody can be a chimera of any of an IgG1 antibody, an IgG2 antibody, an IgG3 antibody, or an IgG4 antibody. In some embodiments, the antibody hinge domain is modified. For example, in one embodiment, the antibody includes the amino acid substitution Ser288Pro. In one embodiment, includes the amino acid substitution Ser288Pro prevents IgG4 Fab arm switching.

The antibody can be a bispecific antibody as described below in more detail. The antibody can be a bifunctional antibody as also described below in more detail.

As described above, the antibody can be generated in the subject upon administration of the composition to the subject. The antibody may have a half-life within the subject. In some embodiments, the antibody may be modified to extend or shorten its half-life within the subject. Such modifications are described below in more detail.

The antibody can be defucosylated as described in more detail below.

### a. Bispecific Antibody

The recombinant nucleic acid sequence can encode a bispecific antibody, a fragment thereof, a variant thereof, or a combination thereof. The bispecific antibody can bind or react with two antigens, for example, two of the antigens described below in more detail. The bispecific antibody can be comprised of fragments of two of the antibodies described herein, thereby allowing the bispecific antibody to bind or react with two desired target molecules, which may include the antigen, which is described below in more detail, a ligand, including a ligand for a receptor, a receptor, including a ligand-binding site on the receptor, a ligand-receptor complex, and a marker.

### b. Bifunctional Antibody

The recombinant nucleic acid sequence can encode a bifunctional antibody, a fragment thereof, a variant thereof, or a combination thereof. The bifunctional antibody can bind or react with the antigen described below. The bifunctional antibody can also be modified to impart an additional functionality to the antibody beyond recognition of and binding to the antigen. Such a modification can include, but is not limited to, coupling to factor H or a fragment thereof. Factor H is a soluble regulator of complement activation and thus, may contribute to an immune response via complement-mediated lysis (CML).

### c. Extension of Antibody Half-Life

As described above, the antibody may be modified to extend or shorten the half-life of the antibody in the subject. The modification may extend or shorten the half-life of the antibody in the serum of the subject.

The modification may be present in a constant region of the antibody. The modification may be one or more amino acid substitutions in a constant region of the antibody that extend the half-life of the antibody as compared to a half-life of an antibody not containing the one or more amino acid substitutions. The modification may be one or more amino acid substitutions in the CH2 domain of the antibody that extend the half-life of the antibody as compared to a half-life of an antibody not containing the one or more amino acid substitutions.

In some embodiments, the one or more amino acid substitutions in the constant region may include replacing a methionine residue in the constant region with a tyrosine residue, a serine residue in the constant region with a threonine residue, a threonine residue in the constant region with a glutamate residue, or any combination thereof, thereby extending the half-life of the antibody.

In other embodiments, the one or more amino acid substitutions in the constant region may include replacing a methionine residue in the CH2 domain with a tyrosine residue, a serine residue in the CH2 domain with a threonine residue, a threonine residue in the CH2 domain with a glutamate residue, or any combination thereof, thereby extending the half-life of the antibody.

### d. Defucosylation

The recombinant nucleic acid sequence can encode an antibody that is not fucosylated (i.e., a defucosylated antibody or a non-fucosylated antibody), a fragment thereof, a variant thereof, or a combination thereof. Fucosylation includes the addition of the sugar fucose to a molecule, for example, the attachment of fucose to N-glycans, O-glycans and glycolipids. Accordingly, in a defucosylated antibody, fucose is not attached to the carbohydrate chains of the constant region. In turn, this lack of fucosylation may improve FcγRIIIa binding and antibody directed cellular cytotoxic (ADCC) activity by the antibody as compared to the fucosylated antibody. Therefore, in some embodiments, the non-fucosylated antibody may exhibit increased ADCC activity as compared to the fucosylated antibody.

The antibody may be modified so as to prevent or inhibit fucosylation of the antibody. In some embodiments, such a modified antibody may exhibit increased ADCC activity as compared to the unmodified antibody. The modification may be in the heavy chain, light chain, or a combination thereof. The modification may be one or more amino acid substitutions in the heavy chain, one or more amino acid substitutions in the light chain, or a combination thereof.

### 5. ANTIGEN

The synthetic antibody is directed to the antigen or fragment or variant thereof. The antigen can be a nucleic acid sequence, an amino acid sequence, a polysaccharide or a combination thereof. The nucleic acid sequence can be DNA, RNA, cDNA, a variant thereof, a fragment thereof, or a combination thereof. The amino acid sequence can be a protein, a peptide, a variant thereof, a fragment thereof, or a combination thereof. The polysaccharide can be a nucleic acid encoded polysaccharide.

The antigen can be from a virus. The antigen can be associated with viral infection. In one embodiment, the antigen can be associated with an HIV infection. In one embodiment, the antigen can be HIV antigen.

In one embodiment, a synthetic antibody of the invention targets two or more antigens. In one embodiment, at least one antigen of a bispecific antibody is selected from the antigens described herein. In one embodiment, the two or more antigens are selected from the antigens described herein.

### a. Viral Antigens

The viral antigen can be a viral antigen or fragment or variant thereof. The virus can be a disease causing virus. The virus can be the Human Immunodeficiency virus.

The antigen may be an HIV viral antigen, or fragment thereof, or variant thereof. The HIV antigen can be from a factor that allows the virus to replicate, infect or survive. Factors that allow HIV to replicate or survive include, but are not limited to structural proteins and non-structural proteins. Such a protein can be an envelope protein or a glycoprotein.

In some embodiments, the HIV antigen can be a subtype A envelope protein, subtype B envelope protein, subtype C envelope protein, subtype D envelope protein, subtype B Nef-Rev protein, Gag subtype A, B, C, or D protein, MPol protein, a nucleic acid or amino acid sequences of Env A, Env B, Env C, Env D, B Nef-Rev, Gag, or any combination thereof. In one embodiment, an envelope protein is an HIV glycoprotein. In one embodiment, the HIV glycoprotein (gp) is HIV gp120, HIV gp41, or gp160.

A synthetic antibody specific for HIV can include a Fab fragment comprising the amino acid sequence of SEQ ID NO:3, which is encoded by the nucleic acid sequence of SEQ ID NO:4, SEQ ID NO: 5, which is encoded by the nucleic acid sequence of SEQ ID NO:6, amino acid sequence of SEQ ID NO:11, which is encoded by the nucleic acid sequence of SEQ ID NO:12, or SEQ ID NO: 13, which is encoded by the nucleic acid sequence of SEQ ID NO:14. The synthetic antibody can comprise the amino acid sequence of SEQ ID NO:1, which is encoded by the nucleic acid sequence of SEQ ID NO:2, the amino acid sequence of SEQ ID NO:7, which is encoded by the nucleic acid sequence of SEQ ID NO:8, or the amino acid sequence of SEQ ID NO:9, which is encoded by the nucleic acid sequence of SEQ ID NO:10.

A synthetic antibody specific for HIV can include a Fab fragment comprising the amino acid sequence of SEQ ID NO:17, which is encoded by the nucleic acid sequence of SEQ ID NO:18, SEQ ID NO: 19, which is encoded by the nucleic acid sequence of SEQ ID NO:20, amino acid sequence of SEQ ID NO:25, which is encoded by the nucleic acid sequence of SEQ ID NO:26, or SEQ ID NO: 27, which is encoded by the nucleic acid sequence of SEQ ID NO:28. The synthetic antibody can comprise the amino acid sequence of SEQ ID NO:15, which is encoded by the nucleic acid sequence of SEQ ID NO:16, the amino acid sequence of SEQ ID NO:21, which is encoded by the nucleic acid sequence of SEQ ID NO:22, or the amino acid sequence of SEQ ID NO:23, which is encoded by the nucleic acid sequence of SEQ ID NO:24.

A synthetic antibody specific for HIV can include a Fab fragment comprising the amino acid sequence of SEQ ID NO:39, which is encoded by the nucleic acid sequence of SEQ ID NO:40, SEQ ID NO: 41, which is encoded by the nucleic acid sequence of SEQ ID NO:42, amino acid sequence of SEQ ID NO:47, which is encoded by the nucleic acid sequence of SEQ ID NO:48, or SEQ ID NO: 49, which is encoded by the nucleic acid sequence of SEQ ID NO:50. The synthetic antibody can comprise the amino acid sequence of SEQ ID NO:37, which is encoded by the nucleic acid sequence of SEQ ID NO:38, the amino acid sequence of SEQ ID NO:43, which is encoded by the nucleic acid sequence of SEQ ID NO:44, the amino acid sequence of SEQ ID NO:45, which is encoded by the nucleic acid sequence of SEQ ID NO:46, the amino acid sequence of SEQ ID NO:51, which is encoded by the nucleic acid sequence of SEQ ID NO:52, or the amino acid sequence of SEQ ID NO:53, which is encoded by the nucleic acid sequence of SEQ ID NO:54.

The synthetic antibody can comprise the amino acid sequence of SEQ ID NO:29, which is encoded by the nucleic acid sequence of SEQ ID NO:30, the amino acid sequence of SEQ ID NO:31, which is encoded by the nucleic acid sequence of SEQ ID NO:32, the amino acid sequence of SEQ ID NO:33, which is encoded by the nucleic acid sequence of SEQ ID NO:34, or the amino acid sequence of SEQ ID NO:35 which is encoded by the nucleic acid sequence of SEQ ID NO:36.

A synthetic antibody specific for HIV can include an Ig comprising the amino acid sequence of SEQ ID NO:1,

The Ig can comprise the amino acid sequence of SEQ ID NO:7, which is encoded by the nucleic acid sequence of SEQ ID NO:8. The Ig can comprise the amino acid sequence of SEQ ID NO: 9, which is encoded by the nucleic acid sequence of SEQ ID NO:10. The Ig can comprise the amino acid sequence of SEQ ID NO:15, which is encoded by the nucleic acid sequence of SEQ ID NO:16. The Ig can comprise the amino acid sequence of SEQ ID NO: 21, which is encoded by the nucleic acid sequence of SEQ ID NO:22. The Ig can comprise the amino acid sequence of SEQ ID NO: 23, which is encoded by the nucleic acid sequence of SEQ ID NO: 24. The Ig can comprise the amino acid sequence of SEQ ID NO: 29, which is encoded by the nucleic acid sequence of SEQ ID NO: 30. The Ig can comprise the amino acid sequence of SEQ ID NO:31, which is encoded by the nucleic acid sequence of SEQ ID NO: 32. The Ig can comprise the amino acid sequence of SEQ ID NO:33, which is encoded by the nucleic acid sequence of SEQ ID NO: 34. The Ig can comprise the amino acid sequence of SEQ ID NO:35, which is encoded by the nucleic acid sequence of SEQ ID NO: 36. The Ig can comprise the amino acid sequence of SEQ ID NO:37, which is encoded by the nucleic acid sequence of SEQ ID NO: 38. The Ig can comprise the amino acid sequence of SEQ ID NO:43, which is encoded by the nucleic acid sequence of SEQ ID NO: 44. The Ig can comprise the amino acid sequence of SEQ ID NO:45, which is encoded by the nucleic acid sequence of SEQ ID NO: 46. The Ig can comprise the amino acid sequence of SEQ ID NO:51, which is encoded by the nucleic acid sequence of SEQ ID NO: 52. The Ig can comprise the amino acid sequence of SEQ ID NO:53, which is encoded by the nucleic acid sequence of SEQ ID NO:54. The Ig can comprise the amino acid sequence of SEQ ID NO:55, which is encoded by the nucleic acid sequence of SEQ ID NO: 56. The Ig can comprise the amino acid sequence of SEQ ID NO:57, which is encoded by the nucleic acid sequence of SEQ ID NO: 58. The Ig can comprise the amino acid sequence of SEQ ID NO:59, which is encoded by the nucleic acid sequence of SEQ ID NO: 60. The Ig can comprise the amino acid sequence of SEQ ID NO:61, which is encoded by the nucleic acid sequence of SEQ ID NO: 62. The Ig can comprise the amino acid sequence of SEQ ID NO:63, which is encoded by the nucleic acid sequence of SEQ ID NO: 64. The Ig can comprise the amino acid sequence of SEQ ID NO:65, which is encoded by the nucleic acid sequence of SEQ ID NO: 66. The Ig can comprise the amino acid sequence of SEQ ID NO:67, which is encoded by the nucleic acid sequence of SEQ ID NO: 68. The Ig can comprise the amino acid sequence of SEQ ID NO:69, which is encoded by the nucleic acid sequence of SEQ ID NO: 70. The Ig can comprise the amino acid sequence of SEQ ID NO:71, which is encoded by the nucleic acid sequence of SEQ ID NO: 72. The Ig can comprise the amino acid sequence of SEQ ID NO:73, which is encoded by the nucleic acid sequence of SEQ ID NO: 74. The Ig can comprise the amino acid sequence of SEQ ID NO:75, which is encoded by the nucleic acid sequence of SEQ ID NO: 76. The Ig can comprise the amino acid sequence of SEQ ID NO:77, which is encoded by the nucleic acid sequence of SEQ ID NO: 78. The Ig can comprise the amino acid sequence of SEQ ID NO:79, which is encoded by the nucleic acid sequence of SEQ ID NO: 80. The Ig can comprise the amino acid sequence of SEQ ID NO:81, which is encoded by the nucleic acid sequence of SEQ ID NO: 82. The Ig can comprise the amino acid sequence of SEQ ID NO:83, which is encoded by the nucleic acid sequence of SEQ ID NO: 84. The Ig can comprise the amino acid sequence of SEQ ID NO:85, which is encoded by the nucleic acid sequence of SEQ ID NO: 86. The Ig can comprise the amino acid sequence of SEQ ID NO:87, which is encoded by the nucleic acid sequence of SEQ ID NO: 88. The Ig can comprise the amino acid sequence of SEQ ID NO:89, which is encoded by the nucleic acid sequence of SEQ ID NO: 90. The Ig can comprise the amino acid sequence of SEQ ID NO:91, which is encoded by the nucleic acid sequence of SEQ ID NO: 92. The Ig can comprise the amino acid sequence of SEQ ID NO:93, which is encoded by the nucleic acid sequence of SEQ ID NO: 94. The Ig can comprise the amino acid sequence of SEQ ID NO:95, which is encoded by the nucleic acid sequence of SEQ ID NO: 96. The Ig can comprise the amino acid sequence of SEQ ID NO:97, which is encoded by the nucleic acid sequence of SEQ ID NO: 98. The Ig can comprise the amino acid sequence of SEQ ID NO:99, which is encoded by the nucleic acid sequence of SEQ ID NO: 100. The Ig can comprise the amino acid sequence of SEQ ID NO:101, which is encoded by the nucleic acid sequence of SEQ ID NO: 102. The Ig can comprise the amino acid sequence of SEQ ID NO:103, which is encoded by the nucleic acid sequence of SEQ ID NO: 104. The Ig can comprise the amino acid sequence of SEQ ID NO:105, which is encoded by the nucleic acid sequence of SEQ ID NO: 106. The Ig can comprise the amino acid sequence of SEQ ID NO:107, which is encoded by the nucleic acid sequence of SEQ ID NO: 108. The Ig can comprise the amino acid sequence of SEQ ID NO:109, which is encoded by the nucleic acid sequence of SEQ ID NO: 110. The Ig can comprise the amino acid sequence of SEQ ID NO:111, which is encoded by the nucleic acid sequence of SEQ ID NO: 112. The Ig can comprise the amino acid sequence of SEQ ID NO:113, which is encoded by the nucleic acid sequence of SEQ ID NO: 114. The Ig can comprise the amino acid sequence of SEQ ID NO:115, which is encoded by the nucleic acid sequence of SEQ ID NO: 116. The Ig can comprise the amino acid sequence of SEQ ID NO:117, which is encoded by the nucleic acid sequence of SEQ ID NO: 118. The Ig can comprise the amino acid sequence of SEQ ID NO:119, which is encoded by the nucleic acid sequence of SEQ ID NO: 120. The Ig can comprise the amino acid sequence of SEQ ID NO:121, which is encoded by the nucleic acid sequence of SEQ ID NO: 122. The Ig can comprise the amino acid sequence of SEQ ID NO:123, which is encoded by the nucleic acid sequence of SEQ ID NO: 124. The Ig can comprise the amino acid sequence of SEQ ID NO:125, which is encoded by the nucleic acid sequence of SEQ ID NO: 126. The Ig can comprise the amino acid sequence of SEQ ID NO:127, which is encoded by the nucleic acid sequence of SEQ ID NO: 128. The Ig can comprise the amino acid sequence of SEQ ID NO:129, which is encoded by the nucleic acid sequence of SEQ ID NO: 130. The Ig can comprise the amino acid sequence of SEQ ID NO:131, which is encoded by the nucleic acid sequence of SEQ ID NO: 132. The Ig can comprise the amino acid sequence of SEQ ID NO:133, which is encoded by the nucleic acid sequence of SEQ ID NO: 134.

### 6. EXCIPIENTS AND OTHER COMPONENTS OF THE COMPOSITION

The composition may further comprise a pharmaceutically acceptable excipient. The pharmaceutically acceptable excipient can be functional molecules such as vehicles, carriers, or diluents. The pharmaceutically acceptable excipient can be a transfection facilitating agent, which can include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

The transfection facilitating agent is a polyanion, polycation, including poly-L-glutamate (LGS), or lipid. The transfection facilitating agent is poly-L-glutamate, and the poly-L-glutamate may be present in the composition at a concentration less than 6 mg/ml. The transfection facilitating agent may also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the composition. The composition may also include a transfection facilitating agent such as lipids, liposomes, including lecithin liposomes or other liposomes known in the art, as a DNA-liposome mixture (see for example W09324640), calcium ions, viral proteins, polyanions, polycations, or nanoparticles, or other known transfection facilitating agents. The transfection facilitating agent is a polyanion, polycation, including poly-L-glutamate (LGS), or lipid. Concentration of the transfection agent in the vaccine is less than 4 mg/ml, less than 2 mg/ml, less than 1 mg/ml, less than 0.750 mg/ml, less than 0.500 mg/ml, less than 0.250 mg/ml, less than 0.100 mg/ml, less than 0.050 mg/ml, or less than 0.010 mg/ml.

The composition may further comprise a genetic facilitator agent as described in U.S. Ser. No. 021,579 filed Apr. 1, 1994, which is fully incorporated by reference.

The composition may comprise DNA at quantities of from about 1 nanogram to 100 milligrams; about 1 microgram to about 10 milligrams; or preferably about 0.1 microgram to about 10 milligrams; or more preferably about 1 milligram to about 2 milligram. In some preferred embodiments, composition according to the present invention comprises about 5 nanogram to about 1000 micrograms of DNA. In some preferred embodiments, composition can contain about 10 nanograms to about 800 micrograms of DNA. In some preferred embodiments, the composition can contain about 0.1 to about 500 micrograms of DNA. In some preferred embodiments, the composition can contain about 1 to about 350 micrograms of DNA. In some preferred embodiments, the composition can contain about 25 to about 250 micrograms, from about 100 to about 200 microgram, from about 1 nanogram to 100 milligrams; from about 1 microgram to about 10 milligrams; from about 0.1 microgram to about 10 milligrams; from about 1 milligram to about 2 milligram, from about 5 nanogram to about 1000 micrograms, from about 10 nanograms to about 800 micrograms, from about 0.1 to about 500 micrograms, from about 1 to about 350 micrograms, from about 25 to about 250 micrograms, from about 100 to about 200 microgram of DNA.

The composition can be formulated according to the mode of administration to be used. An injectable pharmaceutical composition can be sterile, pyrogen free and particulate free. An isotonic formulation or solution can be used. Additives for isotonicity can include sodium chloride, dextrose, mannitol, sorbitol, and lactose. The composition can comprise a vasoconstriction agent. The isotonic solutions can include phosphate buffered saline. The composition can further comprise stabilizers including gelatin and albumin. The stabilizers can allow the formulation to be stable at room or ambient temperature for extended periods of time, including LGS or polycations or polyanions.

### 7. METHOD OF GENERATING THE SYNTHETIC ANTIBODY

The present invention also relates a method of generating the synthetic antibody. The method can include administering the composition to the subject in need thereof by using the method of delivery described in more detail below. Accordingly, the synthetic antibody is generated in the subject or in vivo upon administration of the composition to the subject.

The method can also include introducing the composition into one or more cells, and therefore, the synthetic antibody can be generated or produced in the one or more cells. The method can further include introducing the composition into one or more tissues, for example, but not limited to, skin and muscle, and therefore, the synthetic antibody can be generated or produced in the one or more tissues.

### 8. METHOD OF IDENTIFYING OR SCREENING FOR THE ANTIBODY

The present invention further relates to a method of identifying or screening for the antibody described above, which is reactive to or binds the antigen described above. The method of identifying or screening for the antibody can use the antigen in methodologies known in those skilled in art to identify or screen for the antibody. Such methodologies can include, but are not limited to, selection of the antibody from a library (e.g., phage display) and immunization of an animal followed by isolation and/or purification of the antibody.

### 9. METHOD OF DELIVERY OF THE COMPOSITION

The present invention also relates to a method of delivering the composition to the subject in need thereof. The method of delivery can include, administering the composition to the subject. Administration can include, but is not limited to, DNA injection with and without in vivo electroporation, liposome mediated delivery, and nanoparticle facilitated delivery.

The mammal receiving delivery of the composition may be human, primate, non-human primate, cow, cattle, sheep, goat, antelope, bison, water buffalo, bison, bovids, deer, hedgehogs, elephants, llama, alpaca, mice, rats, and chicken.

The composition may be administered by different routes including orally, parenterally, sublingually, transdermally, rectally, transmucosally, topically, via inhalation, via buccal administration, intrapleurally, intravenous, intraarterial, intraperitoneal, subcutaneous, intramuscular, intranasal intrathecal, and intraarticular or combinations thereof. For veterinary use, the composition may be administered as a suitably acceptable formulation in accordance with normal veterinary practice. The veterinarian can readily determine the dosing regimen and route of administration that is most appropriate for a particular animal. The composition may be administered by traditional syringes, needleless injection devices, “microprojectile bombardment gone guns”, or other physical methods such as electroporation (“EP”), “hydrodynamic method”, or ultrasound.

### a. Electroporation

Administration of the composition via electroporation may be accomplished using electroporation devices that can be configured to deliver to a desired tissue of a mammal, a pulse of energy effective to cause reversible pores to form in cell membranes, and preferable the pulse of energy is a constant current similar to a preset current input by a user. The electroporation device may comprise an electroporation component and an electrode assembly or handle assembly. The electroporation component may include and incorporate one or more of the various elements of the electroporation devices, including: controller, current waveform generator, impedance tester, waveform logger, input element, status reporting element, communication port, memory component, power source, and power switch. The electroporation may be accomplished using an in vivo electroporation device, for example CELLECTRA EP system (Inovio Pharmaceuticals, Plymouth Meeting, PA) or Elgen electroporator (Inovio Pharmaceuticals, Plymouth Meeting, PA) to facilitate transfection of cells by the plasmid.

The electroporation component may function as one element of the electroporation devices, and the other elements are separate elements (or components) in communication with the electroporation component. The electroporation component may function as more than one element of the electroporation devices, which may be in communication with still other elements of the electroporation devices separate from the electroporation component. The elements of the electroporation devices existing as parts of one electromechanical or mechanical device may not limited as the elements can function as one device or as separate elements in communication with one another. The electroporation component may be capable of delivering the pulse of energy that produces the constant current in the desired tissue, and includes a feedback mechanism. The electrode assembly may include an electrode array having a plurality of electrodes in a spatial arrangement, wherein the electrode assembly receives the pulse of energy from the electroporation component and delivers same to the desired tissue through the electrodes. At least one of the plurality of electrodes is neutral during delivery of the pulse of energy and measures impedance in the desired tissue and communicates the impedance to the electroporation component. The feedback mechanism may receive the measured impedance and can adjust the pulse of energy delivered by the electroporation component to maintain the constant current.

A plurality of electrodes may deliver the pulse of energy in a decentralized pattern. The plurality of electrodes may deliver the pulse of energy in the decentralized pattern through the control of the electrodes under a programmed sequence, and the programmed sequence is input by a user to the electroporation component. The programmed sequence may comprise a plurality of pulses delivered in sequence, wherein each pulse of the plurality of pulses is delivered by at least two active electrodes with one neutral electrode that measures impedance, and wherein a subsequent pulse of the plurality of pulses is delivered by a different one of at least two active electrodes with one neutral electrode that measures impedance.

The feedback mechanism may be performed by either hardware or software. The feedback mechanism may be performed by an analog closed-loop circuit. The feedback occurs every 50 μs, 20 μs, 10 μs or 1 μs, but is preferably a real-time feedback or instantaneous (i.e., substantially instantaneous as determined by available techniques for determining response time). The neutral electrode may measure the impedance in the desired tissue and communicates the impedance to the feedback mechanism, and the feedback mechanism responds to the impedance and adjusts the pulse of energy to maintain the constant current at a value similar to the preset current. The feedback mechanism may maintain the constant current continuously and instantaneously during the delivery of the pulse of energy.

Examples of electroporation devices and electroporation methods that may facilitate delivery of the composition of the present invention, include those described in U.S. Pat. No. 7,245,963 by Draghia-Akli, et al., U.S. Patent Pub. 2005/0052630 submitted by Smith, et al., the contents of which are hereby incorporated by reference in their entirety. Other electroporation devices and electroporation methods that may be used for facilitating delivery of the composition include those provided in co-pending and co-owned U.S. patent application Ser. No. 11/874,072, filed Oct. 17, 2007, which claims the benefit under 35 USC 119(e) to U.S. Provisional Applications Ser. Nos. 60/852,149, filed Oct. 17, 2006, and 60/978,982, filed Oct. 10, 2007, all of which are hereby incorporated in their entirety.

U.S. Pat. No. 7,245,963 by Draghia-Akli, et al. describes modular electrode systems and their use for facilitating the introduction of a biomolecule into cells of a selected tissue in a body or plant. The modular electrode systems may comprise a plurality of needle electrodes; a hypodermic needle; an electrical connector that provides a conductive link from a programmable constant-current pulse controller to the plurality of needle electrodes; and a power source. An operator can grasp the plurality of needle electrodes that are mounted on a support structure and firmly insert them into the selected tissue in a body or plant. The biomolecules are then delivered via the hypodermic needle into the selected tissue. The programmable constant-current pulse controller is activated and constant-current electrical pulse is applied to the plurality of needle electrodes. The applied constant-current electrical pulse facilitates the introduction of the biomolecule into the cell between the plurality of electrodes. The entire content of U.S. Pat. No. 7,245,963 is hereby incorporated by reference.

U.S. Patent Pub. 2005/0052630 submitted by Smith, et al. describes an electroporation device which may be used to effectively facilitate the introduction of a biomolecule into cells of a selected tissue in a body or plant. The electroporation device comprises an electro-kinetic device (“EKD device”) whose operation is specified by software or firmware. The EKD device produces a series of programmable constant-current pulse patterns between electrodes in an array based on user control and input of the pulse parameters, and allows the storage and acquisition of current waveform data. The electroporation device also comprises a replaceable electrode disk having an array of needle electrodes, a central injection channel for an injection needle, and a removable guide disk. The entire content of U.S. Patent Pub. 2005/0052630 is hereby incorporated by reference.

The electrode arrays and methods described in U.S. Pat. No. 7,245,963 and U.S. Patent Pub. 2005/0052630 may be adapted for deep penetration into not only tissues such as muscle, but also other tissues or organs. Because of the configuration of the electrode array, the injection needle (to deliver the biomolecule of choice) is also inserted completely into the target organ, and the injection is administered perpendicular to the target issue, in the area that is pre-delineated by the electrodes The electrodes described in U.S. Pat. No. 7,245,963 and U.S. Patent Pub. 2005/005263 are preferably 20 mm long and 21 gauge.

Additionally, contemplated in some embodiments that incorporate electroporation devices and uses thereof, there are electroporation devices that are those described in the following patents: U.S. Pat. No. 5,273,525 issued Dec. 28, 1993, U.S. Pat. No. 6,110,161 issued Aug. 29, 2000, U.S. Pat. No. 6,261,281 issued Jul. 17, 2001, and U.S. Pat. No. 6,958,060 issued Oct. 25, 2005, and U.S. Pat. No. 6,939,862 issued Sep. 6, 2005. Furthermore, patents covering subject matter provided in U.S. Pat. No. 6,697,669 issued Feb. 24, 2004, which concerns delivery of DNA using any of a variety of devices, and U.S. Pat. No. 7,328,064 issued Feb. 5, 2008, drawn to method of injecting DNA are contemplated herein. The above-patents are incorporated by reference in their entirety.

### 10. METHOD OF TREATMENT

Also provided herein is a method of treating, protecting against, and/or preventing disease in a subject in need thereof by generating the synthetic antibody in the subject. The method can include administering the composition to the subject. Administration of the composition to the subject can be done using the method of delivery described above.

In certain embodiments, the invention provides a method of treating protecting against, and/or preventing a HIV infection. In one embodiment, the method treats, protects against, and/or prevents a disease associated with HIV.

Upon generation of the synthetic antibody in the subject, the synthetic antibody can bind to or react with the antigen. Such binding can neutralize the antigen, block recognition of the antigen by another molecule, for example, a protein or nucleic acid, and elicit or induce an immune response to the antigen, thereby treating, protecting against, and/or preventing the disease associated with the antigen in the subject.

The composition dose can be between 1 μg to 10 mg active component/kg body weight/time, and can be 20 μg to 10 mg component/kg body weight/time. The composition can be administered every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. The number of composition doses for effective treatment can be 1, 2, 3, 4, 5, 6, 7, 8, 9, or 10.

### 11. USE IN COMBINATION WITH ANTIRETROVIRALS

The present invention also provides a method of treating, protecting against, and/or preventing disease in a subject in need thereof by administering a combination of the synthetic antibody and a therapeutic antiretroviral agent.

The synthetic antibody and an antiretroviral agent may be administered using any suitable method such that a combination of the synthetic antibody and antiretroviral agent are both present in the subject. In one embodiment, the method may comprise administration of a first composition comprising a synthetic antibody of the invention by any of the methods described in detail above and administration of a second composition comprising an antiretroviral agent less than 1, less than 2, less than 3, less than 4, less than 5, less than 6, less than 7, less than 8, less than 9 or less than 10 days following administration of the synthetic antibody. In one embodiment, the method may comprise administration of a first composition comprising a synthetic antibody of the invention by any of the methods described in detail above and administration of a second composition comprising an antiretroviral agent more than 1, more than 2, more than 3, more than 4, more than 5, more than 6, more than 7, more than 8, more than 9 or more than 10 days following administration of the synthetic antibody. In one embodiment, the method may comprise administration of a first composition comprising an antiretroviral agent and administration of a second composition comprising a synthetic antibody of the invention by any of the methods described in detail above less than 1, less than 2, less than 3, less than 4, less than 5, less than 6, less than 7, less than 8, less than 9 or less than 10 days following administration of the antiretroviral agent. In one embodiment, the method may comprise administration of a first composition comprising an antiretroviral agent and administration of a second composition comprising a synthetic antibody of the invention by any of the methods described in detail above more than 1, more than 2, more than 3, more than 4, more than 5, more than 6, more than 7, more than 8, more than 9 or more than 10 days following administration of the antiretroviral agent. In one embodiment, the method may comprise administration of a first composition comprising a synthetic antibody of the invention by any of the methods described in detail above and a second composition comprising an antiretroviral agent concurrently. In one embodiment, the method may comprise administration of a first composition comprising a synthetic antibody of the invention by any of the methods described in detail above and a second composition comprising an antiretroviral agent concurrently. In one embodiment, the method may comprise administration of a single composition comprising a synthetic antibody of the invention and an antiretroviral agent.

Non-limiting examples of antiretrovirals that can be used in combination with the synthetic antibody of the invention include Nucleoside Reverse Transcriptase Inhibitors (NRTIs), Non-Nucleoside Reverse Transcriptase Inhibitors (NNRTIs), Protease Inhibitors (PIs), Fusion Inhibitors, CCRF Antagonists, Integrase Inhibitors, Post-Attachment Inhibitors, Pharmacokinetic Inhibitors, and any combination thereof. Exemplary antiretrovirals include, but are not limited to abacavir, emtricitabine, lamivudine, tenofovir, zidovudine, efavirenz, etravirine, nevirapine, rilpivirine, atazanavir, darunavir, fosamprenavir, ritonavir, saquinavir, tipranavir, enfuvirtide, maraviroc, dolutegravir, raltegravir, ibalizumab, and cobicistat. The present invention has multiple aspects, illustrated by the following non-limiting examples.

### 12. GENERATION OF SYNTHETIC ANTIBODIES IN VITRO AND EX VIVO

In one embodiment, the synthetic antibody is generated in vitro or ex vivo. For example, in one embodiment, a nucleic acid encoding a synthetic antibody can be introduced and expressed in an in vitro or ex vivo cell. Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New York). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

### 13. USE IN COMBINATION WITH VACCINES

The present invention also provides a method of treating, protecting against, preventing disease, and/or inducing an immune response in a subject in need thereof by administering a combination of the synthetic antibody and a vaccine. In one embodiment, the vaccine is a DNA vaccine.

The synthetic antibody and vaccine may be administered using any suitable method such that a combination of the synthetic antibody and vaccine are both present in the subject. In one embodiment, the method may comprise administration of a first composition comprising a synthetic antibody of the invention by any of the methods described in detail above and administration of a second composition comprising an vaccine less than 1, less than 2, less than 3, less than 4, less than 5, less than 6, less than 7, less than 8, less than 9 or less than 10 days following administration of the synthetic antibody. In one embodiment, the method may comprise administration of a first composition comprising a synthetic antibody of the invention by any of the methods described in detail above and administration of a second composition comprising a vaccine more than 1, more than 2, more than 3, more than 4, more than 5, more than 6, more than 7, more than 8, more than 9 or more than 10 days following administration of the synthetic antibody. In one embodiment, the method may comprise administration of a first composition comprising a vaccine and administration of a second composition comprising a synthetic antibody of the invention by any of the methods described in detail above less than 1, less than 2, less than 3, less than 4, less than 5, less than 6, less than 7, less than 8, less than 9 or less than 10 days following administration of the vaccine. In one embodiment, the method may comprise administration of a first composition comprising a vaccine and administration of a second composition comprising a synthetic antibody of the invention by any of the methods described in detail above more than 1, more than 2, more than 3, more than 4, more than 5, more than 6, more than 7, more than 8, more than 9 or more than 10 days following administration of the vaccine. In one embodiment, the method may comprise administration of a first composition comprising a synthetic antibody of the invention by any of the methods described in detail above and a second composition comprising a vaccine concurrently. In one embodiment, the method may comprise administration of a first composition comprising a synthetic antibody of the invention by any of the methods described in detail above and a second composition comprising a vaccine concurrently. In one embodiment, the method may comprise administration of a single composition comprising a synthetic antibody of the invention and a vaccine.

In one embodiment, the vaccine is a DNA vaccine. In one embodiment, the synthetic antibody is a DMAb.

The DNA vaccine and the DMAb may be administered at the same time or at different times. In one embodiment, the DNA vaccine and the DMAb are administered simultaneously. In one embodiment, the DNA vaccine is administered before the DMAb. In one embodiment, the DMAb is administered before the DNA vaccine.

In certain embodiments, the DNA vaccine is administered 1 or more days, 2 or more days, 3 or more days, 4 or more days, 5 or more days, 6 or more days, 7 or more days, 8 or more days, 9 or more days, 10 or more days, 11 or more days, 12 or more days, 13 or more days, or 14 or more days after the DMAb is administered. In certain embodiments, the DNA vaccine is administered 1 or more weeks, 2 or more weeks, 3 or more weeks, 4 or more weeks, 5 or more weeks, 6 or more weeks, 7 or more weeks, 8 or more weeks, 9 or more weeks, or 10 or more weeks after the DMAb is administered. In certain embodiments, the DNA vaccine is administered 1 or more months, 2 or more months, 3 or more months, 4 or more months, 5 or more months, 6 or more months, 7 or more months, 8 or more months, 9 or more months, 10 or more months, 11 or more months, or 12 or more months after the DMAb is administered.

In certain embodiments, the DMAb is administered 1 or more days, 2 or more days, 3 or more days, 4 or more days, 5 or more days, 6 or more days, 7 or more days, 8 or more days, 9 or more days, 10 or more days, 11 or more days, 12 or more days, 13 or more days, or 14 or more days after the DNA vaccine is administered. In certain embodiments, the DMAb is administered 1 or more weeks, 2 or more weeks, 3 or more weeks, 4 or more weeks, 5 or more weeks, 6 or more weeks, 7 or more weeks, 8 or more weeks, 9 or more weeks, or 10 or more weeks after the DNA vaccine is administered. In certain embodiments, the DMAb is administered 1 or more months, 2 or more months, 3 or more months, 4 or more months, 5 or more months, 6 or more months, 7 or more months, 8 or more months, 9 or more months, 10 or more months, 11 or more months, or 12 or more months after the DNA vaccine is administered.

In certain embodiments, the DMAb and DNA vaccine are administered once. In certain embodiments, the DMAb and/or the DNA vaccine are administered more than once. In certain embodiments, administration of the DMAb and DNA vaccine provides a persistent and systemic immune response.

The composition can comprise 1 or more, 2 or more, 3 or more, 4 or more, 5 or more, 6 or more, 7 or more, 8 or more, 9 or more, or 10 or more DNA vaccines encoding an antigen. The composition may comprise 1 or more, 2 or more, 3 or more, 4 or more, 5 or more, 6 or more, 7 or more, 8 or more, 9 or more, or 10 or more DNA encoded synthetic antibodies or fragments thereof.

### a. DNA Vaccine

The vaccine can be a DNA vaccine, a peptide vaccine, or a combination DNA and peptide vaccine. The DNA vaccine can include a nucleic acid sequence encoding the antigen. The nucleic acid sequence can be DNA, RNA, cDNA, a variant thereof, a fragment thereof, or a combination thereof. The nucleic acid sequence can also include additional sequences that encode linker, leader, or tag sequences that are linked to the antigen by a peptide bond. The peptide vaccine can include an antigenic peptide, an antigenic protein, a variant thereof, a fragment thereof, or a combination thereof. The combination DNA and peptide vaccine can include the above described nucleic acid sequence encoding the antigen and the antigenic peptide or protein, in which the antigenic peptide or protein and the encoded antigen have the same amino acid sequence

The vaccine can induce a humoral immune response in the subject administered the vaccine. The induced humoral immune response can be specific for the antigen. The induced humoral immune response can be reactive with the antigen. The humoral immune response can be induced in the subject administered the vaccine by about 1.5-fold to about 16-fold, about 2-fold to about 12-fold, or about 3-fold to about 10-fold. The humoral immune response can be induced in the subject administered the vaccine by at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, at least about 15.0-fold, at least about 15.5-fold, or at least about 16.0-fold.

The humoral immune response induced by the vaccine can include an increased level of neutralizing antibodies associated with the subject administered the vaccine as compared to a subject not administered the vaccine. The neutralizing antibodies can be specific for the antigen. The neutralizing antibodies can be reactive with the antigen. The neutralizing antibodies can provide protection against and/or treatment of infection and its associated pathologies in the subject administered the vaccine.

The humoral immune response induced by the vaccine can include an increased level of IgG antibodies associated with the subject administered the vaccine as compared to a subject not administered the vaccine. These IgG antibodies can be specific for the antigen. These IgG antibodies can be reactive with the antigen. Preferably, the humoral response is cross-reactive against two or more strains of the antigen. The level of IgG antibody associated with the subject administered the vaccine can be increased by about 1.5-fold to about 16-fold, about 2-fold to about 12-fold, or about 3-fold to about 10-fold as compared to the subject not administered the vaccine. The level of IgG antibody associated with the subject administered the vaccine can be increased by at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, at least about 15.0-fold, at least about 15.5-fold, or at least about 16.0-fold as compared to the subject not administered the vaccine.

The vaccine can induce a cellular immune response in the subject administered the vaccine. The induced cellular immune response can be specific for the antigen. The induced cellular immune response can be reactive to the antigen. Preferably, the cellular response is cross-reactive against two or more strains of the antigen. The induced cellular immune response can include eliciting a CD8⁺ T cell response. The elicited CD8⁺ T cell response can be reactive with the antigen. The elicited CD8⁺ T cell response can be polyfunctional. The induced cellular immune response can include eliciting a CD8⁺ T cell response, in which the CD8⁺ T cells produce interferon-gamma (IFN-γ), tumor necrosis factor alpha (TNF-α), interleukin-2 (IL-2), or a combination of IFN-γ and TNF-α.

The induced cellular immune response can include an increased CD8⁺ T cell response associated with the subject administered the vaccine as compared to the subject not administered the vaccine. The CD8⁺ T cell response associated with the subject administered the vaccine can be increased by about 2-fold to about 30-fold, about 3-fold to about 25-fold, or about 4-fold to about 20-fold as compared to the subject not administered the vaccine. The CD8⁺ T cell response associated with the subject administered the vaccine can be increased by at least about 1.5-fold, at least about 2.0-fold, at least about 3.0-fold, at least about 4.0-fold, at least about 5.0-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, at least about 15.0-fold, at least about 16.0-fold, at least about 17.0-fold, at least about 18.0-fold, at least about 19.0-fold, at least about 20.0-fold, at least about 21.0-fold, at least about 22.0-fold, at least about 23.0-fold, at least about 24.0-fold, at least about 25.0-fold, at least about 26.0-fold, at least about 27.0-fold, at least about 28.0-fold, at least about 29.0-fold, or at least about 30.0-fold as compared to the subject not administered the vaccine.

The induced cellular immune response can include an increased frequency of CD3⁺CD8⁺ T cells that produce IFN-γ. The frequency of CD3⁺CD8⁺IFN-γ⁺ T cells associated with the subject administered the vaccine can be increased by at least about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold as compared to the subject not administered the vaccine.

The induced cellular immune response can include an increased frequency of CD3⁺CD8⁺ T cells that produce TNF-α. The frequency of CD3⁺CD8⁺TNF-α⁺ T cells associated with the subject administered the vaccine can be increased by at least about 2-fold, 3-fold, 4-fold, 5-fold, 6-fold, 7-fold, 8-fold, 9-fold, 10-fold, 11-fold, 12-fold, 13-fold, or 14-fold as compared to the subject not administered the vaccine.

The induced cellular immune response can include an increased frequency of CD3⁺CD8⁺ T cells that produce IL-2. The frequency of CD3⁺CD8⁺IL-2⁺ T cells associated with the subject administered the vaccine can be increased by at least about 0.5-fold, 1.0-fold, 1.5-fold, 2.0-fold, 2.5-fold, 3.0-fold, 3.5-fold, 4.0-fold, 4.5-fold, or 5.0-fold as compared to the subject not administered the vaccine.

The induced cellular immune response can include an increased frequency of CD3⁺CD8⁺ T cells that produce both IFN-γ and TNF-α. The frequency of CD3⁺CD8⁺IFN-γ⁺ TNF-α⁺ T cells associated with the subject administered the vaccine can be increased by at least about 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 110-fold, 120-fold, 130-fold, 140-fold, 150-fold, 160-fold, 170-fold, or 180-fold as compared to the subject not administered the vaccine.

The cellular immune response induced by the vaccine can include eliciting a CD4⁺ T cell response. The elicited CD4⁺ T cell response can be reactive with the desired antigen. The elicited CD4⁺ T cell response can be polyfunctional. The induced cellular immune response can include eliciting a CD4⁺ T cell response, in which the CD4⁺ T cells produce IFN-γ, TNF-α, IL-2, or a combination of IFN-γ and TNF-α.

The induced cellular immune response can include an increased frequency of CD3⁺CD4⁺ T cells that produce IFN-γ. The frequency of CD3⁺CD4⁺IFN-γ⁺ T cells associated with the subject administered the vaccine can be increased by at least about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold as compared to the subject not administered the vaccine.

The induced cellular immune response can include an increased frequency of CD3⁺CD4⁺ T cells that produce TNF-α. The frequency of CD3⁺CD4⁺TNF-α⁺ T cells associated with the subject administered the vaccine can be increased by at least about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, or 22-fold as compared to the subject not administered the vaccine.

The induced cellular immune response can include an increased frequency of CD3⁺CD4⁺ T cells that produce IL-2. The frequency of CD3⁺CD4⁺IL-2⁺ T cells associated with the subject administered the vaccine can be increased by at least about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, 30-fold, 31-fold, 32-fold, 33-fold, 34-fold, 35-fold, 36-fold, 37-fold, 38-fold, 39-fold, 40-fold, 45-fold, 50-fold, 55-fold, or 60-fold as compared to the subject not administered the vaccine.

The induced cellular immune response can include an increased frequency of CD3⁺CD4⁺ T cells that produce both IFN-γ and TNF-α. The frequency of CD3⁺CD4⁺IFN-γ⁺TNF-α⁺ associated with the subject administered the vaccine can be increased by at least about 2-fold, 2.5-fold, 3.0-fold, 3.5-fold, 4.0-fold, 4.5-fold, 5.0-fold, 5.5-fold, 6.0-fold, 6.5-fold, 7.0-fold, 7.5-fold, 8.0-fold, 8.5-fold, 9.0-fold, 9.5-fold, 10.0-fold, 10.5-fold, 11.0-fold, 11.5-fold, 12.0-fold, 12.5-fold, 13.0-fold, 13.5-fold, 14.0-fold, 14.5-fold, 15.0-fold, 15.5-fold, 16.0-fold, 16.5-fold, 17.0-fold, 17.5-fold, 18.0-fold, 18.5-fold, 19.0-fold, 19.5-fold, 20.0-fold, 21-fold, 22-fold, 23-fold 24-fold, 25-fold, 26-fold, 27-fold, 28-fold, 29-fold, 30-fold, 31-fold, 32-fold, 33-fold, 34-fold, or 35-fold as compared to the subject not administered the vaccine.

The vaccine of the present invention can have features required of effective vaccines such as being safe so the vaccine itself does not cause illness or death; is protective against illness resulting from exposure to live pathogens such as viruses or bacteria; induces neutralizing antibody to prevent invention of cells; induces protective T cells against intracellular pathogens; and provides ease of administration, few side effects, biological stability, and low cost per dose.

The vaccine can further induce an immune response when administered to different tissues such as the muscle or skin. The vaccine can further induce an immune response when administered via electroporation, or injection, or subcutaneously, or intramuscularly

### b. Antigen

The antigen of the vaccine may be the same antigen across different subtypes of HIV. The vaccine may comprise 1 or more, 2 or more, 3 or more, 4 or more, or 5 or more DNA sequences encoding a particular protein sequence isolated from HIV subtypes A, B, C, D, or other HIV subtypes, or a combination or variant thereof. The antigen of the vaccine may be the same antigen across different subtypes of HIV. The vaccine may comprise 1 or more, 2 or more, 3 or more, 4 or more, or 5 or more consensus DNA sequences encoding a particular protein sequence isolated from HIV subtypes A, B, C, D, or other HIV subtypes, or a combination or variant thereof. The vaccine may comprise a DNA sequence encoding a particular protein sequence isolated from HIV subtype A, a second DNA sequence encoding a particular protein sequence isolated from HIV subtype B, a third DNA sequence encoding a particular protein sequence isolated from HIV subtype C, a fourth DNA sequence encoding a particular protein sequence isolated from HIV subtype D, or a combination thereof. The vaccine may comprise a consensus DNA sequence encoding a particular protein sequence isolated from HIV subtype A, a second consensus DNA sequence encoding a particular protein sequence isolated from HIV subtype B, a third consensus DNA sequence encoding a particular protein sequence isolated from HIV subtype C, a fourth consensus DNA sequence encoding a particular protein sequence isolated from HIV subtype D, or a combination thereof. The vaccine may comprise a consensus DNA sequence or variant thereof encoding a particular HIV subtype A protein sequence or variant thereof, a second consensus DNA sequence or variant thereof encoding a particular HIV subtype B protein sequence or variant thereof, a third consensus DNA sequence or variant thereof encoding a particular HIV subtype C protein sequence or variant thereof, a fourth consensus DNA sequence or variant thereof encoding a particular HIV subtype D protein sequence or variant thereof.

In some embodiments, the HIV antigen can be a subtype A consensus envelope DNA sequence construct, an IgE leader sequence linked to a consensus sequence for Subtype A envelope protein, or a subtype A consensus Envelope protein sequence.

In other embodiments, the HIV antigen can be a subtype B consensus envelope DNA sequence construct, an IgE leader sequence linked to a consensus sequence for Subtype B envelope protein, or a subtype B consensus Envelope protein sequence.

In still other embodiments, the HIV antigen can be a subtype C consensus envelope DNA sequence construct, an IgE leader sequence linked to a consensus sequence for subtype C envelope protein, or a subtype C consensus envelope protein sequence.

In further embodiments, the HIV antigen can be a subtype D consensus envelope DNA sequence construct, an IgE leader sequence linked to a consensus sequence for Subtype D envelope protein, or a subtype D consensus envelope protein sequence.

In some embodiments, the HIV antigen can be a subtype A Nef-Rev consensus envelope DNA sequence construct, an IgE leader sequence linked to a consensus sequence for Subtype A Nef-Rev protein, or a Subtype A Nef-Rev consensus protein sequence.

In some embodiments, the HIV antigen can be a subtype B Nef-Rev consensus envelope DNA sequence construct, an IgE leader sequence linked to a consensus sequence for Subtype B Nef-Rev protein, or a Subtype B Nef-Rev consensus protein sequence.

In some embodiments, the HIV antigen can be a subtype C Nef-Rev consensus envelope DNA sequence construct, an IgE leader sequence linked to a consensus sequence for Subtype C Nef-Rev protein, or a Subtype C Nef-Rev consensus protein sequence.

In some embodiments, the HIV antigen can be a subtype D Nef-Rev consensus envelope DNA sequence construct, an IgE leader sequence linked to a consensus sequence for Subtype D Nef-Rev protein, or a Subtype D Nef-Rev consensus protein sequence.

In other embodiments, the HIV antigen can be a Gag consensus DNA sequence of subtype A, B, C and D DNA sequence construct, an IgE leader sequence linked to a consensus sequence for Gag consensus subtype A, B, C and D protein, or a consensus Gag subtype A, B, C and D protein sequence.

In still other embodiments, the HIV antigen can be a MPol DNA sequence or a MPol protein sequence. The HIV antigen can be nucleic acid or amino acid sequences of Env A, Env B, Env C, Env D, B Nef-Rev, Gag, MPol, or any combination thereof.

In other embodiments, the HIV antigen may be a DNA sequence or consensus sequence of subtype A, B, C, or D encoding gp140 or consensus gp140 protein. In other embodiments, the HIV antigen may be a DNA sequence or consensus sequence of subtype A, B, C, or D encoding gp140 or consensus gp120 protein. In other embodiments, the HIV antigen gp140 peptide sequence or gp140 consensus peptide sequence of subtype A, B, C, or D. In other embodiments, the HIV antigen gp120 peptide sequence or gp140 consensus peptide sequence of subtype A, B, C, or D.

Examples of DNA vaccines encoding HIV antigens include those described in U.S. Pat. No. 8,168,769 and WO2015/073291, the contents of each are fully incorporated by reference In some embodiments, a DNA vaccine encoding a HIV antigen may include a nucleic acid sequence encoding an amino acid sequence comprising one of SEQ ID NO: 136, SEQ ID NO: 138, SEQ ID NO: 140, SEQ ID NO: 142, SEQ ID NO: 144, SEQ ID NO: 146, SEQ ID NO: 147, SEQ ID NO: 148, SEQ ID NO: 149, SEQ ID NO: 150, SEQ ID NO: 151, or SEQ ID NO: 152. In some embodiments, the DNA vaccine encoding a HIV antigen may include a nucleic acid sequence comprising the sequence of one of SEQ ID NO: 135, SEQ ID NO: 137, SEQ ID NO: 139, SEQ ID NO: 141, SEQ ID NO: 143, or SEQ ID NO: 145.

The antigen can affect a mammal, which can be a human, chimpanzee, dog, cat, horse, cow, mouse, or rat. The antigen can be contained in a protein from a mammal, which can be a human, chimpanzee, dog, cat, horse, cow, pig, sheep, mouse, or rat.

### c. DNA

The composition may comprise the DNA. Also provided herein is a DNA that encodes the antigen as described above. The DNA can include an encoding sequence that encodes the antigen. The DNA can also include additional sequences that encode linker or tag sequences that are linked to the antigen by a peptide bond.

### d. Vector

The composition may comprise a vector that includes the DNA encoding the antigen. The vector can be capable of expressing the antigen. The vector may be an expression construct, which is generally a plasmid that is used to introduce a specific gene into a target cell. Once the expression vector is inside the cell, the protein that is encoded by the gene is produced by the cellular-transcription and translation machinery ribosomal complexes. The plasmid is frequently engineered to contain regulatory sequences that act as enhancer and promoter regions and lead to efficient transcription of the gene carried on the expression vector. The vectors of the present invention express large amounts of stable messenger RNA, and therefore proteins.

The vectors may have expression signals such as a strong promoter, a strong termination codon, adjustment of the distance between the promoter and the cloned gene, and the insertion of a transcription termination sequence and a PTIS (portable translation initiation sequence).

### i. Expression Vectors

The vector may be circular plasmid or a linear nucleic acid vaccine. The circular plasmid and linear nucleic acid are capable of directing expression of a particular nucleotide sequence in an appropriate subject cell. The vector may have a promoter operably linked to the antigen-encoding nucleotide sequence, which may be operably linked to termination signals. The vector may also contain sequences required for proper translation of the nucleotide sequence. The vector comprising the nucleotide sequence of interest may be chimeric, meaning that at least one of its components is heterologous with respect to at least one of its other components. The expression of the nucleotide sequence in the expression cassette may be under the control of a constitutive promoter or of an inducible promoter which initiates transcription only when the host cell is exposed to some particular external stimulus. In the case of a multicellular organism, the promoter can also be specific to a particular tissue or organ or stage of development.

### ii. Circular and Linear Vectors

The vector may be circular plasmid, which may transform a target cell by integration into the cellular genome or exist extrachromosomally (e.g. autonomous replicating plasmid with an origin of replication).

The vector can be pVAX, pcDNA3.0, or provax, or any other expression vector capable of expressing the DNA and enabling a cell to translate the sequence to a antigen that is recognized by the immune system. The vector can be combined with antigen at a mass ratio of between 5:1 and 1:5, or of between 1:1 and 2:1.

Also provided herein is a linear nucleic acid vaccine, or linear expression cassette (“LEC”), that is capable of being efficiently delivered to a subject via electroporation and expressing one or more desired antigens. The LEC may be any linear DNA devoid of any phosphate backbone. The DNA may encode one or more antigens. The LEC may contain a promoter, an intron, a stop codon, a polyadenylation signal. The expression of the antigen may be controlled by the promoter. The LEC may not contain any antibiotic resistance genes and/or a phosphate backbone. The LEC may not contain other nucleic acid sequences unrelated to the desired antigen gene expression.

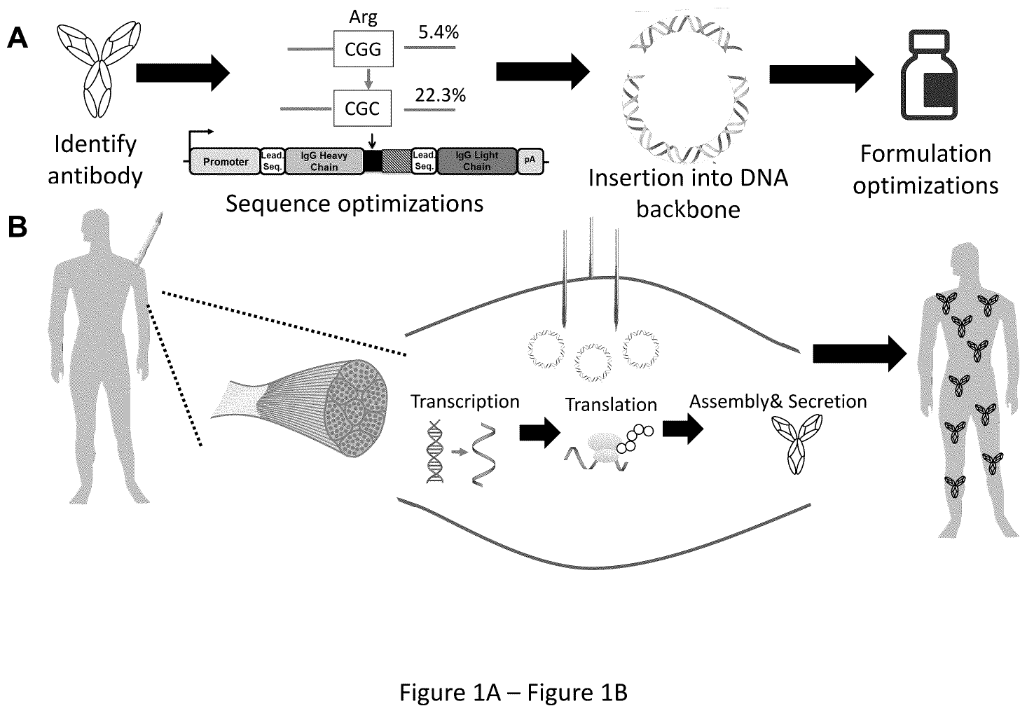

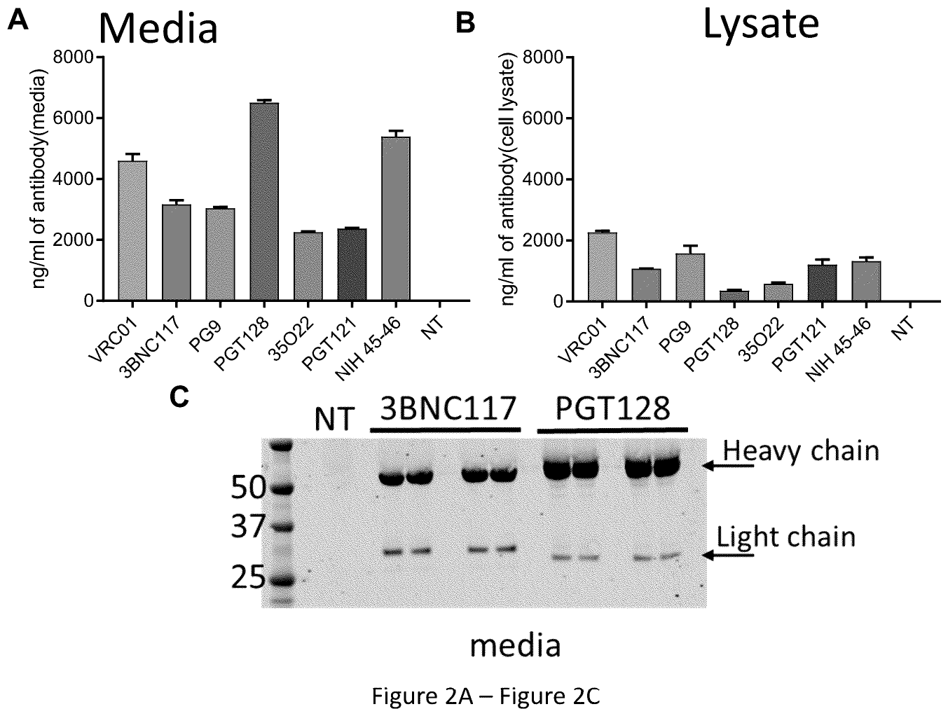

The LEC may be derived from any plasmid capable of being linearized. The plasmid may be capable of expressing the antigen. The plasmid may be pNP (Puerto Rico/34) or pM2 (New Caledonia/99). See FIG. 1. The plasmid may be pVAX, pcDNA3.0, or provax, or any other expression vector capable of expressing the DNA and enabling a cell to translate the sequence to a antigen that is recognized by the immune system.

The LEC may be perM2. The LEC may be perNP. perNP and perMR may be derived from pNP (Puerto Rico/34) and pM2 (New Caledonia/99), respectively. See FIG. 34. The LEC may be combined with antigen at a mass ratio of between 5:1 and 1:5, or of between 1:1 to 2:1.

### iii. Promoter, Intron, Stop Codon, and Polyadenylation Signal

The vector may have a promoter. A promoter may be any promoter that is capable of driving gene expression and regulating expression of the isolated nucleic acid. Such a promoter is a cis-acting sequence element required for transcription via a DNA dependent RNA polymerase, which transcribes the antigen sequence described herein. Selection of the promoter used to direct expression of a heterologous nucleic acid depends on the particular application. The promoter may be positioned about the same distance from the transcription start in the vector as it is from the transcription start site in its natural setting. However, variation in this distance may be accommodated without loss of promoter function.

The promoter may be operably linked to the nucleic acid sequence encoding the antigen and signals required for efficient polyadenylation of the transcript, ribosome binding sites, and translation termination. The promoter may be a CMV promoter, SV40 early promoter, SV40 later promoter, metallothionein promoter, murine mammary tumor virus promoter, Rous sarcoma virus promoter, polyhedrin promoter, or another promoter shown effective for expression in eukaryotic cells.

The vector may include an enhancer and an intron with functional splice donor and acceptor sites. The vector may contain a transcription termination region downstream of the structural gene to provide for efficient termination. The termination region may be obtained from the same gene as the promoter sequence or may be obtained from different genes.

### e. Other Components of Vaccine-Adjuvants, Excipients

The composition may further comprise a pharmaceutically acceptable excipient. The pharmaceutically acceptable excipient can be functional molecules as vehicles, adjuvants, carriers, or diluents. The pharmaceutically acceptable excipient can be a transfection facilitating agent, which can include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

The transfection facilitating agent is a polyanion, polycation, including poly-L-glutamate (LGS), or lipid. The transfection facilitating agent is poly-L-glutamate, and the poly-L-glutamate is may be present in the vaccine at a concentration less than 6 mg/ml. The transfection facilitating agent may also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the genetic construct. The DNA plasmid vaccines may also include a transfection facilitating agent such as lipids, liposomes, including lecithin liposomes or other liposomes known in the art, as a DNA-liposome mixture (see for example W09324640), calcium ions, viral proteins, polyanions, polycations, or nanoparticles, or other known transfection facilitating agents. The transfection facilitating agent is a polyanion, polycation, including poly-L-glutamate (LGS), or lipid. Concentration of the transfection agent in the vaccine is less than 4 mg/ml, less than 2 mg/ml, less than 1 mg/ml, less than 0.750 mg/ml, less than 0.500 mg/ml, less than 0.250 mg/ml, less than 0.100 mg/ml, less than 0.050 mg/ml, or less than 0.010 mg/ml.

The pharmaceutically acceptable excipient can be an adjuvant. The adjuvant can be other genes that are expressed in alternative plasmid or are delivered as proteins in combination with the plasmid above in the vaccine. The adjuvant may be selected from the group consisting of: a-interferon(IFN-α), β-interferon (IFN-β), γ-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHC, CD80,CD86 including IL-15 having the signal sequence deleted and optionally including the signal peptide from IgE. The adjuvant can be IL-12, IL-15, IL-28, CTACK, TECK, platelet derived growth factor (PDGF), TNFα, TNFP, GM-CSF, epidermal growth factor (EGF), IL-1, IL-2, IL-4, IL-5, IL-6, IL-10, IL-12, IL-18, or a combination thereof.

Other genes that can be useful adjuvants include those encoding: MCP-1, MIP-1a, MIP-1p, IL-8, RANTES, L-selectin, P-selectin, E-selectin, CD34, GlyCAM-1, MadCAM-1, LFA-1, VLA-1, Mac-1, pl50.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Flt, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof.

The vaccine may further comprise a genetic vaccine facilitator agent as described in U.S. Ser. No. 021,579 filed Apr. 1, 1994, which is fully incorporated by reference.

The vaccine can be formulated according to the mode of administration to be used. An injectable vaccine pharmaceutical composition can be sterile, pyrogen free and particulate free. An isotonic formulation or solution can be used. Additives for isotonicity can include sodium chloride, dextrose, mannitol, sorbitol, and lactose. The vaccine can comprise a vasoconstriction agent. The isotonic solutions can include phosphate buffered saline. Vaccine can further comprise stabilizers including gelatin and albumin. The stabilizers can allow the formulation to be stable at room or ambient temperature for extended periods of time, including LGS or polycations or polyanions.

### 14. EXAMPLES

The present invention is further illustrated in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1

The studies presented herein demonstrate in vivo production of broadly neutralizing and non-neutralizing antibodies against HIV-1 by novel DNA encoded mAb technology.

As described herein, an optimized, synthetic DNA vector platform (dMAb) to deliver encoding broadly neutralizing antibodies (bNabs) and non-neutralizing antibodies (non-Nabs) against HIV was designed. Plasmids encoding the heavy and light chain of numerous bNabs were designed. Using various optimization strategies including sequence, formulation, structural and delivery modifications, high levels (1-100 μg/ml) of serum bNabs are generated in mice. Antibody levels are detectable in the serum as early as two days after DNA injection with electroporation. Furthermore, these antibodies from the serum retain functional capabilities including binding to Env and neutralization as measured by the TZM-bl neutralization assay. To lower the possibility of viral escape in a clinical scenario, multiple HIV-1 bNabs can be encoded and expressed simultaneously.

### Example 2—DMABs Encoding HIV bnAbs

The studies presented herein demonstrate DNA monoclonal antibodies are able to encode HIV bNabs and non-Nabs.

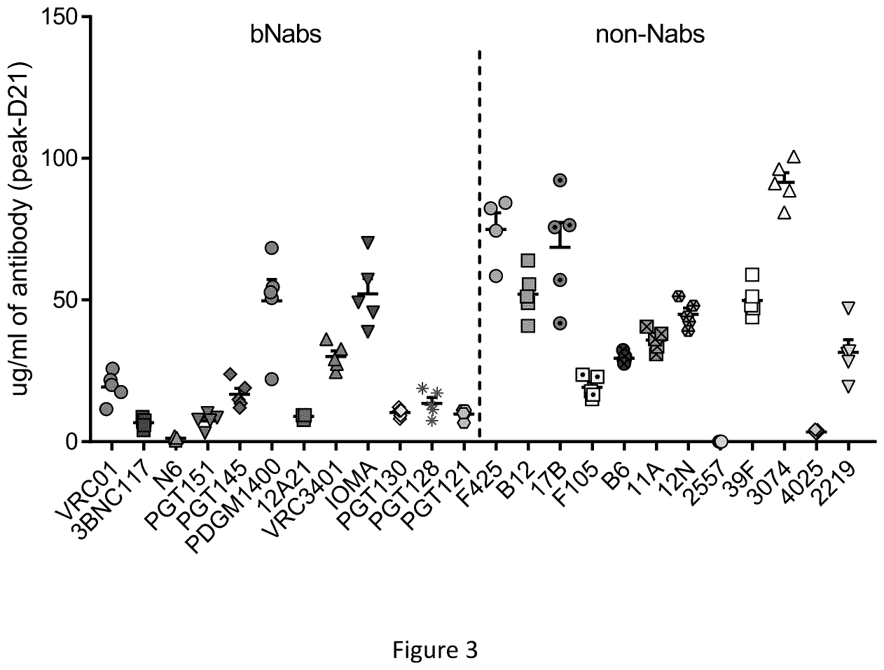

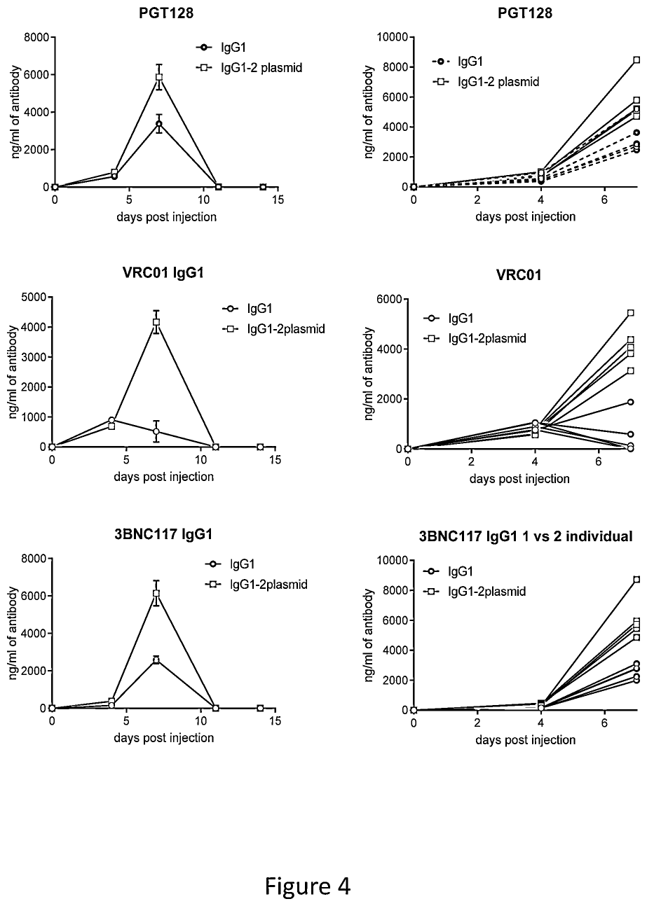

Some HIV dMAbs express at very low levels (FIG. 3) thus we initially looked to determine if dMAb levels could be increases when the heavy and light change were encoded on separate plasmids. Encoding the heavy and light chain on two separate plasmids increases in vivo dMAb production of various HIV bNabs over that of single plasmid injection (FIG. 4).

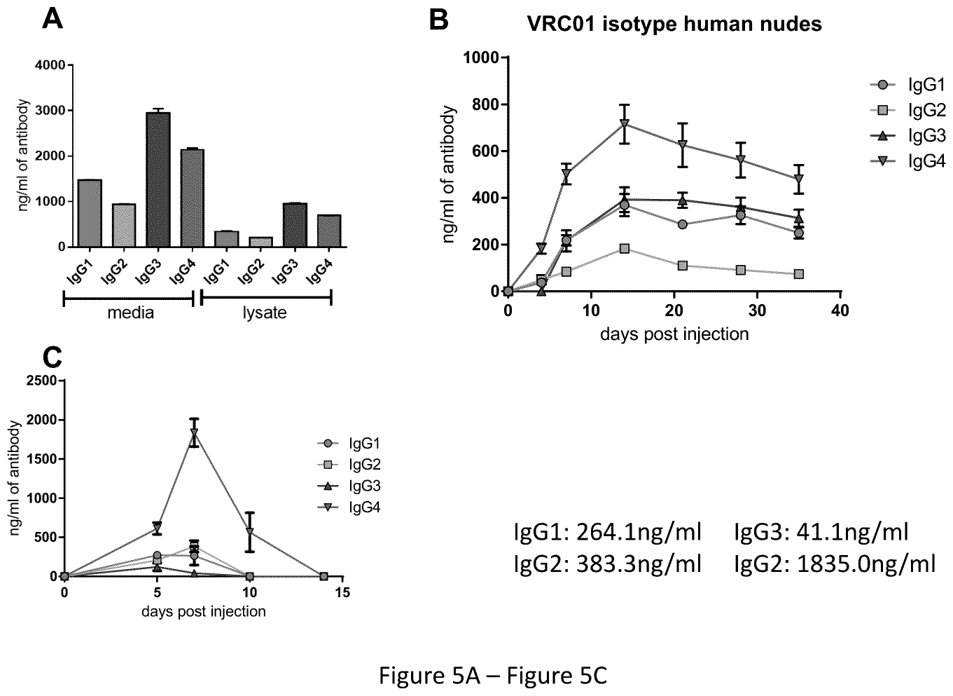

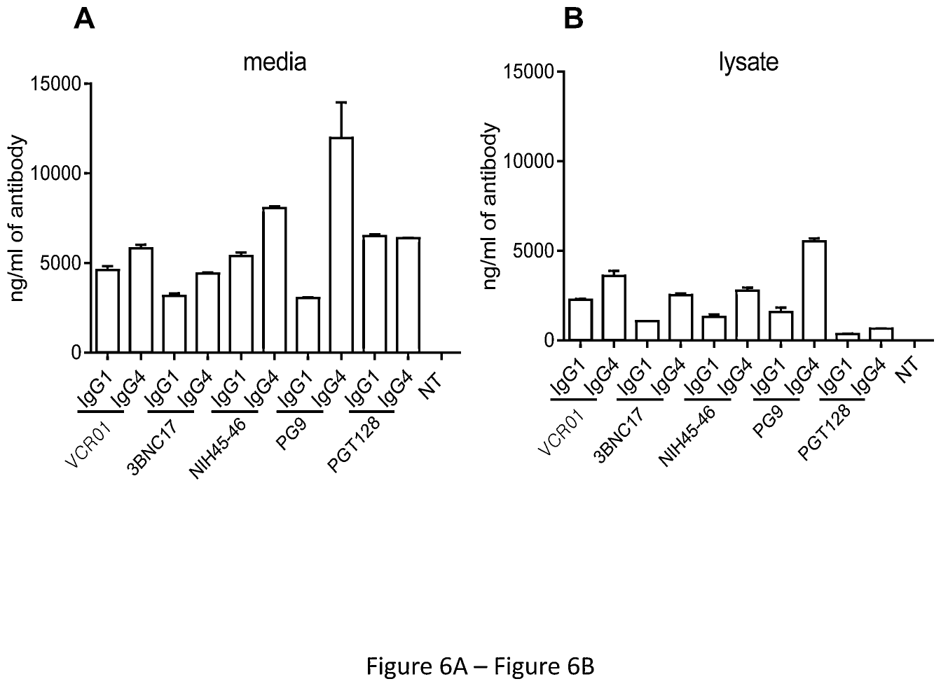

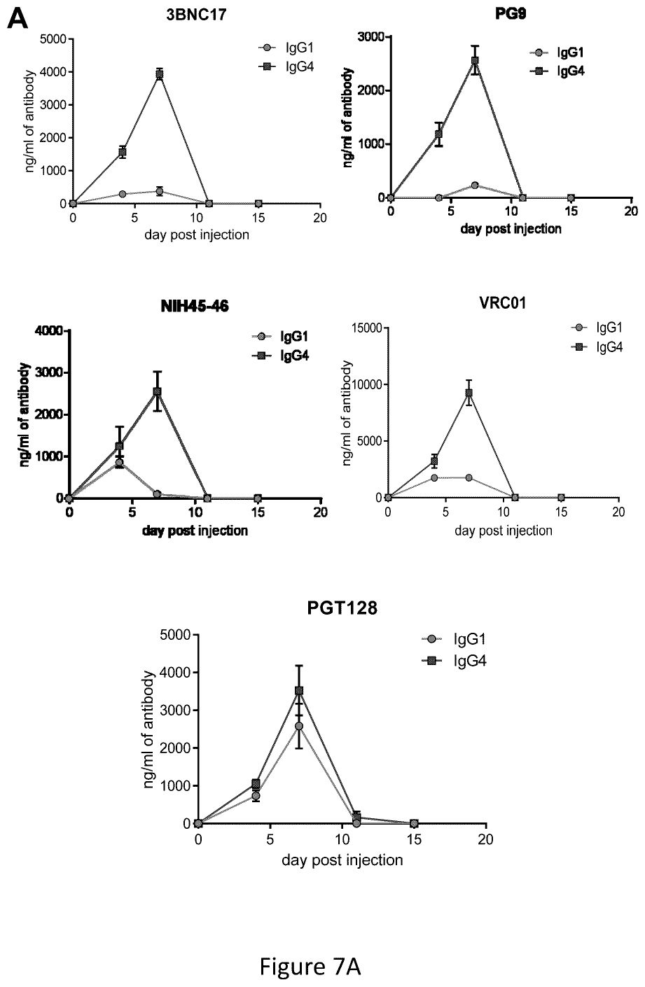

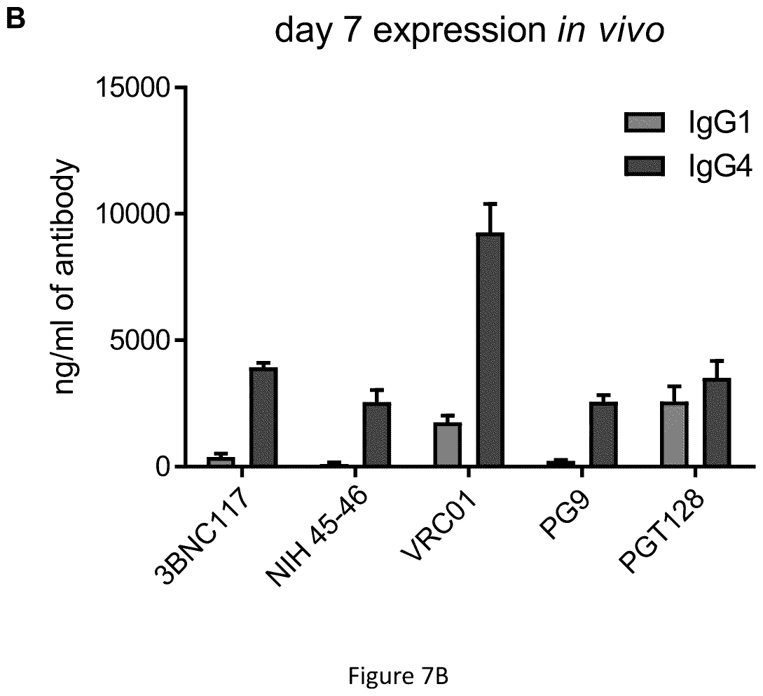

Different IgGs have different effector functions and distributions. Therefore, it was determined whether dMAbs expressing various IgGs have differential expression as well as effector functions. All isotypes of VRCOI express in vitro (FIG. 5A). In nude mice, IgG4 has the highest expression level (FIG. 5B). In BALB/c mice, IgG4 significantly increases VRCOI expression (FIG. 5C). Due to the limited expression in vivo of HIV dMAbs and the increase in VRCOI with IgG4 isotype, we next looked to see IgG4 optimization could be broadly applied to increase the levels of dMAb expression. All IgG4 constructs expressed in vitro in both the cell lysate and media of 293T transfected cells (FIG. 6). Futhermore, IgG4 increased the expression of each of PG9, 3BNC17, NIH 45-46, VRC01, PGT218 over IgG1 (FIGS. 7).

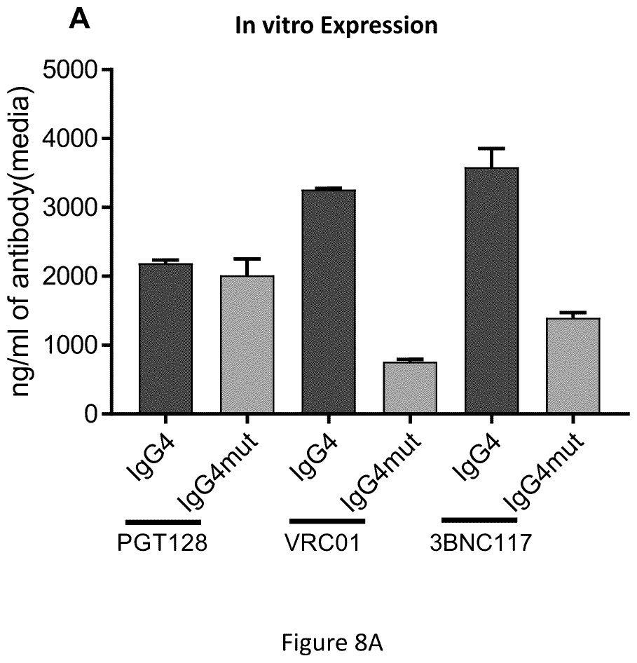

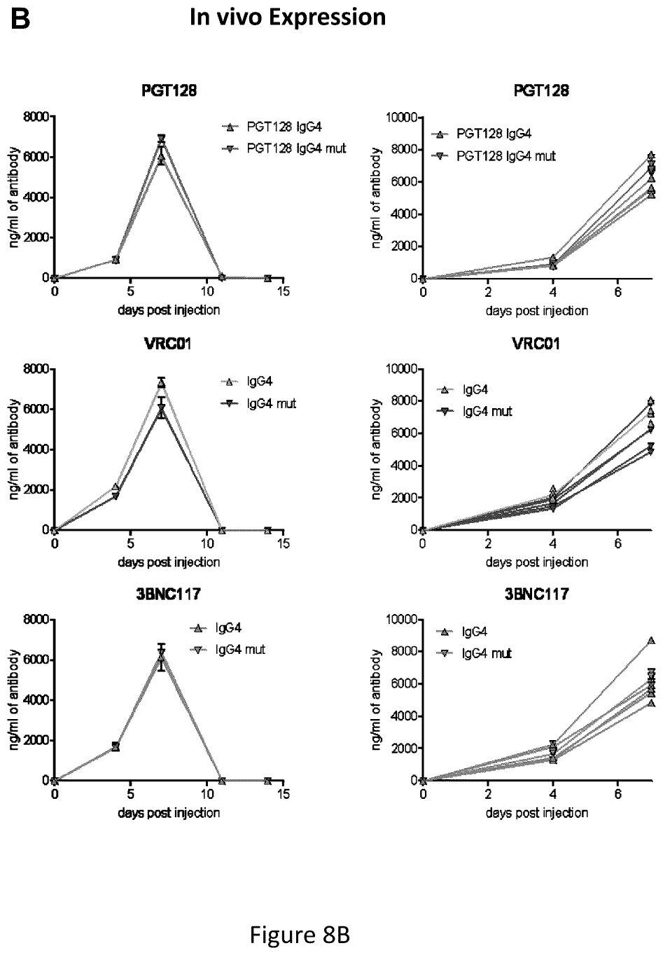

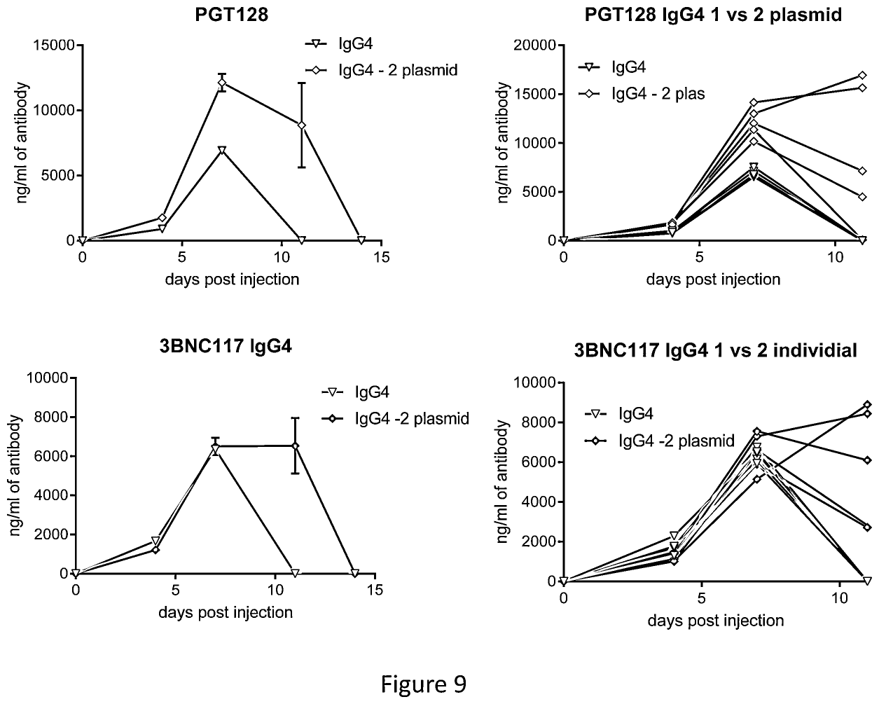

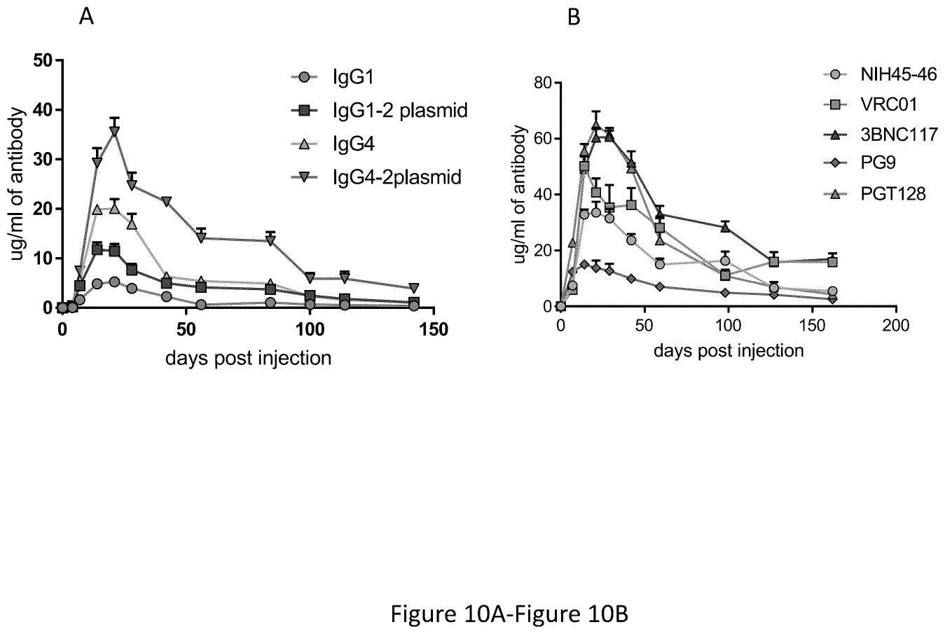

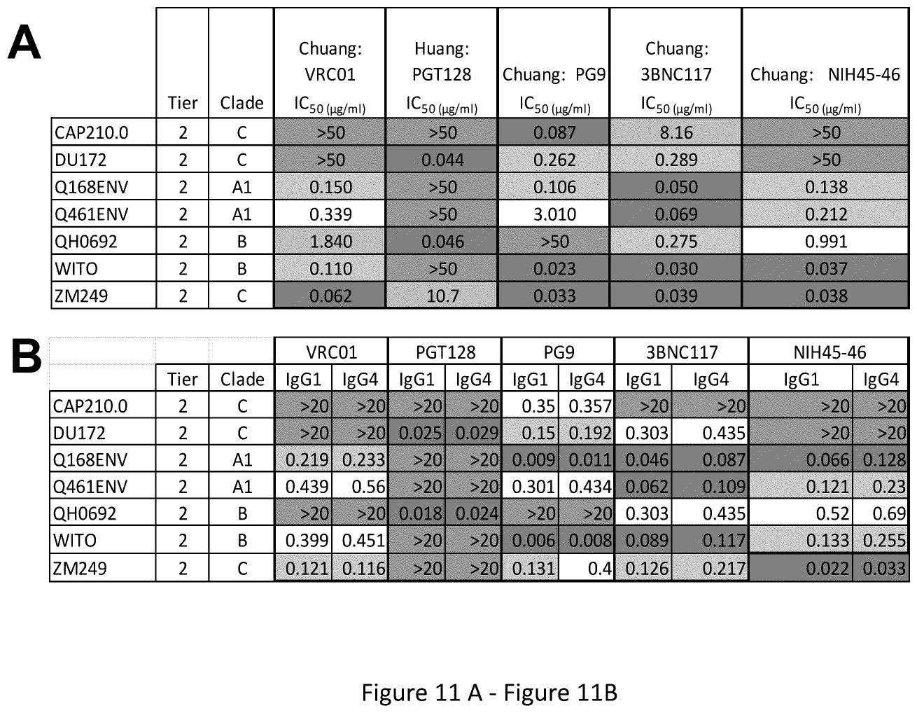

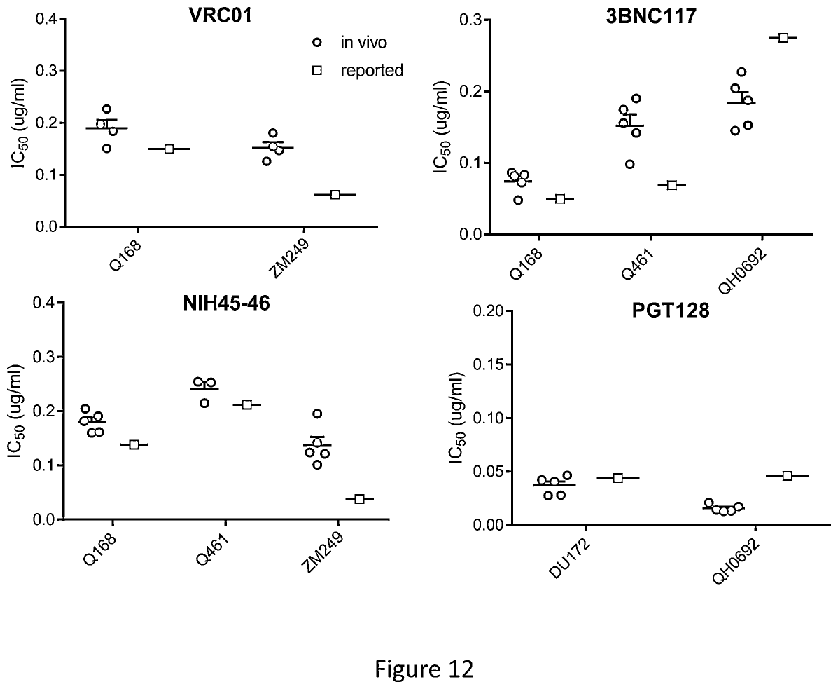

To further increase the level of dMAbs expression, a mutation was made the core-hinge region, Ser228Pro, to block Fab-arm exchange. FIG. 8 demonstrates, that the S228P mutation does not disrupt expression levels. Additionally, encoding the IgG4 heavy and light chain on separate plasmids further increased expression levels and kinetics of HIV dMAbs (FIG. 9). Importantly there was no change in the functionality of these antibodies when testing in the TzmBL assay (FIG. 11 and FIG. 12)

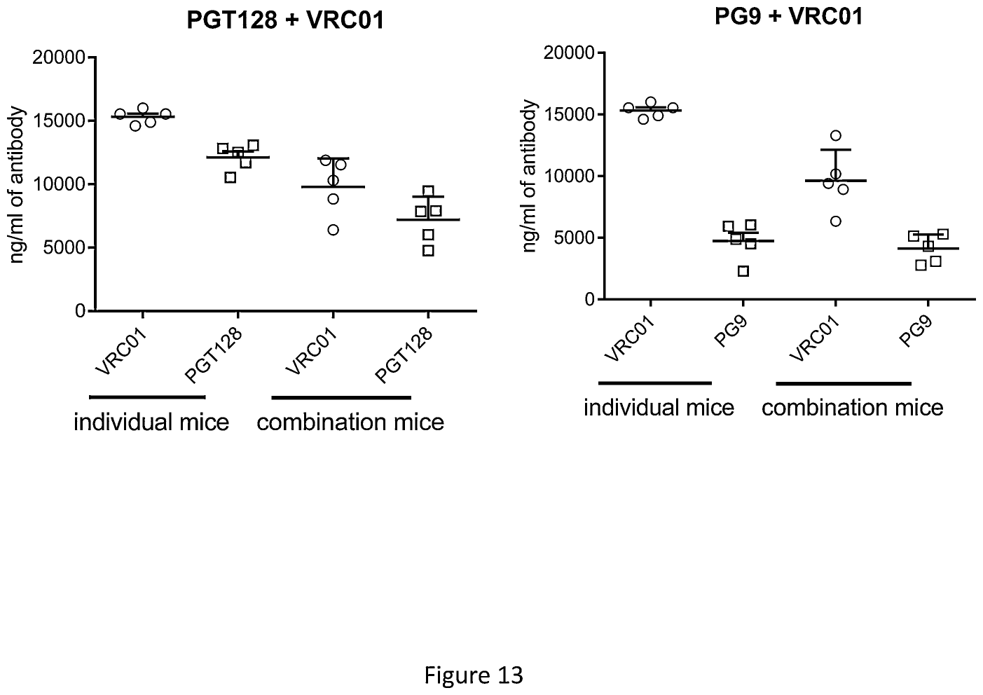

It may be beneficial to express multiple HIV bnAbs to have more than 1 site targeted which may decrease the likelihood of viral escape. We have demonstrated that multiple dMAbs expressing two different HIV bNab can be expressed in a single mouse (FIG. 13).

### Example 3—Engineering HIV-1 Antibodies and Immunoadhesins for Increased In Vivo Production Using DNA Encoded Monoclonal Antibody Technology

The ability of HIV specific broadly neutralizing antibodies (bNabs) to protect or control HIV-1 is well-established in animal models. Though these antibodies are powerful and can prevent infection or control viral loads, the cost of production could severely limit the ability to use these antibodies in the field. Additionally, current vaccine approaches have failed to induce similar antibodies. A novel way to encode and produce bNabs in vivo is described herein using a synthetic DNA encoded monoclonal antibody (dMab) platform.

Engineered plasmids encoding the heavy and light chain of over 20 HIV Envelope (Env) specific antibodies were produced and delivered intramuscularly to mice via electroporation. Initial production levels of many of the antibodies were low; however, using numerous optimization strategies including sequence, formulation, structural modifications and delivery, we can generate high levels (over 80 ug/ml) of serum dMabs in mice. dMabs are detectable in the serum as early as two days after injection and are sustained for over half a year. Additionally, these antibodies retain functional capabilities including binding to Env and neutralization as measured by the TZM-bl neutralization assay. The in vivo produced dMabs have similar neutralization profile and potency as reported with the recombinant purified monoclonal bNabs. To lower the possibility of viral escape, using the dMab platform, we can encode and express multiple HIV-1 bNabs simultaneously in a single mouse. Furthermore, we are exploring the ability to encode and produce bispecific antibodies or other immunoadhesins to increase potency and prevent escape.

The dMab platform represents a novel technique for in vivo antibody production against HIV-1 and possibly other immune targets.

### Example 4—Rapid and Long-Term Immunity Elicited by DNA Encoded Antibody Prophylaxis and DNA Vaccination Against HIV

Vaccination is known to exhibit a lag phase before generation of immunity; thus, there is a gap of time during infection before an immune response is in effect. The following provides specific novel approaches that utilize the benefit of vaccines and the native immune response along with a rapid generation of effective immunity using the DNA synthetic antibodies or dMabs.

An antibody-based prophylaxis/therapy entailing the electroporation mediated delivery of synthetic plasmids, encoding biologically active anti-Human Immunodeficiency virus (HIV) mAb (designated dMAb), is designed and evaluated for anti-viral efficacy as well as for the ability to overcome shortcomings inherent with conventional active vaccination by a novel passive immune-based strategy. One intramuscular injection of the HIV-dMAb produces antibodies in vivo more rapidly than active vaccination with an HIV-DNA vaccine. This dMAb neutralized diverse HIV clinical isolates and protected mice from viral challenge. Combinations of both afford rapid as well as long-lived protection.

A DNA based dMAb strategy induces rapid protection against an emerging viral infection, which can be combined with DNA vaccination providing a uniquely both short term and long-term protection against this emerging infectious disease. These studies have implications for pathogen treatment and control strategies.

### dMAb IgG Quantification and Binding Assays

ELISA assays are performed with sera from subjects administered an HIV-dMAb to quantify expression kinetics and target antigen binding.

### Analysis of dMAb Generated IgG

IgG expression of HIV infected cells are analyzed by western blot. For immunofluorescence analysis HIV infected cells are visually evaluated by confocal microscopy and quantitatively or semi-quantitatively analyzed.

### dMAb DNA Plasmid Administration and In Vivo Analysis

Expression kinetics and functionality were evaluated in subjects following injection of control or HIV-dMAb. For studies that include the DNA vaccine, the HIV-DNA vaccine plasmid is administered.

### Challenge Study

Subjects receive electroporation-enhanced injection of HIV-dMAb or control plasmids. The HIV-DNA vaccine was delivered as described above. After DNA delivery, subjects are challenged with HIV. The animals are monitored for survival and signs of infection. Serum samples are collected for cytokine quantification and other immune analysis. Blood samples are collected from after infection and viremia levels are measured.

### Neutralizing Antibody Analysis

Anti-HIV neutralizing antibody titers from subjects administered HIV-dMAb are determined. Neutralization titers may be calculated as the reciprocal of the highest dilution mediating 100% reduction of the cytopathic effects in the cells.

### Cytokine Quantitative Analysis

Sera is collected from HIV-dMAb, and HIV-DNA vaccine injected subjects as well as HIV challenged subjects. TNF-α, IL-1β and IL-6 sera cytokine levels are measured.

### Anti-HIV dMAbs Design and Confirmation of Expression

The optimized synthetic plasmids constructed from the anti-HIV-neutralizing mAb were designed for the IgG and Fab antibodies. Cells are transfected with either the HIV-IgG plasmid or the HIV-Fab (VL, VH, or combined) plasmids to validate expression in vitro. The HIV-Fab and HIV-IgG expressed antibodies in the muscle that appeared to be properly assembled and biologically functional in vitro.

### In Vivo Expression and Quantification of Anti-HIV dMAb

Following confirmation of in vitro expression, the ability of HIV-Fab or HIV-IgG to produce anti-HIV antibodies in vivo is measured. Both constructs generate mAbs. Subjects are administered either HIV-IgG or HIV-Fab, and sera antibody levels are evaluated through a binding ELISA. Sera collected after injection from both HIV-IgG and HIV-Fab bind to HIV protein but not to an unrelated control antigen. These data indicate that in vivo produced anti-HIV antibodies from HIV-IgG or HIV-Fab constructs have similar biological characteristics to conventionally produced antigen specific antibodies.

### In Vivo Specificity and Broadly Neutralizing Activity in Sera from Anti-HIV dMAb Injected Subjects

The anti-HIV dMAb generated mAbs are tested for binding specificity and anti-HIV neutralizing activity. Sera antibodies bind to HIV-infected cells. There is a strong specificity of the antibody generated from the anti-HIV dMAb plasmid.

Furthermore, the anti-HIV neutralizing activity in sera from subjects that received anti-HIV dMAb is measured against that in HIV strains. Sera from anti-HIV dMAb-injected subjects effectively neutralize HIV isolates, demonstrating that a single injection can produce significant neutralizing levels of human anti-HIV IgG. Thus, antibodies produced in vivo by anti-HIV dMAb constructs have relevant biological activity (i.e., binding and neutralizing activity against HIV).

### Anti-HIV dMAb Injection Protects Mice from Lethal HIV Challenge

To determine whether antibodies generated from anti-HIV dMAb provide protection against early exposure to HIV, groups of 10 subjects receive of a control or anti-HIV dMAb on day 0. Each group subsequently is challenged subcutaneously with virus to mimic natural HIV infection. Subject survival and weight changes are subsequently recorded. Anti-HIV dMAb plasmids confer protective immunity.

The longevity of immune protection is next evaluated. A second group of subjects was challenged with HIV after injection with anti-HIV dMAb, or control plasmid on day 0. Subjects are monitored for survival. Anti-HIV dMAb provides a more durable degree of immune protection.

Anti-HIV dMAb protects subjects from both subcutaneous viral challenge and intranasal viral challenge compared with control-injected subjects, demonstrating that anti-HIV dMAbs can protect against systemic and mucosal infection.

An efficacy study comparing the protective efficacy of anti-HIV dMAb administration vs a HIV-DNA vaccine (HIV-DNA) is next performed. A novel consensus-based DNA vaccine was developed by our laboratory and is capable of providing protection against HIV challenge. The DNA vaccine also induced both measurable cellular immune responses, as well as potent neutralizing antibody responses. Groups of subjects are administered a single injection of anti-HIV dMAb, HIV-DNA, or the pVaxl, followed by viral challenge. Anti-HIV dMAb confers protective immunity more rapidly than the HIV-DNA vaccine.

### Comparison between In Vivo Protective Immunity Conferred by Anti-HIV dMAb Administration and HIV-DNA Vaccination

Next, a long-term HIV challenge protection study was performed following vaccination with the HIV-DNA vaccine or administration of anti-HIV dMAb on day 0. HIV-DNA confers longer protective immunity than anti-HIV dMAb.

### Co-Delivery of Anti-HIV dMAb and the HIV-DNA Vaccine Produces Systemic Humoral Immunity, Cell-Mediated Immunity, and Protection In Vivo

One potential issue of combining antibody delivery with vaccination approaches is that the antibodies can neutralize many traditional vaccines and thus are incompatible platforms. The effect of co-administration of anti-HIV dMAb and HIV-DNA on subject survival in the context of HIV challenge was is evaluated. Subjects are administered at day 0 anti-HIV dMAb and HIV-DNA. Subsequently, some animals are challenged with HIV at day 2 and the others at day 35. Survival in these groups is followed as a function of time. Anti-HIV dMAb mediates protection from infection, with the survival percentage decreasing to approximately 30% by 4 days after challenge in control (pVax1) animals. Both IgG induced by anti-HIV dMAb and HIV-DNA vaccine are detected. Anti-HIV dMAb mediates rapid protection from infection and death after HIV challenge.

Furthermore, T-cell responses induced in subjects injected with Anti-HIV dMAb, HIV-DNA, or anti-HIV dMAb plus HIV-DNA are evaluated. HIV-DNA elicits strong T-cell responses irrespective of co-delivery with anti-HIV dMAb, showing the lack of interference of these approaches. Conversely, animals administered only anti-HIV dMAb do not develop T-cell responses. Both anti-HIV dMAb and HIV-DNA vaccine can be administered simultaneously without reciprocal interference, providing immediate and long-lived protection via systemic humoral and cellular immunity.

### Electroporation-Mediated Delivery of Optimized DNA Plasmids for the In Vivo Rapid Production of Biologically Functional mAbs

Subjects administered anti-HIV dMAbs are fully protected from viral challenge shortly after administration, whereas subjects do not survive infection following a single immunization with HIV-DNA vaccine, owing presumably to an insufficient time to mount protective immunity. However, HIV-DNA provides complete protection after an immunization regimen followed by challenge at later time points. A similar level of protection occurs in subjects administered a single dose of anti-HIV dMAbs, although protection wanes over time. Notably, the co-delivery of anti-HIV dMAbs and HIV-DNA produces rapid and persistent humoral and cellular immunity, suggesting that a combination approach can have additive or synergistic effects. Importantly, co-delivery of anti-HIV dMAbs and HIV-DNA are not antagonistic in terms of the development of short- or long-term protective immune responses.

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

What is claimed is:

1. A nucleic acid molecule encoding one or more synthetic antibodies, wherein the nucleic acid molecule comprises at least one selected from the group consisting of
a) a nucleotide sequence encoding an anti-HIV synthetic antibody;
b) a nucleotide sequence encoding a fragment of an anti-HIV synthetic antibody.

2. The nucleic acid molecule of claim 1, further comprising a nucleotide sequence encoding a cleavage domain.

3. The nucleic acid molecule of claim 1, wherein the nucleic acid molecule comprises a nucleotide sequence encoding an anti-HIV synthetic antibody.

4. The nucleic acid molecule of claim 3, wherein the nucleotide sequence encodes one or more amino acid sequences at least 95% homologous to an amino acid sequence selected from the group consisting of SEQ ID NO:1, SEQ ID NO:3, SEQ ID NO:5, SEQ ID NO:7, SEQ ID NO:9, SEQ ID NO:11, or SEQ ID NO:13, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39 SEQ ID NO:41, SEQ ID NO:43, SEQ ID NO:45, SEQ ID NO:47, SEQ ID NO:49 SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:111, SEQ ID NO:113, SEQ ID NO:115, SEQ ID NO:117, SEQ ID NO:119, SEQ ID NO:121, SEQ ID NO:123, SEQ ID NO:125, SEQ ID NO:127, SEQ ID NO:129, SEQ ID NO:131, and SEQ ID NO:133.

5. The nucleic acid molecule of claim 4, comprising a nucleotide sequence encodes one or more amino acid sequences selected from the group consisting of SEQ ID NO:1, SEQ ID NO:3, SEQ ID NO:5, SEQ ID NO:7, SEQ ID NO:9, SEQ ID NO:11, or SEQ ID NO:13, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39 SEQ ID NO:41, SEQ ID NO:43, SEQ ID NO:45, SEQ ID NO:47, SEQ ID NO:49 SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:111, SEQ ID NO:113, SEQ ID NO:115, SEQ ID NO:117, SEQ ID NO:119, SEQ ID NO:121, SEQ ID NO:123, SEQ ID NO:125, SEQ ID NO:127, SEQ ID NO:129, SEQ ID NO:131, and SEQ ID NO:133.

6. The nucleic acid molecule of claim 3, wherein the nucleic acid molecule comprises a nucleotide sequence at least 95% homologous to a nucleic acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:17, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO: 60, SEQ ID NO:62, SEQ ID NO:64, SEQ ID NO:66, SEQ ID NO:68,SEQ ID NO: 70, SEQ ID NO:72, SEQ ID NO:74, SEQ ID NO:76, SEQ ID NO:78, SEQ ID NO: 80, SEQ ID NO:82, SEQ ID NO:84, SEQ ID NO:86, SEQ ID NO:88, SEQ ID NO: 90, SEQ ID NO:92, SEQ ID NO:94, SEQ ID NO:96, SEQ ID NO:98, SEQ ID NO: 100, SEQ ID NO:102, SEQ ID NO:104, SEQ ID NO:106, SEQ ID NO:108, SEQ ID NO: 110, SEQ ID NO:112, SEQ ID NO:114, SEQ ID NO:116, SEQ ID NO:118, SEQ ID NO: 120, SEQ ID NO:122, SEQ ID NO:124, SEQ ID NO:126, SEQ ID NO:128, SEQ ID NO: 130, SEQ ID NO:132, and SEQ ID NO:134.

7. The nucleic acid molecule of claim 6, wherein the nucleic acid molecule comprises a nucleotide sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:17, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO: 60, SEQ ID NO:62, SEQ ID NO:64, SEQ ID NO:66, SEQ ID NO:68,SEQ ID NO: 70, SEQ ID NO:72, SEQ ID NO:74, SEQ ID NO:76, SEQ ID NO:78, SEQ ID NO: 80, SEQ ID NO:82, SEQ ID NO:84, SEQ ID NO:86, SEQ ID NO:88, SEQ ID NO: 90, SEQ ID NO:92, SEQ ID NO:94, SEQ ID NO:96, SEQ ID NO:98, SEQ ID NO: 100, SEQ ID NO:102, SEQ ID NO:104, SEQ ID NO:106, SEQ ID NO:108, SEQ ID NO: 110, SEQ ID NO:112, SEQ ID NO:114, SEQ ID NO:116, SEQ ID NO:118, SEQ ID NO: 120, SEQ ID NO:122, SEQ ID NO:124, SEQ ID NO:126, SEQ ID NO:128, SEQ ID NO: 130, SEQ ID NO:132, and SEQ ID NO:134.

8. The nucleic acid molecule of claim 3, wherein the nucleic acid molecule comprises a first nucleotide sequence encoding a synthetic anti-HIV heavy chain and a second nucleotide sequence encoding a synthetic anti-HIV light chain.

9. The nucleic acid molecule of claim 8, wherein the first nucleotide sequence encodes an amino acid sequence at least 90% homologous to an amino acid sequence selected from the group consisting of SEQ ID NO:3, SEQ ID NO:11, SEQ ID NO:17, SEQ ID NO:25, SEQ ID NO:39, SEQ ID NO:47, SEQ ID NO:55, SEQ ID NO:59, SEQ ID NO:63, SEQ ID NO:67, SEQ ID NO:71, SEQ ID NO:75, SEQ ID NO:79, SEQ ID NO:83, SEQ ID NO:87, SEQ ID NO:91, SEQ ID NO:95, SEQ ID NO:99, SEQ ID NO:103, SEQ ID NO:107, SEQ ID NO:111, SEQ ID NO:115, SEQ ID NO:119, SEQ ID NO:123, SEQ ID NO:127, and SEQ ID NO:131.

10. The nucleic acid molecule of claim 9, wherein the first nucleotide sequence comprises a nucleotide sequence at least 90% homologous to a nucleotide sequence selected from the group consisting of SEQ ID NO:4, SEQ ID NO:12, SEQ ID NO:18, SEQ ID NO:26, SEQ ID NO:40, SEQ ID NO:48, SEQ ID NO:56, SEQ ID NO:60, SEQ ID NO:64, SEQ ID NO:68, SEQ ID NO:72, SEQ ID NO:76, SEQ ID NO:80, SEQ ID NO:84, SEQ ID NO:88, SEQ ID NO:92, SEQ ID NO:96, SEQ ID NO:100, SEQ ID NO:104, SEQ ID NO:108, SEQ ID NO:112, SEQ ID NO:116, SEQ ID NO:120, SEQ ID NO:124, SEQ ID NO:128, and SEQ ID NO:132.

11. The nucleic acid molecule of claim 8, wherein the second nucleotide sequence encodes an amino acid sequence at least 90% homologous to an amino acid sequence selected from the group consisting of SEQ ID NO:5, SEQ ID NO:13, SEQ ID NO:19, SEQ ID NO:27, SEQ ID NO:41, SEQ ID NO:49, SEQ ID NO:57, SEQ ID NO:61, SEQ ID NO:65, SEQ ID NO:69, SEQ ID NO:73, SEQ ID NO:77, SEQ ID NO:81, SEQ ID NO:85, SEQ ID NO:89, SEQ ID NO:93, SEQ ID NO:97, SEQ ID NO:101, SEQ ID NO:105, SEQ ID NO:109, SEQ ID NO:113, SEQ ID NO:117, SEQ ID NO:121, SEQ ID NO:125, SEQ ID NO:129, and SEQ ID NO:133.

12. The nucleic acid molecule of claim 11, wherein the second nucleotide sequence comprises a nucleotide sequence at least 90% homologous to a nucleotide sequence selected from the group consisting of SEQ ID NO:6, SEQ ID NO:14, SEQ ID NO:20, SEQ ID NO:28, SEQ ID NO:42, SEQ ID NO:50, SEQ ID NO:58, SEQ ID NO:62, SEQ ID NO:66, SEQ ID NO:70, SEQ ID NO:74, SEQ ID NO:76, SEQ ID NO:82, SEQ ID NO:86, SEQ ID NO:90, SEQ ID NO:94, SEQ ID NO:98, SEQ ID NO:102, SEQ ID NO:106, SEQ ID NO:110, SEQ ID NO:114, SEQ ID NO:118, SEQ ID NO:122, SEQ ID NO:126, SEQ ID NO:130, and SEQ ID NO:134.

13. The nucleic acid molecule of clam 8, wherein
the first nucleotide sequence encodes an amino acid sequence at least 90% homologous to SEQ ID NO:3 and the second nucleotide sequence encodes an amino acid sequence at least 90% homologous to SEQ ID NO:5;
the first nucleotide sequence encodes an amino acid sequence at least 90% homologous to SEQ ID NO:11 and the second nucleotide sequence encodes an amino acid sequence at least 90% homologous to SEQ ID NO:13;
the first nucleotide sequence encodes an amino acid sequence at least 90% homologous to SEQ ID NO:17 and the second nucleotide sequence encodes an amino acid sequence at least 90% homologous to SEQ ID NO:19;
the first nucleotide sequence encodes an amino acid sequence at least 90% homologous to SEQ ID NO:25 and the second nucleotide sequence encodes an amino acid sequence at least 90% homologous to SEQ ID NO:27;
the first nucleotide sequence encodes an amino acid sequence at least 90% homologous to SEQ ID NO:39 and the second nucleotide sequence encodes an amino acid sequence at least 90% homologous to SEQ ID NO:41;
the first nucleotide sequence encodes an amino acid sequence at least 90% homologous to SEQ ID NO:47 and the second nucleotide sequence encodes an amino acid sequence at least 90% homologous to SEQ ID NO:49;
the first nucleotide sequence encodes an amino acid sequence at least 90% homologous to SEQ ID NO:55 and the second nucleotide sequence encodes an amino acid sequence at least 90% homologous to SEQ ID NO:57;
the first nucleotide sequence encodes an amino acid sequence at least 90% homologous to SEQ ID NO:59 and the second nucleotide sequence encodes an amino acid sequence at least 90% homologous to SEQ ID NO:61;
the first nucleotide sequence encodes an amino acid sequence at least 90% homologous to SEQ ID NO:63 and the second nucleotide sequence encodes an amino acid sequence at least 90% homologous to SEQ ID NO:65;
the first nucleotide sequence encodes an amino acid sequence at least 90% homologous to SEQ ID NO:67 and the second nucleotide sequence encodes an amino acid sequence at least 90% homologous to SEQ ID NO:69;
the first nucleotide sequence encodes an amino acid sequence at least 90% homologous to SEQ ID NO:71 and the second nucleotide sequence encodes an amino acid sequence at least 90% homologous to SEQ ID NO:73;
the first nucleotide sequence encodes an amino acid sequence at least 90% homologous to SEQ ID NO:75 and the second nucleotide sequence encodes an amino acid sequence at least 90% homologous to SEQ ID NO:77;
the first nucleotide sequence encodes an amino acid sequence at least 90% homologous to SEQ ID NO:79 and the second nucleotide sequence encodes an amino acid sequence at least 90% homologous to SEQ ID NO:81;
the first nucleotide sequence encodes an amino acid sequence at least 90% homologous to SEQ ID NO:83 and the second nucleotide sequence encodes an amino acid sequence at least 90% homologous to SEQ ID NO:85;
the first nucleotide sequence encodes an amino acid sequence at least 90% homologous to SEQ ID NO:87 and the second nucleotide sequence encodes an amino acid sequence at least 90% homologous to SEQ ID NO:89;
the first nucleotide sequence encodes an amino acid sequence at least 90% homologous to SEQ ID NO:91 and the second nucleotide sequence encodes an amino acid sequence at least 90% homologous to SEQ ID NO:93;
the first nucleotide sequence encodes an amino acid sequence at least 90% homologous to SEQ ID NO:95 and the second nucleotide sequence encodes an amino acid sequence at least 90% homologous to SEQ ID NO:97;
the first nucleotide sequence encodes an amino acid sequence at least 90% homologous to SEQ ID NO:99 and the second nucleotide sequence encodes an amino acid sequence at least 90% homologous to SEQ ID NO:101;
the first nucleotide sequence encodes an amino acid sequence at least 90% homologous to SEQ ID NO:103 and the second nucleotide sequence encodes an amino acid sequence at least 90% homologous to SEQ ID NO: 105;
the first nucleotide sequence encodes an amino acid sequence at least 90% homologous to SEQ ID NO: 107 and the second nucleotide sequence encodes an amino acid sequence at least 90% homologous to SEQ ID NO: 109;
the first nucleotide sequence encodes an amino acid sequence at least 90% homologous to SEQ ID NO:111 and the second nucleotide sequence encodes an amino acid sequence at least 90% homologous to SEQ ID NO: 113;
the first nucleotide sequence encodes an amino acid sequence at least 90% homologous to SEQ ID NO: 115 and the second nucleotide sequence encodes an amino acid sequence at least 90% homologous to SEQ ID NO: 117;
the first nucleotide sequence encodes an amino acid sequence at least 90% homologous to SEQ ID NO: 119 and the second nucleotide sequence encodes an amino acid sequence at least 90% homologous to SEQ ID NO: 121;
the first nucleotide sequence encodes an amino acid sequence at least 90% homologous to SEQ ID NO:123 and the second nucleotide sequence encodes an amino acid sequence at least 90% homologous to SEQ ID NO: 125;
the first nucleotide sequence encodes an amino acid sequence at least 90% homologous to SEQ ID NO: 1207 and the second nucleotide sequence encodes an amino acid sequence at least 90% homologous to SEQ ID NO: 129; or the first nucleotide sequence encodes an amino acid sequence at least 90% homologous to SEQ ID NO:131 and the second nucleotide sequence encodes an amino acid sequence at least 90% homologous to SEQ ID NO: 133.

14. The nucleic acid molecule of claim 13, wherein
the first nucleotide sequence comprises a nucleotide sequence at least 90% homologous to SEQ ID NO:4 and the second nucleotide sequence comprises a nucleotide sequence at least 90% homologous to SEQ ID NO:6;
the first nucleotide sequence comprises a nucleotide sequence at least 90% homologous to SEQ ID NO:12 and the second nucleotide sequence comprises a nucleotide sequence at least 90% homologous to SEQ ID NO:14;
the first nucleotide sequence comprises a nucleotide sequence at least 90% homologous to SEQ ID NO:18 and the second nucleotide sequence comprises a nucleotide sequence at least 90% homologous to SEQ ID NO:20;
the first nucleotide sequence comprises a nucleotide sequence at least 90% homologous to SEQ ID NO:26 and the second nucleotide sequence comprises a nucleotide sequence at least 90% homologous to SEQ ID NO:28;
the first nucleotide sequence comprises a nucleotide sequence at least 90% homologous to SEQ ID NO:40 and the second nucleotide sequence comprises a nucleotide sequence at least 90% homologous to SEQ ID NO:42;
the first nucleotide sequence comprises a nucleotide sequence at least 90% homologous to SEQ ID NO:48 and the second nucleotide sequence comprises a nucleotide sequence at least 90% homologous to SEQ ID NO:50;
the first nucleotide sequence comprises a nucleotide sequence at least 90% homologous to SEQ ID NO:56 and the second nucleotide sequence comprises a nucleotide sequence at least 90% homologous to SEQ ID NO:58;
the first nucleotide sequence comprises a nucleotide sequence at least 90% homologous to SEQ ID NO:60 and the second nucleotide sequence comprises a nucleotide sequence at least 90% homologous to SEQ ID NO:62;
the first nucleotide sequence comprises a nucleotide sequence at least 90% homologous to SEQ ID NO:64 and the second nucleotide sequence comprises a nucleotide sequence at least 90% homologous to SEQ ID NO:66;
the first nucleotide sequence comprises a nucleotide sequence at least 90% homologous to SEQ ID NO:68 and the second nucleotide sequence comprises a nucleotide sequence at least 90% homologous to SEQ ID NO:70;
the first nucleotide sequence comprises a nucleotide sequence at least 90% homologous to SEQ ID NO:72 and the second nucleotide sequence comprises a nucleotide sequence at least 90% homologous to SEQ ID NO:74;
the first nucleotide sequence comprises a nucleotide sequence at least 90% homologous to SEQ ID NO:76 and the second nucleotide sequence comprises a nucleotide sequence at least 90% homologous to SEQ ID NO:78;
the first nucleotide sequence comprises a nucleotide sequence at least 90% homologous to SEQ ID NO:80 and the second nucleotide sequence comprises a nucleotide sequence at least 90% homologous to SEQ ID NO:82;
the first nucleotide sequence comprises a nucleotide sequence at least 90% homologous to SEQ ID NO:84 and the second nucleotide sequence comprises a nucleotide sequence at least 90% homologous to SEQ ID NO:86;
the first nucleotide sequence comprises a nucleotide sequence at least 90% homologous to SEQ ID NO:88 and the second nucleotide sequence comprises a nucleotide sequence at least 90% homologous to SEQ ID NO:90;
the first nucleotide sequence comprises a nucleotide sequence at least 90% homologous to SEQ ID NO:92 and the second nucleotide sequence comprises a nucleotide sequence at least 90% homologous to SEQ ID NO:94;
the first nucleotide sequence comprises a nucleotide sequence at least 90% homologous to SEQ ID NO:96 and the second nucleotide sequence comprises a nucleotide sequence at least 90% homologous to SEQ ID NO:98;
the first nucleotide sequence comprises a nucleotide sequence at least 90% homologous to SEQ ID NO:100 and the second nucleotide sequence comprises a nucleotide sequence at least 90% homologous to SEQ ID NO:102;
the first nucleotide sequence comprises a nucleotide sequence at least 90% homologous to SEQ ID NO:104 and the second nucleotide sequence comprises a nucleotide sequence at least 90% homologous to SEQ ID NO: 106;
the first nucleotide sequence comprises a nucleotide sequence at least 90% homologous to SEQ ID NO: 108 and the second nucleotide sequence comprises a nucleotide sequence at least 90% homologous to SEQ ID NO: 110;
the first nucleotide sequence comprises a nucleotide sequence at least 90% homologous to SEQ ID NO:112 and the second nucleotide sequence comprises a nucleotide sequence at least 90% homologous to SEQ ID NO: 114;
the first nucleotide sequence comprises a nucleotide sequence at least 90% homologous to SEQ ID NO: 116 and the second nucleotide sequence comprises a nucleotide sequence at least 90% homologous to SEQ ID NO: 118;
the first nucleotide sequence comprises a nucleotide sequence at least 90% homologous to SEQ ID NO: 120 and the second nucleotide sequence comprises a nucleotide sequence at least 90% homologous to SEQ ID NO: 122;
the first nucleotide sequence comprises a nucleotide sequence at least 90% homologous to SEQ ID NO:124 and the second nucleotide sequence comprises a nucleotide sequence at least 90% homologous to SEQ ID NO: 126;
the first nucleotide sequence comprises a nucleotide sequence at least 90% homologous to SEQ ID NO: 128 and the second nucleotide sequence comprises a nucleotide sequence at least 90% homologous to SEQ ID NO: 130; or
the first nucleotide sequence comprises a nucleotide sequence at least 90% homologous to SEQ ID NO:132 and the second nucleotide sequence comprises a nucleotide sequence at least 90% homologous to SEQ ID NO: 134.

15. The nucleic acid molecule of claim 1, wherein the nucleotide sequence encodes a leader sequence.

16. The nucleic acid molecule of any one of claims 1-15, wherein the nucleic acid molecule comprises an expression vector.

17. A composition comprising the nucleic acid molecule of any one of claims 1-16.

18. The composition of claim 17, further comprising a pharmaceutically acceptable excipient.

19. A composition comprising:
a) a first nucleic acid molecule wherein the nucleic acid molecule encodes a consensus HIV antigen; and
b) a second nucleic acid molecule encoding one or more synthetic antibodies, wherein the nucleic acid molecule comprises at least one selected from the group consisting of
i) a nucleotide sequence encoding an anti-HIV synthetic antibody; and
ii) a nucleotide sequence encoding a fragment of an anti-HIV synthetic antibody.

20. A method of treating a disease in a subject, the method comprising administering to the subject the nucleic acid molecule of any of claims 1-16 or a composition of any of claims 17-19.

21. The method of claim 14, wherein the disease is an HIV infection.

22. A method of inducing an immune response in a subject, the method comprising administering to the subject the nucleic acid molecule of any of claims 1-16 or a composition of any of claims 17-19.

23. A nucleic acid molecule encoding one or more synthetic antibodies, wherein the nucleic acid molecule comprises a nucleotide sequence encoding an IgG4 synthetic antibody, wherein the IgG4 antibody comprises a Ser288Pro amino acid substitution.

24. The nucleic acid molecule of claim 23, wherein the Ser288Pro amino acid substitution prevents IgG4 Fab arm switching.
